SEQUENCE LISTING (4 sequences)

SEQ ID NO: 1 - protein, 264 aa, Homo sapiens
MTKFSSFSLFFLIVGAYMTHVCFNMEIIGGKEVSPHSRPFMASIQYGGHHVCGGVLIDPQWVLTAAHCQYRFTKGQSPTVVLGAHSLSKNEASKQTLEIKKFIPFSRVTSDPQSNDIMLVKLQTAAKLNKHVKMLHIRSKTSLRSGTKCKVTGWGATDPDSLRPSDTLREVTVTVLSRKLCNSQSYYNGDPFITKDMVCAGDAKGQKDSCKGDSGGPLICKGVFHAIVSGGHECGVATKPGIYTLLTKKYQTWIKSNLVPPHTN

SEQ ID NO: 2 - DNA, 1074 bp, Homo sapiens
  source 1..1074: mol_type = genomic DNA
gatcaacacatttcatctgggcttcttaaatctaaatctttaaaatgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgtaagtcattttattaaggaatcaagttctttttcacttgtatcactgatgtatttctaccatgctggttttattctaaataaaatttagaagactcaaaaaaaaaaaaaaaaaaaaaaaaaaaaa

SEQ ID NO: 3 - protein, 247 aa, Homo sapiens [sequence omitted]

SEQ ID NO: 4 - DNA, 4751 bp, Homo sapiens
  source 1..4751: mol_type = genomic DNA
gaattctatattttgagatataccattcctcatagaaaaatttcctcacagaaaatataaaggtggaaacaaatcacaagaatcgaaccatgtagagagacttagttgtcttttaacagaattgggcacgggctgttcagaaacaacaatctttcacatccattataatgatagcattagtgtagtttgtttagcaaatgtttactgcgagcctgttatgtgctgagcctgctatgtaagaagtgtggctctctggacaggagacagaatactaaacaacacaactactgatctttggctgcctggcatgcttcctcacttcatatggtatcagcaatttagcaccacaaacgtcctttagagaccagccctttctcattcttggttctagtggcttgagtagactgaccccactacccaagtggatttgactcctagcaattcattaatctagcccataaatgtcaagtacaggactttattgaagcattcagaaagaggaataggggatgttagaatctctagaaaggaagctatgataataaatgggttgctagatgggtctagtagatggtggccatgctttgttactgccttgtgtattgtgctaccatagccctccccaaactgtactctggctcctggcatttccgtctcttcaaccagatggtcagctctctaagtgaaggagacacatctccaacatgcttggttctagcacaacagaagggctcaaacacatacctgctaaagaaactatcctgatggatttagcagcatggccatgaggcattggcggttctatcactgggaactcaggtttctggtgctccagtacctctactggctgataccacatcctacagttcacttcataggcttgggttcctgctctgggctgaataggtggtccactctgagtcatcagctgtggtgatgatgtggtcactgcatgattctcacacaagcacccagaggacgtcatcaggcagaggcagtgggggtgggcagcatttacagaaaatctgtgatgagacaccacaaaaccagaggggaacatgaagtcactgagcctgctccacctctttcctctcccaagagctaaaagagagcaaggaggaaacaacagcagctccaaccagggcagccttcctgagaagatgcaaccaatcctgcttctgctggccttcctcctgctgcccagggcagatgcaggtgagtgaccgtcttccaacctcggggcccaacccatcccacaggtctcctgccctttctccacattcctgatccatctatctaccaggaatgttctgaactccagctcccattctaccaagaccccccaagtgtgatgctggataagctatcagcaggaatggcagagcagcaggccattctcaagaagagccagtgggtactatcccttccccagagcccacctttgtcacctggagagtaggactttcctagaagtaaatggcagaggatgggaaactagaaaagagaaatattaaattattctagagtaggcctggcttctgtttctgggataagacaggtgcttctctcactgtacttaggagagaaacccagagctcagctgacagcagaattggtacaatcactgtcctcagaacactgttaatgtgtttgctcagtcccattctccaactctgcttttcttccctggcctttggtggctcccctctttccaaggatgaggcactacggcaggccccagcttccctgctttctagaattccaccagcactgctctaccagccctcatccagaggctaactggagccagtccatcatgcagccatgaacatttactgggcacccactacatgtcaggctctaggaaacaggatatgacagtatctagatccctccacttacaccctggccattagaaagcagcactatcctagacaccacaggactcataagggtcttggaaactcacctgaaacaaagcaaagtcaggagaggaatgatcaggagcctctgggatttcactgtccctaagacaggtatgctcgccttcaactacatatggaagaaagatttacagaccaaagtctgctgttcttccctttttcagagcaggaaattgaagccccttcctccaggccactcccaactccaggctatcccaggctcccaaatgcccaggagttctggagccactaagcaggtgcccacccagcagattccatgggtgcccacaagcagacagacttttccttcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaaggtgaggagcagaaaacagcccacaccctcctggaaacactccacagagacccctgccttcttcccaaggagctccctgggctcctgtgaacacacatgccaggaggtctccttagagggtgagaaaagggcagttaagtttgtggagagaggggaaggttggttccagaggtgctgctgaagtaagaaacagcagagtgaccaagcctgccatatttagaactgggggcatactttggcatagaatacaaactgaagcaattccacctgtgtttctagggggaaccgaaccctgagaaacctggtgcaattaccagaattccaattcctggggaccgactgtccttaatttcccctcagctgcagccctgccccagctgtcacctgctcttcactgtctctgggctgtatacactgtgactccacccccatcctcactctgctctctgtgcagctccataaatgtcaccttgggggcccacaatatcaaggaacaggagccgacccagcagtttatccctgtgaaaagagccatcccccatccagcctataatcctaagaacttctccaatgacatcatgctactgcaggtgaggcacactcctgccactcttgctcttcttggtccagttggttccactccccctggaatgccggcccttccctcctttccatcctggcctcttggttagttcctatgcctcagaggagagagggaagattgtgcagccccatcactgtgtcggggcccagaagttcgttggctgacctggactttcttgcctcttccccaccagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaaggtaagactatgcacctgcctggattggctcttgggagaaagatgtttggggaatatctgagacctggagactcaagtagtgggggactccttcacccactagactgtgatatttctctctggaaagagaaaaggggactagactgagctggggagaaattagggcctctgcaaacttaccaagaggccttatggtggatggtgccttctttggaaggatgaatttgcaacactccacccactccaggtcacagatattaggaaactgtgcccatgggggtgcagctaattataaccaggtgtgtcttcagaggctggtacccaacgtggttaatgggctggtcctccatggtggacatcagccctccttgcccacttctgggtccttaaacagccaacggtcccacatacctccgatctcaggatctgggggacatgacggaggctggcccctgggatgaggtgaagcagtaacaatgtccagggccagagcttggcagctggggccaccagcggcctgccctgccctctggtctcccacatgtaggctgtgcaagttggccttttctaaaagggggcttgagatggaagagagggcaggacccggaggagcatcagctcagtccttccactctctattcacagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctcttagctgagtggaaagctggtttcttgtttattcattgaccctcattctcaggcaccacatctgcgctatgcaggccaatgacacaattttgctgttttctgctttctcctctcccctcaccccttgccacctccccaaacccccacatgaagctgatactcagctccttcctatccacaccagtttctccagggcctgccttctgccaaggctgaagctgagcaccatcaggagacaacatggaccactttggtcctggggctttgggtaaacttcttacctccttctccagtgttacattgacagagaaaaaagggataataccatgggacctaactcctcatcccactggggctcctcattctcccctgggcttagtttctctaccctcctctgagctc

(19) United States
(12) Patent Application Publication
(10) Pub. No.: US 2025/0299796 A1
(43) Pub. Date: Sep. 25, 2025

(54) COMPOSITIONS AND METHODS FOR TREATING AND/OR CHARACTERIZING HEMATOLOGICAL MALIGNANCIES AND PRECURSOR CONDITIONS

(71) Applicants: Dana-Farber Cancer Institute, Inc., Boston, MA (US); The General Hospital Corporation, Boston, MA (US)

(72) Inventors: Irene M. Ghobrial, Boston, MA (US); Romanos Sklavenitis-Pistofidis, Jamaica Plain, MA (US); Gad Getz, Cambridge, MA (US)

(21) Appl. No.: 18/682,156

(22) PCT Filed: Aug. 11, 2022
(86) PCT No.: PCT/US2022/074839

§ 371 Date: Feb. 8, 2024

Related U.S. Application Data
(60) Provisional application No. 63/232,015, filed on Aug. 11, 2021.

(51) Int. Cl. G16H 20/17; A61K 31/454; A61K 31/573; C07K 16/28; C12Q 1/6886; G01N 33/569; G01N 33/574

(52) U.S. Cl. CPC G16H 20/17; A61K 31/454; A61K 31/573; C07K 16/2803; C12Q 1/6886; G01N 33/56972; G01N 33/57407; C12Q 2600/106; G01N 2333/96436

(57) ABSTRACT

Provided herein are methods and immune biomarkers that identify progression and treatment options for hematological malignancies (e.g., smoldering multiple myeloma (SMM), monoclonal gammopathy of undetermined significance (MGUS), or multiple myeloma (MM)). Also provided are materials and methods for the prognosis, staging, and monitoring of SMM, MGUS, or MM based on the presence of the immune biomarkers in a sample (e.g., a blood sample or a bone marrow sample), as well as methods for monitoring the progression of SMM, MGUS, or MM, determining the efficacy of a therapeutic agent, determining a treatment for SMM, MGUS (e.g., before progression to MM), or MM, and/or treating SMM, MGUS, or MM. The methods provided herein provide several advantages over invasive biopsies.

### CROSS REFERENCE TO RELATED APPLICATIONS

The present application claims priority to and the benefit of U.S. App. No. 63/232,015, filed Aug. 11, 2021, which is hereby incorporated by reference in its entirety.

### STATEMENT OF RIGHTS TO INVENTIONS MADE UNDER FEDERALLY SPONSORED RESEARCH

This invention was made with government support under grant No. R01 CA205954 awarded by the National Institutes of Health. The government has certain rights in the invention.

### BACKGROUND

Plasma cell dyscrasias are disorders of plasma cells. Multiple Myeloma (MM) is a plasma cell dyscrasia characterized by patchy bone marrow infiltration leading to multiple bone lytic lesions and cytopenias at the time of diagnosis. Bone marrow biopsies are limited in that sampling allows assessment of only one site where the tumor clones and their immune microenvironment can be different from those present in other areas of the bone marrow and may not be reflective of the total disease heterogeneity. It is also a painful procedure for patients and patients with precursor state monoclonal gammopathy of undetermined significance (MGUS) or smoldering multiple myeloma (SMM), who do not have bone marrow biopsies performed regularly, which precludes regular assessment of their progression risk.

Multiple Myeloma (MM) is an incurable plasma cell malignancy that resides in the bone marrow and is preceded by an asymptomatic condition called Smoldering MM (SMM). Approximately half of patients with SMM will progress within the first 5 years from diagnosis, yet not all patients with SMM progress. Therefore, patients with SMM are typically observed until end-organ damage (e.g., kidney failure, bone disease, anemia) occurs, which signifies progression to overt MM, warranting treatment. Identifying the patients who will benefit from early intervention is an unmet clinical need. Identifying the patients who will benefit the most from early intervention remains an unmet clinical need. Likewise, identifying the patients who will benefit from more intensive treatment and for whom treatment may be terminated remain unmet clinical needs.

### SUMMARY

The disclosure provides compositions and methods for characterizing, and treating subjects having or having a propensity to develop smoldering multiple myeloma and related disorders.

In one aspect, the invention provides a method of treating a selected subject having a hematological malignancy or precursor condition, the method involving administering a combination therapy involving administering an immunotherapeutic agent and/or immunomodulatory agent to the subject, where the subject is selected by detecting an increase in GZMK-positive effector memory T-cells in a biological sample of the subject relative to a reference.

In various embodiments, the hematological malignancy or precursor condition is any one or more of plasma cell dyscrasia, a monoclonal gammopathy, monoclonal gammopathy of undermined significance (MGUS), smoldering multiple myeloma (SMM), symptomatic multiple myeloma, Waldenstrom macroglobulinemia (WM), amyloidosis (AL), plasmacytoma syndrome, solitary plasmacytoma of bone, extramedullary plasmacytoma, light chain deposition disease, heavy-chain disease, and B cell or plasma cell neoplasm. In various embodiments, In various embodiments, the immunomodulatory agent is dexamethasone, lenalidomide, pomalidomide, or thalidomide. In various embodiments, In various embodiments, the immunotherapeutic agent is daratumumab, BCMA, GPRC5D, Elotuzumab, a Car T-cell, a bispecific antibody against CD3, an immune checkpoint inhibitor, or a bispecific antibody T-cell engager.

In another aspect, the invention provides a method of treating a selected subject having high risk smoldering multiple myeloma or Monoclonal gammopathy of undetermined significance, the method involving administering a combination therapy containing Elotuzumab, Lenalidomide, and Dexamethasone to the subject, where the subject is selected by detecting an increase in GZMK-positive effector memory T-cells in a biological sample of the subject relative to a reference.

In another aspect, the invention provides a method of selecting a subject having smoldering multiple myeloma for treatment with a combination therapy containing Elotuzumab, Lenalidomide, and Dexamethasone, the method involving detecting GZMK-positive memory T-cells in a biological sample of the subject, where an increase in the number of GZMK-positive memory T-cells in the sample indicates that the subject should be treated with the combination therapy.

In another aspect, the invention provides a method of treating a selected subject having smoldering multiple myeloma, the method involving administering a combination therapy containing Elotuzumab, Lenalidomide, and Dexamethasone to the subject, where the subject is selected by characterizing immune reactivity in a biological sample of the subject prior to treatment, where a patient characterized as immune reactive is selected for treatment with the combination therapy, and a patient characterized as non-immune reactive is selected to receive an alternate therapy.

In another aspect, the invention provides a method of selecting a subject having smoldering multiple myeloma for treatment with a combination therapy containing Elotuzumab, Lenalidomide, and Dexamethasone, the method involving characterizing immune reactivity in a biological sample of the subject prior to treatment, where a patient characterized as immune reactive is selected for treatment with Elotuzumab, Lenalidomide, and Dexamethasone, and a patient characterized as non-immune reactive is selected for alternate therapy.

In another aspect, the invention provides a method for monitoring combination therapy containing an immunotherapeutic or immunomodulatory agent in a subject having a hematological malignancy or precursor condition, the method involving characterizing normalization scores during the course of therapy, where an increase in immune normalization score relative to a baseline normalization score indicates that the combination therapy is effective.

In another aspect, the invention provides a method for characterizing a subject being treated for a hematological malignancy or precursor condition at end of treatment, the method involving characterizing normalization scores at end of treatment, where a significant increase in post-therapy immune normalization score characterizes the subject as having a good prognosis, and no significant change in post-therapy immune normalization score characterizes the subject as having a poor prognosis.

In another aspect, the invention provides a method for selecting a subject being treated for a hematological malignancy or precursor condition for discontinuation of combination therapy, the method involving characterizing immune normalization in a biological sample of the subject, where the combination therapy contains Elotuzumab, Lenalidomide, and Dexamethasone, and where a significant increase in immune normalization score indicates that therapy may be discontinued, where the failure to detect an increase in immune normalization score indicates that combination therapy should be continued or that alternate therapies are indicated.

In various embodiments of the above aspect, a Naïve Bayes classifier is used on a training set containing biological samples from subjects with a hematological malignancy or precursor condition and healthy control subjects, where the input to the classifier is the composition matrix of cell type proportions and the weighted sum of the product of each cell type's proportion is computed to determine a normalization score, where a subject is classified based on the median normalization score. In various embodiments of the above aspect, characterizing post-therapy immune normalization (PIN) involves determining a threshold based on the distribution of change in normalization scores.

In another aspect, the invention provides a panel for characterizing a biological sample from a subject having smoldering multiple myeloma, the panel containing one or more capture molecules, each of which binds a marker polypeptide or a polynucleotide encoding the marker polypeptide, where the marker polypeptides are selected from the following:
a) a GEX-1 panel for characterizing cytokine signaling associated with GZMK-expressing subpopulations of immune cells containing capture molecules that bind XCL1, XCL2, CCL3, and/or CCL4;
b) a GEX-2 panel for characterizing captured naïve T-cells containing capture molecules that bind TCF7, LEF1, TXNIP, IL7R, LTB, and/or NOSIP;
c) a GEX-3 panel for characterizing captured dendritic cells containing capture molecules that bind CLEC10A, FCERIA, CD74, HLA-DRA, and/or HLA-DRB1;
d) a GEX-4 panel for characterizing captured B-cells containing capture molecules that bind CD19, MS4A1, IGHM, IGHD, CD79A, and/or CD79B;
e) a GEX-5 panel for characterizing ferritin heavy and light chain expression containing capture molecules that bind FTH1 and/or FTL;
f) a GEX-6 panel for characterizing pro-inflammatory signaling containing capture molecules that bind IL1B, CXCL8, CXCL2, and/or CEBPD;
g) a GEX-7 panel for characterizing genes induced by interferon-gamma containing capture molecules that bind MX1, STAT1, XAF1, IFI6, IFI44L, and/or ISG15;
h) a GEX-8 panel for characterizing thymosin expression and the alpha and beta chains of the TCR containing capture molecules that bind TMSB4X, TMSB10, TRAC, and/or TRBC1;
i) a GEX-9 panel for characterizing histone 1 gene expression containing capture molecules that bind HIST1H1C, HIST1H1D, and/or HIST1H1E;
j) a GEX-10 panel for characterizing metabolic programming containing capture molecules that bind GLUL, SLC43A2, and/or SLC25A37 and hypoxia-related signaling containing capture molecules that bind HIF1A, TIMP1, and/or NEAT1;
k) a GEX-11 panel for characterizing CD14⁺ monocytes containing capture molecules that bind CD14, MS4A6A, FCN1, and/or LYZ;
l) a GEX-12 panel for characterizing immature B-cells containing capture molecules that bind ACSM3, IGLL1, VPREB1, TCLIA, MME, and/or SOX4;
m) a GEX-13 panel for characterizing pDCs containing capture molecules that bind LILRA4, JCHAIN, SERPINF1, PTGDS, ITM2C, IRF4, and/or IRF8;
n) a GEX-14 panel for characterizing a program within classical monocytes containing capture molecules that bind S100A8, S100A9, S100A12, and/or VCAN;
o) a GEX-15 panel for characterizing Th17 cells and mucosa-associated invariant T-cells (MAITs) containing capture molecules that bind RORA, KLRB1, and/or CCR6;
p) a GEX-16 panel for characterizing interferon type-I signaling containing capture molecules that bind IFIT1, IFIT2, IFIT3, PMAIP1, OASL, TNF, HERC5, and/or ZC3HAV1;
q) a GEX-17 panel for characterizing T-cell surface antigens containing capture molecules that bind CD5, CD6, and/or CD7;
r) a GEX-18 panel for characterizing an AP-1 module containing capture molecules that bind JUN, FOS, and/or FOSB;
s) a GEX-19 panel for characterizing activation markers containing capture molecules that bind DUSP1, TSC22D3, and/or NFKBIA;
t) a GEX-20 panel for characterizing CD16⁺ monocytes containing capture molecules that bind FCGR3A, CDKN1C, SERPINA1, and/or MS4A7;
u) a GEX-21 panel for characterizing cytotoxic T-cells containing capture molecules that bind GZMK, GZMH, NKG7, CD8A, and/or CD8B;
v) a GEX-22 panel for characterizing GZMB⁺ cytotoxic T-cells and NK cells containing capture molecules that bind GZMB, GNLY, SPON2, CLIC3, and/or FGFBP2;
w) a GEX-23 panel for characterizing polynucleotides and polypeptides frequently expressed by myeloid cells containing capture molecules that bind AHNAK, TLR2, and/or CYBB;
x) a GEX-24 panel for characterizing memory CD4⁺ T-cell expression markers containing capture molecules that bind GATA3, CD28, PRDM1, ITGB1, nd/or ANXA1;
Y) a GEX-25/GEX-26 panel for characterizing cytoskeleton-related genes containing capture molecules that bind ACTB, ACTG1, PFN1, and/or CFL.

In various embodiments of the above aspect, the capture molecule is a polypeptide, polynucleotide, or other agent that specifically binds to a marker polypeptide or polynucleotide. In various embodiments of the above aspect, the polypeptide is an antibody or antigenic fragment thereof. In various embodiments of the above aspect, the polynucleotide is a primer or probe that hybridizes to a polynucleotide encoding the marker polypeptide. In various embodiments of the above aspect, the GEX-5 and GEX-6 panels are used to characterize myeloid cells. In various embodiments of the above aspect, the GEX-7 panel is used to characterize T-cells, B-cells, NK cells, and monocytes. In various embodiments of the above aspect, the GEX-8 panel is used to characterize T-cells. In various embodiments of the above aspect, the GEX-9 panel is used to characterize dendritic cells. In various embodiments of the above aspect, the GEX-10 panel is used to characterize myeloid cells. In various embodiments of the above aspect, the GEX-23 panel is used to characterize monocytes. In various embodiments of the above aspect, the GEX-25/GEX-26 panel is used to characterize myeloid cells.

In another aspect, the invention provides a kit including the panel of any previous aspect, and instructions for its use in characterizing a biological sample.

In another aspect, the invention provides a method for characterizing a subject having smoldering multiple myeloma for treatment with a combination therapy including elotuzumab, lenalidomide, and dexamethasone, the method including contacting a biological sample of the subject with the panel of any previous aspect.

In another aspect, the invention provides a method for monitoring therapy in a subject having smoldering multiple myeloma, where the therapy is combination therapy containing elotuzumab, lenalidomide, and dexamethasone, the method involving contacting a biological sample of the subject with the panel of any one previous aspect.

In another aspect, the invention provides a method for analyzing survival, the method involving assessing the mean signature activity of the panel across cells, where mean activity is discretized based on the median.

In various embodiments of any previous aspect or any other aspect of the invention delineated herein, the detecting is by single cell RNA sequencing, targeted single-cell RNA-sequencing, RNA-sequencing, immunohistochemistry, immunofluorescence, flow cytometry, mass cytometry, mass spectrometry, quantitative PCR, immunoblotting, or an imaging-based method. In various embodiments of any previous aspect or any other aspect of the invention delineated herein, the method further involves determining that the human subject has a deletion of the short arm of chromosome 17 (Del17p).

In another aspect, the invention provides a method for characterizing immune dysregulation in bone marrow, involving characterizing blood biomarkers of any one of Tables 1-6 in a biological sample containing blood, serum, plasma, or peripheral blood monocytes.

In another aspect, the invention provides a method for characterizing a hematological malignancy, precursor condition, or other bone marrow disease, the method involving characterizing blood biomarkers of any one of Tables 1-6 in a biological sample containing blood, serum, plasma, or peripheral blood monocytes.

In various embodiments of any previous aspect or any other aspect of the invention delineated herein, the method involves detecting the immune cell composition of blood. In various embodiments of any previous aspect or any other aspect of the invention delineated herein, the immune cells contain plasmacytoid dendritic cells, B cells, and/or T-cells.

In various embodiments of any of the previous aspects, characterizing immune reactivity involves characterizing the similarity or dissimilarity of the subject's immune cell repertoire relative to that of a reference. In various embodiments of any previous aspect or any other aspect of the invention delineated herein, the method further involves detecting an increase in exhaustion marker polypeptides selected from any one or more of the following T cell immunoreceptor with Ig and ITIM domains (TIGIT), Lymphocyte-activation protein 3 (LAG3), Lymphocyte Antigen 9 (LY9) and Killer Cell Lectin Like Receptor G1 (KLRG1), or polynucleotides encoding the polypeptides. In various embodiments of any previous aspect or any other aspect of the invention delineated herein, the method further involves detecting an increase in an Interferon Gamma (IFNG) and/or Tumor Necrosis Factor (TNF) polypeptide, or a polynucleotide encoding the polypeptide(s). In various embodiments of any previous aspect or any other aspect of the invention delineated herein, the method further involves detecting a decrease in a cytotoxicity marker polypeptide selected from any one or more of the following GZMB, FCGR3A, and PRF1, or polynucleotides encoding the polypeptides. In various embodiments of any previous aspect or any other aspect of the invention delineated herein, the method further involves detecting decreased levels of plasmacytoid dendritic cells and Cytokine+CD14+ Monocytes relative to a control, where the decrease indicates that the subject is immune reactive. In various embodiments of any previous aspect or any other aspect of the invention delineated herein, the method further involves detecting T-cell repertoire diversity, where a decrease in diversity is indicative of a poor prognosis and that aggressive therapy for that subject is required. In various embodiments of any previous aspect or any other aspect of the invention delineated herein, detecting T-cell repertoire diversity involves T-cell receptor sequencing and/or characterizing the occurrence of single or dual T-cell clonotypes. In various embodiments of any previous aspect or any other aspect of the invention delineated herein, characterizing immune reactivity involves training a a Naïve Bayes classifier on a training set of bone marrow samples from patients and healthy donors, to predict the presence of malignancy based on the composition of the bone marrow immune microenvironment. In various embodiments of any previous aspect or any other aspect of the invention delineated herein, the method further involves computing a weighted sum score of the product of each cell type's proportion and its corresponding signed importance to the classification, where subjects are classified based on the median score at baseline as reactive or non-reactive. In various embodiments of any previous aspect or any other aspect of the invention delineated herein, subjects classified as reactive are selected for combination therapy, and subjects classified as non-reactive are selected for alternate therapy. In various embodiments of any previous aspect or any other aspect of the invention delineated herein, the method further involves detecting Del17p, where the presence of Del17p is indicative of immune reactivity. In various embodiments of any previous aspect or any other aspect of the invention delineated herein, the method further involves detecting plasmacytoid dendritic cells (pDCs) and cytokine-positive, CD14-positive monocytes in the biological sample, where a lower abundance of pDCs and cytokine-positive, CD14-positive monocytes is indicative that the subject is immune reactive. In various embodiments of any previous aspect or any other aspect of the invention delineated herein, the method further involves detecting a reduction in the level of exhaustion marker polypeptides selected from any one or more of the following TOX, TNFRSF9, TNFSF9, PDCD1, NR4A2, NR4A3 or polynucleotides encoding the marker polypeptides in GZMK+CD8+ TEM cells. In various embodiments of any previous aspect or any other aspect of the invention delineated herein, the method further involves detecting an increase in the level of marker polypeptides IL7R and CD27 associated with long-lived memory effectors or polynucleotides encoding the marker polypeptides; detecting an increase in the level of marker polypeptides associated with terminal effectors: GZMB, GZMH, FCGR3A, FGFBP2, and NKG7 or polynucleotides encoding the marker polypeptides; and/or detecting an increase in the level of marker polypeptides associated with functionality: IFNG and TN or polynucleotides encoding the marker polypeptides. In still other embodiments, the method further involves characterizing GZMB+CD8+ Effector Memory T-cells (TEM) cells from reactive patients to identify an immune reactive profile containing one or more of the following sets of markers: an increase in exhaustion marker polypeptides TIGIT, LAG3, LY9 and KLRG1 or polynucleotides encoding the marker polypeptides; an increase in IFNG and TNF marker polypeptides, or polynucleotides encoding the marker polypeptides, and/or a downregulation of cytotoxicity marker polypeptides GZMB, FCGR3A, and PRF1 or polynucleotides encoding the marker polypeptides, where the immune reactive profile is indicative that the subject has decreased pro-inflammatory myeloid signaling, long-lived GZMK+ effector memory cells of increased potency, and short-lived, exhausted GZMB+ terminal effectors. In still other embodiments, the immune reactive profile is indicative that the subject should be treated with a combination therapy of any of the above aspects. In still other embodiments, the biological sample contains bone marrow or peripheral blood mononuclear cells. In still other embodiments, the biological sample contains CD138-negative bone marrow mononuclear cells or peripheral blood mononuclear cells.

In various embodiments of any of the previous aspects or any other aspect of the invention, the method further involves detecting an increase in CD8+ GZMK-positive effector memory T-cells in a biological sample of the subject. In various embodiments of any of the previous aspects, the reference contains the levels of GZMK-positive effector memory T-cells or the level of GZMK-positive CD8+ effector memory T-cells present in a reference. In various embodiments of any of the previous aspects, a subject having smoldering multiple myeloma, but failing to show an increase in the level of GZMK-positive CD8+ effector memory T-cells in the biological sample relative to a reference is selected to receive an alternate therapy.

In various embodiments of the above aspect, further involving detecting the presence of Del17p in a biological sample of the subject, where such detection indicates a need for more aggressive treatment. In still other embodiments, the methods further involve characterizing the subject's bone marrow microenvironment by detecting an increase in naïve and memory CD4+ T-cells, GZMB+CD8+ effector memory T-cells and CD56dim NK cells, and a reduction in CD14+ monocytes, pDCs and progenitor cells in a biological sample relative to a healthy control. In still other embodiments, detecting at baseline a bone marrow microenvironment that closely resembles the bone marrow microenvironment of a healthy control indicates that the subject should receive an alternate therapeutic regimen. In still other embodiments, detecting at end of treatment a bone marrow microenvironment that closely resembles the bone marrow microenvironment of a healthy control indicates that the therapy was effective and may be discontinued.

Subjects with SMM or MGUS are typically observed until progression, but early treatment may improve outcomes. The data in the Examples section herein include data from a Phase II clinical trial of Elotuzumab, Lenalidomide, and Dexamethasone (“EloLenDex”) in subjects with high-risk SMM. Based on these data, immune biomarkers (see, e.g., Tables 1-6), are used to: identify subjects (e.g., human) with SMM, MGUS, or MM who are likely to benefit from treatment (e.g., treatment before progression from SMM or MGUS to MM for subjects with SMM or MGUS); to identify subjects (e.g., human) with SMM, MGUS, or MM who are likely to benefit from different (e.g., more intensive) treatment regimens; to identify subjects (e.g., human) with MM undergoing immunotherapy who are likely to progress and, thus, are likely to benefit from a different treatment regimen (e.g., a more intensive treatment regimen); to identify subjects (e.g., human) with SMM, MGUS, or MM for whom treatment is likely to result in prolonged biochemical progression free survival, and thus, for whom treatment may be terminated or modified or follow-up may be modified (e.g. changes in the frequency of follow-up assessments or the type of tests performed clinically); to monitor subjects (e.g., human) undergoing treatment for SMM, MGUS, or MM; and to assess the response in subjects (e.g., human) after treatment for SMM, MGUS, or MM.

In particular, the data in the Examples section herein demonstrate that immune biomarkers can be used to identify subjects (e.g., humans) with SMM or MGUS that would benefit from treatment (e.g., before progression to MM for those with SMM or MGUS). For instance, based on the data in the Examples section herein, subjects (e.g., human) with SMM, MGUS, or MM having one or more (e.g., 1, 2, 3, 4, 5, 6, 7, 8, 9, 10, 11) (e.g., prior to treatment for SMM, MGUS, or MM) of the immune biomarkers of Table 1 are predicted to have significantly longer progression-free survival upon treatment (e.g., with immunotherapy) and, thus, are predicted to benefit from treatment (e.g., with immunotherapy or early treatment for SMM or MGUS subjects, i.e., treatment before progression from SMM or MGUS to MM). Additionally, based on the data in the Examples section herein, subjects (e.g., human) with SMM, MGUS, or MM having one or more (e.g., 1, 2, 3, 4, 5, 6, 7, 8, 9, 10, 11) (e.g., prior to treatment for SMM, MGUS, or MM) of the biomarkers of Table 2 are predicted to have significantly shorter progression-free survival upon treatment (e.g., with immunotherapy), and, thus, would benefit from treatment (e.g., with one or more therapeutic agents other than or in addition to immunotherapy or early treatment for SMM or MGUS subjects, i.e., treatment before progression from SMM or MGUS to MM).

The data in the Examples section also demonstrate that immune biomarkers can be used to monitor the response to treatment (e.g., immunotherapy) in a subject (e.g., human) with SMM, MGUS, or MM and determine which subjects with SMM, MGUS, or MM should be treated with a different, e.g., more intensive regimen. For instance, based on the Examples section herein, subjects (e.g., human) with SMM, MGUS, or MM having one or more (e.g., 1, 2, 3, 4, 5, 6, 7) of the immune biomarkers of Table 3 while undergoing treatment (e.g., with immunotherapy) for SMM, MGUS, or MM are predicted to have significantly shorter progression-free survival upon treatment and, thus, are predicted to benefit from a different, e.g., more intensive treatment before progression to MM (e.g., higher dose(s), more dose(s), combination therapy, or a different therapy from that being used). Additionally, based on the Examples section herein, subjects (e.g., human) with SMM, MGUS, or MM having one or more (e.g., 1, 2, 3, 4, 5, 6, 7) of the immune biomarkers of Table 4 while undergoing treatment (e.g., with immunotherapy) for SMM, MGUS, or MM are predicted to have significantly longer progression-free survival upon treatment, and thus, are predicted to benefit from continuing the treatment.

The data in the Examples section also demonstrate that immune biomarkers can be used to monitor response to treatment (e.g., immunotherapy) in subjects (e.g., humans) with SMM, MGUS, MM and to determine which subjects are likely to have prolonged biochemical progression-free survival and, thus, for whom treatment may be terminated or modified (e.g., changes in amount, duration, or type of treatment; changes in the frequency of follow-up assessments or the type of tests performed clinically). For instance, based on the Examples section herein, subjects (e.g., human) with SMM, MGUS, or MM having an immune biomarker from Table 5 after treatment (e.g., with immunotherapy) were predicted to have prolonged biochemical progression-free survival and, thus, to benefit from terminating or modifying (e.g., changing the amount, duration, or type of treatment; changes in the frequency of follow-up assessments or the type of tests performed clinically) the treatment. Additionally, based on the Examples section herein, subjects (e.g., human) with SMM, MGUS, or MM having an immune biomarker from Table 6 after treatment (e.g., with immunotherapy) were predicted to have shortened biochemical progression-free survival and, thus, to benefit from continued or more intensive (e.g., changing the amount, duration, or type of treatment; changes in the frequency of follow-up assessments or the type of tests performed clinically) treatment.

Accordingly, provided herein is a method for identifying a human subject having SMM, MGUS, or MM that would benefit from treatment, the method comprising determining that a sample (e.g., mononuclear cells obtained from a blood sample, CD138-negative (CD138−) mononuclear cells obtained from a bone marrow sample or a blood sample, or a bone marrow tissue section) obtained from the human subject have one or more of (e.g., 1, 2, 3, 4, 5, 6, 7, 8, 9, 10, 11) a biomarker set forth in Table 1 or one or more (e.g., 1, 2, 3, 4, 5, 6, 7, 8, 9, 10, 11) of the biomarkers of Table 2, e.g., one or more (e.g., 1, 2, 3, 4, 5, 6, 7, 8, 9, 10, 11) of (i) an increased or decreased abundance of granzyme K positive (GZMK⁺) T cells relative to a control abundance of GZMK⁺ T cells; (ii) an increased or decreased abundance of GZMK⁺ natural killer (NK) cells relative to a control abundance of GZMK⁺ NK cells; (iii) an increased or decreased abundance of Th17 cells relative to a control abundance of Th17 cells; (iv) an increased or decreased abundance of plasmacytoid dendritic cells (pDCs) relative to a control abundance of pDCs; (v) an increased or decreased abundance of hematopoietic stem cells (HSCs) relative to a control abundance of HSCs; (vi) an increased or decreased abundance of mature B-cells, which include both naïve and memory B-cells, for example, relative to a control abundance of mature B-cells; (vii) an increased or decreased activity of GZMK-associated signaling relative to a control activity of GZMK-associated signaling; (viii) an increased or decreased activity of Th17-associated signaling relative to a control activity of Th17-associated signaling; (ix) an increased or decreased activity of a compositional signature that captures an abundance of mature B-cells relative to a control activity of a compositional signature that captures an abundance of mature B-cells; (x) an increased or decreased activity of a compositional signature that captures an abundance of HSCs relative to a control activity of a compositional signature that captures an abundance of HSCs; and (xi) an increased or decreased activity of an immune reactivity score relative to a control activity of an immune reactivity score. In some instances, the human subject has not or is not undergoing treatment for SMM, MGUS, or MM. In some instances, the human subject has SMM. In some instances, the human subject has high-risk SMM. In some instances, the human subject has MGUS. In some instances, the human subject has MM.

In some instances, the method comprises determining that the sample (e.g., mononuclear cells obtained from a blood sample, CD138-mononuclear cells obtained from a bone marrow sample or a blood sample, or a bone marrow tissue section) obtained from the human subject has one or more (e.g., 1, 2, 3, 4, 5, 6, 7, 8, 9, 10, 11) of the immune biomarkers from Table 1, wherein the sample is obtained prior to treatment. In some instances, the method further comprises (i.e., after the determining) administering to the human subject a treatment for SMM, MGUS, or MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, and a therapeutically effective dose of dexamethasone. In some instances, the treatment for MM, SMM, or MGUS comprises a therapeutically effective dose of elotuzumab, a therapeutically effective dose of lenalidomide, and a therapeutically effective dose of dexamethasone.

In some instances, the method comprises determining that the sample (e.g., mononuclear cells obtained from a blood sample, CD138-mononuclear cells obtained from a bone marrow sample or a blood sample, or a bone marrow tissue section) obtained from the human subject has one or more (e.g., 1, 2, 3, 4, 5, 6, 7, 8, 9, 10, 11) of the immune biomarkers from Table 2, wherein the sample is obtained prior to treatment. In some instances, the method further comprises (i.e., after the determining) administering to the human subject a treatment for SMM, MGUS, or MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, a therapeutically effective dose of dexamethasone, and one or more (e.g., 1, 2, 3) additional therapeutic agents. In some instances, the treatment does not comprise immunotherapy. In some instances, the treatment does not comprise elotuzumab, lenalidomide, and/or dexamethasone. In some instances, the treatment for MM, SMM, or MGUS comprises a therapeutically effective dose of elotuzumab, a therapeutically effective dose of lenalidomide, and a therapeutically effective dose of dexamethasone.

In some instances, (i) the control abundance of GZMK⁺ T cells is a median, mean, first quartile, or third quartile abundance of GZMK⁺ T cells in healthy human subjects or human subjects with MGUS, SMM, or MM; (ii) the control abundance of GZMK-NK cells is a median, mean, first quartile, or third quartile abundance of GZMK⁺ NK cells in healthy human subjects or human subjects with MGUS, SMM, or MM; (iii) the control abundance of Th17 cells is a median, mean, first quartile, or third quartile abundance of Th17 cells in healthy human subjects or human subjects with MGUS, SMM, or MM; (iv) the control abundance of pDCs is a median, mean, first quartile, or third quartile abundance of pDCs in healthy human subjects or human subjects with MGUS, SMM, or MM; (v) the control abundance of HSCs is a median, mean, first quartile, or third quartile abundance of HSCs in healthy human subjects or human subjects with MGUS, SMM, or MM; (vi) the control abundance of mature B-cells is a median, mean, first quartile, or third quartile abundance of mature B-cells in healthy human subjects or human subjects with MGUS, SMM, or MM; (vii) the control activity of GZMK-associated signaling is a median, mean, first quartile, or third quartile activity of GZMK-associated signaling in healthy human subjects or human subjects with MGUS, SMM, or MM; (viii) the control activity of Th17-associated signaling is a median, mean, first quartile, or third quartile activity of Th17-associated signaling in healthy human subjects or human subjects with MGUS, SMM, or MM; (ix) the control activity of a compositional signature that captures an abundance of mature B-cells is a median, mean, first quartile, or third quartile activity of a compositional signature that captures an abundance of mature B-cells in healthy human subjects or human subjects with MGUS, SMM, or MM; (x) the control activity of a compositional signature that captures an abundance of HSCs is a median, mean, first quartile, or third quartile activity of a compositional signature that captures an abundance of HSCs in healthy human subjects or human subjects with MGUS, SMM, or MM; and (xi) the control activity of an immune reactivity score is a median, mean, first quartile, or third quartile activity of an immune reactivity score in healthy human subjects or human subjects with MGUS, SMM, or MM. In some instances, the control is relative to healthy human subjects. In some instances, the control is relative to human subjects with MGUS, SMM, or MM. In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for treating SMM, MGUS, or MM in a human subject, the method comprising administering to the human subject a therapeutically effective amount of a treatment for SMM, MGUS, or MM; wherein a sample (e.g., mononuclear cells from a blood sample, CD138-mononuclear cells from a bone marrow sample or a blood sample, or a bone marrow tissue section) obtained from the human subject has previously been determined to have one or more (e.g., 1, 2, 3, 4, 5, 6, 7, 8, 9, 10, 11) of the biomarkers of Table 1 or one or more (e.g., 1, 2, 3, 4, 5, 6, 7, 8, 9, 10, 11) of the biomarkers of Table 2, e.g., one or more (e.g., 1, 2, 3, 4, 5, 6, 7, 8, 9, 10, 11) of: (i) an increased or decreased abundance of granzyme K positive (GZMK⁺) T cells relative to a control abundance of GZMK⁺ T cells; (ii) an increased or decreased abundance of GZMK⁺ natural killer (NK) cells relative to a control abundance of GZMK⁺ NK cells; (iii) an increased or decreased abundance of Th17 cells relative to a control abundance of Th17 cells; (iv) an increased or decreased abundance of plasmacytoid dendritic cells (pDCs) relative to a control abundance of pDCs; (v) an increased or decreased abundance of hematopoietic stem cells (HSCs) relative to a control abundance of HSCs; (vi) an increased or decreased abundance of mature B-cells relative to a control abundance of mature B-cells; (vii) an increased or decreased activity of GZMK-associated signaling relative to a control activity of GZMK-associated signaling; (viii) an increased or decreased activity of Th17-associated signaling relative to a control activity of Th17-associated signaling; (ix) an increased or decreased activity of a compositional signature that captures an abundance of mature B-cells relative to a control activity of a compositional signature that captures an abundance of mature B-cells; (x) an increased or decreased activity of a compositional signature that captures an abundance of HSCs relative to a control activity of a compositional signature that captures an abundance of HSCs; and (xi) an increased or decreased activity of an immune reactivity score relative to a control activity of an immune reactivity score. In some instances, the sample (e.g., mononuclear cells from a blood sample, CD138-mononuclear cells from a bone marrow sample or a blood sample, or bone marrow tissue section) is obtained from the human prior to treatment for SMM, MGUS, or MM. In some instances, the human subject has SMM. In some instances, the human subject has high-risk SMM. In some instances, the human subject has MGUS. In some instances, the human subject has MM. In some instances, the method is for treating SMM. In some instances, the method is for treating MGUS. In some instances, the method is for treating MM.

In some instances, the method comprises administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein the sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7, 8, 9, 10, 11) of the immune biomarkers from Table 1. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, and a therapeutically effective dose of dexamethasone. In some instances, the treatment comprises a therapeutically effective dose of elotuzumab, a therapeutically effective dose of lenalidomide, and a therapeutically effective dose of dexamethasone.

In some instances, the method comprises administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein the sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7, 8, 9, 10, 11) of the immune biomarkers from Table 2. In some instances, the method comprises obtaining the sample from the human subject and determining the one or more immune biomarkers. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, a therapeutically effective dose of dexamethasone, and one or more (e.g., 1, 2, 3) additional therapeutic agents. In some instances, the treatment does not comprise immunotherapy. In some instances, the treatment comprises a therapeutically effective dose of elotuzumab, a therapeutically effective dose of lenalidomide, and a therapeutically effective dose of dexamethasone.

In some instances, (i) the control abundance of GZMK⁺ T cells is a median, mean, first quartile, or third quartile abundance of GZMK⁺ T cells in healthy human subjects or human subjects with MGUS, SMM, or MM; (ii) the control abundance of GZMK⁺ NK cells is a median, mean, first quartile, or third quartile abundance of GZMK⁺ NK cells in healthy human subjects or human subjects with MGUS, SMM, or MM; (iii) the control abundance of Th17 cells is a median, mean, first quartile, or third quartile abundance of Th17 cells in healthy human subjects or human subjects with MGUS, SMM, or MM; (iv) the control abundance of pDCs is a median, mean, first quartile, or third quartile abundance of pDCs in healthy human subjects or human subjects with MGUS, SMM, or MM; (v) the control abundance of HSCs is a median, mean, first quartile, or third quartile abundance of HSCs in healthy human subjects or human subjects with MGUS, SMM, or MM; (vi) the control abundance of mature B-cells is a median, mean, first quartile, or third quartile abundance of mature B-cells in healthy human subjects or human subjects with MGUS, SMM, or MM; (vii) the control activity of GZMK-associated signaling is a median, mean, first quartile, or third quartile activity of GZMK-associated signaling in healthy human subjects or human subjects with MGUS, SMM, or MM; (viii) the control activity of Th17-associated signaling is a median, mean, first quartile, or third quartile activity of Th17-associated signaling in healthy human subjects or human subjects with MGUS, SMM, or MM; (ix) the control activity of a compositional signature that captures an abundance of mature B-cells is a median, mean, first quartile, or third quartile activity of a compositional signature that captures an abundance of mature B-cells in healthy human subjects or human subjects with MGUS, SMM, or MM; (x) the control activity of a compositional signature that captures an abundance of HSCs is a median, mean, first quartile, or third quartile activity of a compositional signature that captures an abundance of HSCs in healthy human subjects or human subjects with MGUS, SMM, or MM; and (xi) the control activity of an immune reactivity score is a median, mean, first quartile, or third quartile activity of an immune reactivity score in healthy human subjects or human subjects with MGUS, SMM, or MM. In some instances, the control is relative to healthy human subjects. In some instances, the control is relative to human subjects with MGUS, SMM, or MM. In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for monitoring a human subject having SMM, MGUS, or MM undergoing treatment for SMM, MGUS, or MM, the method comprising determining that mononuclear cells obtained from a blood sample, CD138-mononuclear cells obtained from a bone marrow sample, or a bone marrow tissue section obtained from the human subject have one or more (e.g., 1, 2, 3, 4, 5, 6, 7) of the biomarkers in Table 3 or one or more (e.g., 1, 2, 3, 4, 5, 6, 7) of the biomarkers of Table 4, e.g., one or more (e.g., 1, 2, 3, 4, 5, 6, 7) of: (i) an increased or decreased abundance of tissue-resident NK cells relative to a control abundance of tissue-resident NK cells; (ii) an increased or decreased abundance of exhausted GZMK⁺ CD8⁺ T-cells relative to a control abundance of exhausted GZMK⁺ CD8⁺ T-cells; (iii) an increased or decreased abundance of activated CD4⁺ Central Memory T-cells (aCD4⁺ TCMs) relative to a control abundance of aCD4⁺ TCMs; (iv) an increased or decreased activity of a compositional signature corresponding to the abundance of tissue-resident NK cells, exhausted GZMK⁺ CD8⁺ T-cells, and activated CD4⁺ Central Memory T-cells relative to a control activity of the compositional signature corresponding to the abundance of tissue-resident NK cells, exhausted GZMK⁺ CD8⁺ T-cells, and activated CD4⁺ Central Memory T-cells; (v) an increased or decreased activity of a gene expression signature corresponding to the activity of one or more genes selected from the group consisting of AREG, FAM177A1, RGS1, and IL32 relative to a control activity of a gene expression signature corresponding to the activity of the one or more genes selected from the group consisting of AREG, FAM177A1, RGS1, and IL32; (vi) an increased or decreased expression level of one or more genes selected from the group consisting of AREG, FAM177A1, and RGS1 relative to a control expression level of the one or more genes selected from the group consisting of AREG, FAM177A1, and RGS1; and (vii) an increased or decreased expression level of IL32 relative to a control expression level of IL32. In some instances, the human subject has SMM. In some instances, the human subject having SMM has high-risk SMM. In some instances, the human subject has MGUS. In some instances, the human subject has MM.

In some instances, the method comprises determining that the sample (e.g., blood or bone marrow, e.g., mononuclear cells obtained from blood or CD138-mononuclear cells obtained from bone marrow or blood) obtained from the human subject has one or more of (e.g., 1, 2, 3, 4, 5, 6, 7) the immune biomarkers from Table 3. In some instances, the method further comprises administering to the human subject a treatment for MM, SMM, or MGUS. In some instances in which the subject has SMM or MGUS, the method further comprises administering to the human subject a treatment for MM, SMM, or MGUS prior to progression to MM. In some instances, the method further comprises (i.e., after the determining) administering to the human subject a different, e.g., more intensive, treatment (e.g., higher dose(s), more dose(s), combination therapy, or a different therapy from that being used) for SMM, MGUS, or MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab). In some instances, the more intensive treatment comprises a therapeutically effective dose of elotuzumab, a therapeutically effective dose of lenalidomide, and a therapeutically effective dose of dexamethasone. In some instances, the more intensive treatment comprises (a) a therapeutically effective amount of a proteasome inhibitor, an immunomodulatory drug, and a steroid; (b) a therapeutically effective amount of a monoclonal antibody that specifically binds to SLAMF7 or to CD38, a proteasome inhibitor, an immunomodulatory drug, and a steroid; (c) a therapeutically effective amount of autologous stem cell transplantation (ASCT); (d) a therapeutically effective amount of CAR-T cells targeting BCMA; (e) a therapeutically effective amount of a bispecific antibody that specifically binds to BCMA; or (f) a therapeutically effective amount of a monoclonal antibody that specifically binds to SLAMF7 or to CD38, an immunomodulatory drug, and a steroid.

In some instances, the method comprises determining that the sample (e.g., blood or bone marrow, e.g., mononuclear cells obtained from blood or CD138-mononuclear cells obtained from bone marrow or blood) obtained from the human subject has one or more of (e.g., 1, 2, 3, 4, 5, 6, 7) the immune biomarkers from Table 4. In some instances, the method further comprises administering to the human subject a treatment for MM, SMM, or MGUS. In some instances in which the subject has SMM or MGUS, the method further comprises administering to the human subject a treatment for MM, SMM, or MGUS prior to progression to MM. In some instances, the method comprises administering the treatment indefinitely. In some instances, the method comprises administering the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject 1, 2, 3, 4, 5, 6, 7, 8, 9, 10 or more additional doses of the treatment. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab). In some instances, the treatment comprises a therapeutically effective dose of elotuzumab, a therapeutically effective dose of lenalidomide, and a therapeutically effective dose of dexamethasone. In some instances, the treatment comprises (a) a therapeutically effective amount of a proteasome inhibitor, an immunomodulatory drug, and a steroid; (b) a therapeutically effective amount of a monoclonal antibody that specifically binds to SLAMF7 or to CD38, a proteasome inhibitor, an immunomodulatory drug, and a steroid; (c) a therapeutically effective amount of autologous stem cell transplantation (ASCT); (d) a therapeutically effective amount of CAR-T cells targeting BCMA; (e) a therapeutically effective amount of a bispecific antibody that specifically binds to BCMA; or (f) a therapeutically effective amount of a monoclonal antibody that specifically binds to SLAMF7 or to CD38, an immunomodulatory drug, and a steroid.

In some instances, (i) the control abundance of tissue-resident NK cells is a median, mean, first quartile, or third quartile abundance of tissue-resident NK cells in healthy human subjects or human subjects with MGUS, SMM, or MM; (ii) the control abundance of exhausted GZMK⁺ CD8⁺ T-cells is a median, mean, first quartile, or third quartile abundance of exhausted GZMK⁺ CD8⁺ T-cells in healthy human subjects or human subjects with MGUS, SMM, or MM; (iii) the control abundance of activated CD4⁺ Central Memory T-cells (aCD4⁺ TCMs) is a median, mean, first quartile, or third quartile abundance of aCD4⁺ TCMs in healthy human subjects or human subjects with MGUS, SMM, or MM; (iv) the control activity of a compositional signature corresponding to the abundance of tissue-resident NK cells, exhausted GZMK⁺ CD8⁺ T-cells, and activated CD4⁺ Central Memory T-cells is a median, mean, first quartile, or third quartile activity of the compositional signature corresponding to the abundance of tissue-resident NK cells, exhausted GZMK⁺ CD8⁺ T-cells, and activated CD4⁺ Central Memory T-cells in healthy human subjects or human subjects with MGUS, SMM, or MM; (v) the control activity of a gene expression signature corresponding to the activity of one or more genes selected from the group consisting of AREG, FAM177A1, RGS1, and IL32 is a median, mean, first quartile, or third quartile activity of a gene expression signature corresponding to the activity of the one or more genes selected from the group consisting of AREG, FAM177A1, RGS1, and IL32 in healthy human subjects or human subjects with MGUS, SMM, or MM; (vi) the control expression level of one or more genes selected from the group consisting of AREG, FAM177A1, and RGS1 is a median, mean, first quartile, or third quartile expression level of the one or more genes selected from the group consisting of AREG, FAM177A1, and RGS1 in healthy human subjects or human subjects with MGUS, SMM, or MM; and (vii) the control expression level of IL32 is a median, mean, first quartile, or third quartile expression level of IL32 in healthy human subjects or human subjects with MGUS, SMM, or MM. In some instances, the control is relative to healthy human subjects. In some instances, the control is relative to human subjects with MGUS, SMM, or MM. In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for treating SMM, MGUS, or MM in a human subject, the method comprising administering to the human subject a therapeutically effective amount of a treatment for SMM, MGUS, or MM; wherein a sample (e.g., mononuclear cells from a blood sample, CD138-mononuclear cells from a bone marrow sample or a blood sample, or a bone marrow tissue section) obtained from the human subject has previously been determined to have one or more (e.g., 1, 2, 3, 4, 5, 6, 7) of the biomarkers in Table 3 or one or more (e.g., 1, 2, 3, 4, 5, 6, 7) of the biomarkers in Table 4, e.g., one or more (e.g., 1, 2, 3, 4, 5, 6, 7) of: (i) an increased or decreased abundance of tissue-resident NK cells relative to a control abundance of tissue-resident NK cells; (ii) an increased or decreased abundance of exhausted GZMK-CD8⁺ T-cells relative to a control abundance of exhausted GZMK⁺ CD8⁺ T-cells; (iii) an increased or decreased abundance of activated CD4⁺ Central Memory T-cells (aCD4⁺ TCMs) relative to a control abundance of aCD4⁺ TCMs; (iv) an increased or decreased activity of a compositional signature corresponding to the abundance of tissue-resident NK cells, exhausted GZMK⁺ CD8⁺ T-cells, and activated CD4⁺ Central Memory T-cells relative to a control activity of the compositional signature corresponding to the abundance of tissue-resident NK cells, exhausted GZMK⁺ CD8⁺ T-cells, and activated CD4⁺ Central Memory T-cells; (v) an increased or decreased activity of a gene expression signature corresponding to the activity of one or more genes selected from the group consisting of AREG, FAM177A1, RGS1, and IL32 relative to a control activity of a gene expression signature corresponding to the activity of the one or more genes selected from the group consisting of AREG, FAM177A1, RGS1, and IL32; (vi) an increased or decreased expression level of one or more genes selected from the group consisting of AREG, FAM177A1, and RGS1 relative to a control expression level of the one or more genes selected from the group consisting of AREG, FAM177A1, and RGS1; and (vii) an increased or decreased expression level of IL32 relative to a control expression level of IL32. In some instances, the human subject has SMM. In some instances, the human subject has high risk SMM. In some instances, the human subject has MGUS. In some instances, the human subject has MM. In some instances, the method is for treating SMM. In some instances, the method is for treating MGUS. In some instances, the method is for treating MM.

In some instances, the method comprises administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein the sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7) of the immune biomarkers from Table 3, wherein the human subject was undergoing a first treatment for SMM, MGUS, or MM (e.g., immunotherapy) when the sample was obtained from the human subject, and wherein the treatment administered to the human subject is different from the first treatment. In some instances, the first treatment comprises or consists of a treatment for SMM described herein. In some instances, the first treatment comprises or consists of a treatment for MGUS described herein. In some instances, the first treatment comprises or consists of a treatment for MM described herein. In some instances, the first treatment comprises or consists of elotuzumab, lenalidomide, and dexamethasone. In some instances, the first treatment comprises or consists of a triplet therapy (i.e., a combination of a proteasome inhibitor, an immunomodulatory drug, and a steroid, e.g., dexamethasone). In some instances, the first treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab). In some instances, the treatment administered to the human subject is more intensive than the first treatment (e.g., higher dose(s), more dose(s), combination therapy, or a different therapy from that being used). In some instances, the treatment administered to the human subject comprises or consists of a treatment for SMM described herein. In some instances, the treatment administered to the human subject comprises or consists of a treatment for MGUS described herein. In some instances, the treatment administered to the human subject comprises or consists of a treatment for MM described herein. In some instances, the treatment administered to the human subject comprises or consists of elotuzumab, lenalidomide, and dexamethasone. In some instances, the treatment administered to the human subject comprises or consists of a triplet therapy (i.e., a combination of a proteasome inhibitor, an immunomodulatory drug, and a steroid, e.g., dexamethasone). In some instances, the treatment administered to the human subject comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment administered to the human subject treatment comprises or consists of autologous stem cell transplantation (ASCT). In some instances, the treatment administered to the human subject comprises or consists of CAR-T cells targeting BCMA (e.g., Abecma®) [idecabtagene vicleucel]). In some instances, the treatment administered to the human subject comprises or consists of a bispecific antibody targeting BCMA (e.g., an anti-BCMA/anti-CD3 bispecific antibody, e.g., Teclistamab). In some instances, the treatment is administered to the human subject before the SMM or MGUS progresses to MM (e.g., overt MM). In some instances, the treatment comprises or consists of a therapeutically effective dose of elotuzumab, a therapeutically effective dose of lenalidomide, and a therapeutically effective dose of dexamethasone. In some instances, the treatment comprises or consists of (a) a therapeutically effective amount of a proteasome inhibitor, an immunomodulatory drug, and a steroid; (b) a therapeutically effective amount of a monoclonal antibody that specifically binds to SLAMF7 or to CD38, a proteasome inhibitor, an immunomodulatory drug, and a steroid; (c) a therapeutically effective amount of autologous stem cell transplantation (ASCT); (d) a therapeutically effective amount of CAR-T cells targeting BCMA; (e) a therapeutically effective amount of a bispecific antibody that specifically binds to BCMA; or (f) a therapeutically effective amount of a monoclonal antibody that specifically binds to SLAMF7 or to CD38, an immunomodulatory drug, and a steroid.

In some instances, the method comprises administering to the human subject a treatment for SMM, MGUS, or MM; wherein the sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7) of the immune biomarkers from Table 4, wherein the human subject was undergoing the treatment when the sample was obtained from the human subject. In some instances, the method comprises continuing the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject 1, 2, 3, 4, 5, 6, 7, 8, 9, 10 or more additional doses of the treatment. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab).

In some instances, (i) the control abundance of tissue-resident NK cells is a median, mean, first quartile, or third quartile abundance of tissue-resident NK cells in healthy human subjects or human subjects with MGUS, SMM, or MM; (ii) the control abundance of exhausted GZMK⁺ CD8⁺ T-cells is a median, mean, first quartile, or third quartile abundance of exhausted GZMK⁺ CD8⁺ T-cells in healthy human subjects or human subjects with MGUS, SMM, or MM; (iii) the control abundance of activated CD4⁺ Central Memory T-cells (aCD4⁺ TCMs) is a median, mean, first quartile, or third quartile abundance of aCD4⁺ TCMs in healthy human subjects or human subjects with MGUS, SMM, or MM; (iv) the control activity of a compositional signature corresponding to the abundance of tissue-resident NK cells, exhausted GZMK⁺ CD8⁺ T-cells, and activated CD4⁺ Central Memory T-cells is a median, mean, first quartile, or third quartile activity of the compositional signature corresponding to the abundance of tissue-resident NK cells, exhausted GZMK⁺ CD8⁺ T-cells, and activated CD4⁺ Central Memory T-cells in healthy human subjects or human subjects with MGUS, SMM, or MM; (v) the control activity of a gene expression signature corresponding to the activity of one or more genes selected from the group consisting of AREG, FAM177A1, RGS1, and IL32 is a median, mean, first quartile, or third quartile activity of a gene expression signature corresponding to the activity of the one or more genes selected from the group consisting of AREG, FAM177A1, RGS1, and IL32 in healthy human subjects or human subjects with MGUS, SMM, or MM; (vi) the control expression level of one or more genes selected from the group consisting of AREG, FAM177A1, and RGS1 is a median, mean, first quartile, or third quartile expression level of the one or more genes selected from the group consisting of AREG, FAM177A1, and RGS1 in healthy human subjects or human subjects with MGUS, SMM, or MM; and (vii) the control expression level of IL32 is a median, mean, first quartile, or third quartile expression level of IL32 in healthy human subjects or human subjects with MGUS, SMM, or MM. In some instances, the control is relative to healthy human subjects. In some instances, the control is relative to human subjects with MGUS, SMM, or MM. In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for identifying a human subject having SMM, MGUS, or MM that would benefit from termination or modification of treatment for SMM, MGUS, or MM, the method comprising determining that CD138-mononuclear cells obtained from a bone marrow sample or blood sample, mononuclear cells obtained from a blood sample, or a bone marrow tissue section obtained from the human subject have an immune cell composition similar to a control immune cell composition, wherein the control immune cell composition is an immune cell composition for a panel (e.g., 1, 2, 3, 4, 5, 6, 7, 8, 9, 10, 15, 20, 25, 30, 35, or 40 or more) of healthy human subjects. In some instances, the human subject has SMM. In some instances, the human subject having SMM has high-risk SMM. In some instances, the human subject has MGUS. In some instances, the human subject has MM.

In some instances, the method comprises determining that a sample (e.g., blood or bone marrow, e.g., mononuclear cells obtained from blood or CD138-mononuclear cells obtained from bone marrow or blood) obtained from the human subject has an immune biomarker from Table 5. In some instances, the method comprises obtaining the sample from the human subject. In some instances, the sample is obtained from the human subject within one day, within one week, within one month, within two months, within three months, within six months, or within one year of the last dose of the treatment (e.g., immunotherapy and optionally a steroid, e.g., elotuzumab, lenalidomide, and dexamethasone). In some instances, the method further comprises (i.e., after the determining) terminating the treatment. In some instances, the method comprises terminating the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method further comprises (i.e., after the determining) modifying the treatment (e.g., changes in amount, duration, or type of treatment; changes in the frequency of follow-up assessments or the type of tests performed clinically). In some instances, the modifying of the treatment is to reduce treatment (e.g., reduce the amount, reduce the duration, reduce the doses). For example, in some instances, the method comprises decreasing the dose of the treatment. In some instances, the method comprises decreasing the frequency of the dose of the medication. In some instances, the method comprises decreasing the frequency of follow-up assessments. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab).

In some instances, the method comprises determining that the sample (e.g., blood or bone marrow, e.g., mononuclear cells obtained from blood or CD138-mononuclear cells obtained from bone marrow or blood) obtained from the human subject has an immune biomarker from Table 6. In some instances, the method comprises obtaining the sample from the human subject. In some instances, the sample is obtained from the human subject within one day, within one week, within one month, within two months, within three months, within six months, or within one year of the last dose of the treatment (e.g., immunotherapy and optionally a steroid, e.g., elotuzumab, lenalidomide, and dexamethasone). In some instances, the method further comprises (i.e., after the determining) administering to the human subject a different, e.g., more intensive, treatment (e.g., higher dose(s), more dose(s), combination therapy, or a different therapy from that being used) for SMM, MGUS, or MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab).

In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for treating SMM, MGUS, or MM in a human subject (e.g., in a human subject who has undergone treatment), the method comprising: (a) administering to the human subject a therapeutically effective amount of a treatment for SMM, MGUS, or MM; (b) determining that mononuclear cells obtained from a sample (e.g., a blood sample, CD138-mononuclear cells obtained from a bone marrow sample or a blood sample, or a bone marrow tissue section) obtained from the human subject has an immune cell composition that is similar to a control immune cell composition (i.e., a biomarker of Table 5), wherein the control immune cell composition is an immune cell composition for a panel (e.g., 1, 2, 3, 4, 5, 6, 7, 8, 9, 10, 15, 20, 25, 30, 35, or 40 or more) of healthy human subjects, and (c) discontinuing or modifying the administering of the treatment for SMM, MGUS, or MM. Also provided herein is a method for treating SMM, MGUS, or MM in a human subject who has undergone treatment (e.g., received 1, 2, 3, or more doses) for SMM, MGUS, or MM (e.g., immunotherapy), the method comprising terminating (discontinuing) the treatment or administering to the human subject a modified treatment (e.g., different amount, duration, or type of treatment, different frequency of follow-up assessments, different type of tests performed clinically), wherein a sample (e.g., blood or bone marrow, e.g., mononuclear cells obtained from blood or CD138-mononuclear cells obtained from bone marrow or blood) obtained from the human subject has previously been determined to have an immune biomarker from Table 5, and wherein the sample was obtained from the human subject after (e.g., within one day, within one week, within one month, within two months, within three months, within six months, or within one year of) the last dose of the treatment. In some instances, the human subject has SMM. In some instances, the human subject having SMM has high-risk SMM. In some instances, the human subject has MGUS. In some instances, the human subject has MM. In some instances, the method is for treating SMM. In some instances, the method is for treating MGUS. In some instances, the method is for treating MM. In some instances, the method comprises obtaining the sample from the human subject. In some instances, the method comprises (i.e., after the determining) terminating the treatment. In some instances, the method comprises terminating the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject a modified treatment (e.g., different amount, duration, or type of treatment, different frequency of follow-up assessments, different type of tests performed clinically). In some instances, the modified treatment is a reduced treatment (e.g., reduced amount, reduced duration, reduce number of doses). For example, in some instances, the method comprises administering to the human subject a decreased dose of the treatment. In some instances, the method comprises administering to the human subject a decreased frequency of the dose of the medication. In some instances, the method comprises decreasing the frequency of follow-up assessments. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab). In some instances, the modified treatment is a treatment for SMM described herein. In some instances, the modified treatment is a treatment for MGUS described herein. In some instances, the modified treatment is a treatment for MM described herein. In some instances, the modified treatment comprises or consists of a triplet therapy (i.e., a combination of a proteasome inhibitor, an immunomodulatory drug, and a steroid, e.g., dexamethasone). In some instances, the modified treatment comprises or consists of elotuzumab, lenalidomide, and dexamethasone. In some instances, the modified treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the modified treatment comprises or consists of autologous stem cell transplantation (ASCT). In some instances, the treatment comprises or consists of CAR-T cells targeting BCMA (e.g., Abecma® [idecabtagene vicleucel]). In some instances, the modified treatment comprises or consists of a bispecific antibody targeting BCMA (e.g., an anti-BCMA/anti-CD3 bispecific antibody, e.g., Teclistamab). In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Also provided herein is a method for treating SMM, MGUS, or MM in a human subject (e.g., in a human subject who has undergone treatment), the method comprising: (a) administering to the human subject a therapeutically effective amount of a treatment for SMM, MGUS, or MM; (b) determining that mononuclear cells obtained from a sample (e.g., a blood sample, CD138-mononuclear cells obtained from a bone marrow sample or a blood sample, or a bone marrow tissue section) obtained from the human subject has an immune cell composition that is different from a control immune cell composition (i.e., a biomarker of Table 6), wherein the control immune cell composition is an immune cell composition for a panel (e.g., 1, 2, 3, 4, 5, 6, 7, 8, 9, 10, 15, 20, 25, 30, 35, or 40 or more) of healthy human subjects, and (c) administering to the human subject a different, e.g. more intensive, treatment (e.g., higher dose(s), more dose(s), combination therapy, or a different therapy from that being used). Also provided herein is a method for treating SMM, MGUS, or MM in a human subject who has undergone treatment (e.g., received 1, 2, 3, or more doses) for SMM, MGUS, or MM (e.g., immunotherapy), the method comprising administering to the human subject a different, e.g. more intensive, treatment (e.g., higher dose(s), more dose(s), combination therapy, or a different therapy from that being used), wherein a sample (e.g., blood or bone marrow, e.g., mononuclear cells obtained from blood or CD138-mononuclear cells obtained from bone marrow or blood) obtained from the human subject has previously been determined to have an immune biomarker from Table 6, and wherein the sample was obtained from the human subject after (e.g., within one day, within one week, within one month, within two months, within three months, within six months, or within one year of) the last dose of the treatment. In some instances, the method is for treating SMM. In some instances, the method is for treating MGUS. In some instances, the method is for treating MM. In some instances, the method comprises obtaining the sample from the human subject. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab). In some instances, the determining is by single cell RNA sequencing, targeted single-cell RNA-sequencing, RNA-sequencing, immunohistochemistry, immunofluorescence, flow cytometry, mass cytometry, mass spectrometry, or an imaging-based method. In some instances, the method further comprises determining that the human subject has one or more genetic biomarkers described herein. In some instances, the method further comprises determining that the subject has a deletion of the short arm of chromosome 17 (Del17p).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “combination therapy” is meant any treatment that comprises the administration of two or more agents serially or concurrently. In one embodiment, the combination therapy comprises 2, 3 or more immunomodulatory agents and/or immunotherapeutic agents. In another embodiment, the combination therapy comprises Lenalidomide, Dexamethasone, and one or more other immunotherapeutic or immunomodulatory agents. In yet another embodiment, the combination therapy comprises Elotuzumab, Lenalidomide, Dexamethasone, or analogs thereof. In other embodiments, the combination therapy comprises Lenalidomide, Dexamethasone and a CAR-T cell, a bispecific antibody, steroid, or another immunomodulatory agent selected from a monoclonal antibody that specifically binds to, e.g., SLAMF7 or CD38, a proteasome inhibitor, an immunomodulatory drug, and a steroid, e.g., dexamethasone). In embodiments, the administration is separated by minutes (e.g., 5, 10, 20, 30, 40, 50 minutes), hours (e.g., 1, 2, 3, 6, 12, 18, 24 hours), days (e.g., 1, 2, 3, 5, 7), or even weeks (e.g., 1, 2, 3, 4, 6, 8).

By “Granzyme K (GZMK) polypeptide” is meant a GZMK protein or fragment thereof, having serine protease activity and having at least about 85% amino acid sequence identity to GenBank Accession No. AAH35802.1. An exemplary GZMK amino acid sequence from Homo Sapiens is provided below (GenBank: AAH35802.1).

| | |
|---|---|
| 1 | MTKFSSFSLF FLIVGAYMTH VCFNMEIIGG KEVSPHSRPF MASIQYGGHH VCGGVLIDPQ |
| 61 | WVLTAAHCQY RFTKGQSPTV VLGAHSLSKN EASKQTLEIK KFIPFSRVTS DPQSNDIMLV |
| 121 | KLQTAAKLNK HVKMLHIRSK TSLRSGTKCK VTGWGATDPD SLRPSDTLRE VTVTVLSRKL |
| 181 | CNSQSYYNGD PFITKDMVCA GDAKGQKDSC KGDSGGPLIC KGVFHAIVSG GHECGVATKP |
| 241 | GIYTLLTKKY QTWIKSNLVP PHTN. |

By “Granzyme K (GZMK) polynucleotide” is meant a nucleic acid molecule encoding a GZMK polypeptide, as well as the introns, exons, and regulatory sequences associated with its expression, or fragments thereof. In embodiments, a GZMK polynucleotide is the genomic sequence, mRNA, or gene associated with and/or required for GZMK expression. An exemplary GZMK nucleotide sequence from Homo Sapiens is provided below (GenBank: BC035802.1):

| | |
|---|---|
| 1 | gatcaacaca tttcatctgg gcttcttaaa tctaaatctt taaaatgact aagttttctt |
| 61 | ccttttctct gtttttccta atagttgggg cttatatgac tcatgtgtgt ttcaatatgg |
| 121 | aaattattgg agggaaagaa gtgtcacctc attccaggcc atttatggcc tccatccagt |
| 181 | atggcggaca tcacgtttgt ggaggtgttc tgattgatcc acagtgggtg ctgacagcag |
| 241 | cccactgcca atatcggttt accaaaggcc agtctcccac tgtggtttta ggcgcacact |
| 301 | ctctctcaaa gaatgaggcc tccaaacaaa cactggagat caaaaaattt ataccattct |
| 361 | caagagttac atcagatcct caatcaaatg atatcatgct ggttaagctt caaacagccg |
| 421 | caaaactcaa taaacatgtc aagatgctcc acataagatc caaaacctct cttagatctg |
| 481 | gaaccaaatg caaggttact ggctggggag ccaccgatcc agattcatta agaccttctg |
| 541 | acaccctgcg agaagtcact gttactgtcc taagtcgaaa actttgcaac agccaaagtt |
| 601 | actacaacgg cgaccctttt atcaccaaag acatggtctg tgcaggagat gccaaaggcc |
| 661 | agaaggattc ctgtaagggt gactcagggg gccccttgat ctgtaaaggt gtcttccacg |
| 721 | ctatagtctc tggaggtcat gaatgtggtg ttgccacaaa gcctggaatc tacaccctgt |
| 781 | taaccaagaa ataccagact tggatcaaaa gcaaccttgt cccgcctcat acaaattaag |
| 841 | ttacaaataa ttttattgga tgcacttgct tcttttttcc taatatgctc gcaggttaga |
| 901 | gttgggtgta agtaaagcag agcacatatg gggtccattt ttgcacttgt aagtcatttt |
| 961 | attaaggaat caagttcttt ttcacttgta tcactgatgt atttctacca tgctggtttt |
| 1021 | attctaaata aaatttagaa gactcaaaaa aaaaaaaaaa aaaaaaaaaa aaaa |

By “Granzyme B (GZMB) polypeptide” is meant a GZMB protein or fragment thereof, having serine protease activity and having at least about 85% amino acid sequence identity to GenBank Accession No. AAA75490.1. An exemplary GZMB amino acid sequence from Homo Sapiens is provided below (GenBank: AAA75490.1):

| | |
|---|---|
| 1 | MQPILLLLAF LLLPRADAGE IIGGHEAKPH SRPYMAYLMI WDQKSLKRCG GFLIQDDFVL |
| 61 | TAAHCWGSSI NVTLGAHNIK EQEPTQQFIP VKRAIPHPAY NPKNFSNDIM LLQLERKAKR |
| 121 | TRAVQPLRLP SNKAQVKPGQ TCSVAGWGQT APLGKHSHTL QEVKMTVQED RKCESDLRHY |
| 181 | YDSTIELCVG DPEIKKTSFK GDSGGPLVCN KVAQGIVSYG RNNGMPPRAC TKVSSFVHWI |
| 241 | KKTMKRY |

By “Granzyme K (GZMB) polynucleotide” is meant a nucleic acid molecule encoding a GZMB polypeptide, as well as the introns, exons, and regulatory sequences associated with its expression, or fragments thereof. In embodiments, a GZMB polynucleotide is the genomic sequence, mRNA, or gene associated with and/or required for GZMB expression. An exemplary GZMB nucleotide sequence from Homo Sapiens is provided below (GenBank: M28879.1):

| | |
|---|---|
| 1 | gaattctata ttttgagata taccattcct catagaaaaa tttcctcaca gaaaatataa |
| 61 | aggtggaaac aaatcacaag aatcgaacca tgtagagaga cttagttgtc ttttaacaga |
| 121 | attgggcacg ggctgttcag aaacaacaat ctttcacatc cattataatg atagcattag |
| 181 | tgtagtttgt ttagcaaatg tttactgcga gcctgttatg tgctgagcct gctatgtaag |
| 241 | aagtgtggct ctctggacag gagacagaat actaaacaac acaactactg atctttggct |
| 301 | gcctggcatg cttcctcact tcatatggta tcagcaattt agcaccacaa acgtccttta |
| 361 | gagaccagcc ctttctcatt cttggttcta gtggcttgag tagactgacc ccactaccca |
| 421 | agtggatttg actcctagca attcattaat ctagcccata aatgtcaagt acaggacttt |
| 481 | attgaagcat tcagaaagag gaatagggga tgttagaatc tctagaaagg aagctatgat |
| 541 | aataaatggg ttgctagatg ggtctagtag atggtggcca tgctttgtta ctgccttgtg |
| 601 | tattgtgcta ccatagccct ccccaaactg tactctggct cctggcattt ccgtctcttc |
| 661 | aaccagatgg tcagctctct aagtgaagga gacacatctc caacatgctt ggttctagca |
| 721 | caacagaagg gctcaaacac atacctgcta aagaaactat cctgatggat ttagcagcat |
| 781 | ggccatgagg cattggcggt tctatcactg ggaactcagg tttctggtgc tccagtacct |
| 841 | ctactggctg ataccacatc ctacagttca cttcataggc ttgggttcct gctctgggct |
| 901 | gaataggtgg tccactctga gtcatcagct gtggtgatga tgtggtcact gcatgattct |
| 961 | cacacaagca cccagaggac gtcatcaggc agaggcagtg ggggtgggca gcatttacag |
| 1021 | aaaatctgtg atgagacacc acaaaaccag aggggaacat gaagtcactg agcctgctcc |
| 1081 | acctctttcc tctcccaaga gctaaaagag agcaaggagg aaacaacagc agctccaacc |
| 1141 | agggcagcct tcctgagaag atgcaaccaa tcctgcttct gctggccttc ctcctgctgc |
| 1201 | ccagggcaga tgcaggtgag tgaccgtctt ccaacctcgg ggcccaaccc atcccacagg |
| 1261 | tctcctgccc tttctccaca ttcctgatcc atctatctac caggaatgtt ctgaactcca |
| 1321 | gctcccattc taccaagacc ccccaagtgt gatgctggat aagctatcag caggaatggc |
| 1381 | agagcagcag gccattctca agaagagcca gtgggtacta tcccttcccc agagcccacc |
| 1441 | tttgtcacct ggagagtagg actttcctag aagtaaatgg cagaggatgg gaaactagaa |
| 1501 | aagagaaata ttaaattatt ctagagtagg cctggcttct gtttctggga taagacaggt |
| 1561 | gcttctctca ctgtacttag gagagaaacc cagagctcag ctgacagcag aattggtaca |
| 1621 | atcactgtcc tcagaacact gttaatgtgt ttgctcagtc ccattctcca actctgcttt |
| 1681 | tcttccctgg cctttggtgg ctcccctctt tccaaggatg aggcactacg gcaggcccca |
| 1741 | gcttccctgc tttctagaat tccaccagca ctgctctacc agccctcatc cagaggctaa |
| 1801 | ctggagccag tccatcatgc agccatgaac atttactggg cacccactac atgtcaggct |
| 1861 | ctaggaaaca ggatatgaca gtatctagat ccctccactt acaccctggc cattagaaag |
| 1921 | cagcactatc ctagacacca caggactcat aagggtcttg gaaactcacc tgaaacaaag |
| 1981 | caaagtcagg agaggaatga tcaggagcct ctgggatttc actgtcccta agacaggtat |
| 2041 | gctcgccttc aactacatat ggaagaaaga tttacagacc aaagtctgct gttcttccct |
| 2101 | ttttcagagc aggaaattga agccccttcc tccaggccac tcccaactcc aggctatccc |
| 2161 | aggctcccaa atgcccagga gttctggagc cactaagcag gtgcccaccc agcagattcc |
| 2221 | atgggtgccc acaagcagac agacttttcc ttcaggggag atcatcgggg gacatgaggc |
| 2281 | caagccccac tcccgcccct acatggctta tcttatgatc tgggatcaga agtctctgaa |
| 2341 | gaggtgcggt ggcttcctga tacaagacga cttcgtgctg acagctgctc actgttgggg |
| 2401 | aaggtgagga gcagaaaaca gcccacaccc tcctggaaac actccacaga gacccctgcc |
| 2461 | ttcttcccaa ggagctccct gggctcctgt gaacacacat gccaggaggt ctccttagag |
| 2521 | ggtgagaaaa gggcagttaa gtttgtggag agaggggaag gttggttcca gaggtgctgc |
| 2581 | tgaagtaaga aacagcagag tgaccaagcc tgccatattt agaactgggg gcatactttg |
| 2641 | gcatagaata caaactgaag caattccacc tgtgtttcta gggggaaccg aaccctgaga |
| 2701 | aacctggtgc aattaccaga attccaattc ctggggaccg actgtcctta atttcccctc |
| 2761 | agctgcagcc ctgccccagc tgtcacctgc tcttcactgt ctctgggctg tatacactgt |
| 2821 | gactccaccc ccatcctcac tctgctctct gtgcagctcc ataaatgtca ccttgggggc |
| 2881 | ccacaatatc aaggaacagg agccgaccca gcagtttatc cctgtgaaaa gagccatccc |
| 2941 | ccatccagcc tataatccta agaacttctc caatgacatc atgctactgc aggtgaggca |
| 3001 | cactcctgcc actcttgctc ttcttggtcc agttggttcc actccccctg gaatgccggc |
| 3061 | ccttccctcc tttccatcct ggcctcttgg ttagttccta tgcctcagag gagagaggga |
| 3121 | agattgtgca gccccatcac tgtgtcgggg cccagaagtt cgttggctga cctggacttt |
| 3181 | cttgcctctt ccccaccagc tggagagaaa ggccaagcgg accagagctg tgcagcccct |
| 3241 | caggctacct agcaacaagg cccaggtgaa gccagggcag acatgcagtg tggccggctg |
| 3301 | ggggcagacg gcccccctgg gaaaacactc acacacacta caagaggtga agatgacagt |
| 3361 | gcaggaagat cgaaagtgcg aatctgactt acgccattat tacgacagta ccattgagtt |
| 3421 | gtgcgtgggg gacccagaga ttaaaaagac ttcctttaag gtaagactat gcacctgcct |
| 3481 | ggattggctc ttgggagaaa gatgtttggg gaatatctga gacctggaga ctcaagtagt |
| 3541 | gggggactcc ttcacccact agactgtgat atttctctct ggaaagagaa aaggggacta |
| 3601 | gactgagctg gggagaaatt agggcctctg caaacttacc aagaggcctt atggtggatg |
| 3661 | gtgccttctt tggaaggatg aatttgcaac actccaccca ctccaggtca cagatattag |
| 3721 | gaaactgtgc ccatgggggt gcagctaatt ataaccaggt gtgtcttcag aggctggtac |
| 3781 | ccaacgtggt taatgggctg gtcctccatg gtggacatca gccctccttg cccacttctg |
| 3841 | ggtccttaaa cagccaacgg tcccacatac ctccgatctc aggatctggg ggacatgacg |
| 3901 | gaggctggcc cctgggatga ggtgaagcag taacaatgtc cagggccaga gcttggcagc |
| 3961 | tggggccacc agcggcctgc cctgccctct ggtctcccac atgtaggctg tgcaagttgg |
| 4021 | ccttttctaa aagggggctt gagatggaag agagggcagg acccggagga gcatcagctc |
| 4081 | agtccttcca ctctctattc acagggggac tctggaggcc ctcttgtgtg taacaaggtg |
| 4141 | gcccagggca ttgtctccta tggacgaaac aatggcatgc ctccacgagc ctgcaccaaa |
| 4201 | gtctcaagct ttgtacactg gataaagaaa accatgaaac gctactaact acaggaagca |
| 4261 | aactaagccc ccgctgtaat gaaacacctt ctctggagcc aagtccagat ttacactggg |
| 4321 | agaggtgcca gcaactgaat aaatacctct tagctgagtg gaaagctggt ttcttgttta |
| 4381 | ttcattgacc ctcattctca ggcaccacat ctgcgctatg caggccaatg acacaatttt |
| 4441 | gctgttttct gctttctcct ctcccctcac cccttgccac ctccccaaac ccccacatga |
| 4501 | agctgatact cagctccttc ctatccacac cagtttctcc agggcctgcc ttctgccaag |
| 4561 | gctgaagctg agcaccatca ggagacaaca tggaccactt tggtcctggg gctttgggta |
| 4621 | aacttcttac ctccttctcc agtgttacat tgacagagaa aaaagggata ataccatggg |
| 4681 | acctaactcc tcatcccact ggggctcctc attctcccct gggcttagtt tctctaccct |
| 4741 | cctctgagct c |

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “administer” is meant the direct application of an agent to a subject. In embodiments, a medication is administered by ingestion, inhalation, infusion, injection, or any other means, whether self-administered or administered by a clinician or other medical professional.

By “agent” is meant any small molecule chemical compound, antibody, nucleic acid molecule, or polypeptide, or fragments thereof. Exemplary agents useful in the methods disclosed herein include, but are not limited to, immunotherapeutic agents and immunomodulatory agents. Other agents include anti-SLAMF7 antibody, Elotuzumab, Lenalidomide, Dexamethasone.

By “alteration” is meant a change in the structure, expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels. In embodiments, alterations in expression, level, or abundance are detected in any of the markers delineated herein (e.g., Tables 1-6, the Examples, and the claims).

By “alternate therapy” is meant a therapeutic regimen that differs in one or more aspects from the combination therapy provided in the clinical trial described in the Examples herein below. In another embodiment, an alternate therapy comprises an increase in the duration or dosage of Lenalidomide, and Dexamethasone, Elotuzumab or another immunotherapeutic agent. In some embodiments, the alternate therapy comprises or consists of a triplet therapy (i.e., a combination of a proteasome inhibitor, an immunomodulatory drug, and a steroid, e.g., dexamethasone). In some instances, the alternate therapy comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the alternate treatment comprises or consists of autologous stem cell transplantation (ASCT). In some instances, the alternate treatment comprises or consists of CAR-T cells targeting BCMA (e.g., Abecma® [idecabtagene vicleucel]). In some instances, the alternate treatment comprises or consists of a bispecific antibody targeting BCMA (e.g., an anti-BCMA/anti-CD3 bispecific antibody, e.g., Teclistamab.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. In embodiments, the disclosure provides for the use of Elotuzumab, Lenalidomide, and Dexamethasone.

By “capture molecule” is meant an agent that specifically binds to an analyte. Capture molecules are used in, for example, ligand binding assays, immune assays, and protein or nucleic acid molecule capture assays. In one embodiment, a biological sample is characterized using a capture array, in which reagents, which are usually antibodies, but can also be alternative protein scaffolds, peptides or nucleic acid aptamers, are used to detect target molecules in mixtures, such as biological samples (e.g., blood, plasma or tissue extracts). In diagnostics, capture arrays are used to carry out multiple immunoassays in parallel, both testing for several analytes in individual blood/sera for example and testing many blood/serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, optionally in a multiplex array format against multiple protein targets.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected. In embodiments, the present disclosure provides for the detection of markers (e.g., those listed in Tables 1-6, the Examples, or the claims) useful in characterizing SMM.

By “disease” is meant any condition or disorder that damages or interferes with the normal function of a cell, tissue, or organ. In some instances, the disease is a hemotological malignancy or a precursor condition. Examples of diseases include plasma cell dyscrasias (e.g., a monoclonal gammopathy), such as monoclonal gammopathy of undermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. Further non-limiting examples of diseases include any B cell or plasma cell neoplasm.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In embodiments, an effective amount is sufficient to inhibit progression of SMM to MM, to increase survival, to modulate (i.e., increase, decrease) an immune response, or to otherwise improve, treat, or stabilize the subject's condition.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “immune dysregulation” is meant a disruption in the normal functioning of the immune system. Such disruption may be characterized using any one or more of the methods described herein (e.g., by measuring the level of markers of Tables 1B-6 in a biological sample of a subject, measuring the proportion of immune cells in a biological sample of a subject, measuring the immune cell repertoire present in in a biological sample of a subject.

By “immune cell repertoire” is meant the diversity of immune cells present in a subject. In some embodiments, a subject may show about 45, 50, 55, 60, or 65 different immune cell types (e.g., CD4+ T-cells, stem cells, pDCs, proinflammatory myeloid cells, or any of the cell types characterized in FIG. 4. In an embodiment, T-cell repertoire diversity is measured using TCR sequencing, Chao index, Shannon entropy, Pielou's evenness, Simpson index, Gini-Simpson index, Gini index, Hill numbers, Renyi entropy, richness (i.e., the total number of unique clonotypes).

By “immune reactive” is meant characterized by an activated immune response. In an embodiment, an immune reactive patient shows an increase in more potent, long-lived GZMK⁺ CD8⁺ TEM cells. In an embodiment, an immune reactive subject shows lower proportions of pro-inflammatory myeloid cells and pDCs. In another embodiment, an immune reactive subject displays markers listed in Table 1B.

By “increase” is meant to alter positively by at least 5% relative to a reference. An increase may be by 5%, 10%, 25%, 30%, 50%, 75%, or even by 100%. In some embodiments, the increase is an increase in progression free survival relative to the progression free survival of untreated subject having SMM. In other embodiments, an increase is in an increase in the level, expression, or activity of a marker described herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “negative” the context of a polypeptide or polynucleotide marker is meant undetectable or virtually undetectable by conventional means known in the art and/or described herein. In embodiments, a sample is “negative” for a marker when the level of marker in the sample falls below a defined level.

By “positive” in the context of a polypeptide or polynucleotide marker is meant detectable by conventional means known in the art and/or described herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “marker” is meant any analyte, including protein or polynucleotides having an alteration in expression, level, or activity that is associated with a disease, disorder, or state. Markers useful for characterizing SMM include, but are not limited to, those included in Tables 1B-6, the marker profiles defined by Gex-1 through Gex-26, those polypeptide and polynucleotide markers listed in the Examples, as well as those included in the claims. In embodiments, exemplary markers include, but are not limited to the following polypeptides and polynucleotides encoding them: ABCB1, ACSM3, ACTB, ACTG1, ADA, ADAM33, AHNAK, Amplq, ANXA1, AVP, Axl, AZU1, APOC1, APOE, BCL2, BEX1, BEX2, BLVRB, B3GAT1, CA1, CA2, CCL5, CCL3, CCL4, CD1C, CD2, CD3D, CD4, CD5, CD6, CD7, CD8, CD8A, CD8B, CD74, CD83, complement factor Clq (CIQA, CIQB, CIQC, SELENOP, SDC3), CCR4, CD14, CD16, CD19, CD38, CD27, CD28, CD33, CD34, CD40, CD69, CD79A, CD79B, CD86, CD160, CD164, CDKN1C, CCR6, CCR7, CEBPD, CFL, CLIC3, CSF2RB, CLEC10A, CLEC9A, C1orf54, IDO1, CADM1, COL1A1, COLIA2, CCL2, CCL3, CRHBP, CLMK, CDIC, CLC, CMC1, CSFIR, CTLA4, CYBB, CXCR5, CXCL8 (encoding IL-8), CXCL2, CXCL12, LEPR, KITLG, VCAM1, CX3CR1, CXCR3, CXCR4, Dellp, DNTT, DUSP1, ENC1, ELANE, EPCAM, EPOR, FAS, FCERIA, FCER2, FCGR3A, FCN1, FGFBP2, FCRL5, FLT3, FOS, FOSB, FOXP3, GATA1, GATA2, GLUL, GNLY, GPIBB, GZMA, GZMB, GZMH, GZMK, GATA3, GP9, HDC, HERC5, HIFIA, HISTIHIC, HISTIHID, HISTIHIE, HLA-DRA, HLA-DRB1, HLF, HNRNPLL, IGHM, IGHD, IGLL1, IGLLIIL2RA, IL7R, IFI6, IFI44L, IFNG, interferon type I (IFN-I), IFIT1, IFIT2, IFIT3, IKZF2, IKZF3 (which encodes Aiolos), IL4R, ILIB, IL3RA, ISG15, IFI6, IFI44L, IFI6, IFI44L, IL7R, IRF4, IRF8, ISG15, ITAG2B, ITGAX, ITGB1, ITM2C, IRF4, IRF8, JUN, JUNB, KLRB1, TSC22D3, IGLL1, JCHAIN, KLRC1, KLF1, KLRG1, KRAS, KRTI, LAG3, LEF1, LILRA4, LILRB4, LINC0185LY9, LTK, LTB, LYZ, LY86, LYAR, MEF2C, MME, MPO, MPP6, MS4A1, MS4A2, MS4A6A, MS4A3, MS4A7, MS4A1, MX1, MZB, MZB1, NCAMI, NEAT1, NFKB1, NFKBIA, NFKB2, NKG, NKG7, NOSIP, NR4A2, NR4A3, OASL, PAX5, PD-1, PDCD1, PLD4, PRG2, PLEK, PPBP, PFN1, PF4, PRF1, PRTN3, PRDM1, PTGDS, PMAIP1, RNASE2, RAG1, RAG2, RHOC, RORA, RTKN2, RUNX2, SLC43A2, SLC25A37, SIGLEC6, S100A8, S100A9, S100A12, S100A10, SELL, SIP receptors, SLC4A10, SPON2, SPRY2, SOX4, SOX5, TCLIA, STAT1, SERPINA1, SERPINF1, TBX21, TCF7 (encoding Tcf-1), Th17 and Th1 TIGIT, TIMP1, TLR2, TMSB4X, TMSB10, TRAC, TRBCITNF, TSC22D3, TOX, TOX2, TPSAB1, TPSB2, TNFRSF4, TNFRSFIB, TNFRSF9, TP53 SNV, TRBC2, TRGV9, TRDV2, TCLIA, TXNIP, VCAN, VPREB1, XAF1, XCL1, XCL2, and ZC3HAV1. In other embodiments, a marker comprises the abundance of cells in a biological sample of a subject, e.g., T-cells (including Tregs, Th1, Th2, and Th17 cells), granzyme B (GZMB)-expressing CD8⁺ T-cells, and CD56_{dim} NK cells, CD14⁺ Monocytes, Plasmacytoid Dendritic Cells (pDCs), AXL⁺ SIGLEC6⁺ dendritic cells (AS-DCs), progenitor cells, and immature B-cells, mucosa-associated invariant T-cells (MAITs), memory B-cells, cDC2, and pDC subpopulations, and terminal effector cells. In other embodiments, a marker is T-cell receptor (TCR) repertoire diversity and/or T cell receptor clonality.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference is a healthy subject or a subject having untreated SMM. In other embodiments, a “reference” is a numeric value based on an a statistical calculation, for example, a numeric value based on the average value of an analyte present in a population of normal patients or patients having a disease, such as a hematologic malignancy or precursor condition (e.g., SMM, MGUS, MM), or the distribution of a marker in a population of subjects with a hematologic malignancy or precursor condition (e.g., SMM, MGUS, MM).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. A reference sequence can be the genome of a healthy cell or a portion thereof. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “remission” is meant a subject having substantially no signs or symptoms of disease (e.g., smoldering multiple myeloma, monoclonal gammopathy of undetermined significance, or multiple myeloma). In embodiments, a multiple myeloma subject in remission shows little-to-no signs or symptoms of multiple myeloma and/or shows signs or symptoms of multiple myeloma similar to those observed in a healthy subject and/or a subject having a non-active multiple myeloma (e.g., MGUS or SMM).

By “subject” is meant an animal. The animal can be a mammal. The mammal can be a human or non-human mammal, such as a bovine, equine, canine, ovine, rodent, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DESCRIPTION OF THE DRAWINGS

FIGS. 1A-1F show a genomic dissection of response to early treatment with EloLenDex (anti-SLAMF7 antibody, Elotuzumab, Lenalidomide, Dexamethasone). FIG. 1A is a Kaplan-Meier curve of Progression-Free Survival (PFS) in the E-PRISM cohort. Only clinical progression events (CRAB) were considered for this analysis. FIG. 1B is a scatter plot of Cancer Cell Fractions (CCF) at baseline (x axis) and end of treatment (y axis) for progressor patient #21 (in grey: mutational drivers and copy number variations (CNV) associated with risk of progression). FIG. 1C is a scatter plot of cancer cell fractions at baseline (x axis) and end of treatment (y axis) for progressor patient #51 (in grey: mutational drivers and copy number variations associated with risk of progression). FIG. 1D is the genomic landscape of the E-PRISM cohort at baseline. FIG. 1E is a Univariate Cox regression forest plot of genomic variables present in at least 3 individuals. FIG. 1F is a Kaplan-Meier curve of PFS in the E-PRISM cohort, stratified based on the presence of Del17p.

FIGS. 2A-2L show bone marrow and peripheral blood immune cell populations in the E-PRISM cohort. FIG. 2A is a Uniform Manifold Approximation and Projection plot (UMAP) showing embedding of T-cells. FIG. 2B is a heatmap of gene expression markers (Mean Z-score of normalized expression) in T-cells. FIG. 2C is an UMAP showing embedding of NK cells. FIG. 2D is a heatmap of gene expression markers (Mean Z-score of normalized expression) in NK cells. FIG. 2E is a UMAP showing embedding of B-cells. FIG. 2F is a heatmap of gene expression markers (Mean Z-score of normalized expression) in B-cells. FIG. 2G is an UMAP showing embedding of Monocytes. FIG. 2H is a heatmap of gene expression markers (Mean Z-score of normalized expression) in Monocytes. FIG. 21 is an UMAP showing embedding of Dendritic cells. FIG. 2J is a heatmap of gene expression markers (Mean Z-score of normalized expression) in Dendritic cells. FIG. 2K is an UMAP showing embedding of progenitor cells. FIG. 2L is a heatmap of gene expression markers (Mean Z-score of normalized expression) in progenitor cells.

FIGS. 3A-3F show a comprehensive profiling of changes in BM immune cell composition and T-cell receptor repertoire in patients with high-risk SMM. FIG. 3A is a volcano plot of proportion changes in the bone marrow of patients with HRSMM (n=35) compared to healthy donors (NBM, n=22). P-values were computed with Wilcoxon's rank-sum test and corrected using the Benjamini-Hochberg approach. Cell types with a q-value of <0.2 were marked with stars. FIG. 3B is a plot comparing bone marrow T-cell receptor (TCR) repertoire diversity, as assessed by the Chao index, between patients with HRSMM (n=14) and healthy individuals (NBM, n=11) given different numbers of downsampled cells. Each data point represents the average diversity estimate across 100 random samples of the given size for a single sample. The range of diversity estimates across all iterations for each sample is visualized in error bars (dotted line). P-values were computed with Wilcoxon's rank-sum tests. FIG. 3C is a bar plot showing the proportion of T-cell clonotypes (y axis) in a given baseline patient sample (P) or sample from a healthy donor (HD) that were determined to belong to one of four clone size categories (Rare: ≤1%; Small: >1% and <5%; Medium: ≥5% and <10%; Large: ≥10%) through iterative (n=100) downsampling of 100 cells. The average proportion per clone size category was visualized and the standard deviation of that proportion across iterations was depicted in solid-line error bars. FIG. 3D provides two UMAPs showing embedding of healthy donor Normal Bone Marrow (NBM) and patient bone marrow (BM) T-cells at baseline with matched TCR data. T-cells that belonged to rare clonotypes (with a frequency of ≤1%) are shown in light grey, while T-cells that belonged to expanded clonotypes (with a frequency of >1%) are shown in darker shading. FIG. 3E is a barplot showing the proportion (y axis) of clonotypes in a given T-cell subtype across all patients (Patient BM) or healthy donors (NBM) that belonged to one of the four clone size categories. For each T-cell subtype, 100 cells were randomly sampled 100 times from all patients or healthy donors, and the proportion of expanded (1-Rare) clonotypes was compared between patients and healthy donors using Wilcoxon's rank-sum tests. FIG. 3F is a volcano plot (x axis: log₂ fold-change of normalized expression, y axis: −log₁₀ (q-value)) highlighting genes that are highly expressed in expanded GZMB™ CD8⁺ TEM cells (n=3,338, X>0) compared to rare GZMB⁺ CD8⁺ TEM cells (n=2,216, X<0).

FIGS. 4A-4K show that immune reactivity at baseline and post-therapy immune normalization are associated with significantly longer Progression-Free Survival in patients with high-risk SMM under treatment. FIG. 4A is a barplot showing the importance of each cell type towards the classification, as computed based on a Naïve Bayes classifier. FIG. 4B is a Kaplan-Meier curve of progression-free survival in the E-PRISM cohort, stratified based on the median normalization score (Reactivity⁺: least normal-like; Reactivity−: most normal-like). FIG. 4C is a barplot showing the distribution of different risk stages based on the 20-2-20 criteria according to reactivity status. FIG. 4D is a barplot showing the frequency of Del17p in patients stratified based on their reactivity status. P-value was computed with a Fisher's exact test. FIG. 4E is a boxplot comparing the abundance of Cytokine⁺ CD14⁺-Monocytes and pDCs between patients classified as reactive or not. P-values were computed with Wilcoxon's rank-sum test. FIG. 4F is a volcano plot showing genes that are differentially expressed between GZMK⁺ CD8⁺ TEM cells of reactive patients compared to non-reactive. FIG. 4G is a volcano plot showing genes that are differentially expressed between GZMB⁺ CD8⁺ TEM cells of reactive patients compared to non-reactive. FIG. 4H is a boxplot showing the distribution of normalization scores in patient bone marrow samples drawn at baseline (BL), cycle 9 day 1 (C9D1), or end of treatment (EOT), and in healthy donor bone marrow samples (NBM). P-values were computed using a paired t-test for paired patient samples drawn at baseline and EOT or Wilcoxon's rank-sum test for comparisons between patient samples and healthy donors. FIG. 4I is a histogram of the distribution of change in normalization scores between baseline bone marrow samples and end of treatment (EOT) bone marrow samples from the same patients. The dashed line corresponds to the threshold used to determine the presence of post-therapy immune normalization (PIN). FIG. 4J is a boxplot showing paired normalization scores at baseline (BL) and end of treatment (EOT) samples from patients with HRSMM. Solid lines connect samples from patients classified as positive for post-therapy immune normalization (PIN⁺); dashed lined connect samples from patients classified as PIN-negative (PIN−). FIG. 4K is a Kaplan-Meier curve of progression-free survival in the E-PRISM cohort, stratified based on post-therapy immune normalization (PIN) status.

FIGS. 5A-5K show Granzyme-K (GZMK)-positive CD8⁺ TEM cells, Memory B-cells, pDCs and pro-inflammatory monocytes may be associated with response to therapy in patients with high-risk SMM. FIG. 5A is a boxplot visualizing the relative abundance of GZMK⁺ CD8⁺ TEM and GZMB⁺ CD8⁺ TEM out of all cytotoxic T-cells (i.e., their sum) in patient bone marrow compared to samples from healthy donors (NBM). P-values were computed with Wilcoxon's rank-sum test. FIG. 5B is a boxplot showing the abundance of GZMK CD8⁺ TEM cells, as measured by CyTOF, in patient bone marrow samples drawn at baseline (BL) and end of treatment (EOT). FIG. 5C is a barplot showing the proportion of clonotypes belonging to each of four clone size categories per cytotoxic T-cell subtype in patient peripheral blood samples drawn at baseline (BL) or end of treatment (EOT). P-values were computed with Wilcoxon rank-sum tests, as cells were sampled across individuals. FIG. 5D is a volcano plot showing genes that are differentially expressed between GZMK⁺ CD8⁺ TEM and GZMB⁺ CD8⁺ TEM cells from patient bone marrow samples drawn at baseline. FIG. 5E is a boxplot comparing the abundance of GZMK⁺ CD8⁺ TEM cells between paired bone marrow (BM) and peripheral blood (PB) samples drawn at baseline from patients with high-risk SMM. Solid lines connect samples that are enriched in the BM, while dashed lines connect samples that are enriched in the PB. The p-value was computed using a paired t-test. FIG. 5F is a scatter plot showing the positive correlation between the proportion of CD8⁺ T-cells expressing PD-1 by CyTOF and the proportion of CD8⁺ T-cells expressing GZMK by CyTOF. A regression line was fitted (solid) and the correlation coefficient and p-value were computed using Pearson's approach. FIG. 5G is a Kaplan-Meier curve of progression-free survival in the E-PRISM cohort, stratified based on the median abundance of GZMK⁺ CD8⁺ TEM cells in patient bone marrow samples at baseline. FIG. 5H is a boxplot showing the proportion of naïve B-cells (NBC), resting memory B-cells (BRM), marginal zone B-cells (MZB), and effector memory B-cells (BEM) in patients with immunoparesis (IP), patients without IP (No IP), and healthy donors (NBM). FIG. 5I is a heatmap of mean z-scored gene expression (GEX) signature activity in cells assigned to those signatures through non-negative matrix factorization. FIG. 5J is a UMAP showing embedding of lymphocytes and antigen-presenting cells, greyed by the log-scaled activity of signature GEX-6. FIG. 5K is a forest plot showing the effect of mean baseline GEX-6 and GEX-13 signature activity in the bone marrow on progression-free survival. The hazard ratio, 95% confidence interval, and p-value were computed using Cox proportional hazards regression.

FIGS. 6A-6G show peripheral blood-based immune profiling accurately detects alterations in immune cell composition and T-cell receptor repertoire diversity in the bone marrow. FIG. 6A is a boxplot showing the Jensen-Shannon divergence between matched bone marrow and peripheral blood samples, compared to unmatched bone marrow and peripheral blood samples. The p-value was computed using Wilcoxon's rank-sum test. FIG. 6B is a heatmap of Pearson's correlation coefficient (r) between bone marrow immune cell abundance and the first 10 principal components. The x axis was sorted in decreasing order of PC1. The top panel shows the Log₂ fold-change in abundance between bone marrow samples from patients with HRSMM (n=35) and those from healthy donors (n=22). Stars correspond to pairs with significant (<0.05) correlation. FIG. 6C is a two-dimensional density plot of bone marrow (BM) and peripheral blood (PB) samples from patients (BM: n=26, PB: n=29) or healthy donors (BM: n=22, PB: 10) according to PC1 (x axis) and PC2 (y axis). FIG. 6D is a volcano plot of proportion changes in the peripheral blood of patients with HRSMM (n=29) compared to healthy donors (NPB, n=10). P-values were computed with Wilcoxon's rank-sum test and corrected using the Benjamini-Hochberg approach. Cell types with a q-value of <0.2 were marked with stars. FIG. 6E is a series of boxplots comparing peripheral blood T-cell receptor (TCR) repertoire diversity, as assessed by the Chao index, between patients with HRSMM (n=22) and healthy individuals (NPB, n=10) given different numbers of downsampled cells. Each data point represents the average diversity estimate across 100 random samples of the given size for a single sample. The range of diversity estimates across all iterations for each sample is visualized in error bars (dotted line). P-values were computed with Wilcoxon's rank-sum tests. FIG. 6F is a forest plot showing the effect of mean baseline GEX-13 signature activity in the peripheral blood on progression-free survival. The hazard ratio, 95% confidence interval, and p-value were computed using Cox proportional hazards regression. FIG. 6G is a confusion matrix showing the accuracy of a Naïve Bayes classifier trained to identify the presence of SMM based on bone marrow samples from patients and healthy donors in detecting the presence of SMM in peripheral blood samples.

FIG. 7A is an E-PRISM trial schema.

FIG. 7B is a CONSORT diagram of the E-PRISM study.

FIG. 8A is a treatment-related Grade 2 adverse events with at least 10% frequency and all Grade 3-5 events.

FIG. 8B is a series of Kaplan-Meier curves for Overall Survival (OS) and Progression-Free Survival (PFS) in the E-PRISM cohort and by arm.

FIG. 9A is a Kaplan-Meier curve of Progression-Free Survival (PFS) in the E-PRISM cohort, stratified based on the “20-2-20” criteria.

FIG. 9B is a Kaplan-Meier curve of Progression-Free Survival (PFS) in the Lenalidomide arm of the ECOG cohort, stratified based on the “20-2-20” criteria.

FIGS. 10A-10B are boxplots comparing the inter-replicate Jensen-Shannon divergence of immune cell composition, compared to cross-replicate estimates for (FIG. 10A) technical replicates (i.e., two cell vials from the same sample were thawed and libraries were prepared using the same technology) (n=5), and (FIG. 10B) technology replicates (i.e., two cell vials from the same sample were thawed and libraries were prepared using different technology for each replicate: 3′-end or 5′-end library preparation) (n=4).

FIG. 11 is a scatterplot of immune cell composition principal components 1 (PC1, x axis) and 2 (PC2, y axis), demonstrating the absence of an observable batch effect due to the samples' tissue of origin or library preparation technology (n=176).

FIG. 12 is a series of scatterplots of cell type proportions as measured by single-cell RNA-sequencing (y axis) or CyTOF (x axis) performed on CD138-negative bone marrow immune cells (n=17). Dashed black lines correspond to the y=x diagonal. Correlation coefficients and p-values were computed with Pearson's approach.

FIGS. 13A-13C are boxplots of (FIG. 13A) T-cells, (FIG. 13B) Monocyte, and (FIG. 13C) B-cell proportions in healthy individuals (NBM, n=22), patients with HRSMM in the E-PRISM cohort (n=26), and non-trial patients with HRSMM (n=9). Cell types with adjusted p-values<0.1 have been annotated with brackets and their corresponding adjusted p-value.

FIG. 14 is a confusion matrix visualizing the performance of the disease classifier on a testing set of BM samples from patients and healthy donors (n=16).

FIG. 15A is a boxplot comparing mean GZMK expression levels in T-cells of untreated patients (BL) compared to patients at end of treatment (EOT).

FIG. 15B is a scatterplot of the proportion of CD8⁺ T-cells that were positive for PD-1 (x axis), and the proportion of CD8⁺ T-cells and were positive for both PD-1 and GZMK (y axis) by CyTOF.

FIG. 15C is a Kaplan-Meier curve of progression-free survival in the E-PRISM cohort, stratified based on the mean expression of GZMK across all T-cells.

FIG. 16A is a Kaplan-Meier curve of biochemical progression-free survival in the E-PRISM cohort, stratified based on the presence or absence of immunoparesis (abnormally low levels of serum immunoglobulin affecting at least one uninvolved isotype).

FIG. 16B is a Kaplan-Meier curve of biochemical progression-free survival in the E-PRISM cohort, stratified based on the median abundance of marginal zone B-cells (MZB).

FIG. 16C is a Kaplan-Meier curve of biochemical progression-free survival in the E-PRISM cohort, stratified based on the median abundance of effector memory B-cells (BEM).

FIG. 16D is a boxplot comparing B-cell proportions, as assessed by CyTOF, between HRSMM patients with or without immunoparesis (IP).

FIG. 16E is a volcano plot showing genes that are differentially expressed between memory B-cells in patients with HRSMM and those in healthy individuals (NBM).

FIG. 17 is a scatterplot of the number of signatures extracted (K, x axis) and the corresponding value of the objective function (y axis) for each of our NMF runs (n=30). On the top axis is a histogram of the frequency of runs supporting a given K (mode=26). On the right axis is a density plot of the objective function values across all runs in medium grey. The selected run, which has the lowest objective among runs with K equal to the distribution's mode (i.e., K=26), is highlighted in medium grey.

FIG. 18A is a boxplot showing the activity of gene expression signature GEX-6 across lymphocytes and antigen-presenting cells.

FIG. 18B is a boxplot comparing the abundance of Cytokine⁺ CD14⁺ Monocytes between matched BM and PB patient samples drawn at baseline.

FIG. 18C is a boxplot comparing the activity of gene expression signatures GEX-13 and GEX-6 between patients with HRSMM and healthy donors. Each dot corresponds to the mean signature activity for the particular individual.

FIG. 19 is a boxplot showing the abundance of CD16⁺ monocytes in patient bone marrow (BM), patient peripheral blood (PB), healthy donor bone marrow (NBM), and healthy donor peripheral blood (NPB). P-values were computed using a paired t-test for matched patient BM and PB samples, and Wilcoxon's rank-sum test for unmatched healthy donor BM and PB samples.

FIG. 20 is a Kaplan-Meier curve of patients with newly diagnosed Multiple Myeloma stratified based on the average TCR repertoire diversity, as assessed through the Shannon index.

### DETAILED DESCRIPTION

The disclosure features compositions and methods for diagnosing, characterizing, treating or selecting for treatment a subject having smoldering multiple myeloma (SMM), monoclonal gammopathy of undetermined significance (MGUS), or another condition that has propensity to develop into multiple myeloma, wherein the methods of treatment are selected based on characterizing a biological sample of the subject.

The invention is based, at least in part, on several discoveries. First, that the similarity of a patient's immune microenvironment to that of healthy donors may have prognostic relevance at diagnosis and post-treatment; second, that GZMK+CD8+ effector memory T-cells may be associated with response to treatment; and third, that striking similarities exist between immune alterations observed in the bone marrow (BM) and peripheral blood (PB), suggesting that peripheral blood-based immune profiling may have diagnostic and prognostic utility. Such discoveries are founded, at least in part, on results obtained in a clinical trial of a combination therapy comprising immunotherapeutics and immunomodulatory agents (e.g., Elotuzumab, Lenalidomide, and Dexamethasone), which was used to treat subjects having smoldering multiple myeloma. In embodiments, the disclosure provides methods for selecting subjects having or having a propensity to develop SMM for treatment with immunotherapeutics and immunomodulatory agents (e.g., Elotuzumab, Lenalidomide, and Dexamethasone) Elotuzumab, Lenalidomide, and Dexamethasone.

### Use of Markers to Select Subjects for Treatment

Subjects with monoclonal gammopathy of undetermined significance (MGUS) or smoldering multiple myeloma (SMM) are typically observed until progression, but early treatment may improve outcomes. The data in the Examples section herein include data from a Phase II clinical trial of Elotuzumab, Lenalidomide, and Dexamethasone in subjects with high-risk SMM showing that early immunotherapy can be safe and effective. Moreover, single-cell RNA-sequencing on bone marrow (BM) and peripheral blood (PB) samples from subjects and healthy donors can provide a comprehensive characterization of alterations in immune cell composition and T-cell receptor repertoire diversity in patients. Importantly, the similarity of a patient's immune microenvironment to that of healthy donors may have prognostic relevance at diagnosis and post-treatment, and GZMK⁺ CD8⁺ effector memory T-cells may be associated with response to treatment. Lastly, similarities between immune alterations can be observed in the BM and PB. PB-based immune profiling may have diagnostic and prognostic utility.

Here, a Phase II trial of the immunotherapeutic anti-SLAMF7 antibody, Elotuzumab, in combination with LenDex (EloLenDex, E-PRISM study) was conducted, to determine the utility and safety of early immunotherapy in patients with high-risk SMM. Moreover, correlative DNA sequencing studies were performed on 34 BM samples at baseline, and single-cell RNA-sequencing and TCR-sequencing studies on 149 serial BM and PB samples from patients and healthy donors to identify genomic and immune biomarkers for optimal patient selection and monitoring of response to treatment.

Based on these data, markers (e.g., immune markers), such as the abundance of particular cells (e.g., mature B-cells, Th17 cells and GZMK⁺ T and NK cells), are used to identify and select subjects (e.g., human) with SMM, MGUS, or MM for treatment (e.g., treatment before progression from SMM or MGUS to MM for subjects with SMM or MGUS), to identify subjects (e.g., human) with SMM, MGUS, or MM who are likely to benefit from different, e.g., more intensive, treatment regimens, subjects (e.g., human) with MM undergoing immunotherapy who are likely to progress and, thus, are likely to benefit from a different treatment regimen (e.g., a more intensive treatment regimen), and to identify subjects (e.g., human) with SMM, MGUS, or MM for whom treatment is likely to result in prolonged biochemical progression free survival, and thus, for whom treatment may be terminated or modified or follow-up may be modified (e.g. changes in the frequency of follow-up assessments or the type of tests performed clinically).

The data in the Examples section herein demonstrate that markers described herein can be used to identify and/or select subjects (e.g., humans) with SMM or MGUS that would benefit from early treatment (i.e., before progression to MM). For instance, based on the data in the Examples section herein, subjects (e.g., human) with SMM, MGUS, or MM having one or more (e.g., 1, 2, 3, 4, 5, 6, 7, 8, 9, 10, 11) (e.g., prior to treatment for SMM, MGUS, or MM) of the markers of Table 1 are predicted to have significantly longer progression-free survival upon treatment (e.g., with immunotherapy) and, thus, are predicted to benefit from treatment (e.g., with immunotherapy or early treatment for SMM or MGUS subjects, i.e., treatment before progression from SMM or MGUS to MM). Such subjects would be selected for therapy (e.g., for treatment with an immunomodulator or immunotherapeutic agent). Additionally, based on the data in the Examples section herein, subjects (e.g., human) with SMM, MGUS, or MM having one or more (e.g., 1, 2, 3, 4, 5, 6, 7, 8, 9, 10, 11) (e.g., prior to treatment for SMM, MGUS, or MM) of the markers of Table 2 are predicted to have significantly shorter progression-free survival upon treatment (e.g., with immunotherapy), and, thus, would benefit from treatment (e.g., with one or more therapeutic agents other than or in addition to immunotherapy or early treatment for SMM or MGUS subjects, i.e., treatment before progression from SMM or MGUS to MM). Furthermore, the data in the Examples section also demonstrate that patients with MM can also present with an increased immune reactivity and, thus, are also more likely to respond to treatment with, e.g., immunotherapy.

The data in the Examples section also demonstrate that markers can be used to monitor the response to treatment (e.g., immunotherapy) in a subject (e.g., human) with SMM, MGUS, or MM and determine which subjects with SMM, MGUS, or MM should be treated with a different, e.g., more intensive regimen. For instance, based on the Examples section herein, subjects (e.g., human) with SMM, MGUS, or MM having one or more (e.g., 1, 2, 3, 4, 5, 6, 7) of the biomarkers of Table 3 while undergoing treatment (e.g., with immunotherapy) for SMM, MGUS, or MM are predicted to have significantly shorter progression-free survival upon treatment and, thus, are predicted to benefit from a different, e.g., more intensive treatment before progression to MM (e.g., higher dose(s), more dose(s), combination therapy, or a different therapy from that being used). Additionally, based on the Examples section herein, subjects (e.g., human) with SMM, MGUS, or MM having one or more (e.g., 1, 2, 3, 4, 5, 6, 7) of the markers of Table 4 while undergoing treatment (e.g., with immunotherapy) for SMM, MGUS, or MM are predicted to have significantly longer progression-free survival upon treatment, and thus, are predicted to benefit from continuing the treatment. Non-limiting examples of alternate therapies include: a triplet therapy (i.e., a combination of a proteasome inhibitor, an immunomodulatory drug, and a steroid, e.g., dexamethasone), a quadruplet therapy (i.e., a combination of (i) a monoclonal antibody that specifically binds to, e.g., SLAMF7, CD38, (ii) a proteasome inhibitor, (iii) an immunomodulatory drug, and (iv) a steroid, e.g., dexamethasone), autologous stem cell transplantation (ASCT), CAR-T cells targeting B-cell maturation antigen (BCMA) (e.g., Abecma® [idecabtagene vicleucel]); and bispecific antibodies targeting BCMA (e.g., anti-BCMA/anti-CD3 bispecific antibodies (e.g., Teclistamab)).

The data in the Examples section also demonstrate that markers (e.g., immune markers) can be used to monitor response to treatment (e.g., immunotherapy) in subjects (e.g., humans) with SMM, MGUS, MM and to determine which subjects are likely to have prolonged biochemical progression-free survival and, thus, for whom treatment may be terminated or modified (e.g., changes in amount, duration, or type of treatment; changes in the frequency of follow-up assessments or the type of tests performed clinically). For instance, based on the Examples section herein, subjects (e.g., human) with SMM, MGUS, or MM having an immune biomarker from Table 5 after treatment (e.g., with immunotherapy) were predicted to have prolonged biochemical progression-free survival and, thus, to benefit from terminating or modifying (e.g., changing the amount, duration, or type of treatment; changes in the frequency of follow-up assessments or the type of tests performed clinically) the treatment. Additionally, based on the Examples section herein, subjects (e.g., human) with SMM, MGUS, or MM having an immune biomarker from Table 6 after treatment (e.g., with immunotherapy) were predicted to have shortened biochemical progression-free survival and, thus, to benefit from continued or more intensive (e.g., changing the amount, duration, or type of treatment; changes in the frequency of follow-up assessments or the type of tests performed clinically) treatment.

The data in the Examples section also demonstrate the concordance of the markers in bone marrow samples and blood samples. These data reveal that these markers (e.g., immune markers) can be evaluated using blood samples rather than invasive bone marrow samples. Thus, the markers (e.g., immune markers) identified herein may be evaluated in a blood sample or a bone marrow sample from a subject (e.g., human) according to the methods described herein. This is in contrast to solid malignancies, where significant discrepancies have been observed between immune data collected from the tissue infiltrate and peripheral blood (see, e.g., Chuah S. and Chew V., 2020, J. Immunother. Cancer, 8 (1): e000363).

The data in the Examples section also demonstrate that immune reactivity (as determined by one or more (e.g., 1, 2, 3, 4, 5, 6, 7, 8, 9, 10, 11) immune biomarkers described herein) is still present in subjects with overt MM. Thus, the markers described herein may also be used for subjects with overt MM, in addition to subjects with MGUS or SMM.

Thus, this disclosure describes novel markers to select subjects for therapy, to identify progression and treatment response of SMM, MGUS, or MM in subjects (e.g., humans) and to identify and/or select subjects (e.g., humans) who would benefit from particular treatments, termination of treatment, or modification of treatment (e.g., changes in amount, duration, or type of treatment; changes in the frequency of follow-up assessments or the type of tests performed clinically). More specifically, the present disclosure provides materials and methods for the subject selection, prognosis, staging, monitoring, and treatment of SMM, MGUS, or MM based on the presence of the biomarkers in a bone marrow sample or a blood sample. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate. This disclosure also provides methods for monitoring the progression of SMM, MGUS, or MM, determining the efficacy of a therapeutic agent (e.g., as determined by likelihood of progression-free survival), and/or determining a therapy for SMM, MGUS, or MM. This disclosure also provides methods for treating SMM (e.g., high risk SMM), MGUS, or MM in a subject (e.g., human) based on the presence of one or more (e.g., 1, 2, 3, 4, 5, 6, 7, 8, 9, 10, 11) biomarkers in a bone marrow sample or blood sample.

### Smoldering Multiple Myeloma, Monoclonal Gammopathy of Undetermined Significance (MGUS), and Multiple Myeloma

Multiple myeloma (MM; also known as plasma cell myeloma, myelomatosis, or Kahler's disease) is a cancer of plasma cells, a type of white blood cell normally responsible for producing antibodies. In MM, collections of abnormal plasma cells accumulate in the bone marrow, where they interfere with the production of normal blood cells.

Recent studies have shown that MM is consistently preceded by a precursor state such as monoclonal gammopathy of undetermined significance (MGUS) or smoldering multiple myeloma (SMM) (Landgren et al., 2009 Blood 113:5412-5417; Weiss et al., 2009 Blood 113:5418-5422). MGUS is characterized by blood M protein <30 g/L, bone marrow plasma cells <10%, and no myeloma-related organ or tissue impairment. MGUS is observed for progression, but is typically not treated. SMM is characterized by blood M protein >30 g/L, bone marrow plasma cells >10%, and myeloma-related organ or tissue impairment. SMM is typically observed and not treated. The presence of serum M-protein >2 g/dL together with involved to uninvolved sFLC ratio >20 and bone marrow plasma cell infiltration >20% (see, e.g., Mateos et al., Blood Cancer Journal, 10 (102): (2020); Rajkumar S V, et al., Blood 125:3069-3075 (2015), which are incorporated by reference herein in their entirety) are independent risk factors of progression that can be used to identify subjects with SMM at high risk of progression. High risk for progression can also be assessed separately or in combination by the Kyle et al. model, the PETHEMA model, or the “20-2-20” model.

Some subjects rapidly progress from SMM to overt MM (progressors) with a rate of progression of 70% over 5 years, while others remain indolent with minimal progression (non-progressors) over the same time period (Landgren, 2013 Hematology: ASH Education Book 1:478-487). Biological factors that distinguish progressors and non-progressors in MGUS/SMM are not well known (Ghobrial et al, 2014 Blood 124:3380-8). The current prognostic factors used to assess progression are based on tumor burden markers including the level of monoclonal spike, free light chains, and/or percent of plasma cells in the bone marrow.

As used herein, unless otherwise indicated, MM refers to any stage of MM except for MGUS and SMM. Thus, stages of MM include MM defined by the presence of myeloma-defining events, as defined by the International Myeloma Working Group (such as bone marrow infiltration >=60%, FLC ratio >=100 and more than 1 focal lesion on MRI) (see, e.g., Rajkumar S V, American Society of Clinical Oncology Educational Book 2016:36, e418-e423, which is incorporated by reference herein in its entirety), and plasma cell leukemia (PCL; the most aggressive plasma cell disorder), but do not include MGUS and SMM. Thus, in some instances, a subject is considered to have progressed to MM when said subject has the presence of one or more myeloma-defining event or plasma cell leukemia. MM is characterized by the presence of plasma cells ≥10% in bone marrow or in any quantity in other tissues (plasmacytoma) and at least one myeloma-defining event (see, e.g., Rajkumar S V, American Society of Clinical Oncology Educational Book 2016:36, e418-e423, which is incorporated by reference herein in its entirety). Thus, in some instances, a subject is considered to have progressed to MM when said subject has hypercalcemia (e.g., a serum calcium level greater than 0.25 mmol/L above the upper limit of normal or a level that is greater than 2.75 mmol/L), renal or kidney problems (e.g., a creatinine greater than 173 mmol/L), anemia (e.g., a low hemoglobin level, e.g., 2 g/dL below the lower limit of normal or a hemoglobin level that is less than 10 g/dL), and/or bone pain or lesions (e.g., lytic lesions, osteoporosis, or compression fraction of the spine). Other myeloma-defining events indicating a subject has progressed to MM include: more than 60% of the cells in the bone marrow are plasma cells, symptomatic hyperviscosity of the blood, amyloidosis, repeated serious bacterial infections (i.e., more than 2 episodes in a 12 month time-frame), bone lesions seen on MRI or PET-CT imaging, and an involved-to-uninvolved free light chain ratio of greater than 100 (based on serum testing), with an absolute value greater than 100 mg/L or 10 mg/dL. Methods of staging MM are known in the art (see, e.g., the International Staging System for Multiple Myeloma (Greipp et al., Journal of Clinical Oncology, 2005, 23 (15): 3412-3420, which is incorporated by reference herein in its entirety, and any updates thereto)).

MM is typically treated immediately. PCL can evolve from an existing case of multiple myeloma as part of the terminal phase of the disease and characterized by plasma cells accounting for more than 20% of cells in the peripheral blood with an absolute plasma cell count of more than 2×10⁹/L. Treatment for SMM, MGUS, or MM includes, for example, the following therapeutic agents: an immunomodulating agent (e.g., Empliciti™ [elotuzumab], Thalomid® [thalidomide], Pomalyst™ [pomalidomide], or Revlimid® [lenalidomide]), a proteasome inhibitor (e.g., Velcade® [bortezomib], Ninlaro™ [ixazomib] or Kyprolis™ [carfilzomib]), a chemotherapy agent (e.g., Doxil® [doxorubicin], cyclophosphamide, etoposide, liposomal doxorubicin, melphalan, melphalan flufenamide, bendamustine), a histone deacetylase (e.g., Farydak™ [panobinostat]), a monoclonal antibody against CD38 (e.g., Darzalex™ [daratumumab], Sarclisa™ [isatuximab]), an antibody against SLAMF7 (e.g., Empliciti™ [elotuzumab]), an antibody-drug conjugate (e.g., Blenrep™ [belantamab mafodotin-blmf]), a nuclear export inhibitor (e.g., Xpovio [selinexor]), a steroid (e.g., a corticosteroid, e.g., dexamethasone or prednisone), a bisphosphonate (for individuals with osteolytic lesions, osteoporosis, or osteopenia), a CAR-T cell therapy such as CAR-T cells for BCMA (e.g., Abecma® [idecabtagene vicleucel]) or GPRC5D), multispecific antibodies (e.g., targeting BCMA), and any combination thereof. Therapeutic agents for treatment of SMM, MGUS, or MM include any therapeutic agent approved (e.g., by the US Food and Drug Administration or the European Medicines Agency), or any combination thereof, for the treatment of SMM, MGUS, or MM. In some instances, a treatment used in the methods described herein comprises a therapeutically effective amount of one or more (e.g., 1, 2, 3, 4) therapeutic agents used to treat MM. In some instances, a treatment used in the methods described herein comprises a therapeutically effective amount of one or more (e.g., 1, 2, 3, 4) therapeutic agents used to treat SMM. In some instances, a treatment used in the methods described herein comprises a therapeutically effective amount of one or more (e.g., 1, 2, 3, 4) therapeutic agents used to treat MGUS. In addition, any therapeutic agent may be used alone or in combination with other therapies. In some instances, the treatment comprises bortezomib, lenalidomide, and dexamethasone. In some instances, the treatment comprises bortezomib, lenalidomide, dexamethasone, and daratumumab. In some instances, the treatment comprises autologous stem cell transplantation (ASCT). In some instances, the treatment comprises CAR-T cells for BCMA. In some instances, the treatment comprises an immunotherapy. In some instances, the treatment comprises an immunotherapy and a proteasome inhibitor, optionally also CAR-T cells for BCMA. In some instances, the treatment comprises an immunotherapy and an immunomodulating agent. In some instances, the treatment comprises an immunotherapy, an immunomodulating agent, and a proteasome inhibitor, optionally also CAR-T cells for BCMA. In some instances, the treatment comprises an immunotherapy and a steroid. In some instances, the treatment comprises an immunotherapy, an immunomodulating agent, and a steroid, optionally also CAR-T cells for BCMA. In some instances, the treatment comprises an immunotherapy, an immunomodulating agent, a steroid, and a proteasome inhibitor. In some instances, the treatment comprises an immunotherapy, an immunomodulating agent, a steroid, a proteasome inhibitor, and CAR-T cells for BCMA. For instance, a subject (e.g., human) described herein (e.g., a subject having SMM, MGUS, or MM) may be administered an immunotherapy (e.g., an anti-SLAMF7 antibody, e.g., elotuzumab), an immunomodulatory imide drug (e.g., lenalidomide), and a steroid (e.g., dexamethasone). In some instances, a subject (e.g., human) described herein (e.g., a subject having SMM, MGUS, or MM) may be administered elotuzumab, lenalidomide, and dexamethasone.

Given that MM is always preceded by a well-defined precursor state, and given the ease of access to primary patient samples (peripheral blood samples and bone marrow samples), MM can represent one of the best models of cancer to determine biomarkers of tumor progression in early premalignant conditions. This disclosure provides molecular biomarkers of SMM, MGUS useful for prognosis, treatment, and/or staging of SMM, MGUS, or MM that will significantly impact the clinical care of patients having SMM, MGUS, or MM.

### Methods for Detecting Multiple Myeloma

To date, the gold standard for characterizing MM disease state has involved a bone marrow biopsy. The present disclosure provides a non-invasive method for characterizing the disease state of a patient. The methods of the invention are suitable for use alone, or if desired, may be used in concert with one or more of the following conventional diagnostic methods.

Traditionally, the initial evaluation of a suspected hemotological malignancy (e.g., a monoclonal gammopathy) includes both serum and urine protein electrophoresis with immunofixation to identify and quantify the M protein. The majority of patients are expected to have a detectable M protein, but approximately 1-3% can present with a non-secretory myeloma that does not produce light or heavy chains. True non-secretory myeloma is thus rare, not least because, with the availability of serum free light chain testing, it is recognized that M protein is present. The most common M protein is IgG, followed by IgA, and light-chain-only disease. IgD and IgE are relatively uncommon and can be more difficult to diagnose because their M spikes are often very small. Up to 20% of patients will produce only light chains, which may not be detectable in the serum because they pass through the glomeruli and are excreted in the urine. The present invention provides methods that can also be used to detect and/or characterize a monoclonal gammopathy in a patient.

A standard evaluation of a documented monoclonal gammopathy includes a complete blood count with differential, calcium, serum urea nitrogen, and creatinine. Serum free light chain testing is also a useful diagnostic test (Piehler A. P. et al, Clin. Chem., 54:1823-30 (2008)). Bone disease is best assessed by skeletal survey. Bone scans are not a sensitive measure of myelomatous bone lesions because the radioisotope is poorly taken up by lytic lesions in MM, as a result of osteoblast inhibition. Magnetic resonance imaging (MRI) is useful for the evaluation of solitary plasmacytoma of bone and for the evaluation of paraspinal and epidural components. 18F-FDG Positron Emission Tomography (PET)/CT scans are more sensitive in the detection of active lesions in the whole body (Fonti R. et al., J. Nucl. Med., 49:195-200 (2008)). A bone marrow aspiration and biopsy are helpful to quantify the plasma cell infiltrate and adds important prognostic information with cytogenetic evaluation, including fluorescent in situ hybridization (FISH). Additional prognostic information can be obtained with serum B2-microglobulin (B2M) and C-reactive protein (CRP).

The criteria for the diagnosis of MM, SMM, and MGUS are detailed in Table 1 below. Distinction among these disease states informs treatment decisions and prognostic recommendations.

TABLE 1A
Conventional criteria for the diagnosis of MM, SMM, and MGUS

| Disorder | Disease definition |
|---|---|
| MGUS | Serum monoclonal protein level <3 g/dL, |
| | bone marrow plasma cells 10%, and absence |
| | of end-organ damage, such as lytic bone |
| | lesions, anemia, hypercalcemia, or renal |
| | failure, that can be attributed to a plasma |
| | cell proliferative disorder. |
| SMM | Serum monoclonal protein (IgG or IgA) |
| | level ≥3 g/dL and/or bone marrow plasma |
| | cells .10%, absence of end-organ damage, |
| | such as lytic bone lesions, anemia, |
| | hypercalcemia, or renal failure that can be |
| | attributed to a plasma cell proliferative |
| | disorder. |
| | Alternatively or additionally, SMM may be |
| | defined by the presence or absence of |
| | biomarkers (e.g., Myeloma Defining Event |
| | biomarkers), by bone-marrow plasma cell |
| | infiltration ≥60%, by serum-free light chain |
| | ratio ≥100, and/or by >1 focal lesion in the |
| | skeleton on magnetic resonance imaging |
| | analysis (see, Rajkumar SV, et al. |
| | International Myeloma Working Group |
| | updated criteria for the diagnosis of multiple |
| | myeloma. Lancet Oncol 15, e538-548 |
| | (2014)). In some instances, SMM may be |
| | associated with organ damage. |
| MM | Bone marrow plasma cells ≥10%, presence |
| | of serum and/or urinary monoclonal protein |
| | (except in patients with true nonsecretory |
| | multiple myeloma), plus evidence of lytic |
| | bone lesions, anemia, hypercalcemia, or |
| | renal failure that can be attributed to the |
| | underlying plasma cell proliferative |
| | disorder. |

Conventional staging systems involve the following. The most widely used myeloma staging system since 1975 has been the Durie-Salmon, in which the clinical stage of disease is based on several measurements including levels of M protein, serum hemoglobin value, serum calcium level, and the number of bone lesions. The International Staging System (ISS), developed by the International Myeloma Working Group is now also widely used (Greipp P R. Et al, J. Clin. Oncol, 23:3412-20 (2005)). ISS is based on two prognostic factors: serum levels of B2M and albumin, and is comprised of three stages: B2M 3.5 mg/L and albumin 3.5 g/dL (median survival, 62 months; stage I); B2M<3.5 mg/L and albumin<3.5 g/dL or B2M 3.5 to <5.5 mg/L (median survival, 44 months; stage II); and B2M 5.5 mg/L (median survival, 29 months; stage III). With an increased understanding of the biology of myeloma, other factors have been shown to correlate well with clinical outcome and are now commonly used. For example, cytogenetic abnormalities as detected by FISH techniques have been shown to identify patient populations with very different outcomes. For instance, loss of the long arm of chromosome 13 is found in up to 50% of patients and, when detected by metaphase chromosome analysis, is associated with poor prognosis. In addition, a hypodiploid karyotyped t (4;14), and—17pl3.1 is typically associated with poor outcome, while the t (11; 14) and hypodiploidy are associated with improved survival (Kyrtsonis M. C. et al., Semin. Hematol, 46:110-7, (2009)).

### Markers

As described above, the present disclosure involves, determining that a sample (e.g., blood or bone marrow, e.g., mononuclear cells obtained from blood or CD138-mononuclear cells obtained from bone marrow or blood) from a human subject having SMM, MGUS, or MM has one or more (e.g., 1, 2, 3, 4, 5, 6, 7, 8, 9, 10, 11) markers set forth in Tables 1-6 or the Examples section herein. In some instances, the subject having SMM, MGUS, or MM is not undergoing treatment for SMM, MGUS, or MM or has not undergone treatment for SMM, MGUS, or MM (see, e.g., Tables 1 and 2). In some instances, the subject having SMM, MGUS, or MM is undergoing treatment (e.g., has received/is receiving one or more (e.g., 1, 2, 3) doses of one or more (e.g., 1, 2, 3, 4) therapeutic agents for the treatment of SMM, MGUS, or MM, e.g., immunotherapy and optionally a steroid, e.g., elotuzumab, lenalidomide, and dexamethasone) (see, e.g., Tables 3 and 4). In some instances, the human subject having SMM, MGUS, or MM has undergone treatment (e.g., has received one or more (e.g., 1, 2, 3) doses of one or more (e.g., 1, 2, 3, 4) therapeutic agents for the treatment of SMM, MGUS, or MM, e.g., immunotherapy and optionally a steroid, e.g., elotuzumab, lenalidomide, and dexamethasone) (see, e.g., Tables 5 and 6). In some instances, the subject has SMM.

In some instances, the subject having SMM has high-risk SMM. In some instances, the subject having SMM has high-risk SMM based on the Rajkumar et al., Blood 125:3069-3075 (2015) criteria. In some instances, the subject having SMM has high-risk SMM based on the “20-2-20” criteria. In some instances, the subject having SMM has low- or intermediate-risk SMM based on the “20-2-20” criteria. In some cases, the subject having SMM has high-risk SMM based on the Rajkumar et al., criteria and the “20-2-20” criteria (Mateos et al., Blood Cancer Journal, 10 (102): (2020)). In some instances, the subject has MGUS. In some instances, the human subject has MM (e.g., smoldering). The level of the biomarker can be determined by any method known in the art (e.g., single-cell RNA sequencing, targeted single-cell RNA sequencing, immunohistochemistry, flow cytometry, mass cytometry, or an imaging-based method).

TABLE 1B
Markers prior to treatment (e.g., with immunotherapy, Elotuzumab,
Lenalidomide, Dexamethasone) useful in subject selection for
treatment, and/or predictive of significantly longer progression-
free survival upon treatment (e.g., with immunotherapy)

| Marker ID | Marker |
|---|---|
| 1i | an increase in the proportion of GZMK⁺ T cells present in a biological |
| | sample relative to a control proportion of GZMK⁺ T cells present in a |
| | reference |
| 1ii | an increased proportion of GZMK⁺ NK cells relative to a control |
| | proportion of GZMK⁺ NK cells |
| 1iii | an increased proportion of Th17 cells relative to a control proportion of |
| | Th17 cells |
| 1iv | a decreased proportion of pDCs relative to a control proportion of |
| | pDCs |
| 1v | a decreased proportion of HSCs relative to a control proportion of |
| | HSCs |
| 1vi | an increased proportion of mature B-cells relative to a control |
| | proportion of mature B-cells |
| 1vii | an increased activity of GZMK-associated signaling relative to a control |
| | activity of GZMK-associated signaling |
| 1viii | an increased activity of Th17-associated signaling relative to a control |
| | activity of Th17-associated signaling |
| 1ix | an increased activity of a compositional signature that captures an |
| | abundance of mature B-cells relative to a control activity of a |
| 1x | a decreased activity of a compositional signature that captures a |
| | proportion of HSCs relative to a control activity of a compositional |
| | signature that captures a proportion of HSCs |
| 1xi | An alteration (e.g., increased, decreased) activity of an immune |
| | reactivity score relative to a control activity of an immune reactivity |

TABLE 2
Markers prior to treatment predictive of significantly
shorter progression-free survival upon treatment

| Biomarker ID | Biomarker |
|---|---|
| 2i | a decreased proportion of GZMK⁺ T cells relative to a control |
| | proportion of GZMK⁺ T cells |
| 2ii | a decreased proportion of GZMK⁺ NK cells relative to a control |
| | proportion of GZMK⁺ NK cells |
| 2iii | a decreased proportion of Th17 cells relative to a control proportion of |
| | Th17 cells |
| 2iv | an increased proportion of pDCs relative to a control proportion of |
| | pDCs |
| 2v | an increased proportion of HSCs relative to a control proportion of |
| | HSCs |
| 2v | a decreased proportion of mature B-cells relative to a control proportion |
| | of mature B-cells |
| 2vii | a decreased activity of GZMK-associated signaling relative to a control |
| | activity of GZMK-associated signaling |
| 2viii | a decreased activity of Th17-associated signaling relative to a control |
| | activity of Th17-associated signaling |
| 2ix | a decreased activity of a compositional signature that captures an |
| | proportion of mature B-cells relative to a control activity of a |
| | compositional signature that captures an proportion of mature B-cells |
| 2x | an increased activity of a compositional signature that captures an |
| | proportion of HSCs relative to a control activity of a compositional |
| | signature that captures an proportion of HSCs |
| 2xi | a decreased activity of an immune reactivity score relative to a control |
| | activity of an immune reactivity score |

In some instances of the biomarkers of Table 1 or Table 2, the B-cells are mature naïve B-cells. In some instances of the biomarkers of Table 1 or Table 2, the B-cells are mature memory B-cells. In some instances of the biomarkers of Table 1 or Table 2, the B-cells are mature naïve B-cells and mature memory B-cells.

TABLE 3
Markers during treatment (e.g., with immunotherapy) predictive of significantly
shorter progression-free survival upon treatment (e.g., with immunotherapy)

| Marker ID | Marker |
|---|---|
| 3i | an increased proportion of tissue-resident NK cells |
| 3ii | an increased proportion of exhausted GZMK⁺ CD8⁺ T-cells |
| 3iii | an increased proportion of activated CD4⁺ Central Memory T-cells |
| | (aCD4⁺ TCMs) |
| 3iv | an increased activity of a compositional signature corresponding to the |
| | proportion of tissue-resident NK cells, exhausted GZMK⁺ CD8⁺ T-cells, |
| | and activated CD4⁺ Central Memory T-cells |
| 3v | an increased activity of a gene expression signature corresponding to |
| | the activity of one or more (e.g., 1, 2, 3, 4) genes selected from the |
| | group consisting of AREG, FAM177A1, RGS1, and IL32 |
| 3vi | an increased expression level of one or more (e.g., 1, 2, 3) genes |
| | selected from the group consisting of AREG, FAM177A1, and RGS1 |
| 3vii | a decreased expression level of IL32 |

TABLE 4
Markers during treatment (e.g., with immunotherapy) predictive of significantly
longer progression-free survival upon treatment (e.g., with immunotherapy)

| Biomarker ID | Biomarker |
|---|---|
| 4i | a decreased proportion of tissue-resident NK cells |
| 4ii | a decreased proportion of exhausted GZMK⁺ CD8⁺ T-cells |
| 4iii | a decreased proportion of activated CD4⁺ Central Memory T-cells |
| | (aCD4⁺ TCMs) |
| 4iv | a decreased activity of a compositional signature corresponding to the |
| | proportion of tissue-resident NK cells, exhausted GZMK⁺ CD8⁺ T- |
| | cells, and activated CD4⁺ Central Memory T-cells |
| 4v | a decreased activity of a gene expression signature corresponding to the |
| | activity of one or more (e.g., 1, 2, 3, 4) genes selected from the group |
| | consisting of AREG, FAM177A1, RGS1, and IL32 |
| 4vi | a decreased expression level of one or more (e.g., 1, 2, 3) genes selected |
| | from the group consisting of AREG, FAM177A1, and RGS1 |
| 4vii | an increased expression level of IL32 |

TABLE 5
Markers after treatment (e.g., with immunotherapy)
predictive of significantly longer progression-free
survival upon treatment (e.g., with immunotherapy)
Biomarker

Immune cell composition similar to a control immune cell composition
Increase in immune normalization score relative to immune normalization
at baseline (e.g., prior to therapy)

TABLE 6
Markers after treatment (e.g., with immunotherapy)
predictive of significantly shorter progression-free
survival upon treatment (e.g., with immunotherapy)
Biomarker

Immune cell composition similar to a control immune cell composition

The Examples section herein provides exemplary descriptions of immune cell compositions considered to be similar to healthy human subjects.

In some instances with respect to the markers of Tables 5 and 6, the immune cell composition comprises the proportion of one or more (e.g., 1, 2, 3, 4, 5) of T-cells, B-cells, NK cells, monocytes, and stem cells. In some instances, the immune cell composition comprises the proportion of T-cells. In some instances, the immune cell composition comprises the proportion of B-cells. In some instances, the B-cells are mature naïve B-cells. In some instances, the B-cells are mature memory B-cells. In some instances, the B-cells are mature naïve B-cells and mature memory B-cells. In some instances, the immune cell composition comprises the proportion of NK cells. In some instances, the immune cell composition comprises the proportion of monocytes. In some instances, the immune cell composition comprises the proportion of stem cells.

The markers delineated herein, including those presented in Tables 1-6 can be measured in a sample obtained from the human subject having SMM, MGUS, or MM. In some instances, the sample comprises or consists of mononuclear cells from a blood sample from the human subject. In some instances, the sample comprises or consists of CD138-mononuclear cells from a bone marrow sample or a blood sample from the human subject. In some instances, the sample comprises or consists of a bone marrow tissue section from the human subject. In some cases, aspiration is used to obtain a bone marrow sample. In some cases a biopsy is performed to obtain a bone marrow sample. In some instances, the sample is a bone marrow aspirate. In some cases, the sample is a fresh sample. In some cases, the sample is a frozen sample. In some cases, the sample is a tissue section sample (e.g., FFPE bone marrow tissue sample). The level of the marker can be determined by any method known in the art (e.g., single-cell RNA sequencing, targeted single-cell RNA sequencing, immunohistochemistry, flow cytometry, mass cytometry, or an imaging-based method).

In some instances, the level of the marker is increased relative to a control level of the marker if the level of the marker is at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, at least 25-fold, at least 50-fold, at least 75-fold, or at least 100-fold higher, or at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 1,500%, at least 2,000%, at least 2,500%, at least 3,000%, at least 3,500%, at least 4,000%, at least 4,500%, or at least 5,000% higher than the control level of the marker. In some instances, the level of the marker is decreased relative to a control level of the marker if the level of the marker is at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, at least 25-fold, at least 50-fold, at least 75-fold, or at least 100-fold lower, or at least 10%, at least 20%, at least 30%, at least 40%, at least 50%, at least 60%, at least 70%, at least 80%, at least 90%, at least 95%, at least 97%, or 100% lower than the control level of the marker. In some instances, the control level of the marker is the level of the marker in a corresponding sample (e.g., same tissue type as the test sample) from a healthy human subject (e.g., a human subject of similar age who does not have SMM, MGUS, or MM).

In some instances with respect to the marker of Table 5, the immune cell composition is similar to the control immune cell composition if the level of one or more (e.g., 1, 2, 3, 4, 5) types of immune cells (e.g., T-cells, B-cells, NK cells, monocytes, stem cells) in the immune cell composition is within 1%, 2%, 3%, 4%, 5%, 10%, 15%, 20%, or 25% of the level of the one or more types of immune cells in the control immune cell composition. In some instances with respect to the marker of Table 5, the immune cell composition is similar to the control immune cell composition if the proportion of one or more (e.g., 1, 2, 3, 4, 5) cell type (e.g., T-cells, B-cells, NK cells, monocytes, and stem cells) in the immune cell composition positively correlate with those in the control immune cell composition. In some instances with respect to the marker of Table 6, the immune cell composition is dissimilar to the control immune cell composition if the level of one or more (e.g., 1, 2, 3, 4, 5) types of immune cells (e.g., T-cells, B-cells, NK cells, monocytes, stem cells) in the immune cell composition is not within 1%, 2%, 3%, 4%, 5%, 10%, 15%, 20%, or 25% of the level of the one or more types of immune cells in the control immune cell composition. In some instances with respect to the marker of Table 6, the immune cell composition is dissimilar to the control immune cell composition if the proportion of one or more (e.g., 1, 2, 3, 4, 5) cell type (e.g., T-cells, B-cells, NK cells, monocytes, and stem cells) in the immune cell composition does not positively correlate with those in the control immune cell composition.

In some instances, the control level of the marker is the median level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the control level of the marker is the mean level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the control level of the marker is the first quartile level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the control level of the marker is the third quartile level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM.

In some instances, the marker is defined as a correlation coefficient, computed between immune cell proportions in human subjects with SMM or MGUS and immune cell proportions in a panel (e.g., 1, 2, 3, 4, 5, 6, 7, 8, 9, 10, 15, 20, 25, 30, 35, or 40 or more) of healthy human subjects, which is higher than the median level in a panel (e.g., 1, 2, 3, 4, 5, 6, 7, 8, 9, 10, 15, 20, 25, 30, 35, or 40 or more) of human subjects with SMM or MGUS. In some instances, the marker is defined as a correlation coefficient, computed between immune cell proportions in human subjects with SMM or MGUS and immune cell proportions in a panel (e.g., 1, 2, 3, 4, 5, 6, 7, 8, 9, 10, 15, 20, 25, 30, 35, or 40 or more) of healthy human subjects, which is higher than the mean level in a panel (e.g., 1, 2, 3, 4, 5, 6, 7, 8, 9, 10, 15, 20, 25, 30, 35, or 40 or more) of human subjects with SMM or MGUS. In some instances, the marker is defined as a correlation coefficient, computed between immune cell proportions in human subjects with SMM or MGUS and immune cell proportions in a panel (e.g., 1, 2, 3, 4, 5, 6, 7, 8, 9, 10, 15, 20, 25, 30, 35, or 40 or more) of healthy human subjects, which is higher than the first quartile level in a panel (e.g., 1, 2, 3, 4, 5, 6, 7, 8, 9, 10, 15, 20, 25, 30, 35, or 40 or more) of human subjects with SMM or MGUS. In some instances, the marker is defined as a correlation coefficient, computed between immune cell proportions in human subjects with SMM or MGUS and immune cell proportions in a panel (e.g., 1, 2, 3, 4, 5, 6, 7, 8, 9, 10, 15, 20, 25, 30, 35, or 40 or more) of healthy human subjects, which is higher than the third quartile level in a panel (e.g., 1, 2, 3, 4, 5, 6, 7, 8, 9, 10, 15, 20, 25, 30, 35, or 40 or more) of human subjects with SMM or MGUS. In some instances, the marker is defined by the presence of a significant correlation (e.g., p<0.05, p<0.01, FDR<0.1, FDR<0.05, or FDR<0.01), computed between immune cell proportions in human subjects with SMM or MGUS and immune cell proportions in a panel (e.g., 1, 2, 3, 4, 5, 6, 7, 8, 9, 10, 15, 20, 25, 30, 35, or 40 or more) of healthy human subjects. In some instances, the marker is defined based on a weighted sum of immune cell proportions (i.e., the sum of the products of immune cell proportions and their corresponding weights) and its level compared to the median of healthy human subjects or the median of human subjects with SMM, MM or MGUS. In some instances, the marker is defined based on the change in the human subject's weighted sum of immune cell proportions post-treatment (i.e, whether it increases, decreases or remains unchanged).

Methods of determining the proportion of a particular cell type (e.g., GZMK⁺ T cells, GZMK⁺ NK cells, Th17 cells, pDCs, HSCs, mature B-cells, tissue-resident NK [trNK] cells, exhausted GZMK⁺ CD8⁺ T-cells, activated CD4⁺ Central Memory T-cells [aCD4⁺ TCMs]) in a sample (e.g., bone marrow or blood) obtained from a subject having SMM or MGUS are known in the art and described herein. Non-limiting examples of methods that may be used to determine the proportion of a particular cell type include single-cell RNA sequencing, targeted single-cell RNA-sequencing, RNA-sequencing, immunohistochemistry, immunofluorescence, flow cytometry, mass cytometry, mass spectrometry, and imaging-based methods, such as imaging cytometry. In some instances, for methods done in bulk, such as RNA sequencing or mass spectrometry, the methods are coupled with computation deconvolution approaches to estimate the proportion of different cell types in the sample. Non-limiting examples of deconvolution approaches include EPIC (Racle & Gfeller, Methods Mol Biol. 2020, 2120:233-248) and CIBERSORT (Chen et al., Methods Mol Biol. 2018; 1711:243-259). In some instances, the proportion of a particular cell type is determined using single-cell RNA sequencing. Gene expression markers known in the art can be used to annotate cell types. See, e.g., the materials and methods in the Examples section below. In some instances, GZMK⁺ T cells are annotated by expression of CD3D, CD8A, CD8B, and GZMK. In some instances, GZMK⁺ NK cells are annotated by expression of NKG7, CD56, XCL2, SELL, and GZMK. In some instances, Th17 cells are annotated by expression of CD3D, CD4, RORA, RORC, KLRB1, CXCR6, CCR6, and GZMK. In some instances, pDCs are annotated by expression of MZB1, LILRA4, and CLEC4C. In some instances, HSCs are annotated by expression of CD34 and CDK6. In some instances, mature B-cells are annotated by expression of CD19, MS4A1, IGHM, IGHD, CD27, IGHG1, IGHG2, IGHG3, IGHA1, and IGHA2. In some instances, trNK cells are annotated by expression of CD69, ICAMI, CD160, AREG, FAM177A1, RGS1, RGS2, CXCR4, IFRD1, and NR4A2. In some instances, exhausted GZMK⁺ CD8⁺ T-cells cells are annotated by expression of CD3D, CD8A, CD8B, GZMK, TOX, XCL2, CCL3, CCL4, TIGIT, and PDCD1. In some instances, aCD4⁺ TCMs cells are annotated by expression of CD3D, CD4, JUN, FOS, DUSP1, TSC22D3, NFKBIA, and NR4A2. In some instances, the proportion of a particular cell type is determined in a bone marrow sample (e.g., CD138-mononuclear cells obtained from a bone marrow sample) from a human subject having SMM or MGUS. In some instances, the proportion of a particular cell type is determined in a blood sample (e.g., CD138-mononuclear cells or mononuclear cells obtained from a blood sample) from a human subject having SMM or MGUS. In some instances, the proportion of a particular cell type is determined in a population of PBMCs from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the activity of GZMK-associated signaling are known in the art and described herein. For instance, the activity of GZMK-associated signaling may be determined by determining the level (e.g., mRNA or protein level) of GZMK and/or one or more (e.g., 1, 2, 3, 4, 5, 6, 7) mRNAs/proteins expressed in T-cells and NK cells. For example, in some instances, the expression level (e.g., mRNA or protein level) of one or more (e.g., 1, 2, 3, 4, 5, 6, 7) of GZMK, XCL2, CMC1, XCL1, SELL, CCL3 and CCL4 is evaluated to determine the activity of GZMK-associated signaling. In some instances, a sample (e.g., bone marrow or blood) from a human subject having SMM or MGUS is determined to have an increased activity of GZMK-associated signaling if there is an increased level (e.g., mRNA or protein) of GZMK and/or an increased level (e.g., mRNA or protein) of one or more (e.g., 1, 2, 3, 4, 5, 6) proteins (e.g., XCL2, CMC1, XCL1, SELL, CCL3, CCL4) expressed in T-cells and NK cells. Non-limiting examples of methods that may be used to determine the activity of GZMK-associated signaling (e.g., the level, e.g., the mRNA or protein level, of GZMK and/or an mRNA/protein expressed in GZMK⁺ T-cells and NK cells) include single-cell RNA sequencing, targeted single-cell RNA-sequencing, RNA-sequencing, mass spectrometry, immunohistochemistry, immunofluorescence, flow cytometry, mass cytometry, quantitative real-time PCR (qPCR), western blots, and imaging-based methods, such as imaging cytometry. In some instances, the activity of GZMK-associated signaling is determined using single-cell RNA sequencing. In some instances, the activity of GZMK-associated signaling is determined in a bone marrow sample (e.g., CD138-mononuclear cells obtained from a bone marrow sample) from a human subject having SMM or MGUS. In some instances, the activity of GZMK-associated signaling is determined in a blood sample (e.g., mononuclear cells, e.g., CD138-mononuclear cells, obtained from a blood sample) from a human subject having SMM or MGUS. In some instances, the activity of GZMK-associated signaling is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the activity of Th17-associated signaling are known in the art and described herein. The activity of Th17-associated signaling may be determined by determining the level (e.g., mRNA or protein level) of one or more (e.g., 1, 2, 3, 4, 5) proteins expressed by type 17 T-cells. For example, in some instances, the expression level (e.g., mRNA or protein level) of one or more (e.g., 1, 2, 3, 4, 5) of KLRB1, RORA, RORC, CXCR6 and CCR6 is evaluated to determine the activity of Th17-associated signaling. In some instances, a sample (e.g., bone marrow or blood) from a human subject having SMM or MGUS is determined to have an increased activity of Th17-associated signaling if there is an increased level (e.g., mRNA or protein) of one or more (e.g., 1, 2, 3, 4, 5) proteins (e.g., KLRB1, RORA, RORC, CXCR6, CCR6) expressed in T-cells. Non-limiting examples of methods that may be used to determine the activity of Th17-associated signaling (e.g., the level, e.g., the mRNA or protein level, of a protein expressed by type 17 T-cells) include single-cell RNA sequencing, targeted single-cell RNA-sequencing, RNA-sequencing, quantitative real-time PCR (qPCR), immunohistochemistry, immunofluorescence, flow cytometry, mass cytometry, mass spectrometry, western blot, and imaging-based methods, such as imaging cytometry. In some instances, the activity of Th17-associated signaling is determined using single-cell RNA sequencing. In some instances, the activity of Th17-associated signaling is determined in a bone marrow sample (e.g., a population of CD138-mononuclear cells in a bone marrow sample) from a human subject having SMM. In some instances, the activity of Th17-associated signaling is determined in a blood sample (e.g., mononuclear cells, e.g., CD138-mononuclear cells, obtained from a blood sample) from a human subject having SMM or MGUS. In some instances, the activity of Th17-associated signaling is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the activity of a compositional signature capturing the proportion of mature B-cells (Naïve and/or Memory) and hematopoietic stem cells are described herein. Computationally, a matrix deconvolution or dimensionality reduction approach, such as Principal Component Analysis or Non-Negative Matrix Factorization, can be applied on a proportion matrix with cell types in rows and samples in columns to produce a matrix of compositional signatures and their activity in each sample. Non-limiting examples of methods that may be used to produce a matrix of immune cell proportions and determine the activity of a compositional signature capturing the proportion of mature B-cells or hematopoietic stem cells using one of the computational analyses described above include single-cell RNA sequencing, targeted single-cell RNA-sequencing, RNA-sequencing, immunohistochemistry, immunofluorescence, flow cytometry, mass cytometry, mass spectrometry, and imaging-based methods, such as imaging cytometry. In some instances, the activity of a compositional signature capturing the proportion of mature B-cells or hematopoietic stem cells is determined using single-cell RNA sequencing. In some instances, the activity of a compositional signature capturing the proportion of mature B-cells or hematopoietic stem cells is determined in a bone marrow sample (e.g., a population of CD138-mononuclear cells in a bone marrow sample) from a human subject having SMM or MGUS. In some instances, the activity of a compositional signature capturing the proportion of mature B-cells or hematopoietic stem cells is determined in a blood sample (e.g., mononuclear cells, e.g., CD138-mononuclear cells, in a blood sample) from a human subject having SMM or MGUS. In some instances, the activity of a compositional signature capturing the proportion of mature B-cells or hematopoietic stem cells is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining an immune reactivity score are described herein. Immune reactivity is defined relative to the immune cell composition of healthy human subjects and captures the degree of dissimilarity between human subjects with SMM or MGUS and healthy human subjects. In some instances, the immune reactivity score is computed as the sum of compositional signature activity, whereby each signature is weighed by its importance in classifying a sample of CD138-mononuclear cells from bone marrow or blood or mononuclear cells from blood of a human subject having SMM or MGUS as normal or malignant. In some instances, the distinction between normal and malignant is based off of a Naïve Bayes classifier (e.g., trained on normal and malignant samples that is able to look at the compositional signatures or immune cell proportions and identify which sample is normal and which is malignant) and a weighted sum of immune cell proportions. This is described in the Example section. In some instances, the immune reactivity score is computed as the correlation coefficient and/or its corresponding p-value between the immune composition of a sample of CD138-mononuclear cells from bone marrow or blood or mononuclear cells from blood of a human subject having SMM or MGUS and the immune composition of a control (e.g., a panel of healthy human subjects). Non-limiting examples of methods that may be used to produce a matrix of immune cell proportions and determine the immune reactivity score include single-cell RNA sequencing, targeted single-cell RNA-sequencing, RNA-sequencing, immunohistochemistry, immunofluorescence, flow cytometry, mass cytometry, mass spectrometry, and imaging-based methods, such as imaging cytometry. Non-limiting examples of computational methods that may be used to summarize a matrix of immune cell proportions into an immune reactivity score per sample include non-negative matrix factorization, principal component analysis, Pearson's correlation, and Spearman's correlation. In some instances, the immune reactivity score is determined using single-cell RNA sequencing. In some instances, the immune reactivity score is determined in a bone marrow sample (e.g., a population of CD138-mononuclear cells in a bone marrow sample) from a human subject having SMM or MGUS. In some instances, the immune reactivity score is determined in a blood sample (e.g., a population of CD138-mononuclear cells in a blood sample or mononuclear cells in a blood sample) from a human subject having SMM or MGUS. In some instances, the immune reactivity score is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the activity of a compositional signature corresponding to the proportion of tissue-resident NK cells, exhausted GZMK⁺ CD8⁺ T-cells, and activated CD4⁺ Central Memory T-cells are known in the art and described herein. Computationally, a matrix deconvolution or dimensionality reduction approach, such as Principal Component Analysis or Non-Negative Matrix Factorization, can be applied on a proportion matrix with cell types in rows and samples in columns to produce a matrix of compositional signatures and their activity in each sample. Non-limiting examples of methods that may be used to produce a matrix of immune cell proportions and to determine the activity of a compositional signature corresponding to the proportion of tissue-resident NK cells, exhausted GZMK⁺ CD8⁺ T-cells, and activated CD4⁺ Central Memory T-cells include single-cell RNA sequencing, targeted single-cell RNA-sequencing, RNA-sequencing, immunohistochemistry, immunofluorescence, flow cytometry, mass cytometry, and imaging-based methods, such as imaging cytometry. In some instances, the activity of a compositional signature corresponding to the proportion of tissue-resident NK cells, exhausted GZMK⁺ CD8⁺ T-cells, and activated CD4⁺ Central Memory T-cells is determined using single-cell RNA sequencing. In some instances, the activity of a compositional signature corresponding to the proportion of tissue-resident NK cells, exhausted GZMK⁺ CD8⁺ T-cells, and activated CD4⁺ Central Memory T-cells is determined in a bone marrow sample (e.g., CD138-mononuclear cells obtained from bone marrow) from a human subject having SMM or MGUS. In some instances, the activity of a compositional signature corresponding to the proportion of tissue-resident NK cells, exhausted GZMK⁺ CD8⁺ T-cells, and activated CD4⁺ Central Memory T-cells is determined in a blood sample (e.g., mononuclear cells, e.g., CD138-mononuclear cells, obtained from blood) from a human subject having SMM or MGUS. In some instances, the activity of a compositional signature corresponding to the proportion of tissue-resident NK cells, exhausted GZMK⁺ CD8⁺ T-cells, and activated CD4⁺ Central Memory T-cells is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the activity of a gene expression signature corresponding to the activity of one or more (e.g., 1, 2, 3, 4) genes, e.g., selected from the group consisting of AREG, FAM177A1, RGS1, and IL32 are known in the art and described herein. Computationally, the gene expression levels of the above-mentioned genes for every sample can be averaged, summed or z-scored and then averaged or summed, or summarized in any other way to create an expression signature score per sample. Alternatively, unsupervised or semi-supervised matrix deconvolution methods can be employed on matrices of gene expression, where a large number of genes have been profiled in order to detect gene expression signatures and their activity in each sample de novo or quantify the activity of a known signature in each sample. Non-limiting examples of methods that may be used to produce a gene expression matrix at the mRNA or protein level and to determine the activity of a gene expression signature corresponding to the activity of one or more (e.g., 1, 2, 3, 4) genes, e.g., selected from the group consisting of AREG, FAM177A1, RGS1, and IL32 include single-cell RNA sequencing, targeted single-cell RNA-sequencing, RNA-sequencing, quantitative real-time PCR (qPCR), immunohistochemistry, immunofluorescence, flow cytometry, mass cytometry, mass spectrometry, western blot, and imaging-based methods, such as imaging cytometry. In some instances, the activity of a gene expression signature corresponding to the activity of one or more (e.g., 1, 2, 3, 4) genes, e.g., selected from the group consisting of AREG, FAM177A1, RGS1, and IL32 is determined using single-cell RNA sequencing. In some instances, the activity of a gene expression signature corresponding to the activity of one or more (e.g., 1, 2, 3, 4) genes, e.g., selected from the group consisting of AREG, FAM177A1, RGS1, and IL32 is determined in a bone marrow sample (e.g., CD138-mononuclear cells obtained from bone marrow) from a human subject having SMM or MGUS. In some instances, the activity of a gene expression signature corresponding to the activity of one or more (e.g., 1, 2, 3, 4) genes, e.g., selected from the group consisting of AREG, FAM177A1, RGS1, and IL32 is determined in a blood sample (e.g., mononuclear cells, e.g., CD138-mononuclear cells, obtained from blood) from a human subject having SMM or MGUS. In some instances, the activity of a gene expression signature corresponding to the activity of one or more (e.g., 1, 2, 3, 4) genes, e.g., selected from the group consisting of AREG, FAM177A1, RGS1, and IL32 is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

Methods of determining the expression level (e.g., mRNA or protein) of genes (e.g., AREG, FAM177A1, RGS1, IL32, KLRB1, RORA, RORC, CXCR6, CCR6, GZMK, XCL2, CMC1, XCL1, SELL, CCL3, CCL4) are known in the art and described herein. The expression level of a gene (e.g., AREG, FAM177A1, RGS1, IL32, KLRB1, RORA, RORC, CXCR6, CCR6, GZMK, XCL2, CMC1, XCL1, SELL, CCL3, CCL4) may be determined by determining the mRNA level of the gene. The expression level of a gene (e.g., AREG, FAM177A1, RGS1, IL32, KLRB1, RORA, RORC, CXCR6, CCR6, GZMK, XCL2, CMC1, XCL1, SELL, CCL3, CCL4) may be determined by determining the protein level of the gene. Non-limiting examples of methods that may be used to determine the expression level (e.g., mRNA or protein) of a gene (e.g., AREG, FAM177A1, RGS1, IL32, KLRB1, RORA, RORC, CXCR6, CCR6, GZMK, XCL2, CMC1, XCL1, SELL, CCL3, CCL4) include single-cell RNA sequencing, targeted single-cell RNA-sequencing, RNA-sequencing, quantitative real-time PCR (qPCR), immunohistochemistry, immunofluorescence, flow cytometry, mass cytometry, mass spectrometry, western blot, and imaging-based methods, such as imaging cytometry. In some instances, the expression level of a gene (e.g., AREG, FAM177A1, RGS1, IL32, KLRB1, RORA, RORC, CXCR6, CCR6, GZMK, XCL2, CMC1, XCL1, SELL, CCL3, CCL4) is determined using single-cell RNA sequencing. In some instances, the expression level of a gene is determined in a bone marrow sample (e.g., CD138-mononuclear cells obtained from bone marrow) from a human subject having SMM or MGUS. In some instances, the expression level of a gene is determined in a blood sample (e.g., mononuclear cells, e.g., CD138-mononuclear cells, obtained from blood) from a human subject having SMM or MGUS. In some instances, the expression level of a gene is determined in a population of PBMCs isolated from a blood sample from a human subject having SMM or MGUS. The samples may be fresh or frozen samples. In some instances, the sample is a tissue section (e.g., bone marrow section) sample (e.g., Formalin-Fixed Paraffin-Embedded (FFPE) tissue specimen). In some instances, the sample is a bone marrow aspirate.

In some instances, one or more (e.g., 1, 2, 3, 4, 5, 6, 7, 8, 9, 10, 15) of the above markers or any other marker delineated herein are evaluated in combination with one or more (e.g., 1, 2, 3, 4, 5, 6, 7, 8, 9, 10, 15) genetic markers. A genetic marker is any mutation to a gene associated with a plasma cell dyscrasia or associated with susceptibility to a plasma cell dyscrasia. For example, a genetic marker can be a genetic abnormality in any gene associated with MM or associated with susceptibility to MM. Many types of genetic markers are known in the art and may include mutations to a chromosome and/or mutations to the genetic sequence. Genetic markers are shown herein using standard mutation nomenclature (den Dunnen and Anonarakis, 2000 Human Mutation 15:7-12). For example, the nomenclature of t (A;B) indicates a translocation which joins chromosomes shown in the parentheses. For example, a p. indicates a substitution in the protein with the wild type amino acid appearing before the residue number and the mutated amino acid following the residue number. An asterisk (*) in place of the second amino acid indicates a stop codon has been introduced.

In some instances, one or more (e.g., 1, 2, 3, 4, 5, 6, 7, 8, 9, 10, 15) of the above markers or any other marker delineated herein are evaluated in combination with one or more (e.g., 1, 2, 3, 4, 5, 6, 7, 8, 9, 10, 15) genetic markers described in US Patent Application Publication No. 2018-0305766-A1, which is incorporated by reference herein in its entirety. In some instances, one or more (e.g., 1, 2, 3, 4, 5, 6, 7, 8, 9, 10, 15) genetic markers are selected from: (i) one or more (e.g., 1, 2, 3, 4, 5) translocations selected from the group consisting of t (4;14), t (6;14), t (11;14), t (14;16), and t (14;20); (ii) one or more (e.g., 1, 2, 3, 4, 5, 6, 7) copy number variations (CNVs) selected from the group consisting of a 1q21 amplification, a 1p32 deletion, a 6q deletion, a 13q deletion, a 14q deletion, a 16q deletion, and a 17p deletion; (iii) one or more (e.g., 1, 2, 3, 4, 5) single nucleotide variations (SNVs) in one or more (e.g., 1, 2, 3, 4, 5, 10, 15) genes selected from the group consisting of KRAS, NRAS, BRAF, IRF4, FAM46C, DIS3, MAX, IGLL5, TRAF3, DUSP2, TCL1A, TRAF2, CYLD, LTB, HISTIHIE, BCL7A, SP140, NFKBIA, EGR1, PRKD2, RB1, TGDS, PTPN11, FUBP1, RPL5, RPL10, FGFR3, SAMHD1, ACTG1, HISTIHIB, NFKB2, KMT2B, KLHL6, RASA2, PIM1, PRDM1, SETD2, BHLHE41, BTG1, CCND1, RPRD1B, HIST1H1D, ZNF292, RFTN1, CDKNIB, XBP1, IRF1, POTI, ZFP36L1, TET2, ARID2, KDM6A, EP300, ARIDIA, NCOR1, HUWE1, SF3B1, CDKN2C, ATM, NF1, CREBBP, DNMT3A, MAF, MAFB, KDM5C, UBR5, IDH1, IKBKB, MYC, CDK8, and CXCR4; (iv) one or more (e.g., 1, 2, 3, 4, 5) SNVs selected from the group consisting of KRAS (p.G12D), KRAS (p.Q61H), NRAS (p.G12D), BRAF (p.G469R), and IRF4 (p.L116R); (v) one or more (e.g., 1, 2, 3, 4, 5) SNVs in one or more (e.g., 1, 2, 3, 4, 5) genes of the MAPK pathway (e.g., KRAS, NRAS, BRAF, PTPN11); (vi) presence of deletion of the short arm of chromosome 17 (Del17p), (vii) one or more (e.g., 1, 2, 3, 4, 5) SNVs in one or more (e.g., 1, 2, 3, 4, 5) genes of the DNA repair pathway (e.g., TP53, ATM, ATR); (viii) presence of a translocation and/or copy number amplification or SNV in Myc. In some instances, the one or more genetic markers comprises or consists of Del17p. In some instances, the one or more genetic markers comprises or consists of one or more (e.g., 1, 2, 3, 4, 5) mutations in one or more (e.g., 1, 2, 3, 4, 5) genes of the MAPK pathway (e.g., one or more of KRAS, NRAS, BRAF, and PTPN11). In some instances, the one or more genetic markers comprises or consists of one or more (e.g., 1, 2, 3, 4, 5) mutations in one or more (e.g., 1, 2, 3, 4, 5) genes of the DNA repair pathway (e.g., one or more of TP53, ATM, and ATR). In some instances, the one or more genetic markers comprises or consists of one or more (e.g., 1, 2, 3, 4, 5) translocations involving the heavy chain of immunoglobulin gene (IgH) and partner genes such as CCND1, CCND3, MAF, MAFB, WHSCI or FGFR3. In some instances, the one or more genetic markers comprises or consists of one or more (e.g., 1, 2, 3, 4, 5) chromosomal alterations, such as Del13q, Dellp, Del14q, Del16q, Amplq, Trisomy 9, Trisomy 11, hyperdiploidy. In some instances, the one or more genetic markers comprises or consists of one or more (e.g., 1, 2, 3, 4, 5) mutations in one or more (e.g., 1, 2, 3, 4, 5) genes that have driver activity in MM (e.g., one or more of KRAS, NRAS, BRAF, IRF4, FAM46C, DIS3, MAX, IGLL5, TRAF3, DUSP2, TCL1A, TRAF2, CYLD, LTB, HISTIHIE, BCL7A, SP140, NFKBIA, EGR1, PRKD2, RB1, TGDS, PTPN11, FUBP1, RPL5, RPL10, FGFR3, SAMHD1, ACTG1, HIST1H1B, NFKB2, KMT2B, KLHL6, RASA2, PIM1, PRDM1, SETD2, BHLHE41, BTG1, CCND1, RPRD1B, HISTIHID, ZNF292, RFTN1, CDKNIB, XBP1, IRF1, POTI, ZFP36L1, TET2, ARID2, KDM6A, EP300, ARIDIA, NCOR1, HUWE1, SF3B1, CDKN2C, ATM, NF1, CREBBP, DNMT3A, MAF, MAFB, KDM5C, UBR5, IDH1, IKBKB, MYC, CDK8, and CXCR4).

### Methods of Selecting Subjects for Treatment and Methods of Treatment

Markers can be used to select subjects (e.g., humans) with SMM or MGUS that would benefit from early treatment (i.e., before progression to MM). Patients with MM can also present with an increased immune reactivity and, thus, are also more likely to respond to treatment with, e.g., immunotherapy. Thus, prior to undergoing treatment for SMM, MGUS, or MM, subjects (e.g., human) with SMM, MGUS, or MM having one or more (e.g., 1, 2, 3, 4, 5, 6, 7, 8, 9, 10, 11) of the markers from Table 1 are selected for treatment, and are predicted to have significantly longer progression-free survival upon treatment (e.g., with immunotherapy) and are, thus, predicted to benefit from treatment (e.g., treatment for MM subjects or early treatment for SMM or MGUS subjects, i.e., treatment before progression from SMM or MGUS to MM). Accordingly, provided herein is a method for identifying a human subject having SMM, MGUS, or MM that would benefit from treatment, the method comprising determining that a sample (e.g., mononuclear cells obtained from a blood sample, CD138-mononuclear cells obtained from a bone marrow sample or a blood sample, or a bone marrow tissue section) obtained from the human subject has one or more (e.g., 1, 2, 3, 4, 5, 6, 7, 8, 9, 10, 11) of the immune markers from Table 1, wherein the sample is obtained prior to treatment. In some instances, the method comprises obtaining the sample from the human subject. In some instances, the human subject has not or is not currently undergoing treatment for SMM, MGUS, or MM at the time the sample is obtained from the human subject. In some instances, the method comprises determining that the sample has markers 1i, 1ii, and 1vii (see Table 1). In some instances, the method comprises determining that the sample has markers liii and Iviii (see Table 1). In some instances, the method comprises determining that the sample has markers 1vi and 1ix (see Table 1). In some instances, the method comprises determining that the sample has markers 1i-1vi and 1x (see Table 1). In some instances, the method comprises determining that the sample has markers 1ix and 1x (see Table 1). In some instances, the method comprises determining that the sample has markers 1i-1xi (see Table 1). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method further comprises (i.e., after the determining) administering to the human subject a treatment for SMM, MGUS, or MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, and a therapeutically effective dose of dexamethasone.

Prior to undergoing treatment for SMM, MGUS, or MM, subjects (e.g., human) with SMM, MGUS, or MM having one or more (e.g., 1, 2, 3, 4, 5, 6, 7, 8, 9, 10, 11) of the immune markers from Table 2 are predicted to have significantly shorter progression-free survival upon treatment with, e.g., immunotherapy. Thus, human subjects with SMM, MGUS, or MM having one or more (e.g., 1, 2, 3, 4, 5, 6, 7, 8, 9, 10, 11) of the immune markers from Table 2 are predicted to benefit from treatment (e.g., early treatment for SMM or MGUS subjects, i.e., treatment before progression from SMM or MGUS to MM) with one or more therapeutic agents (e.g., one or more therapeutic agents for the treatment of SMM, MGUS, or MM) in addition to or instead of immunotherapy. Accordingly, provided herein is a method for identifying a human subject having SMM, MGUS, or MM that would benefit from treatment, the method comprising determining that a sample (e.g., mononuclear cells obtained from a blood sample, CD138-mononuclear cells obtained from a bone marrow sample or a blood sample, or a bone marrow tissue section) obtained from the human subject has one or more (e.g., 1, 2, 3, 4, 5, 6, 7, 8, 9, 10, 11) of the immune markers from Table 2, wherein the sample is obtained prior to treatment. In some instances, the method comprises obtaining the sample from the human subject. In some instances, the human subject has not or is not currently undergoing treatment for SMM, MGUS, or MM at the time the sample is obtained from the human subject. In some instances, the method comprises determining that the sample has markers 2i, 2ii, and 2vii (see Table 2). In some instances, the method comprises obtaining the sample from the human subject. In some instances, the method comprises determining that the sample has markers 2iii and 2viii (see Table 2). In some instances, the method comprises determining that the sample has markers 2vi and 2ix (see Table 2). In some instances, the method comprises determining that the sample has markers 2i-2vi and 2x (see Table 2). In some instances, the method comprises determining that the sample has markers 2ix and 2x (see Table 2). In some instances, the method comprises determining that the sample has markers 2i-2xi (see Table 2). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method further comprises (i.e., after the determining) administering to the human subject a treatment for SMM, MGUS, or MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, a therapeutically effective dose of dexamethasone, and one or more (e.g., 1, 2, 3) additional therapeutic agents. In some instances, the treatment does not comprise immunotherapy. In some instances, the treatment does not comprise elotuzumab, lenalidomide, and/or dexamethasone.

The markers from Tables 1 and 2 are also useful in methods of treating SMM, MGUS, or MM in human subjects. For instance, as described above, subjects (e.g., human) with SMM, MGUS, or MM having one or more (e.g., 1, 2, 3, 4, 5, 6, 7, 8, 9, 10, 11) of the immune markers from Table 1 prior to treatment (e.g., with immunotherapy) are predicted to have significantly longer progression-free survival upon treatment (e.g., with immunotherapy) and, thus, are predicted to benefit from treatment (e.g., early treatment for SMM or MGUS subjects, i.e., treatment before progression from SMM or MGUS to MM). Accordingly, provided herein is a method for treating SMM, MGUS, or MM in a human subject, the method comprising administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein a sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7, 8, 9, 10, 11) of the immune markers from Table 1. In some instances, the method is for treating SMM. In some instances, the method is for treating MGUS. In some instances, the method is for treating MM. In some instances, the method comprises obtaining the sample from the human subject and determining the one or more immune biomarkers. In some instances, the human subject has not or is not currently undergoing treatment for SMM, MGUS, or MM at the time the sample was determined to have the one or more markers. In some instances, the sample has previously been determined to have markers 1i, 1ii, and 1vii (see Table 1). In some instances, the sample has previously been determined to have markers 1iii and 1viii (see Table 1). In some instances, the sample has previously been determined to have markers 1vi and 1ix (see Table 1). In some instances, the method comprises determining that the sample has markers 1i-1vi and 1x (see Table 1). In some instances, the sample has previously been determined to have markers 1ix and 1x (see Table 1). In some instances, the sample has previously been determined to have markers 1i-1xi (see Table 1). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, and a therapeutically effective dose of dexamethasone.

Additionally, as described above, subjects (e.g., human) with SMM, MGUS, or MM having one or more (e.g., 1, 2, 3, 4, 5, 6, 7, 8, 9, 10, 11) of the markers from Table 2 prior to treatment (e.g., with immunotherapy) are predicted to have significantly shorter progression-free survival upon treatment (e.g., with immunotherapy) and, thus, are predicted to benefit from treatment (e.g., early treatment for SMM or MGUS subjects, i.e., treatment before progression from SMM or MGUS to MM) with one or more therapeutic agents (e.g., one or more therapeutic agents for the treatment of SMM, MGUS, or MM) in addition to or instead of immunotherapy. Accordingly, provided herein is a method for treating SMM, MGUS, or MM in a human subject, the method comprising administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein a sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7, 8, 9, 10, 11) of the immune markers from Table 2. In some instances, the method is for treating SMM. In some instances, the method is for treating MGUS. In some instances, the method is for treating MM. In some instances, the method comprises obtaining the sample from the human subject and determining the one or more immune markers. In some instances, the sample has previously been determined to have markers 2i, 2ii, and 2vii (see Table 2). In some instances, the sample has previously been determined to have markers 2iii and 2viii (see Table 2). In some instances, the sample has previously been determined to have markers 2vi and 2ix (see Table 2). In some instances, the method comprises determining that the sample has markers 2i-2vi and 2x (see Table 2). In some instances, the sample has previously been determined to have markers 2ix and 2x (see Table 2). In some instances, the sample has previously been determined to have markers 2i-2xi (see Table 2). In some instances, the human subject has not or is not currently undergoing treatment for SMM, MGUS, or MM at the time the sample was determined to have the one or more markers. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances in which the human subject has SMM or MGUS, the treatment is administered to the human subject before the SMM or MGUS progresses to MM (e.g., overt MM). In some instances, the treatment is a treatment for SMM described herein. In some instances, the treatment is a treatment for MGUS described herein. In some instances, the treatment is a treatment for MM described herein. In some instances, the treatment is a triplet therapy (e.g., as described herein). In some instances, the treatment is a quadruplet therapy (e.g., as described herein). In some instances, the treatment is a therapeutically effective dose of elotuzumab, a therapeutically effective dose of lenalidomide, a therapeutically effective dose of dexamethasone, and one or more (e.g., 1, 2, 3) additional therapeutic agents. In some instances, the treatment does not comprise immunotherapy.

### Methods of Monitoring Subjects During Treatment and Methods of Treatment

Markers described herein can be used to monitor the response to treatment (e.g., immunotherapy) in a subject (e.g., human) with SMM, MGUS, or MM and to select those subjects with SMM, MGUS, or MM that should be treated with a different therapeutic regimen (e.g., alternate agent, alternate dose).

For instance, subjects (e.g., human) with SMM, MGUS, or MM having one or more (e.g., 1, 2, 3, 4, 5, 6, 7) of the immune markers from Table 3 while undergoing treatment (e.g., immunotherapy) for SMM, MGUS, or MM are predicted to have significantly shorter progression-free survival upon treatment (e.g., with immunotherapy). Thus, human subjects with SMM, MGUS, or MM having one or more (e.g., 1, 2, 3, 4, 5, 6, 7) of the immune markers from Table 3 while undergoing treatment (e.g., immunotherapy) are predicted to benefit from a different, e.g., more intensive, treatment (e.g., higher dose(s), more dose(s), combination therapy, or a different therapy from that being used). Accordingly, provided herein is a method for monitoring a human subject having SMM, MGUS, of MM undergoing treatment for SMM, MGUS, or MM, the method comprising determining that a sample (e.g., blood or bone marrow, e.g., mononuclear cells obtained from blood or CD138-mononuclear cells obtained from bone marrow or blood) obtained from the human subject has one or more of (e.g., 1, 2, 3, 4, 5, 6, 7) the immune markers from Table 3. In some instances, the method is for treating SMM. In some instances, the method is for treating MGUS. In some instances, the method is for treating MM. In some instances, the method comprises obtaining the sample from the human subject. In some instances, the sample is obtained while the human subject is undergoing the treatment for SMM, MGUS, or MM (e.g., has received/is receiving one or more (e.g., 1, 2, 3) doses of the treatment of SMM, MGUS, or MM, e.g., immunotherapy and optionally a steroid, e.g., elotuzumab, lenalidomide, and dexamethasone). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method further comprises (i.e., after the determining) administering to the human subject a different, e.g., more intensive, treatment (e.g., higher dose(s), more dose(s), combination therapy, or a different therapy from that being used) for SMM, MGUS, or MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the alternate therapy comprises or consists of a bispecific antibody targeting BCMA (e.g., an anti-BCMA/anti-CD3 bispecific antibody, e.g., Teclistamab).

Markers described herein can be used to monitor the response to treatment (e.g., immunotherapy) in a subject (e.g., human) with SMM, MGUS, or MM and determine which subjects with SMM, MGUS, or MM should be treated with the same treatment (e.g., immunotherapy).

For instance, subjects (e.g., human) with SMM, MGUS, or MM having one or more (e.g., 1, 2, 3, 4, 5, 6, 7) of the markers from Table 4 while undergoing treatment (e.g., immunotherapy) for SMM, MGUS, or MM are predicted to have significantly longer progression-free survival upon treatment (e.g., with immunotherapy). Thus, human subjects with SMM, MGUS, or MM having one or more (e.g., 1, 2, 3, 4, 5, 6, 7) of the immune markers from Table 4 while undergoing treatment (e.g., immunotherapy) are predicted to benefit from continuation of the treatment. Accordingly, provided herein is a method for monitoring a human subject having SMM or MGUS undergoing treatment for SMM, MGUS, or MM, the method comprising determining that a sample (e.g., blood or bone marrow, e.g., mononuclear cells obtained from blood or CD138-mononuclear cells obtained from bone marrow or blood) obtained from the human subject has one or more of (e.g., 1, 2, 3, 4, 5, 6, 7) the immune markers from Table 4. In some instances, the method comprises obtaining the sample from the human subject. In some instances, the sample is obtained while the human subject is undergoing the treatment for SMM, MGUS, or MM (e.g., has received/is receiving one or more (e.g., 1, 2, 3) doses of the treatment of SMM, MGUS, or MM, e.g., immunotherapy and optionally a steroid, e.g., elotuzumab, lenalidomide, and dexamethasone). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the method comprises administering the treatment indefinitely. In some instances, the method comprises administering the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject 1, 2, 3, 4, 5, 6, 7, 8, 9, 10 or more additional doses of the treatment. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab).

The immune markers from Tables 3 and 4 are also useful in methods of treating SMM, MGUS, or MM in human subjects. For example, as described above, subjects (e.g., human) with SMM, MGUS, or MM having one or more (e.g., 1, 2, 3, 4, 5, 6, 7) of the immune markers from Table 3 while undergoing treatment (e.g., with immunotherapy), are predicted to have significantly shorter progression-free survival, and thus, are predicted to benefit from a different, e.g., more intensive, treatment (e.g., higher dose(s), more dose(s), combination therapy, or a different therapy from that being used). Accordingly, provided herein is a method for treating SMM, MGUS, or MM in a human subject, the method comprising administering to the human subject a therapeutically effective amount of a treatment for SMM, MGUS, or MM (e.g., immunotherapy, e.g., immunotherapy in combination with steroid, e.g., elotuzumab, lenalidomide, and dexamethasone); wherein a sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7) of the immune markers from Table 3, wherein the human subject was undergoing a first treatment for SMM, MGUS, or MM (e.g., immunotherapy) when the sample was obtained from the human subject, and wherein the treatment administered to the human subject is different from the first treatment. In some instances, the method is for treating SMM. In some instances, the method is for treating MGUS. In some instances, the method is for treating MM. In some instances, the method comprises obtaining the sample from the human subject. In some instances, the human subject was undergoing treatment (e.g., had received/was receiving one or more (e.g., 1, 2, 3) doses of one or more (e.g., 1, 2, 3, 4) therapeutic agents for the treatment of SMM, MGUS, or MM, e.g., immunotherapy and optionally a steroid, e.g., elotuzumab, lenalidomide, and dexamethasone) at the time the sample was obtained from the human subject. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the first treatment comprises or consists of a treatment for SMM described herein. In some instances, the first treatment comprises or consists of a treatment for MGUS described herein. In some instances, the first treatment comprises or consists of a treatment for MM described herein. In some instances, the first treatment comprises or consists of elotuzumab, lenalidomide, and dexamethasone. In some instances, the first treatment comprises or consists of a triplet therapy (i.e., a combination of a proteasome inhibitor, an immunomodulatory drug, and a steroid, e.g., dexamethasone). In some instances, the first treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab). In some instances, the treatment administered to the human subject is more intensive than the first treatment (e.g., higher dose(s), more dose(s), combination therapy, or a different therapy from that being used). In some instances, the treatment administered to the human subject comprises or consists of a treatment for SMM described herein. In some instances, the treatment administered to the human subject comprises or consists of a treatment for MGUS described herein. In some instances, the treatment administered to the human subject comprises or consists of a treatment for MM described herein. In some instances, the treatment administered to the human subject comprises or consists of elotuzumab, lenalidomide, and dexamethasone. In some instances, the treatment administered to the human subject comprises or consists of a triplet therapy (i.e., a combination of a proteasome inhibitor, an immunomodulatory drug, and a steroid, e.g., dexamethasone). In some instances, the treatment administered to the human subject comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment administered to the human subject treatment comprises or consists of autologous stem cell transplantation (ASCT). In some instances, the treatment administered to the human subject comprises or consists of CAR-T cells targeting BCMA (e.g., Abecma® [idecabtagene vicleucel]). In some instances, the treatment administered to the human subject comprises or consists of a bispecific antibody targeting BCMA (e.g., an anti-BCMA/anti-CD3 bispecific antibody, e.g., Teclistamab). In some instances, the treatment is administered to the human subject before the SMM or MGUS progresses to MM (e.g., overt MM).

With respect to the immune markers from Table 4, as described above, subjects (e.g., human) with SMM, MGUS, or MM having one or more (e.g., 1, 2, 3, 4, 5, 6, 7) of the immune markers from Table 4 while undergoing treatment (e.g., with immunotherapy), are predicted to have significantly longer progression-free survival, and thus, are predicted to benefit from continuing treatment. Accordingly, provided herein is a method for treating SMM, MGUS, or MM in a human subject, the method comprising administering to the human subject a treatment for SMM, MGUS, or MM; wherein a sample (e.g., blood or bone marrow, e.g., mononuclear cells obtained from blood or CD138-mononuclear cells obtained from bone marrow or blood) obtained from the human subject has previously been determined to have one or more (e.g., 1, 2, 3, 4, 5, 6, 7) of the immune markers from Table 4, wherein the human subject was undergoing the treatment when the sample was obtained from the human subject. In some instances, the method is for treating SMM. In some instances, the method is for treating MGUS. In some instances, the method is for treating MM. In some instances, the method comprises obtaining the sample from the human subject. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method comprises continuing the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject 1, 2, 3, 4, 5, 6, 7, 8, 9, 10 or more additional doses of the treatment. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab).

Methods of Assessing Response after Treatment and Methods of Treatment

Markers described herein can be used to characterize and/or determine the response to treatment (e.g., immunotherapy) in a subject (e.g., human) with SMM, MGUS, or MM who has undergone or is undergoing (e.g., received 1, 2, 3, or more doses of a treatment for SMM, MGUS, or MM, e.g., immunotherapy) treatment for SMM, MGUS, MM and determine which subjects with SMM, MGUS, or MM are predicted to have prolonged or shortened biochemical progression-free survival. Biochemical progression free survival includes both clinical and biochemical progression. In some instances, biochemical progression free survival comprises (i) a significant increase in tumor burden (e.g., as determined by M-spike levels or free light chain (FLC) ratio) during treatment (ii) in the absence of a myeloma-defining event (e.g., as described above).

Markers described herein can be used identify subjects (e.g., humans) with SMM, MGUS, or MM who have undergone (e.g., received 1, 2, 3, or more doses) treatment for SMM, MGUS, or MM (e.g., immunotherapy) that would benefit from termination or modification of treatment. For instance, subjects (e.g., human) with SMM, MGUS, or MM having an immune marker from Table 5 after undergoing treatment (e.g., immunotherapy) for SMM, MGUS, or MM are predicted to have significantly longer biochemical progression-free survival upon treatment (e.g., with immunotherapy). Thus, human subjects with SMM, MGUS, or MM having an immune marker from Table 5 after undergoing treatment (e.g., within one day, within one week, within one month, within two months, within three months, within six months, or within one year of the last dose of the treatment (e.g., immunotherapy and optionally a steroid, e.g., elotuzumab, lenalidomide, and dexamethasone)) are predicted to benefit from termination of the treatment or modification of the treatment (e.g., changes in amount, duration, or type of treatment; changes in the frequency of follow-up assessments or the type of tests performed clinically). Accordingly, provided herein is a method for identifying a human subject having SMM, MGUS, or MM who has undergone (e.g., received 1, 2, 3, or more doses) treatment for SMM, MGUS, or MM (e.g., immunotherapy) that would benefit from terminating or modifying (e.g., changes in amount, duration, or type of treatment; changes in the frequency of follow-up assessments or the type of tests performed clinically) the treatment, the method comprising determining that a sample (e.g., blood or bone marrow, e.g., mononuclear cells obtained from blood or CD138-mononuclear cells obtained from bone marrow or blood) obtained from the human subject has an immune marker from Table 5. In some instances, the method comprises obtaining the sample from the human subject. In some instances, the sample is obtained from the human subject within one day, within one week, within one month, within two months, within three months, within six months, or within one year of the last dose of the treatment (e.g., immunotherapy and optionally a steroid, e.g., elotuzumab, lenalidomide, and dexamethasone). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method further comprises (i.e., after the determining) terminating the treatment. In some instances, the method comprises terminating the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method further comprises (i.e., after the determining) modifying the treatment (e.g., changes in amount, duration, or type of treatment; changes in the frequency of follow-up assessments or the type of tests performed clinically). In some instances, the modifying of the treatment is to reduce treatment (e.g., reduce the amount, reduce the duration, reduce the doses). For example, in some instances, the method comprises decreasing the dose of the treatment. In some instances, the method comprises decreasing the frequency of the dose of the medication. In some instances, the method comprises decreasing the frequency of follow-up assessments. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab).

Subjects (e.g., human) with SMM, MGUS, or MM having an immune marker from Table 6 after undergoing treatment (e.g., immunotherapy) for SMM, MGUS, or MM are predicted to have significantly shorter progression-free survival upon treatment (e.g., with immunotherapy). Thus, human subjects with SMM, MGUS, or MM having an immune marker from Table 6 after undergoing treatment (e.g., within one day, within one week, within one month, within two months, within three months, within six months, or within one year of the last dose of the treatment (e.g., immunotherapy and optionally a steroid, e.g., elotuzumab, lenalidomide, and dexamethasone)) are predicted to benefit from a different, e.g., more intensive, treatment (e.g., higher dose(s), more dose(s), combination therapy, or a different therapy from that being used)). Accordingly, provided herein is a method for identifying a human subject having SMM, MGUS, or MM who has undergone (e.g., received 1, 2, 3, or more doses) treatment for SMM, MGUS, or MM (e.g., immunotherapy) that would benefit from a different, e.g., more intensive, treatment (e.g., higher dose(s), more dose(s), combination therapy, or a different therapy from that being used), the method comprising determining that a sample (e.g., blood or bone marrow, e.g., mononuclear cells obtained from blood or CD138-mononuclear cells obtained from bone marrow or blood) obtained from the human subject has an immune marker from Table 6. In some instances, the method comprises obtaining the sample from the human subject. In some instances, the sample is obtained from the human subject within one day, within one week, within one month, within two months, within three months, within six months, or within one year of the last dose of the treatment (e.g., immunotherapy and optionally a steroid, e.g., elotuzumab, lenalidomide, and dexamethasone). In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method further comprises (i.e., after the determining) administering to the human subject a different, e.g., more intensive, treatment (e.g., higher dose(s), more dose(s), combination therapy, or a different therapy from that being used) for SMM, MGUS, or MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab).

The immune markers from Tables 5 and 6 are also useful in methods of treating SMM, MGUS, or MM in human subjects. For example, as described above, subjects (e.g., human) with SMM, MGUS, or MM having a marker from Table 5 after undergoing treatment (e.g., with immunotherapy), are predicted to have significantly longer biochemical progression-free survival, and thus, are predicted to benefit from terminating or modifying the treatment (e.g., changes in amount, duration, or type of treatment; changes in the frequency of follow-up assessments or the type of tests performed clinically). Accordingly, provided herein is a method for treating SMM, MGUS, or MM in a human subject who has undergone treatment (e.g., received 1, 2, 3, or more doses) for SMM, MGUS, or MM (e.g., immunotherapy), the method comprising terminating the treatment or administering to the human subject a modified treatment (e.g., different amount, duration, or type of treatment, different frequency of follow-up assessments, different type of tests performed clinically), wherein a sample (e.g., blood or bone marrow, e.g., mononuclear cells obtained from blood or CD138-mononuclear cells obtained from bone marrow or blood) obtained from the human subject has previously been determined to have an immune marker from Table 5, and wherein the sample was obtained from the human subject after (e.g., within one day, within one week, within one month, within two months, within three months, within six months, or within one year of) the last dose of the treatment. In some instances, the method is for treating SMM. In some instances, the method is for treating MGUS. In some instances, the method is for treating MM. In some instances, the method comprises obtaining the sample from the human subject. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances, the method comprises (i.e., after the determining) terminating the treatment. In some instances, the method comprises terminating the treatment indefinitely. In some instances, the method comprises terminating the treatment for a period of time (e.g., at least 1 month, at least 2 months, at least 3 months, at least 4 months, at least 5 months, at least 6 months, at least 1 year, at least 2 years, at least 5 years). In some instances, the method comprises (i.e., after the determining) administering to the human subject a modified treatment (e.g., different amount, duration, or type of treatment, different frequency of follow-up assessments, different type of tests performed clinically). In some instances, the modified treatment is a reduced treatment (e.g., reduced amount, reduced duration, reduce number of doses). For example, in some instances, the method comprises administering to the human subject a decreased dose of the treatment. In some instances, the method comprises administering to the human subject a decreased frequency of the dose of the medication. In some instances, the method comprises decreasing the frequency of follow-up assessments. In some instances, the treatment is a treatment for SMM described herein. In some instances, the treatment is a treatment for MGUS described herein. In some instances, the treatment is a treatment for MM described herein. In some instances, the treatment comprises or consists of a triplet therapy (i.e., a combination of a proteasome inhibitor, an immunomodulatory drug, and a steroid, e.g., dexamethasone). In some instances, the treatment comprises or consists of elotuzumab, lenalidomide, and dexamethasone. In some instances, the treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the treatment comprises or consists of autologous stem cell transplantation (ASCT). In some instances, the treatment comprises or consists of CAR-T cells targeting BCMA (e.g., Abecma® [idecabtagene vicleucel]). In some instances, the treatment comprises or consists of a bispecific antibody targeting BCMA (e.g., an anti-BCMA/anti-CD3 bispecific antibody, e.g., Teclistamab). In some instances, the modified treatment is a treatment for SMM described herein. In some instances, the modified treatment is a treatment for MGUS described herein. In some instances, the modified treatment is a treatment for MM described herein. In some instances, the modified treatment comprises or consists of a triplet therapy (i.e., a combination of a proteasome inhibitor, an immunomodulatory drug, and a steroid, e.g., dexamethasone). In some instances, the modified treatment comprises or consists of elotuzumab, lenalidomide, and dexamethasone. In some instances, the modified treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the modified treatment comprises or consists of autologous stem cell transplantation (ASCT). In some instances, the treatment comprises or consists of CAR-T cells targeting BCMA (e.g., Abecma® [idecabtagene vicleucel]). In some instances, the modified treatment comprises or consists of a bispecific antibody targeting BCMA (e.g., an anti-BCMA/anti-CD3 bispecific antibody, e.g., Teclistamab).

As described above, subjects (e.g., human) with SMM, MGUS, or MM having a marker from Table 6 after undergoing treatment (e.g., with immunotherapy), are predicted to have significantly shorter biochemical progression-free survival, and thus, are predicted to benefit from a different, e.g., more intensive, treatment (e.g., higher dose(s), more dose(s), combination therapy, or a different therapy from that being used)). Accordingly, provided herein is a method for treating SMM, MGUS, or MM in a human subject who has undergone treatment (e.g., received 1, 2, 3, or more doses) for SMM, MGUS, or MM (e.g., immunotherapy), the method comprising administering to the human subject a different, e.g. more intensive, treatment (e.g., higher dose(s), more dose(s), combination therapy, or a different therapy from that being used), wherein a sample (e.g., blood or bone marrow, e.g., mononuclear cells obtained from blood or CD138-mononuclear cells obtained from bone marrow or blood) obtained from the human subject has previously been determined to have an immune marker from Table 6, and wherein the sample was obtained from the human subject after (e.g., within one day, within one week, within one month, within two months, within three months, within six months, or within one year of) the last dose of the treatment. In some instances, the method is for treating SMM. In some instances, the method is for treating MGUS. In some instances, the method is for treating MM. In some instances, the method comprises obtaining the sample from the human subject. In some instances, the human subject has SMM. In some instances, the human subject having SMM has high-risk SMM. In some instances, the human subject having SMM has high-risk SMM based on the Rajkumar et al., Blood 125:3069-3075 (2015) criteria. In some instances, the human subject having SMM has high-risk SMM based on the “20-2-20” criteria. In some instances, the human subject having SMM has low- or intermediate-risk SMM based on the “20-2-20” criteria. In some cases, the human subject having SMM has high-risk SMM based on the Rajkumar et al., criteria and the “20-2-20” criteria (Mateos et al., Blood Cancer Journal, 10 (102): (2020)). In some instances, the human subject has MGUS. In some instances, the human subject has MM. In some instances in which the human subject has SMM or MGUS, the different (e.g., more intensive) treatment is administered to the human subject before the SMM or MGUS progresses to MM (e.g., overt MM). In some instances, the more intensive treatment is a treatment for SMM described herein. In some instances, the more intensive treatment is a treatment for MGUS described herein. In some instances, the more intensive treatment is a treatment for MM described herein. In some instances, the more intensive treatment comprises or consists of a triplet therapy (i.e., a combination of a proteasome inhibitor, an immunomodulatory drug, and a steroid, e.g., dexamethasone). In some instances, the more intensive treatment comprises or consists of elotuzumab, lenalidomide, and dexamethasone. In some instances, the more intensive treatment comprises or consists of a quadruplet therapy (i.e., a combination of a monoclonal antibody that specifically binds to, e.g., SLAMF7 or CD38, a proteasome inhibitor, an immunomodulatory drug, and a steroid, e.g., dexamethasone). In some instances, the more intensive treatment comprises or consists of autologous stem cell transplantation (ASCT). In some instances, the more intensive treatment comprises or consists of CAR-T cells targeting BCMA (e.g., Abecma® [idecabtagene vicleucel]). In some instances, the more intensive treatment comprises or consists of a bispecific antibody targeting BCMA (e.g., an anti-BCMA/anti-CD3 bispecific antibody, e.g., Teclistamab).

### Subject Management

In certain embodiments, the methods of the invention involve managing subject treatment based on disease status (e.g., complete remission, partial remission, resistant disease, stable disease) or based on characterization of a biological sample (e.g., PBMCs, bone marrow) from the subject for polypeptide or polynucleotide markers delineated herein. Such management includes referral, for example, to a qualified specialist (e.g., an oncologist). In one embodiment, if a physician makes a diagnosis of a multiple myeloma (MM), then a certain regime of treatment, such as prescription or administration of therapeutic agent might follow. Alternatively, a diagnosis of non-cancer might be followed with further testing to determine a specific disease that the patient might be suffering from or to determine whether a multiple myeloma in the subject has progressed (e.g., from one state in the development of a multiple myeloma to another, such as from MGUS to SMM or from SMM to MM). In some embodiments, subject management involves monitoring of multiple myeloma (MM) status in the patient during combination therapy through regular (e.g., weekly, monthly, yearly, etc.) characterization of biological samples (e.g., PBMCs, bone marrow) from the patient. Also, if the diagnostic test gives an inconclusive result on disease status, further tests may be called for.

Additional embodiments of the invention relate to the communication of assay results or diagnoses or both to technicians, physicians, or patients. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

The disease state or treatment of a patient having SMM can be monitored using the methods and compositions of this invention. In one embodiment, the response of a patient to a treatment can be monitored using the methods and compositions of this invention. Such monitoring may be useful, for example, in assessing the efficacy of a particular treatment in a patient. Treatments amenable to monitoring using the methods of the invention include, but are not limited to, chemotherapy, radiotherapy, immunotherapy, and surgery.

### Hardware and Software

The present disclosure also relates to a computer system involved in carrying out the methods of the disclosure relating to both computations and sequencing. The methods described herein, analyses can be performed on general-purpose or specially-programmed hardware or software. One can then record the results (e.g., characterization of SMM in a biological sample) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In aspects, software used to analyze the data can include code that applies an algorithm to the analysis of the results. The software also can also use input data (e.g., sequence) to characterize a biological sample (e.g., PBMCs, bone marrow).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in characterizing a biological sample (e.g., bone marrow, PBMCs) from a subject. Kits of the disclosure may include one or more containers comprising an agent for enriching/isolating and/or characterization of SMM and/or for treatment of a multiple myeloma (MM). In some embodiments, the kits further include instructions for use in accordance with the methods of this disclosure. In some embodiments, these instructions comprise a description of use of the agent to enrich/isolate and/or characterize a biological sample of the subject and/or use of the agent for treatment of a smoldering multiple myeloma (MM). In some embodiments, the instructions comprise a description of how to isolate polynucleotides from a sample and/or to characterize the sample. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

The invention will be further described in the following examples, which do not limit the scope of the invention described in the claims.

### EXAMPLES

Subjects with Smoldering Multiple Myeloma (SMM) are typically observed until progression, but early treatment may improve outcomes. The data in this Examples section include data from a Phase II clinical trial of Elotuzumab, Lenalidomide and Dexamethasone, in subjects with high-risk SMM. These data indicate that immune biomarkers (see, e.g., Tables 1-6) can be used to predict treatment outcome, determine treatments, monitor progression, and monitor treatment response. Biomarkers were also detectable in the subjects' blood; thus, minimally invasive immune profiling for prognostication and monitoring is feasible.

Moreover, single-cell RNA-sequencing on 149 bone marrow (BM) and peripheral blood (PB) samples from patients and healthy donors was performed and provided a comprehensive characterization of alterations in immune cell composition and T-cell receptor repertoire diversity in patients. Importantly, it was shown that the similarity of a patient's immune microenvironment to that of healthy donors may have prognostic relevance at diagnosis and post-treatment, and GZMK CD8⁺ effector memory T-cells may be associated with response to treatment. Lastly, striking similarities between immune alterations observed in the BM and PB were demonstrated and that PB-based immune profiling may have diagnostic and prognostic utility.

### Example 1: Early Treatment with Elotuzumab, Lenalidomide and Dexamethasone (EloLenDex) is Safe and Effective in Patients with High-Risk Smoldering Multiple Myeloma

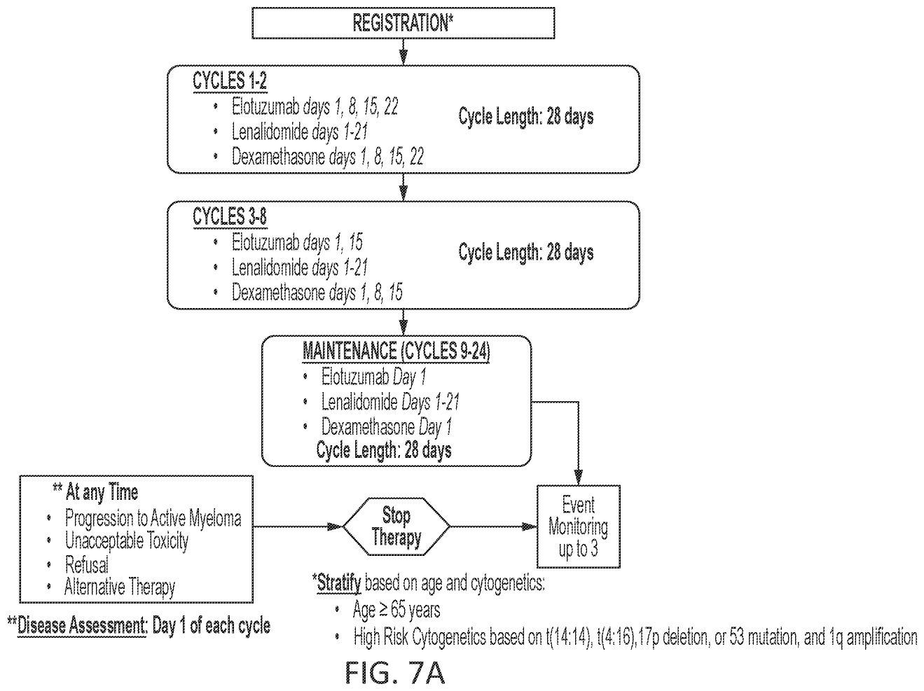

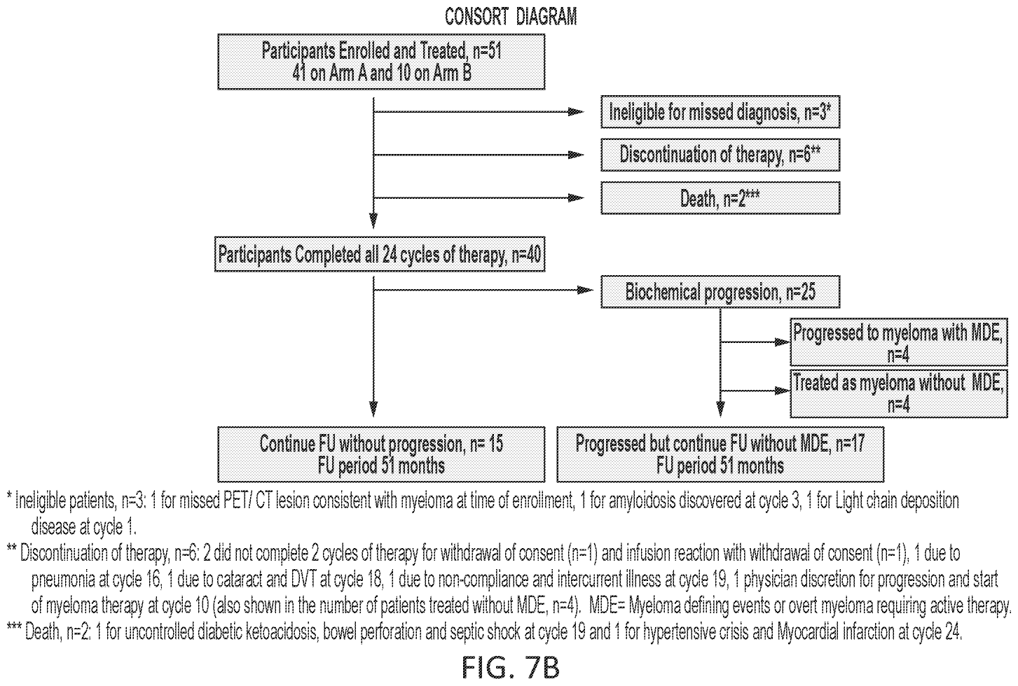

A Phase II trial of Elotuzumab, Lenalidomide, Dexamethasone was conducted in patients with high-risk smoldering multiple myeloma (SMM) (E-PRISM, n=51). Enrollment in E-PRISM was based on the high-risk SMM criteria described by Rajkumar, which comprised all risk factors shown to confer a high risk of progression and are thus more inclusive. The primary objective was to determine the proportion of patients with high-risk SMM who were progression-free at 2 years post-treatment. Patients were initially randomized 1:1 to Arm A (EloLenDex, n=40) or Arm B (EloLen, n=11), but accrual to Arm B was halted early, as the exposure to steroids was similar in both arms due to the premedication requirement for Elotuzumab infusion (Online Methods). Treatment was planned for 24 cycles followed by observation until progression to overt multiple myeloma (MM). Stem cell collection was allowed for eligible patients. The trial schema and CONSORT diagram are shown in FIGS. 7A and 7B, respectively.

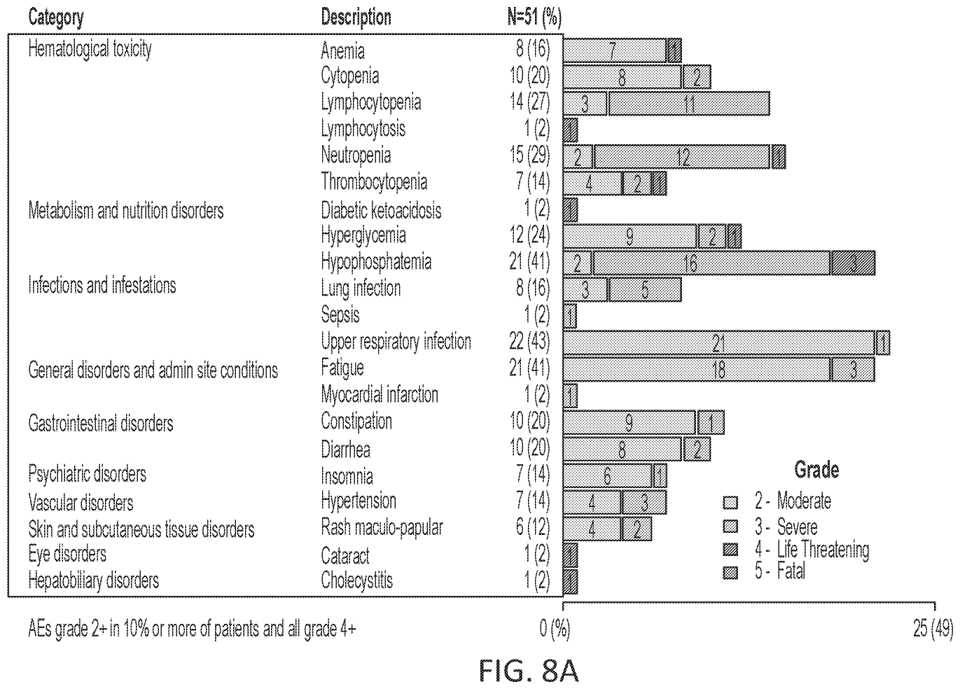

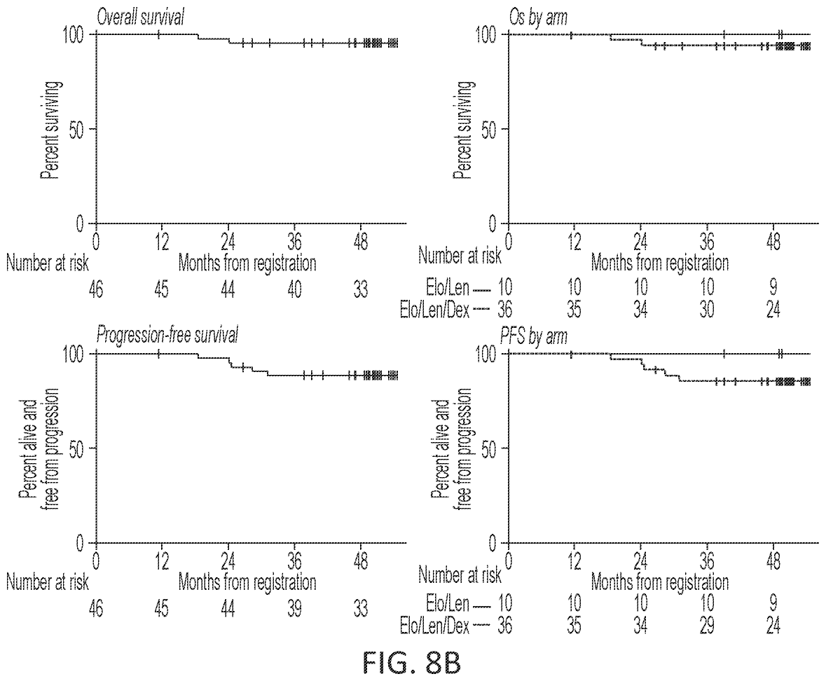

The most common toxicities were manageable FIG. 8A). Median follow-up for all 51 patients was 50 months (range 2-67). Six patients had progression-free survival (PFS) events defined as progressive disease to myeloma-defining events (n=4) or death (n=2).

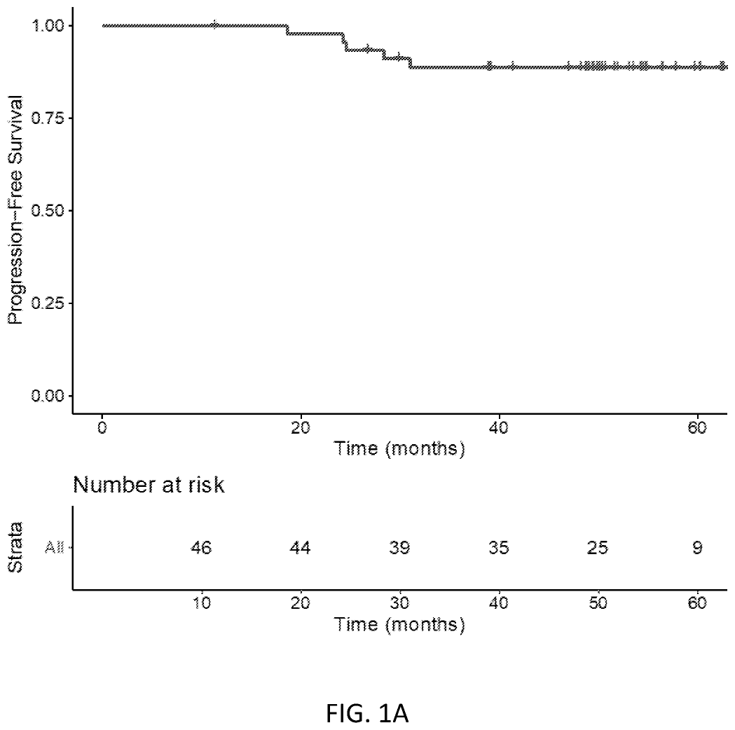

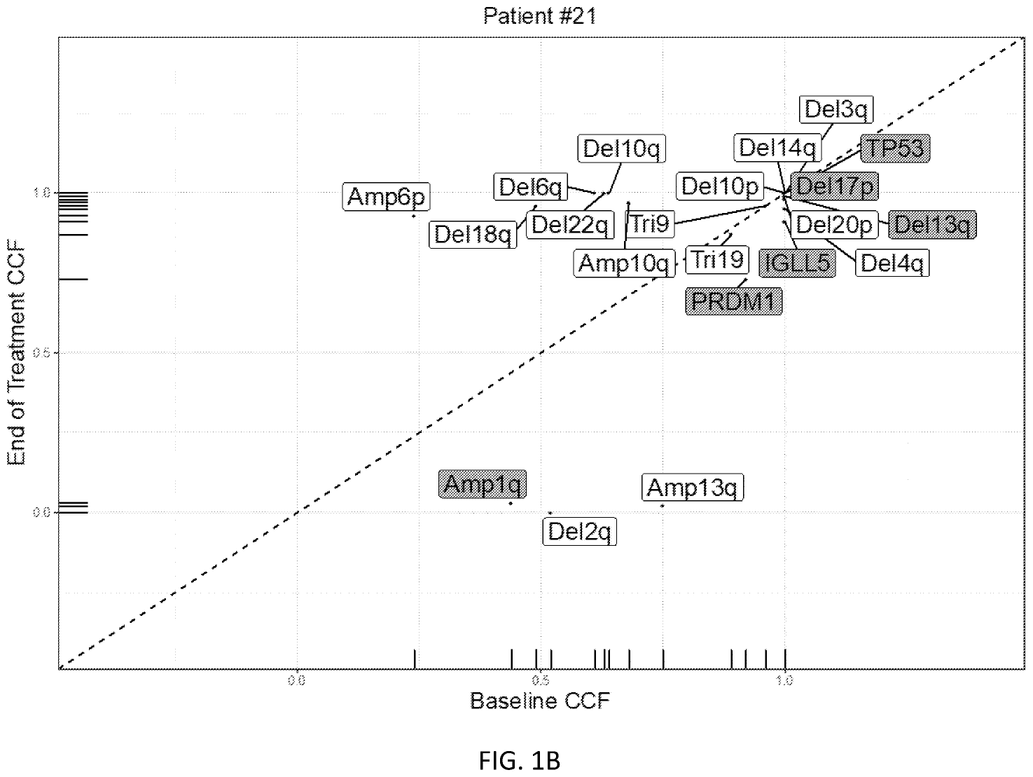

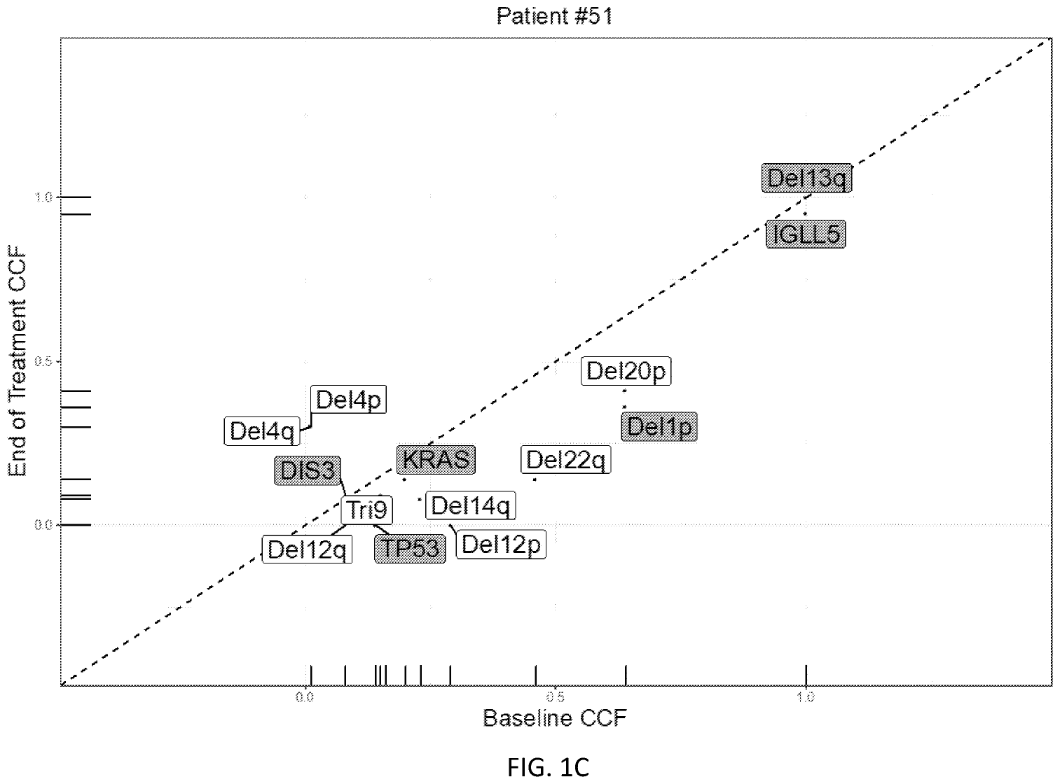

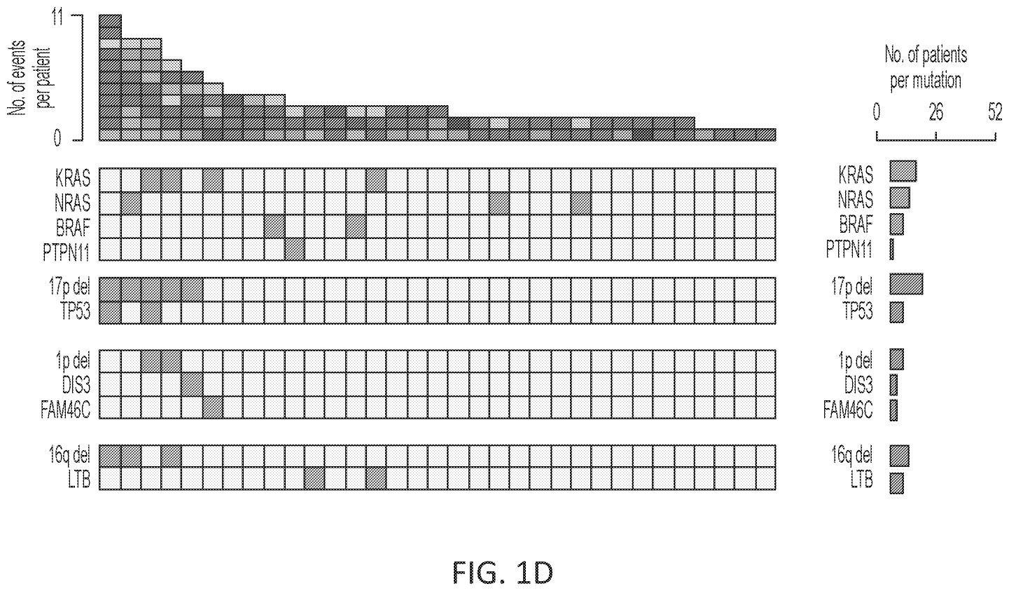

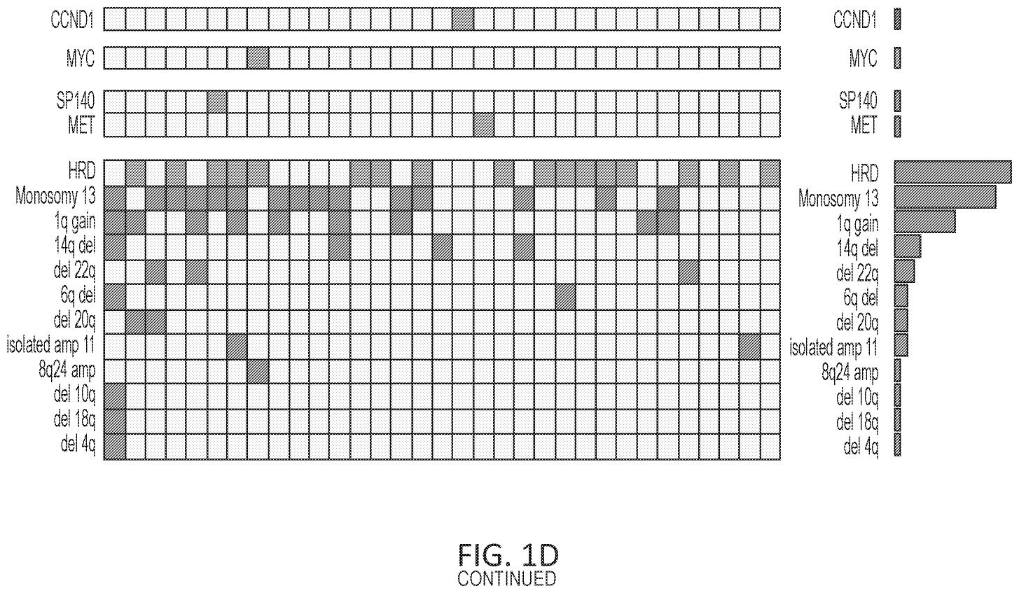

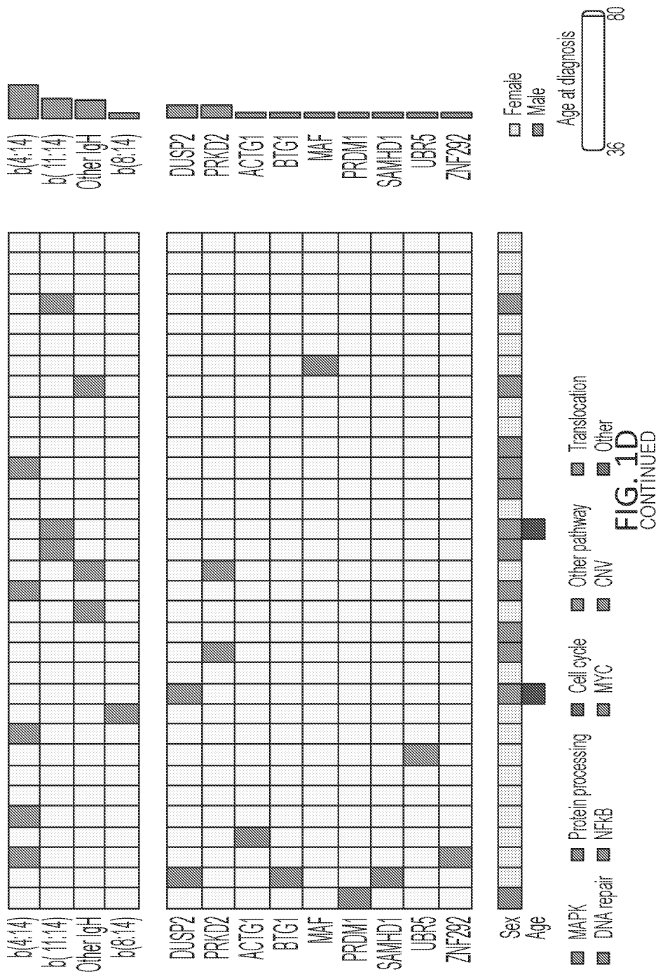

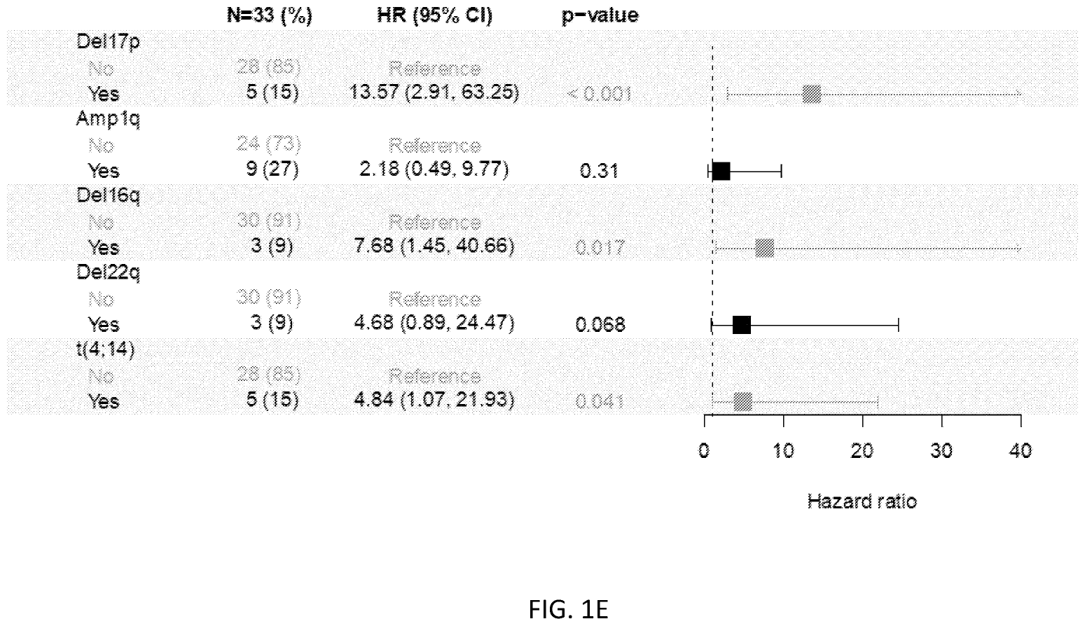

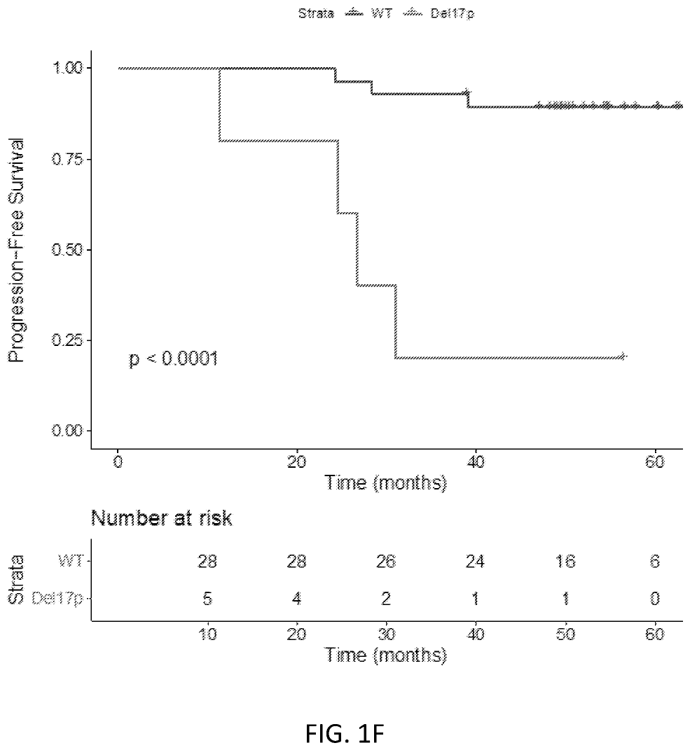

Median PFS and overall survival (OS) in the 46 patients who were eligible for the study and received at least two full doses of treatment were not reached; PFS was 88.7% at 48 months (90% CI, 81.2-96.9%) and OS at 48 months was 95.6% (90% CI, 90.6-100%) (FIG. 8B). The Kaplan-Meier curve of progression free survival in eligible patients who received at least two doses is depicted in FIG. 1A.

The overall response rate (partial response (PR) or better) for these patients was 87% (n=40). Three patients (7%) experienced a best overall response of stringent Complete Response (CR), 1 patient (2%) achieved CR, 14 patients (30%) achieved VGPR, 22 patients (48%) achieved PR, and 6 patients (13%) achieved marginal response (MR). Overall, early treatment with EloLenDex was deemed safe and effective in patients with high-risk SMM.

### Example 2: Genomic Predictors of Response to Early Therapeutic Intervention

Three main categories of progression were observed in our cohort: those who developed overt myeloma (n=4), those who were treated based on evolving biomarkers and the physician's decision (n=4), and those who presented biochemical progression (n=19). In two of the four individuals who developed overt MM and had available samples for testing, whole-exome sequencing (WES) was performed on bone marrow (BM) CD138⁺ cells taken at baseline (BL) and end of treatment (EOT), to assess whether early treatment had selected for aggressive subclones with high-risk genetic abnormalities (Online Methods). Both individuals had high-risk genomic features at baseline: patient #21 had Del17p and a TP53 single nucleotide variant (SNV), while patient #51 had t (4;14), Dellp, a TP53 and a KRAS SNV. Selection of aggressive subclones was not observed in these patients (FIGS. 1B and 1C). In patient #21, several large-scale copy number variants (CNVs) were enriched post-treatment, but none are known to be associated with an increased risk of progression. In both patients, a contraction of potentially aggressive subclones were observed that harbor Amplq (patient #21) and Dellp and a TP53 SNV (patient #51), suggesting that in certain instances, early treatment can alter the tumor's clonal dynamics.

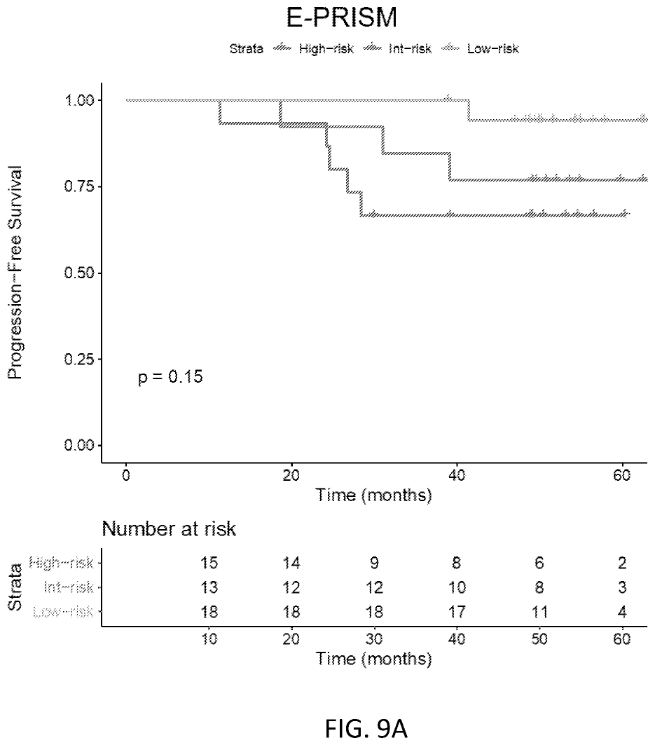

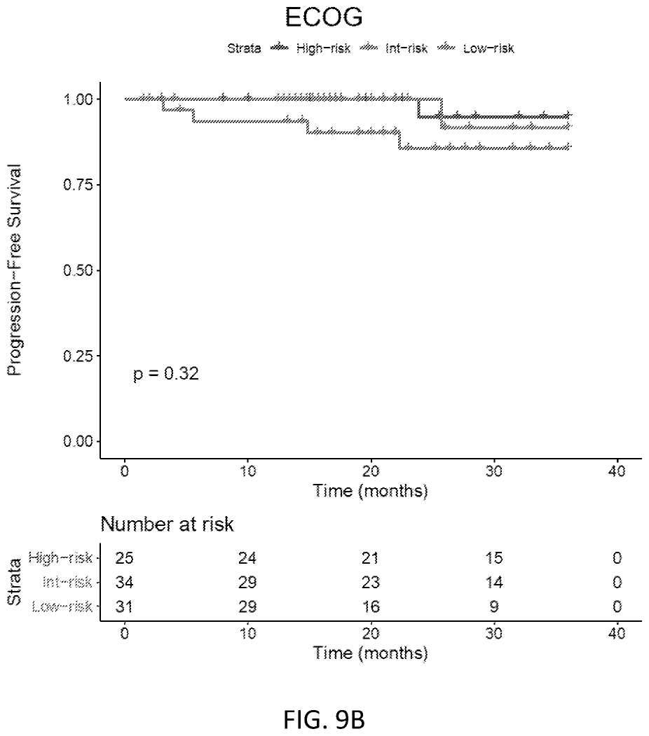

A total of 17 patients did not progress to date, suggesting that our patient selection can improve further to identify patients with high-risk SMM who are more likely to benefit from early immunotherapy. The 20-2-20 model was not a significant predictor of response in our cohort (FIG. 9A). Consistent with this result, 20-2-20 was not a significant predictor of response in the treatment arm of a Phase III randomized trial of Lenalidomide vs Observation in patients with high-risk SMM either (FIG. 9B). This stratification model relies on tumor burden metrics, such as bone marrow infiltration and M-spike, to infer disease aggressiveness. It is thus possible that in the context of immunomodulation, tumor burden does not adequately predict response to therapy.

Next, WES (n=31) and deep targeted sequencing (DTS) (n=3) was performed on BM samples collected at baseline to detect coding genetic alterations and explore their association with patient outcome (FIG. 1D). At the univariate level, testing events were present in at least 3 individuals, Del17p, t (4;14), and Del16q were significant predictors of PFS, and Del17p was an independent predictor of progression at the multivariate level (FIGS. 1E and 1F). These results suggested that patients with SMM and Del17p may need more intensive regimens to prolong PFS, a hypothesis that requires validation in a randomized trial.

### Example 3: Comprehensive Characterization of Changes in the BM Immune Cell Composition and T-Cell Receptor Repertoire of Patients with High-Risk SMM

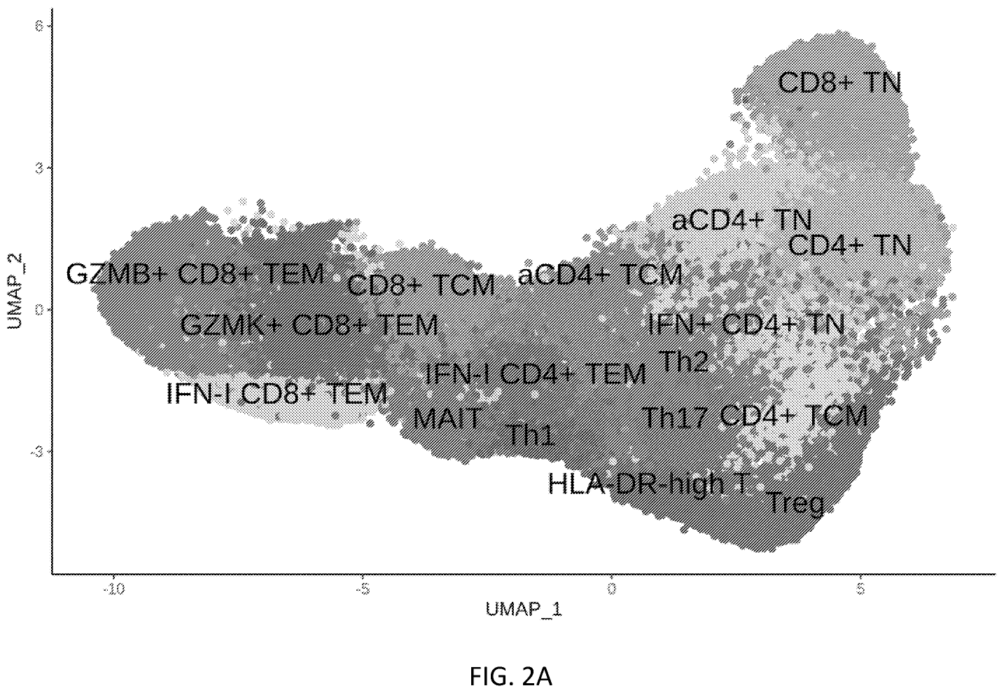

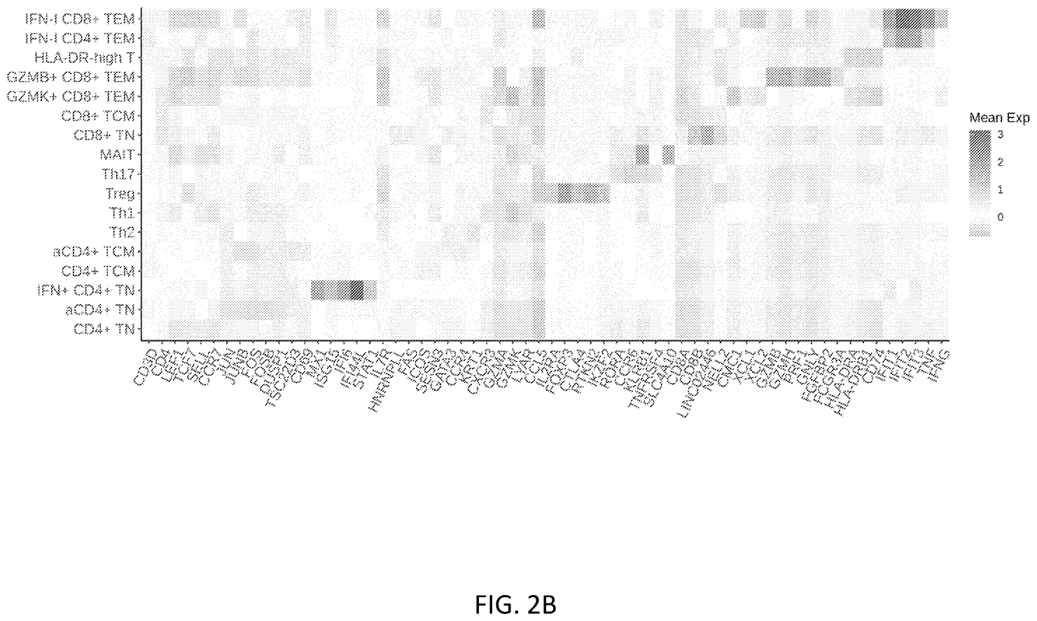

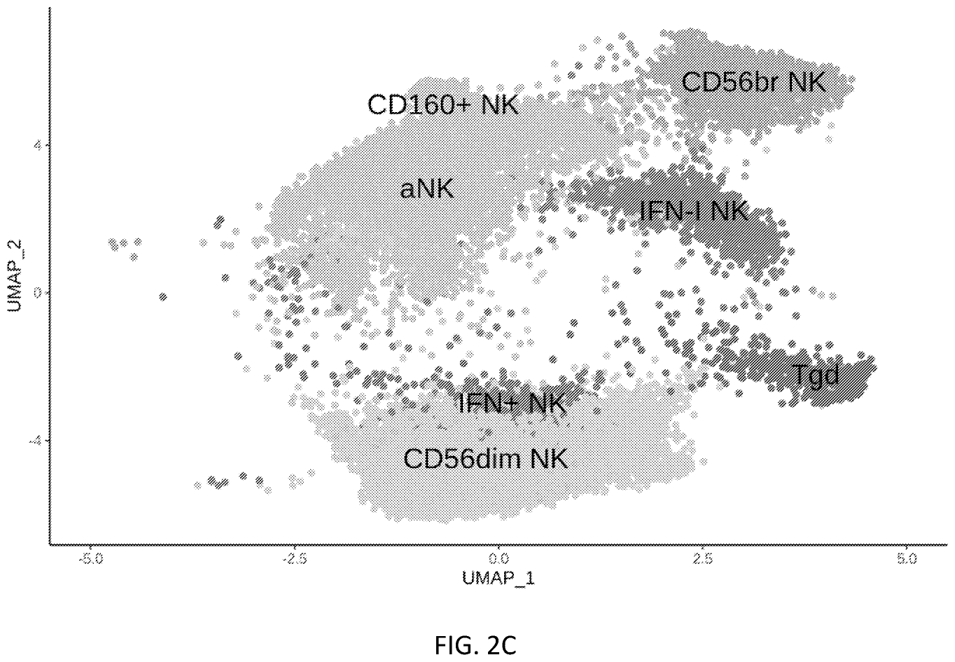

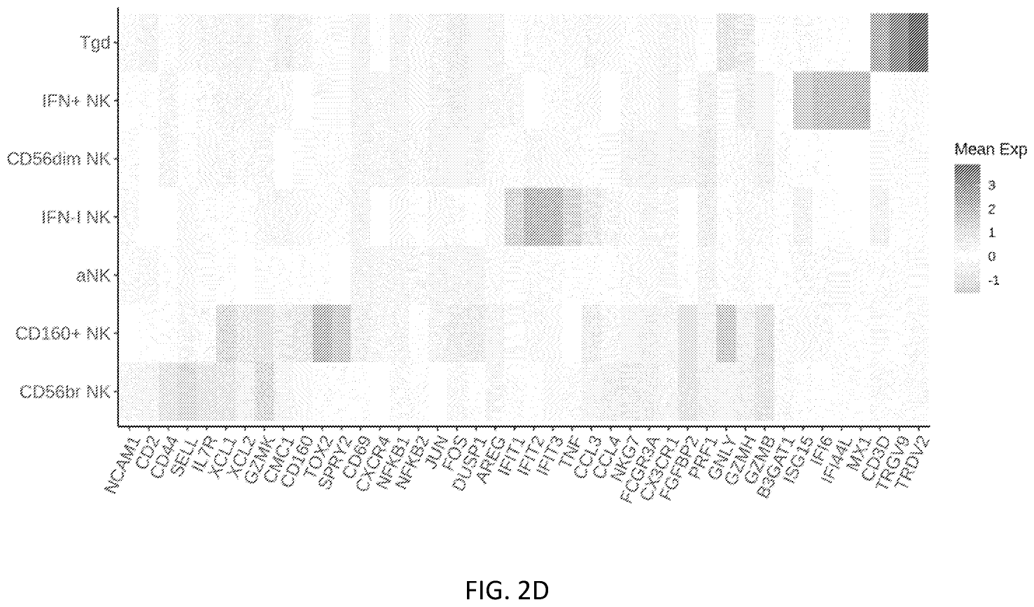

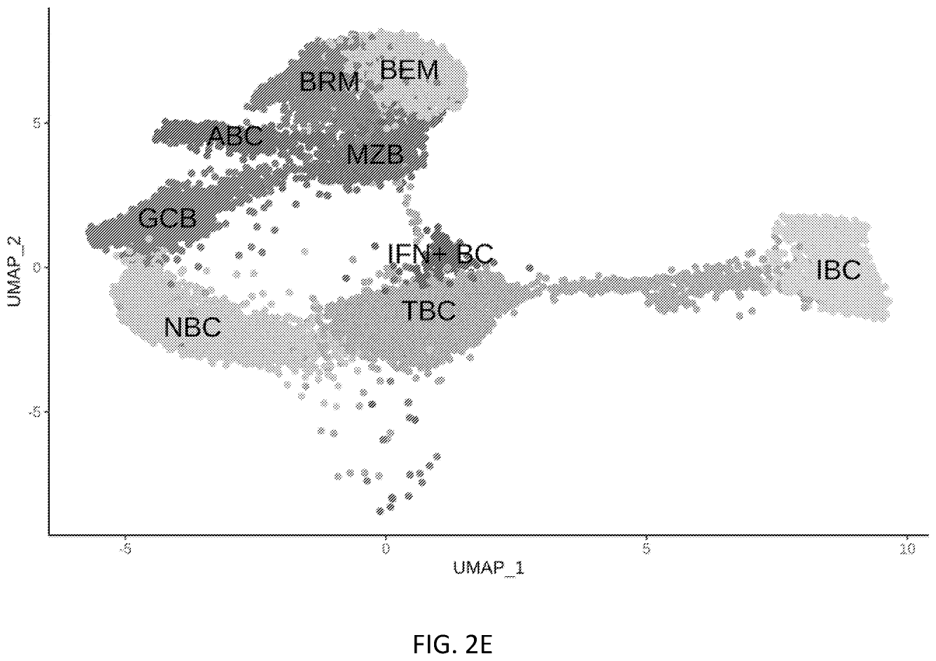

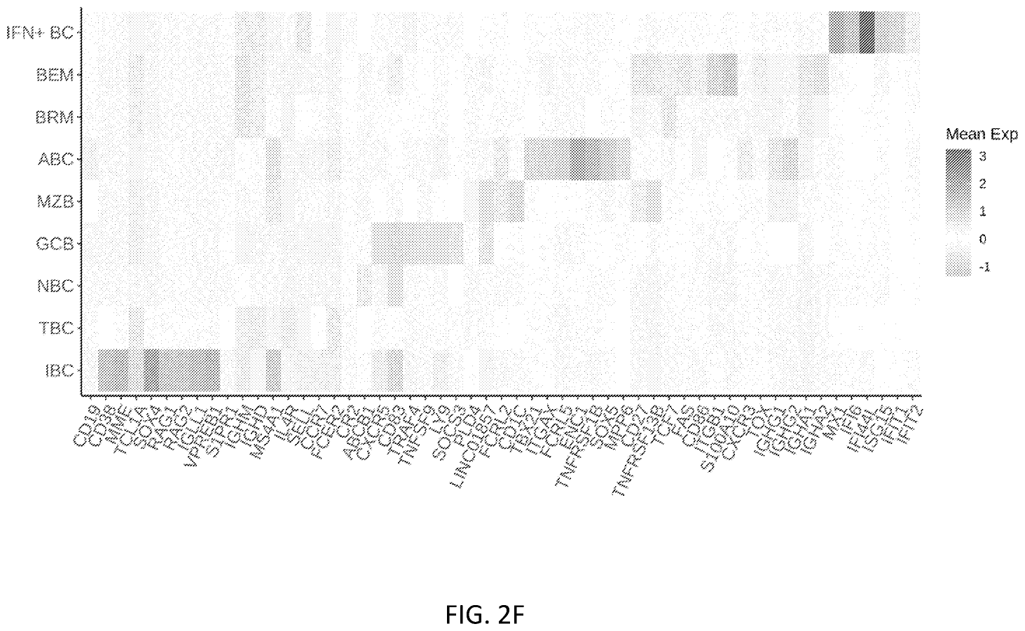

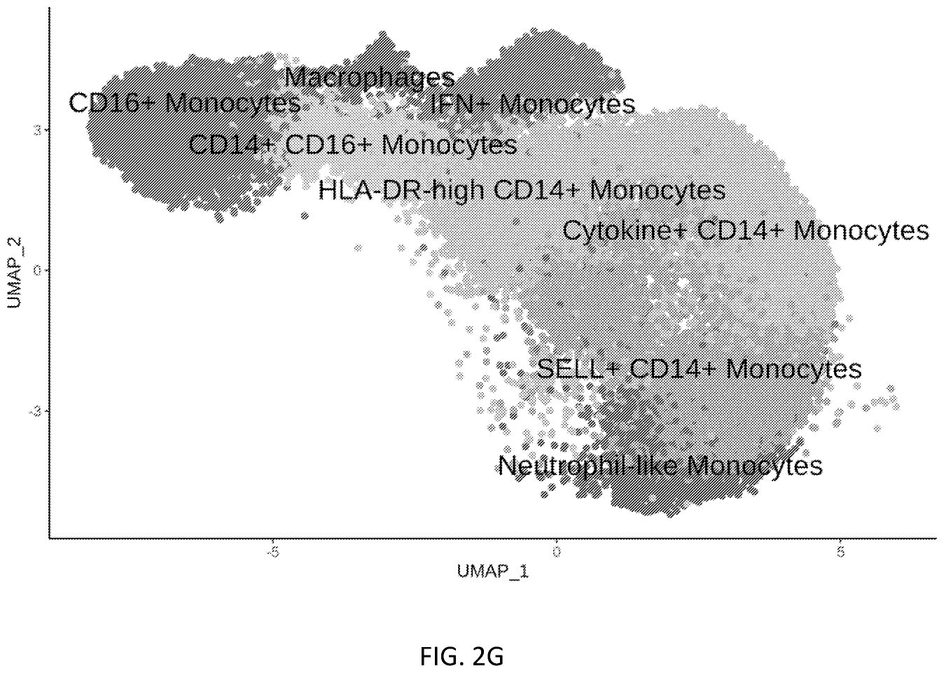

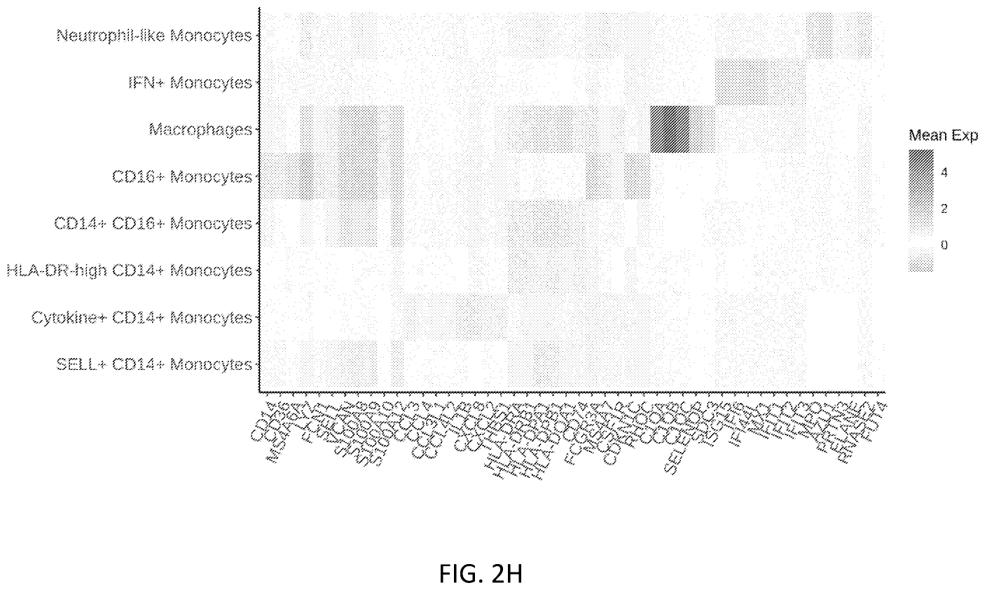

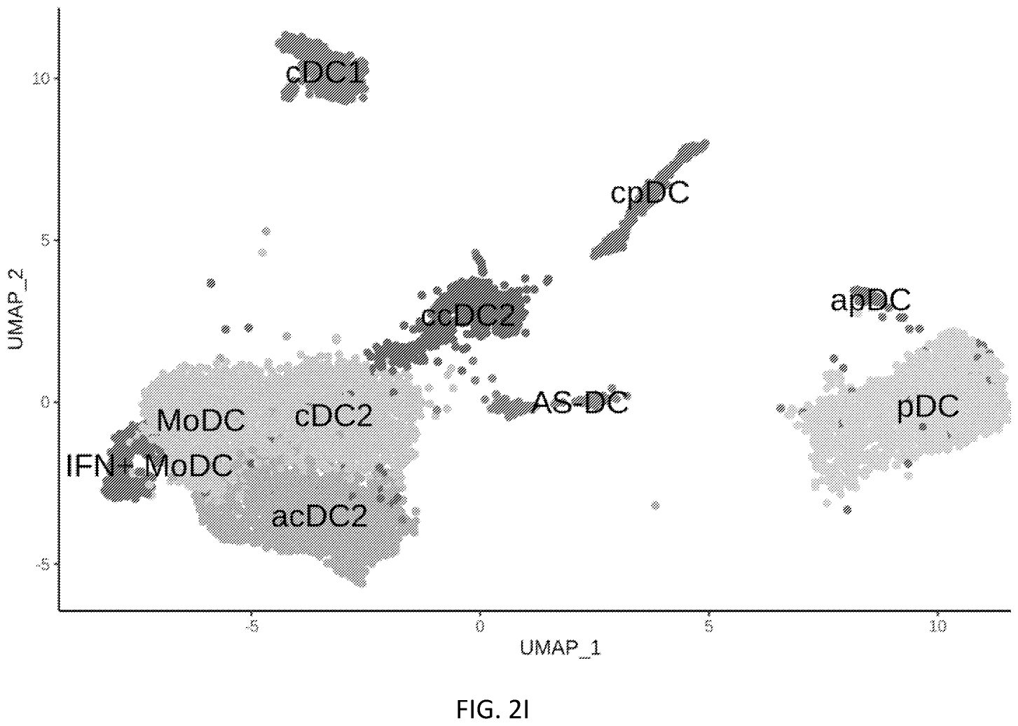

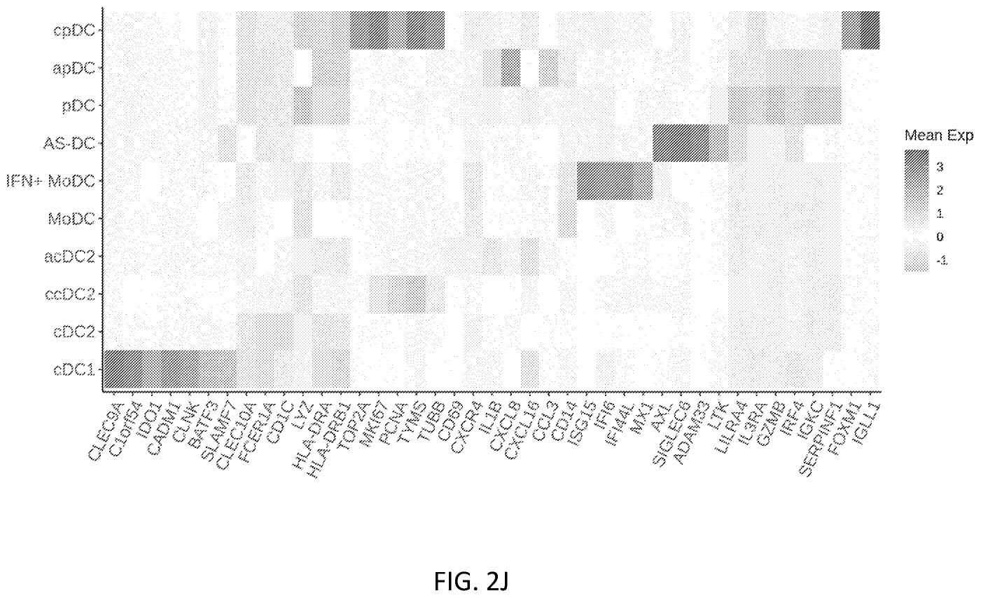

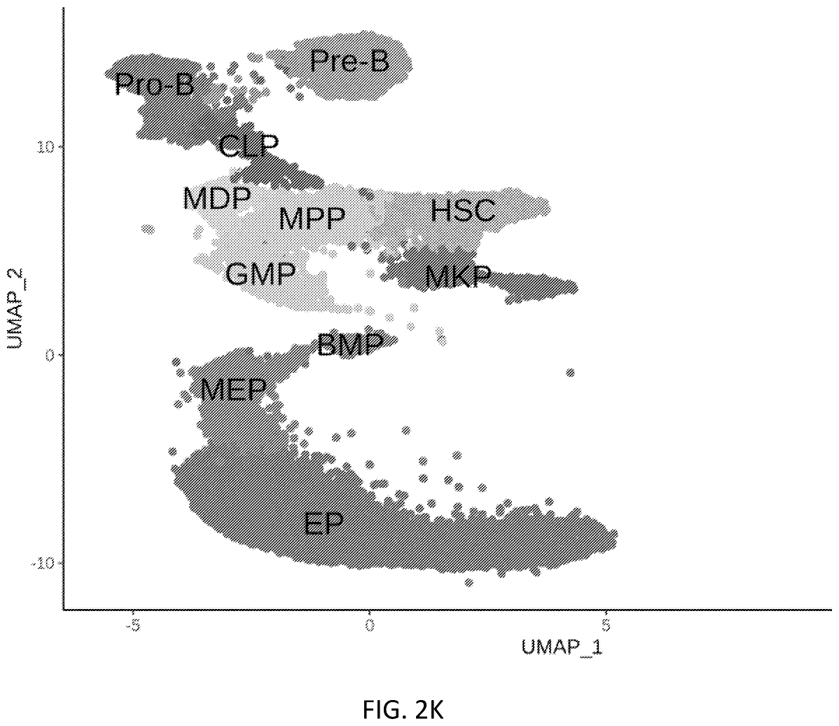

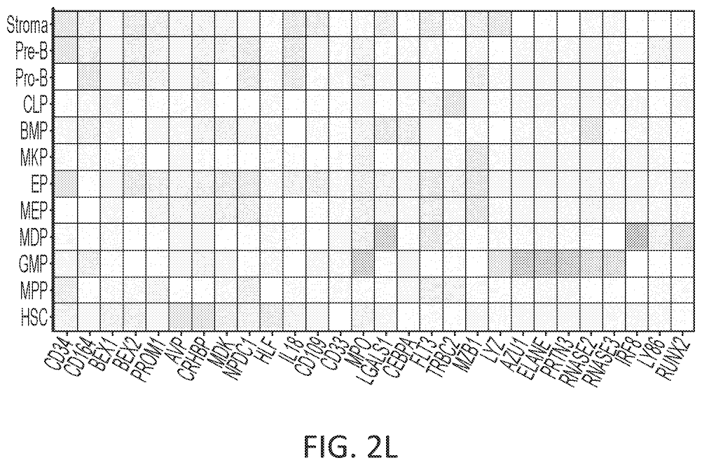

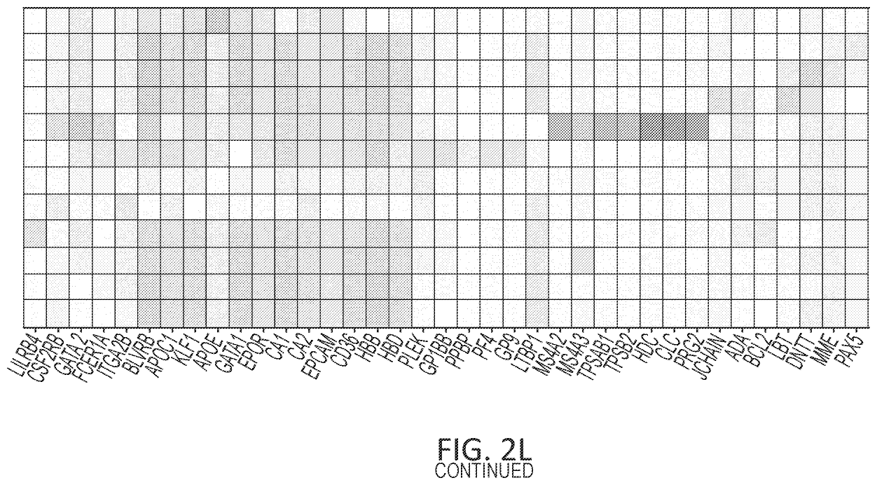

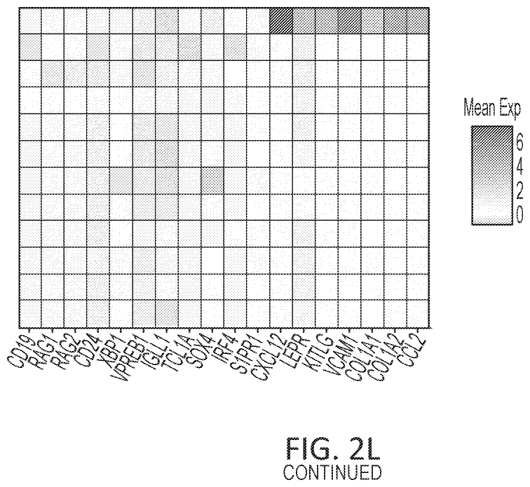

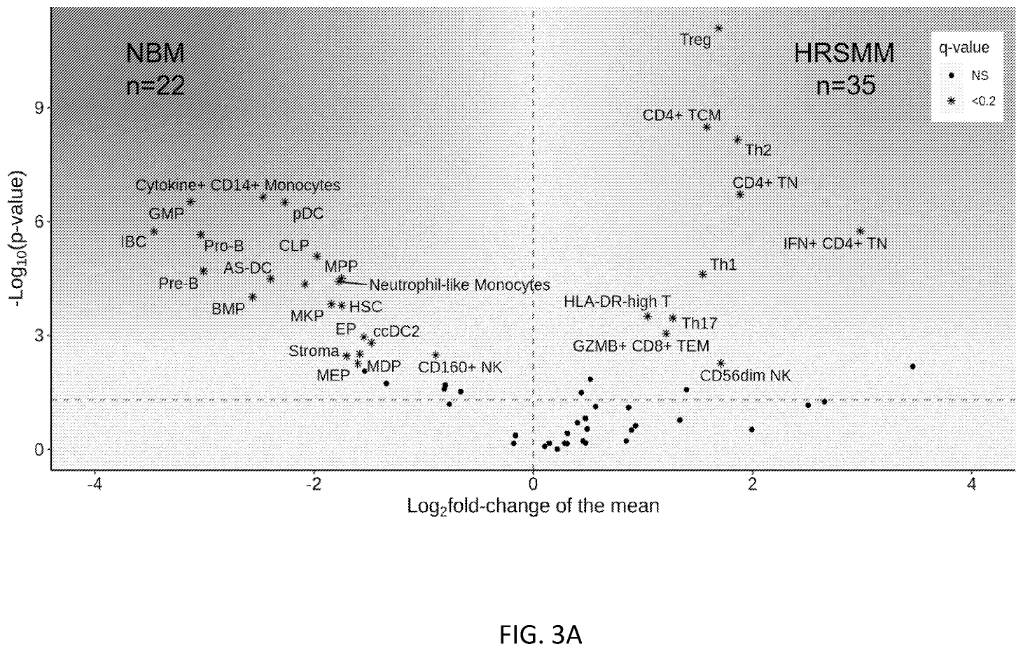

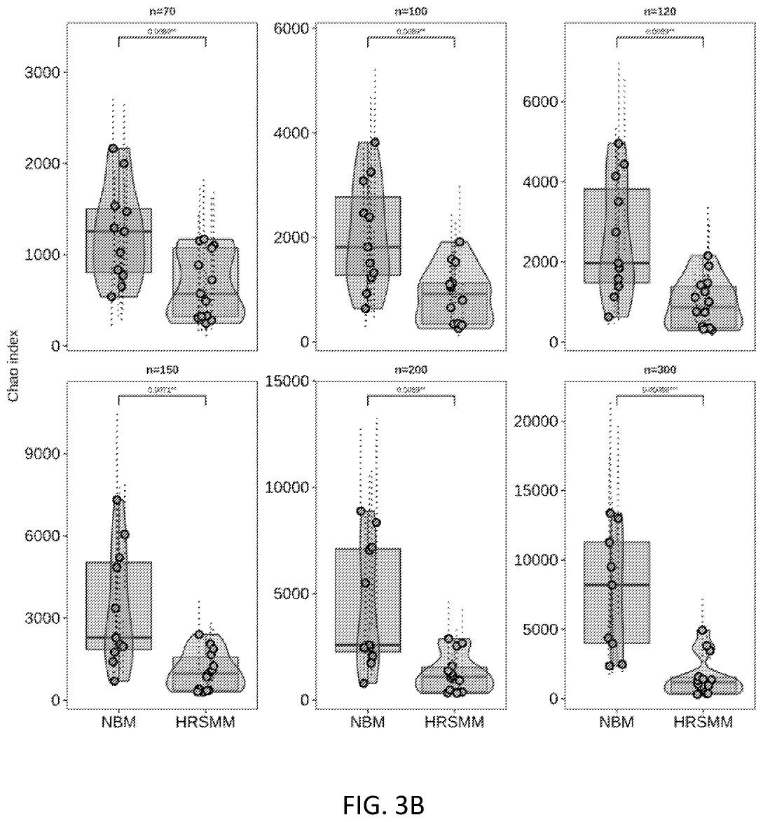

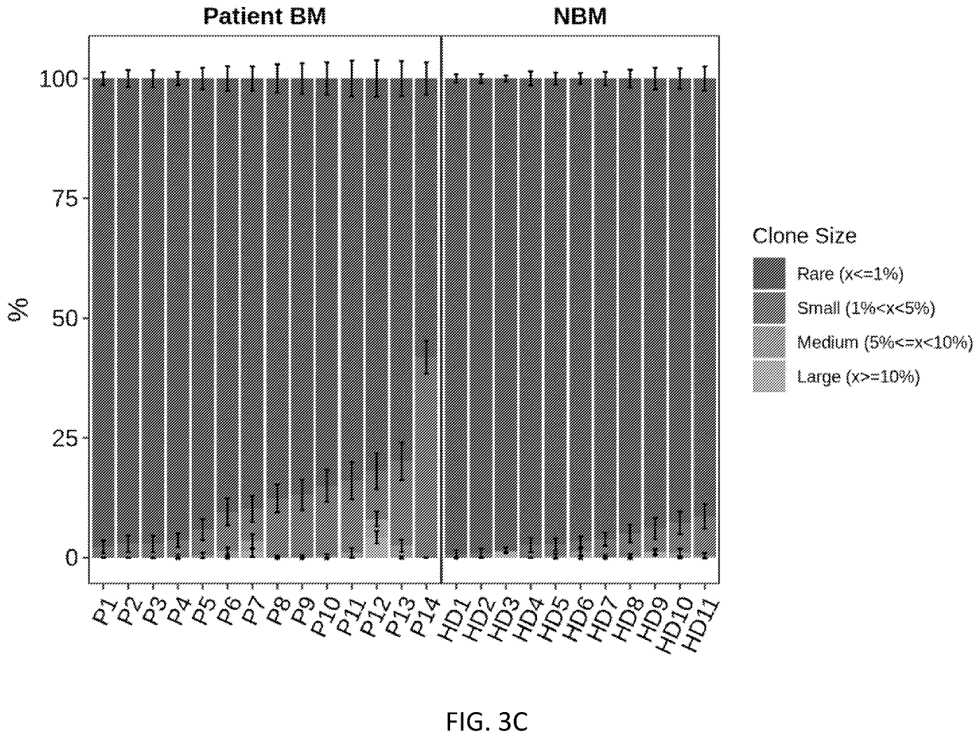

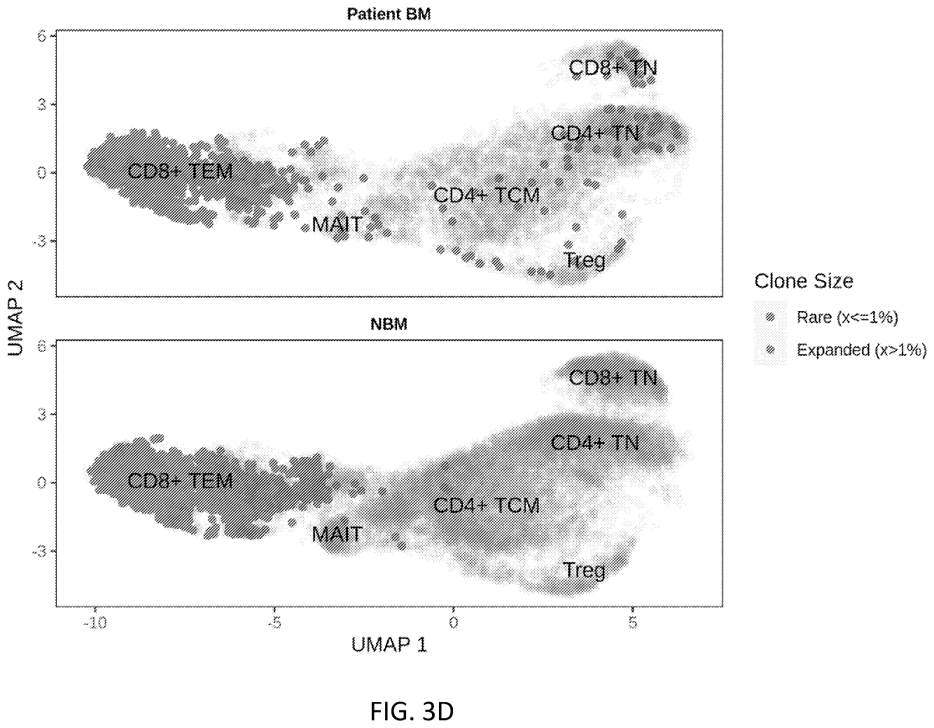

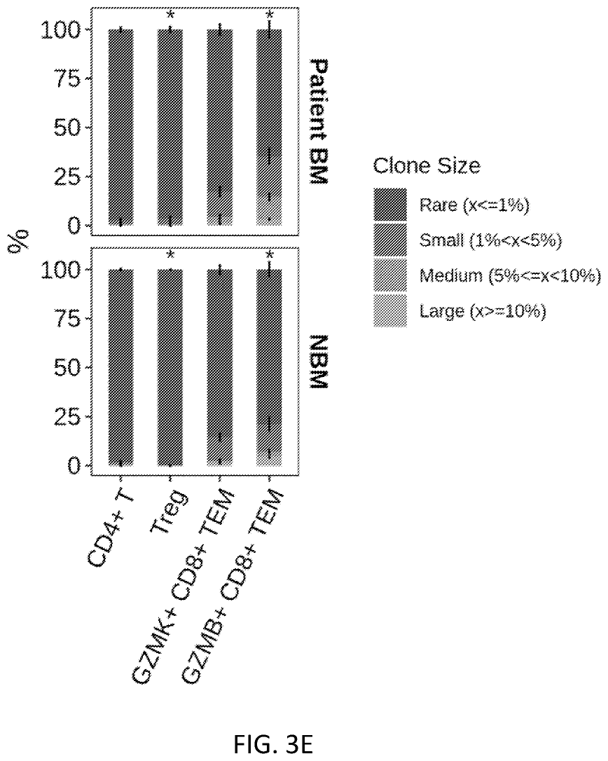

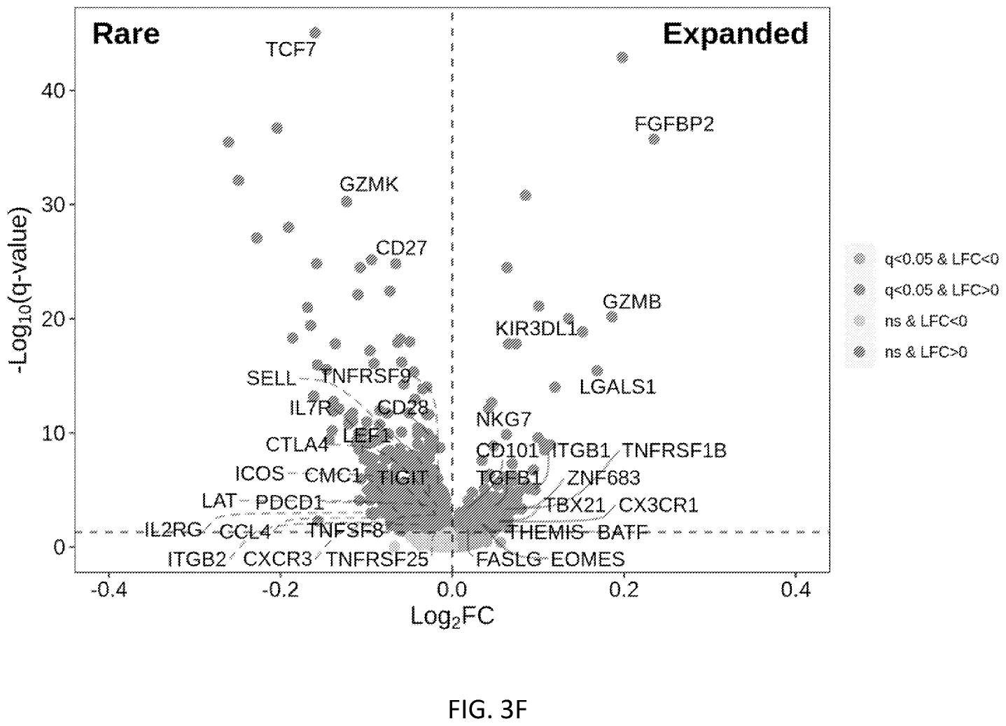

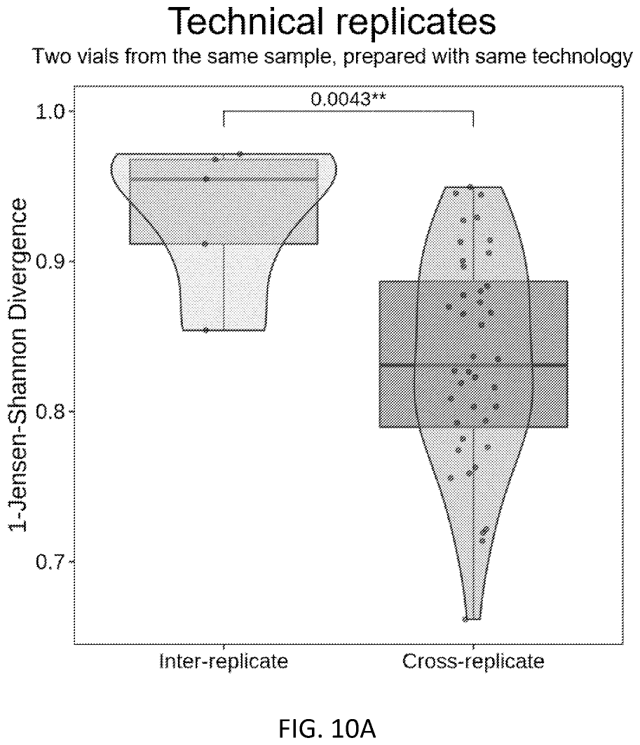

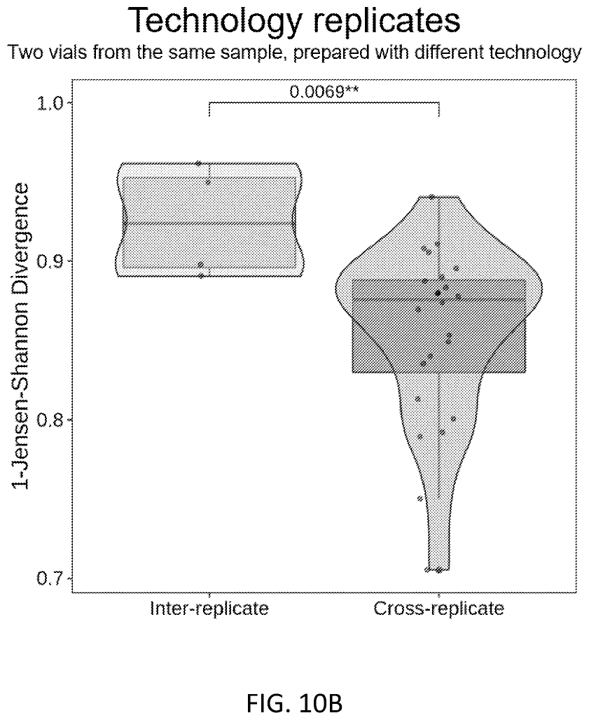

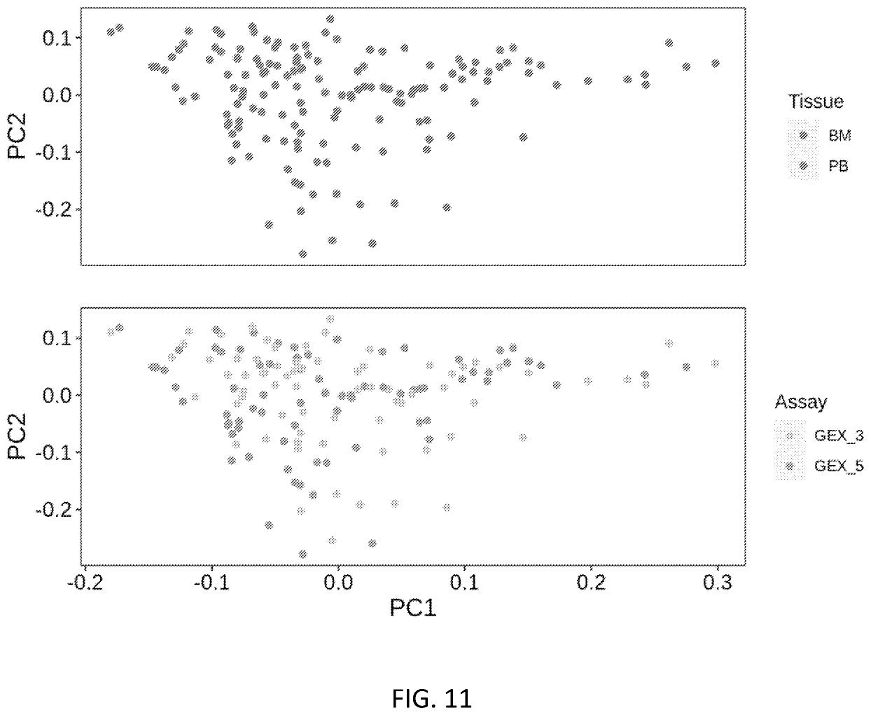

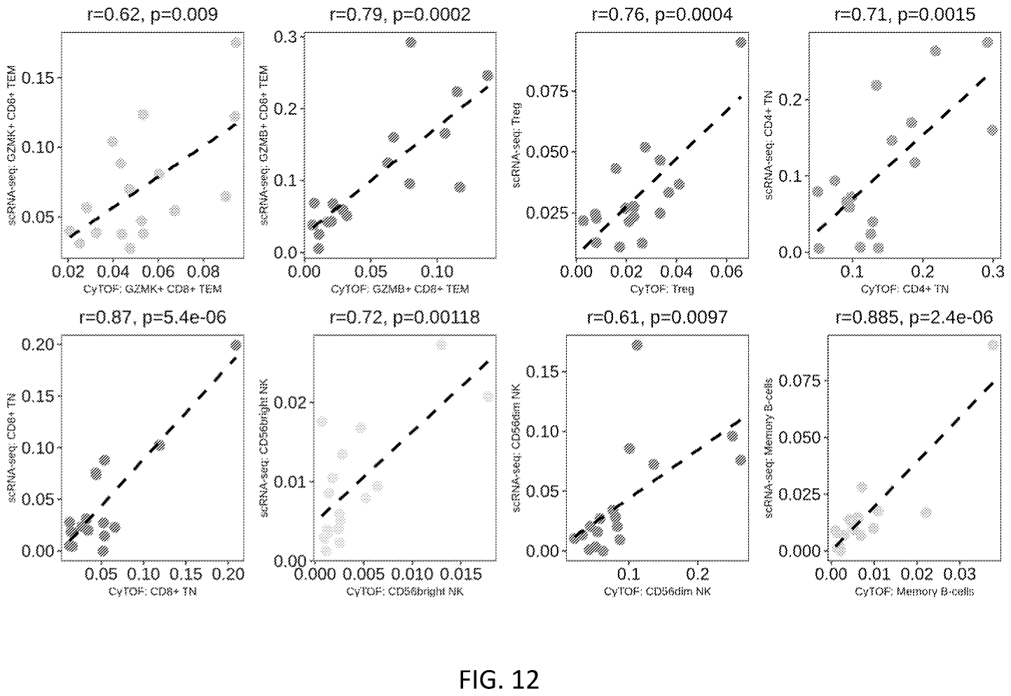

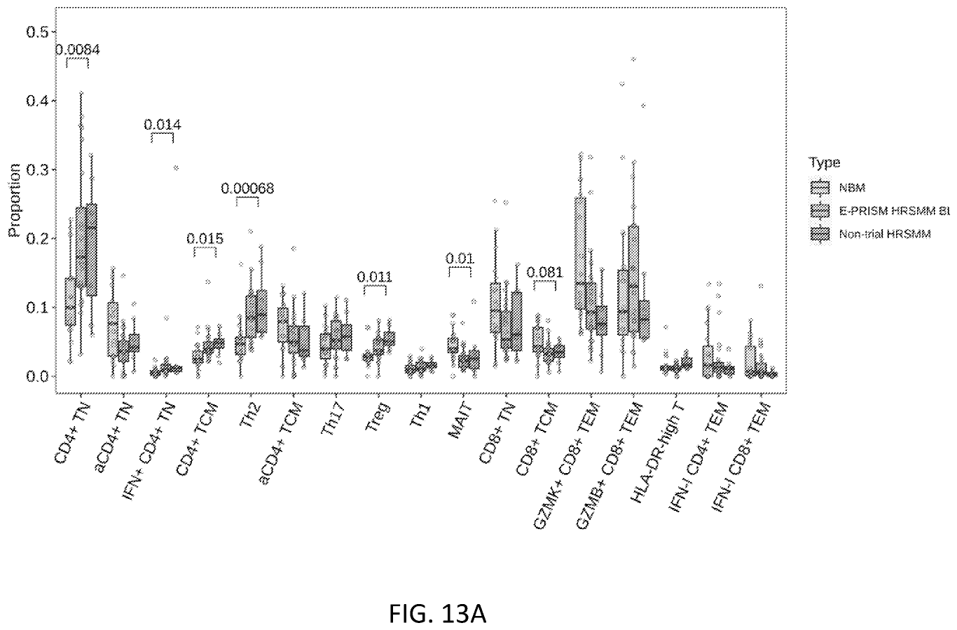

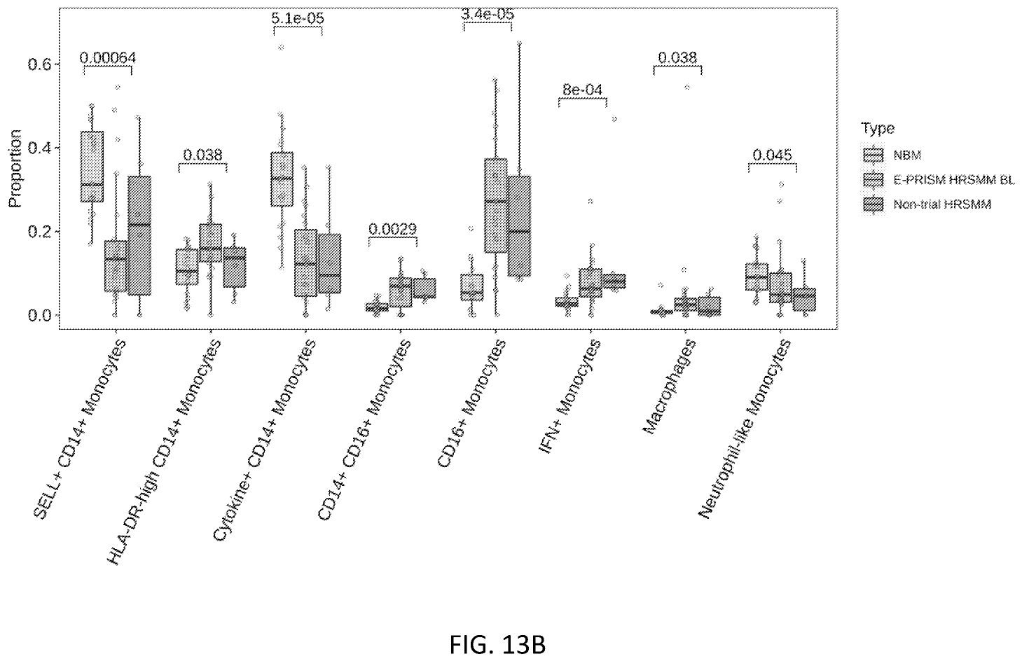

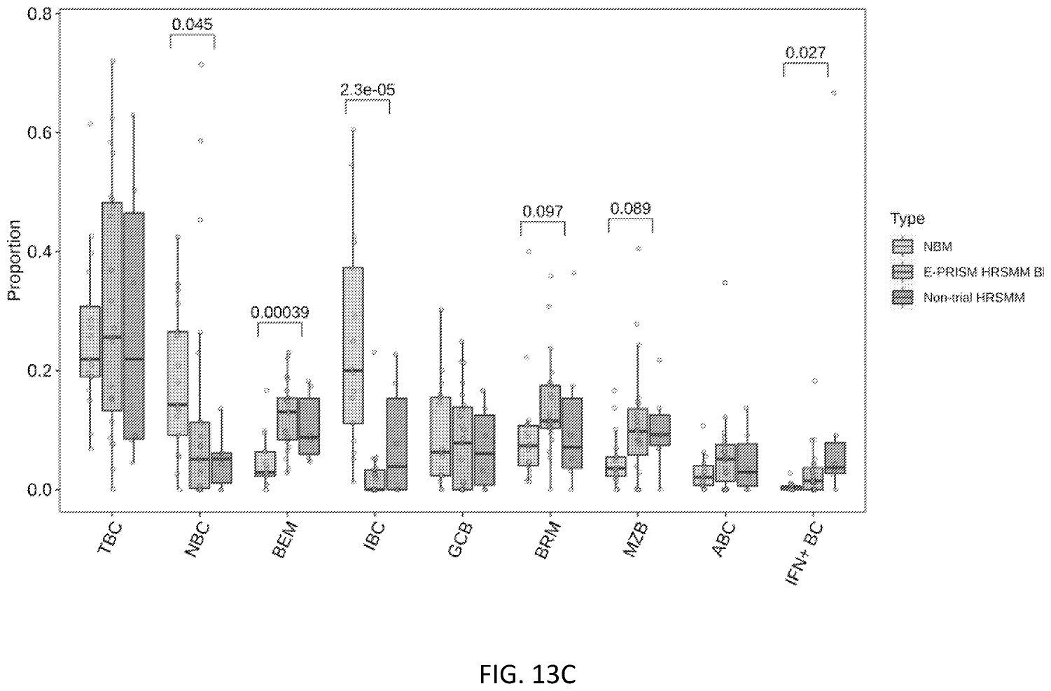

Next, experiments were undertaken to explore whether immune profiling can help to identify patients with high-risk SMM who are more likely to benefit from immunotherapy. Single-cell RNA-sequencing was performed on 211,374 immune cells from 149 samples of 34 patients enrolled in E-PRISM [60 CD138-negative BM mononuclear cell samples at baseline (n=28), cycle 9, Day 1 (C9D1, n=16) and EOT (n=16), and 68 peripheral blood mononuclear cell (PBMC) samples at baseline (n=33), C9D1 (n=14), and EOT (n=21)], and 21 age-matched healthy donors [BM samples (n=11) and PBMCs (n=10)] (Online Methods). E-PRISM data was also integrated with our cohort of non-trial patients with MGUS (n=6), low-risk SMM (LRSMM, n=3), high-risk SMM (HRSMM, n=9), newly diagnosed MM (NDMM, n=9) and healthy donors (n=11) 15, reaching a total of 187 samples and 231,608 cells (FIGS. 2A-2L). To assess the reproducibility of the present cell type quantification, two cell vials were thawed for each of 9 samples; for five of them, both replicate libraries were prepared using the same technology (3′- or 5′-end sequencing) (technical replicates); for four of them, each replicate library was prepared with a different technology (technology replicates). Across all cases, the inter-replicate divergence of immune cell composition was significantly lower compared to cross-replicate estimates, suggesting that the quantification of immune cell composition is reproducible across technical replicates and library technologies (FIGS. 10A and 10B). Furthermore, in principle component analysis (PCA) of immune cell composition, BM and PB samples mixed well, as did samples prepared with 3′-end or 5′-end technology demonstrating successful integration across tissues of origin or library preparation technology (FIG. 11). To explore whether cross-platform reproducibility of potential composition-based biomarkers would be feasible, cytometry by time-of-flight (CyTOF) was performed on 17 BM samples (10 drawn at baseline, and 7 drawn at EOT) with matched single-cell RNA-sequencing data and observed significant positive correlation between protein-level and RNA-level abundance estimates (FIG. 12). By comparing the BM immune cell composition of patients with HRSMM (n=35) and healthy donors (n=22), a significant increase in the abundance of naïve and memory CD4⁺ T-cells (including Tregs, Th1, Th2, and Th17 cells), granzyme B (GZMB)-expressing CD8⁺ T-cells, and CD56_{dim} NK cells in patients, and a significant decrease in the abundance of CD14⁺ Monocytes, Plasmacytoid Dendritic Cells (pDCs), AXL⁺ SIGLEC6⁺ dendritic cells (AS-DCs), progenitor cells, and immature B-cells (FIG. 3A) was observed. Furthermore, a relative decrease in the abundance of mucosa-associated invariant T-cells (MAITs) was observed in patients, a shift from CD14⁺ to CD16⁺ monocyte subpopulations, and a bias towards memory B-cells (FIGS. 13A-13C).

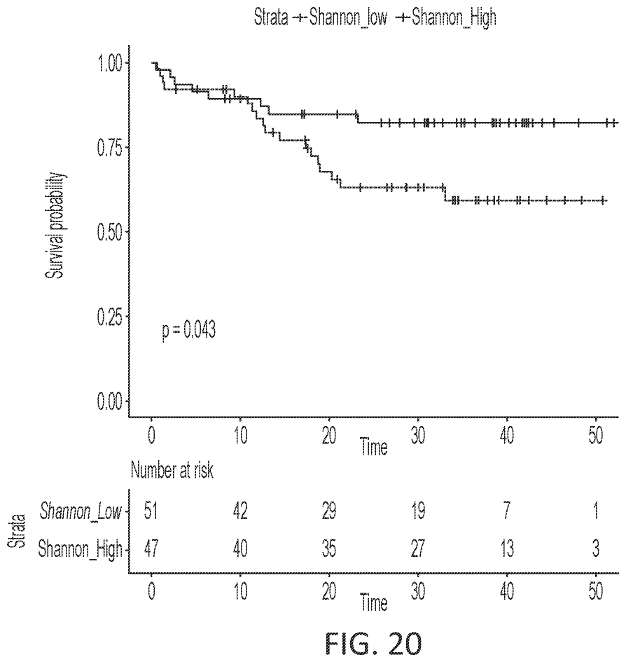

Next, changes in the T-cell receptor (TCR) repertoire of patients was characterized as compared to healthy donors and single-cell TCR-sequencing was performed on 86 samples with available single-cell RNA-sequencing data. Specifically, 65 BM and PB patient samples drawn before and after therapy were profiled [BM: 14 at BL, 2 at C9D1, 2 at EOT; PB: 22 at BL, 8 at C9D1, 17 at EOT], and 21 samples from healthy donors [BM: 11; PB: 10]. To control for variability in T-cell numbers, a range of T-cell numbers (n=75-500) from each sample were downsampled for 100 iterations, and averaged repertoire diversity estimates across iterations. Independent of the number of T-cells sampled, patient BM TCR repertoires were significantly less diverse compared to those of healthy donors, as assessed by the Chao index (FIG. 3B). The observed decrease in repertoire diversity appeared to be driven by broad low-level clonal expansion, rather than a targeted expansion of a few large clones dominating the repertoire (FIG. 3C). Similar to healthy donors, clonal expansion was primarily observed in cytotoxic populations, however Tregs and GZMB⁺ CD8⁺ TEM cells from patient BM were more likely to be part of an expanded clone compared to those from healthy donor BM (FIGS. 3D and 3E). This finding is consistent with the expansion of Tregs and GZMB⁺ CD8⁺ TEM cells observed in patient BM and suggests that these cells may be expanding in an antigen-specific manner. To characterize the phenotype of expanded GZMB⁺ CD8⁺ TEM cells, expression levels between clonally expanded and rare GZMB⁺ CD8⁺ TEM cells were compared across patients. Clonally expanded cells had a more potent terminal effector phenotype, with decreased expression of genes associated with long-lived memory subpopulations, and decreased levels of exhaustion markers (FIG. 3F). Next, to assess the impact of decreased TCR repertoire diversity on patient outcome, bulk TCR-sequencing was performed on PBMC-derived DNA from 100 patients with newly diagnosed MM. Significantly shorter overall survival in patients with lower repertoire diversity was observed (FIG. 20), indicating that the alteration we observed in patients with SMM may in fact contribute to patient outcome.

### Example 4: Immune Reactivity at Baseline and Post-Therapy Immune Normalization are Associated with Significantly Longer Progression Free Survival in Patients with High-Risk SMM Under Treatment

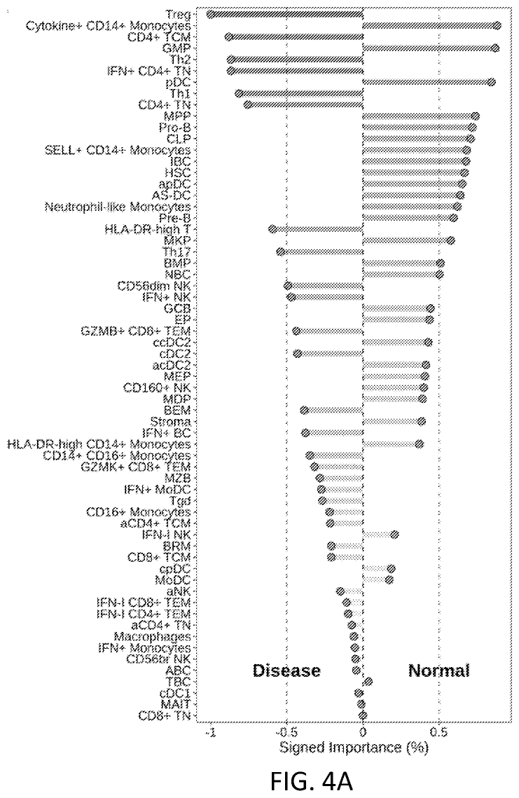

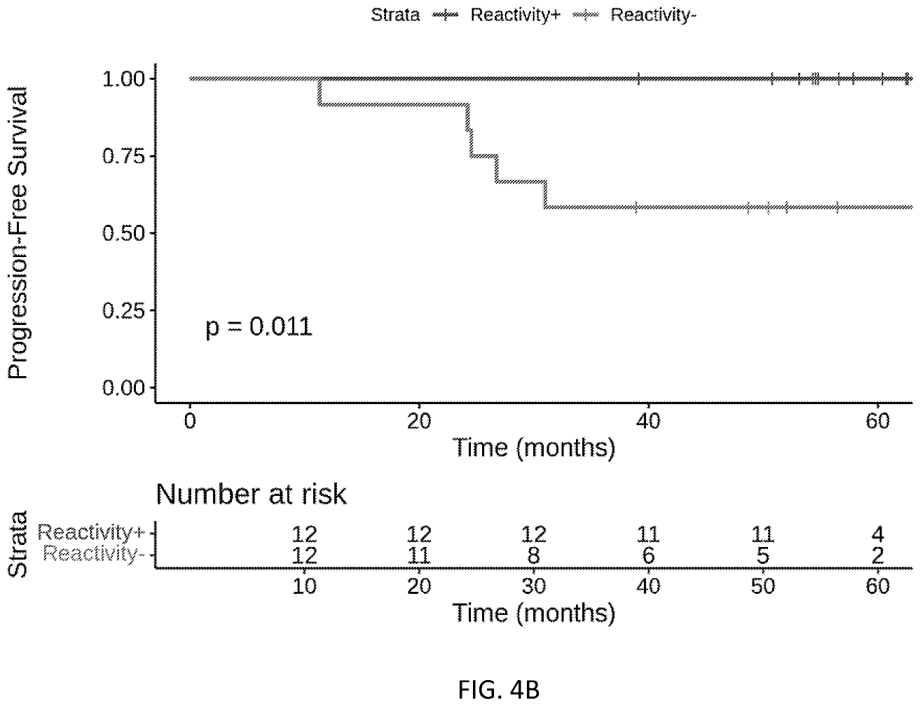

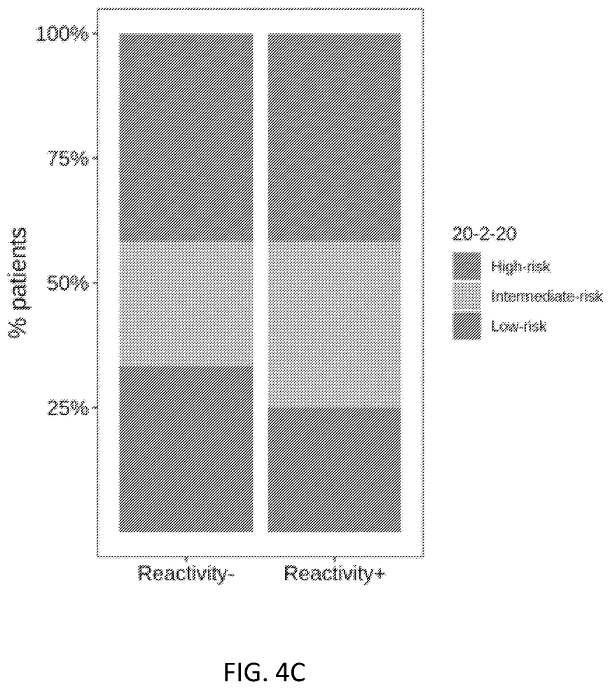

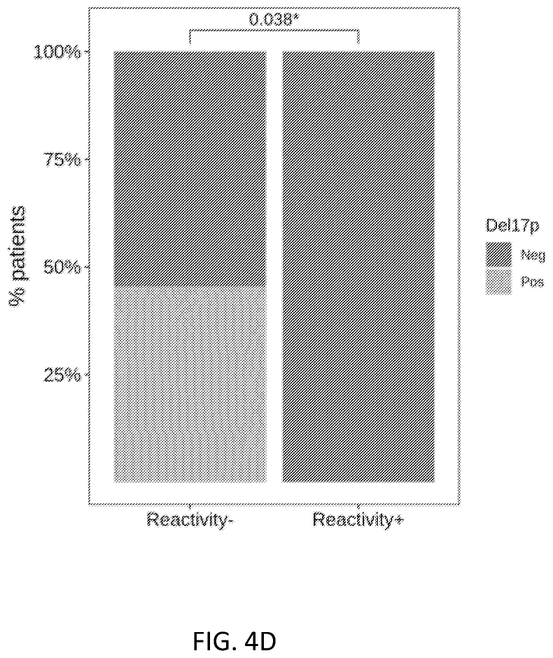

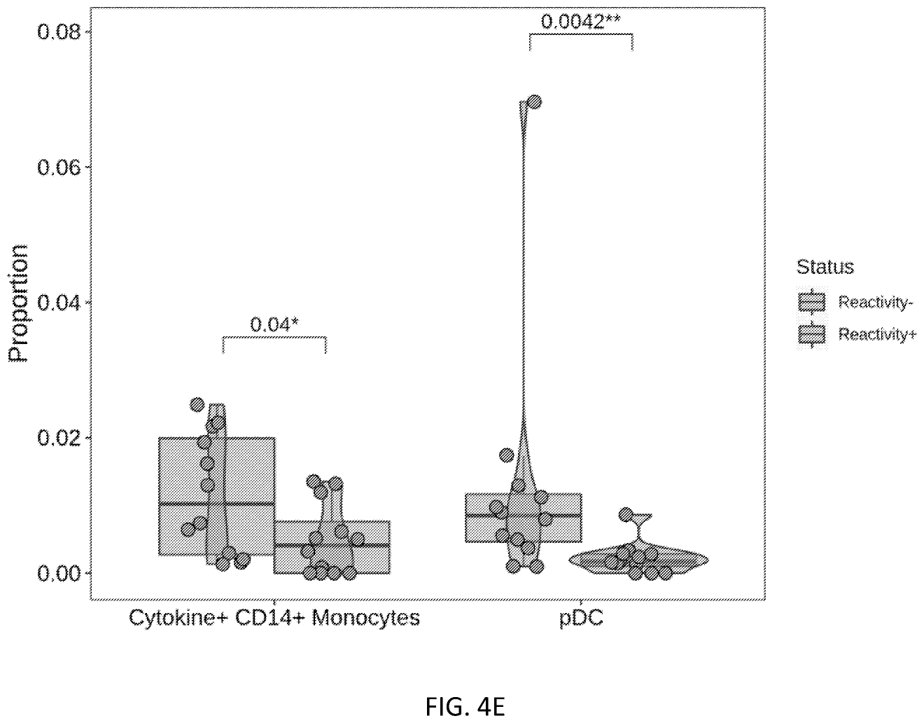

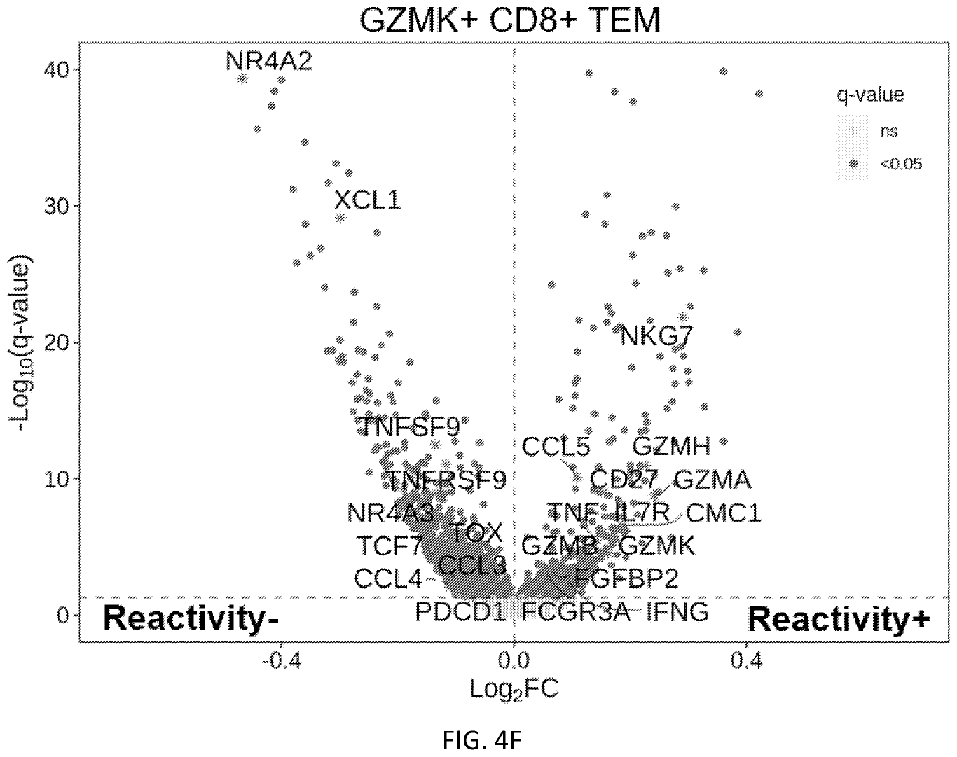

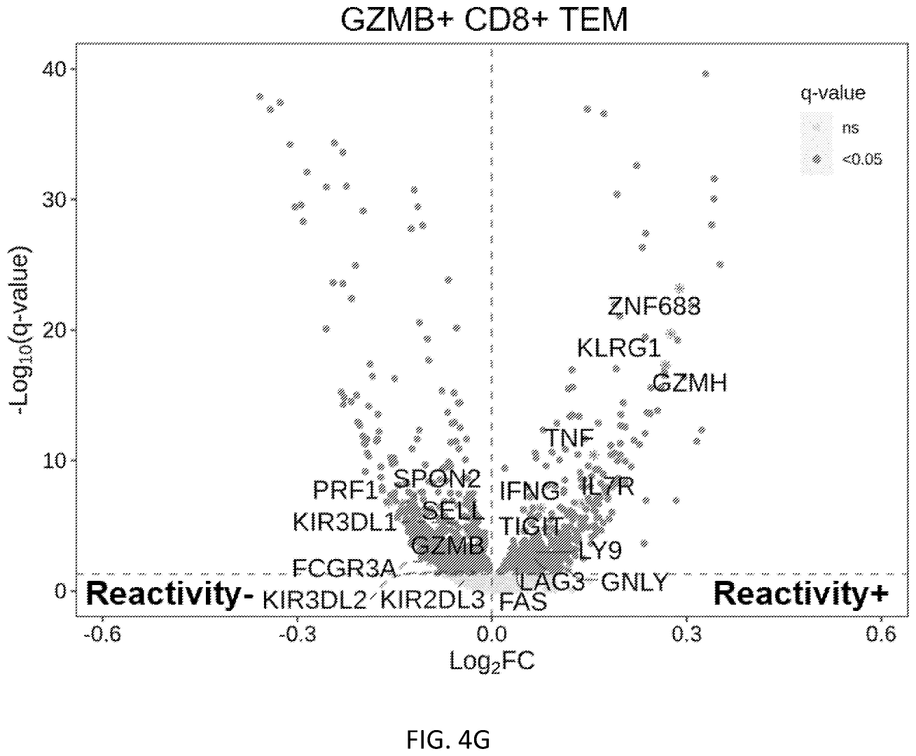

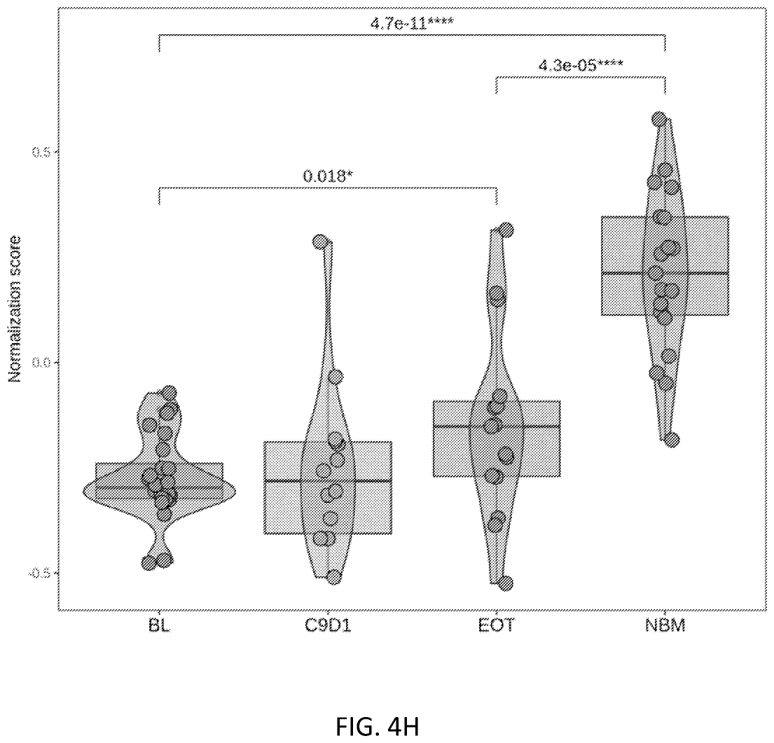

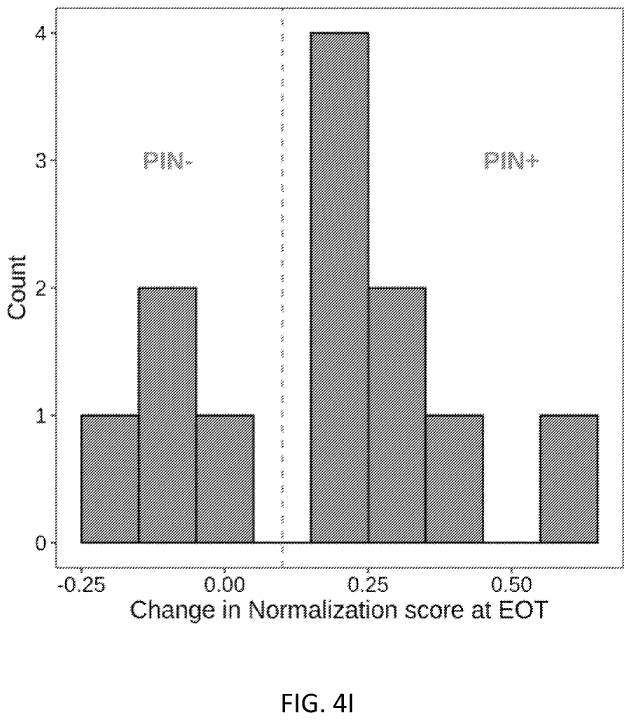

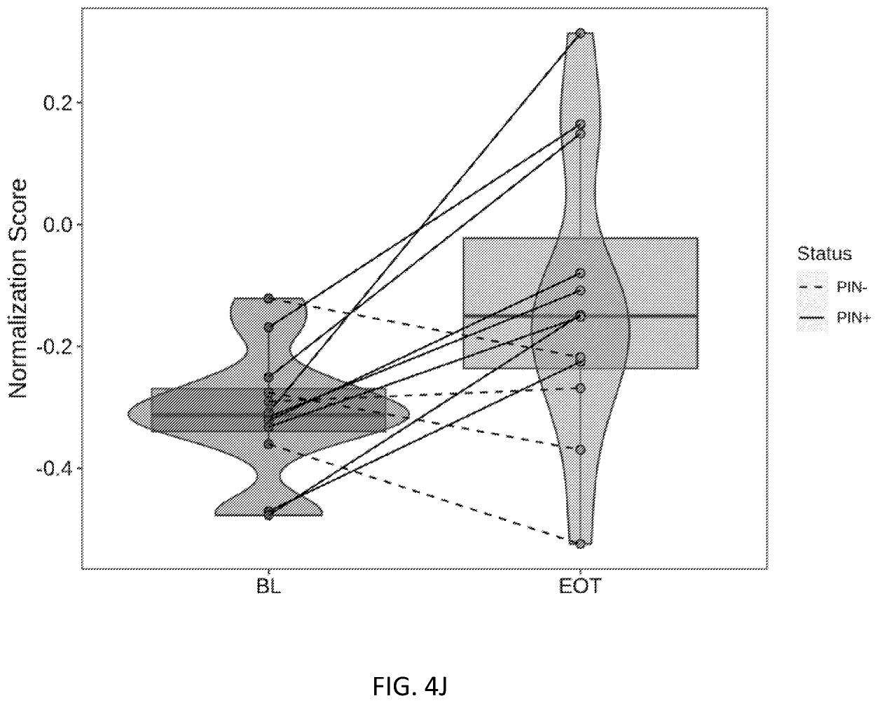

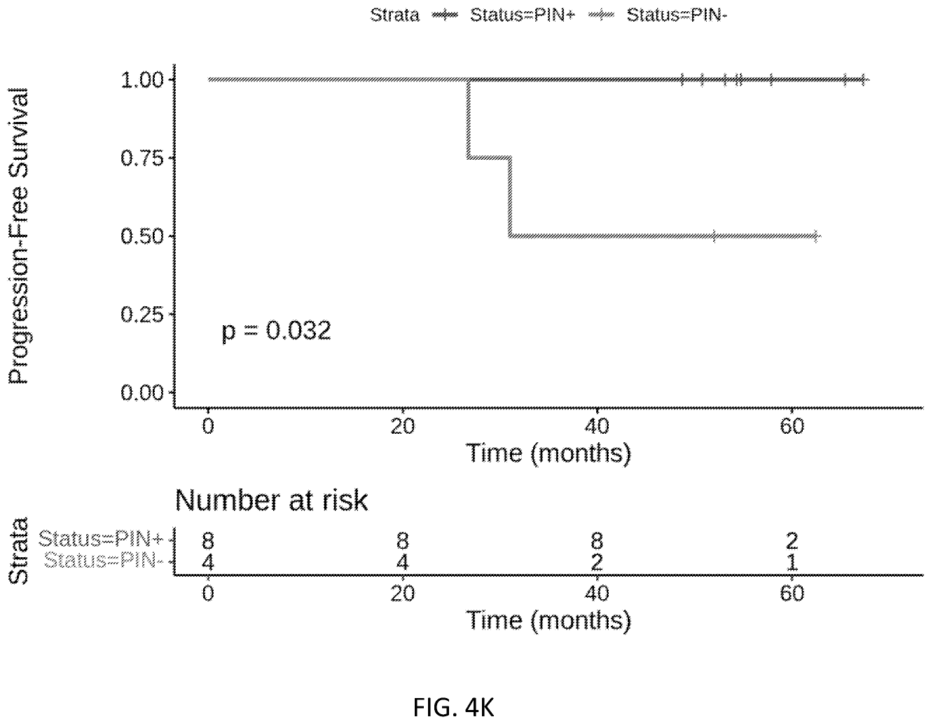

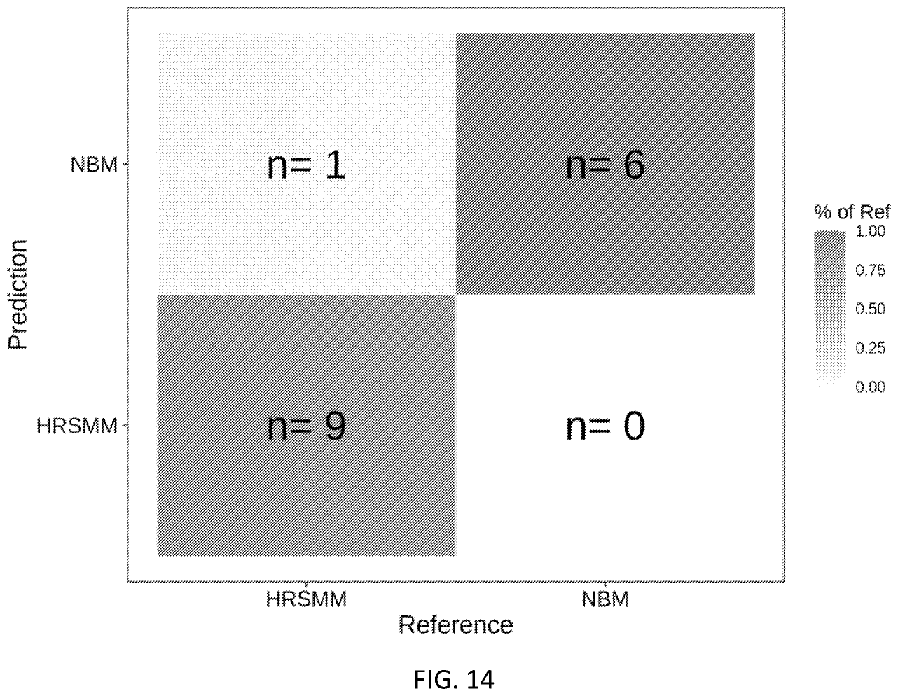

To test whether the similarity of a patient's BM immune cell composition to that of healthy donors could be harnessed for prognostication a Naïve Bayes classifier with 10-fold cross-validation and 100 iterations was trained on a training set of 41 BM samples from patients and healthy donors, to predict the presence of malignancy based on the composition of the bone marrow immune microenvironment; the classifier achieved 94% accuracy in the testing set (n=16, FIG. 14). A weighted sum score of the product of each cell type's proportion was then computed and its corresponding signed importance to the classification, and classified patients based on the median score at baseline as reactive (least normal-like) or non-reactive (most normal-like) (FIG. 4A). Patients who were classified as reactive at baseline had significantly longer PFS (FIG. 4B). Immune reactivity was independent of the patients' risk stage, but was significantly associated with the presence of Del17p, although patients with Del17p were only a fraction of the non-reactive group (FIGS. 4C and 4D). Plasmacytoid DCs and the pro-inflammatory Cytokine⁺ CD14⁺ Monocytes, which express genes related to neutrophil chemotaxis and acute inflammation, were among the top 3 cell types with the highest importance for a sample to be classified as normal-like. As expected, reactive patients had significantly lower abundance of pDCs and Cytokine⁺ CD14⁺ Monocytes (FIG. 4E). By comparing gene expression levels between reactive and non-reactive patients, a significant downregulation of exhaustion markers was observed (TOX, TNFRSF9, TNFSF9, PDCD1, NR4A2, NR4A3) in GZMK⁺ CD8⁺ TEM cells, along with an upregulation of markers associated with long-lived memory effectors (II.7R and (D) 27), markers associated with terminal effectors (GZMB, GZMH, FCGR3A, FGFBP2, NKG7) and markers associated with functionality (IFNG, TNF), a profile consistent with long-lived memory effector cells of increased potency (FIG. 4F). Moreover, in GZMB⁺ CD8⁺ TEM cells from reactive patients, an upregulation of exhaustion markers was observed (TIGIT, LAG3, LY9) and KLRG1, along with higher levels of IFNG and TNF and a downregulation of cytotoxicity markers (GZMB, FCGR3A, PRF1), a profile consistent with short-lived, exhausted terminal effector cells (FIG. 4G). Collectively, these results indicate that immune reactivity captures a patient subpopulation with decreased pro-inflammatory myeloid signaling, long-lived GZMK⁺ effector memory cells of increased potency, and short-lived, exhausted GZMB⁺ terminal effectors.

On average, patients showed significantly higher normalization scores at end of treatment, compared to baseline (FIG. 4H). Using a threshold of 0.1 based on the distribution of change in normalization scores at EOT, a state of post-therapy immune normalization (PIN) was defined and four patients who showed no or minimal increase in their normalization score at EOT as PIN-negative (FIGS. 41 and 4J) were classified. Patients who achieved post-therapy immune normalization (PIN) at end of treatment (PIN) had significantly longer progression free survival (FIG. 4K), indicating that post-therapy immune normalization may be important for prognostication. Of note, both patients who had Del17p in this subcohort were classified as PIN-negative.

### Example 5: Higher Abundance of Granzyme K-Expressing CD8⁺ T-Cells is Associated with Longer Progression Free Survival in Patients with High-Risk SMM Under Treatment

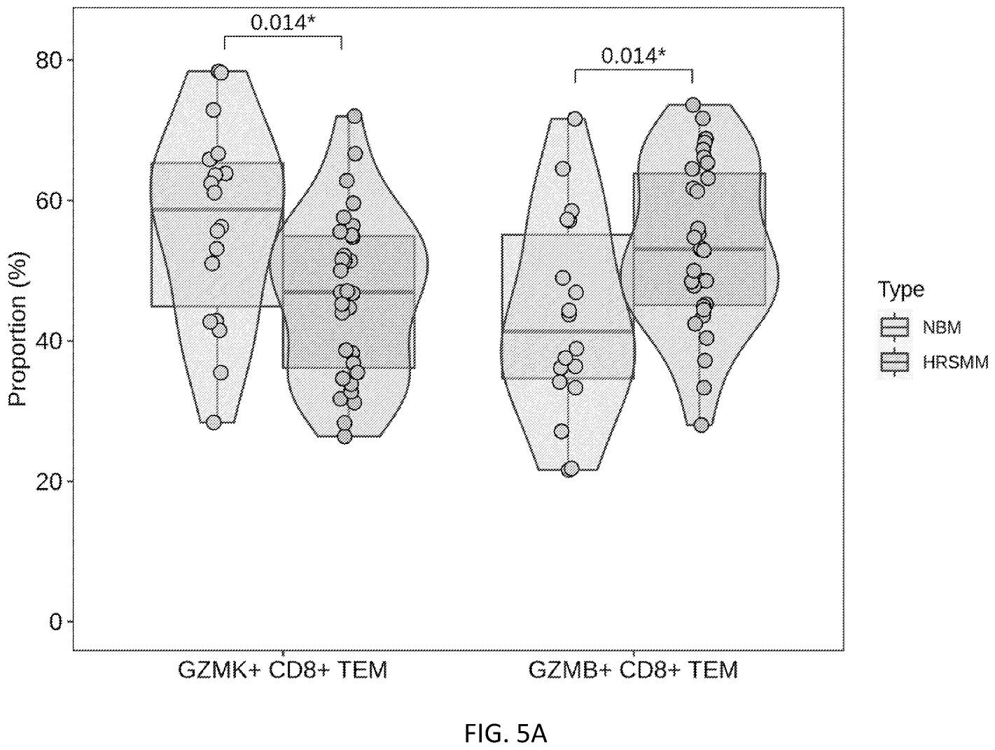

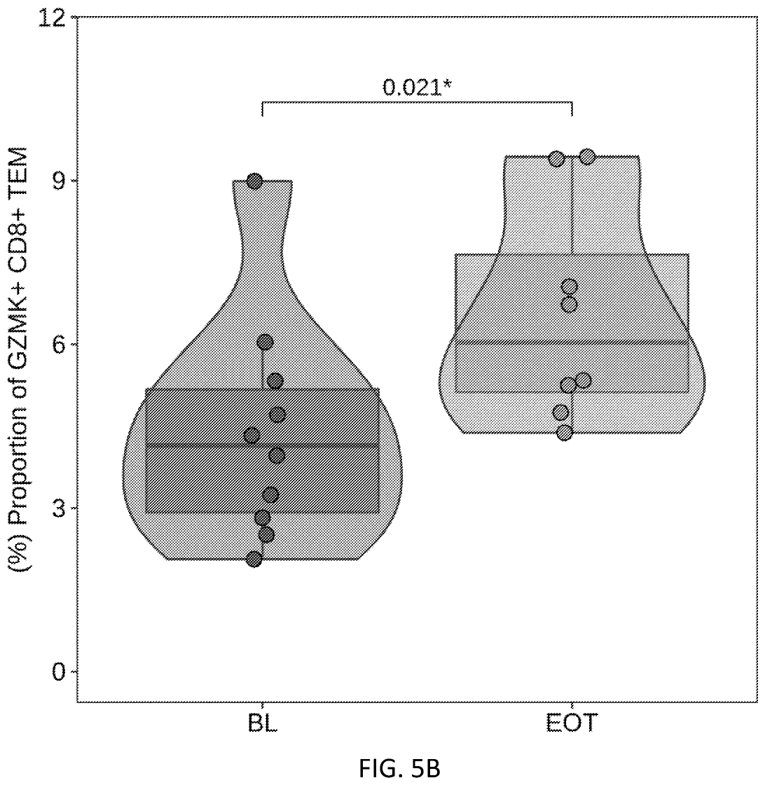

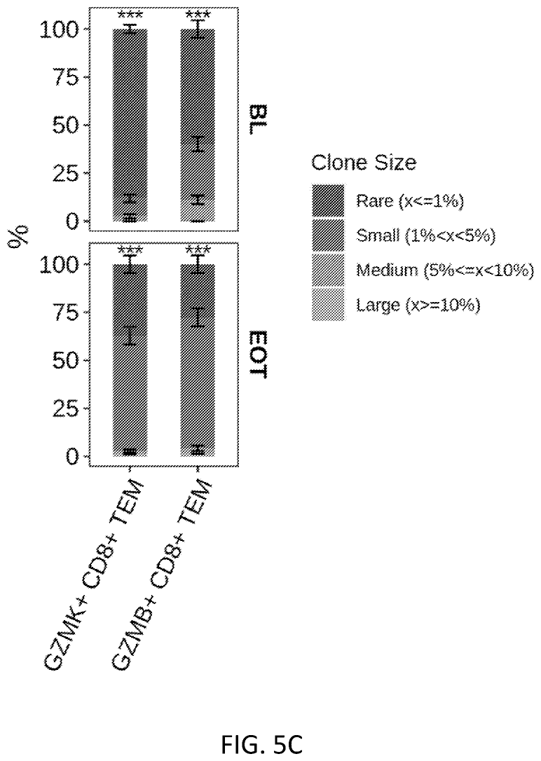

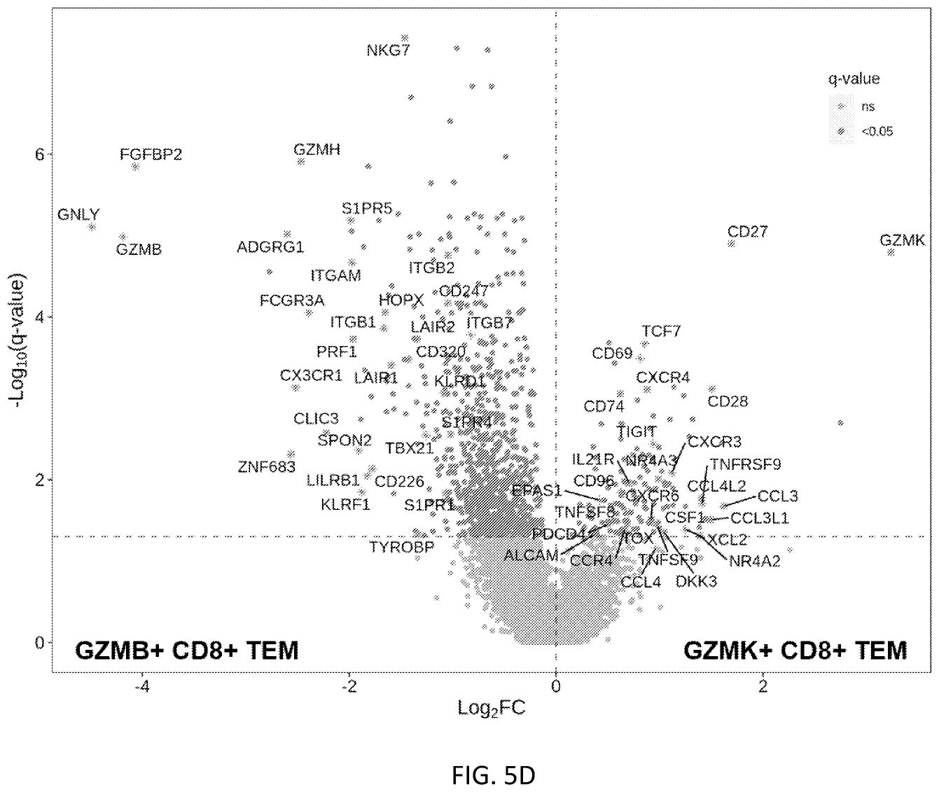

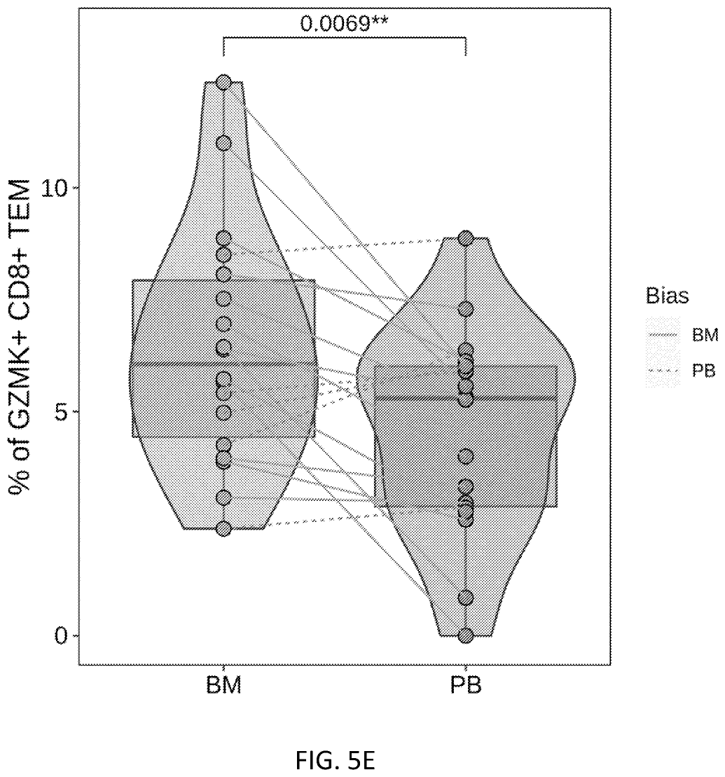

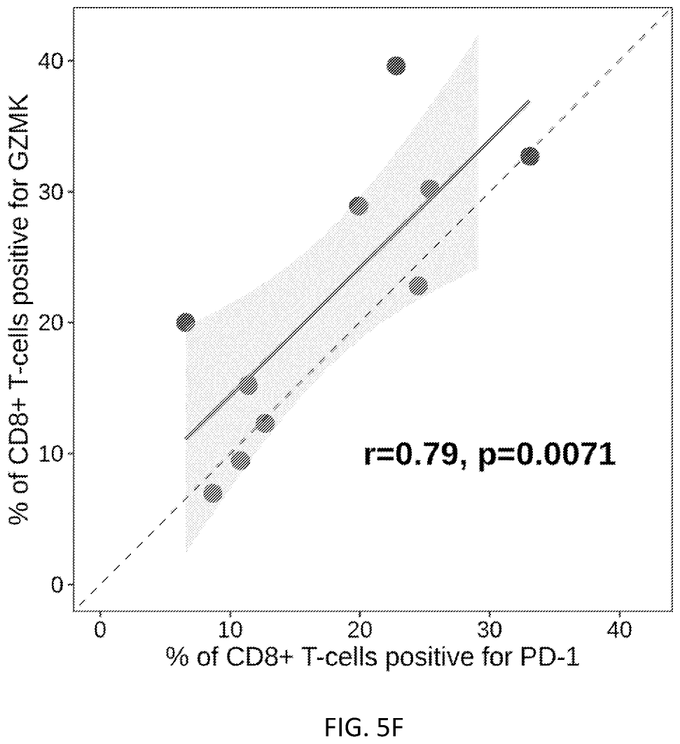

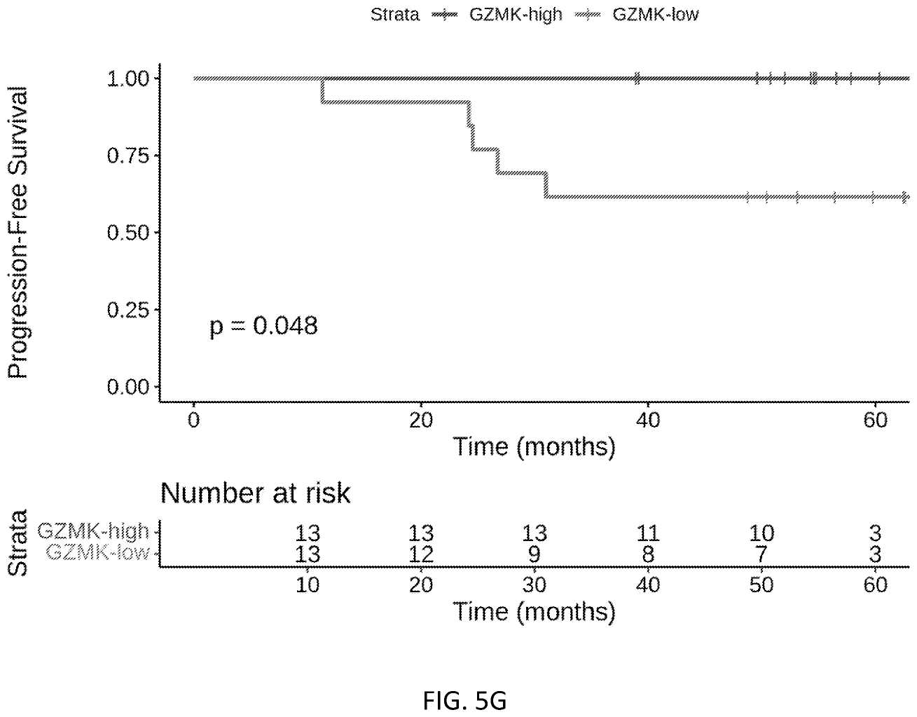

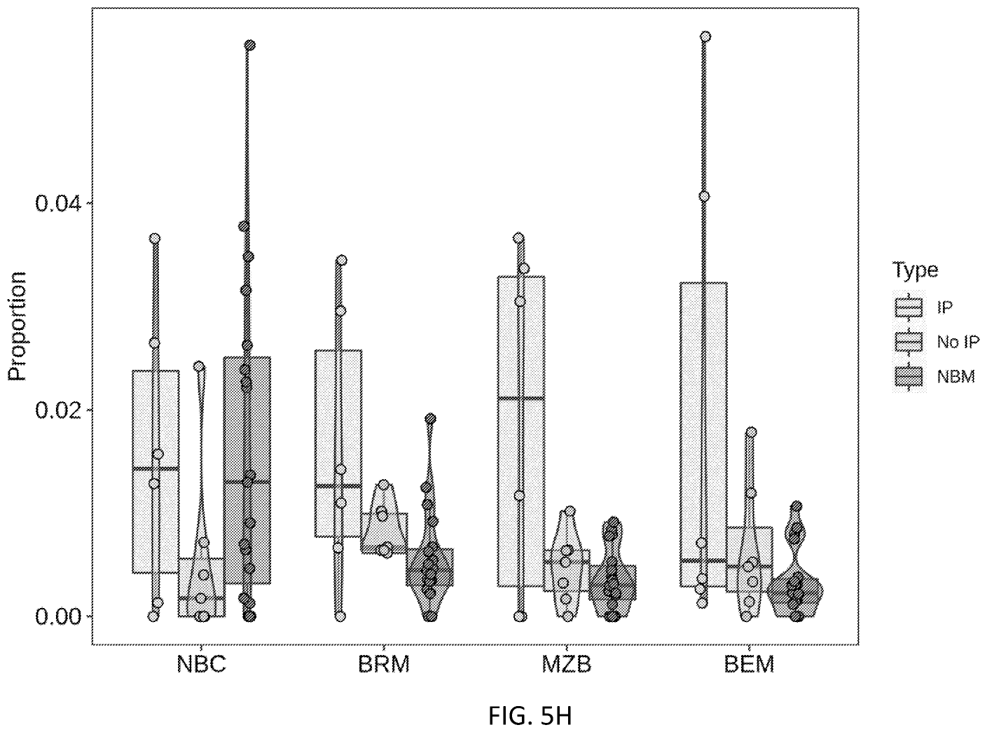

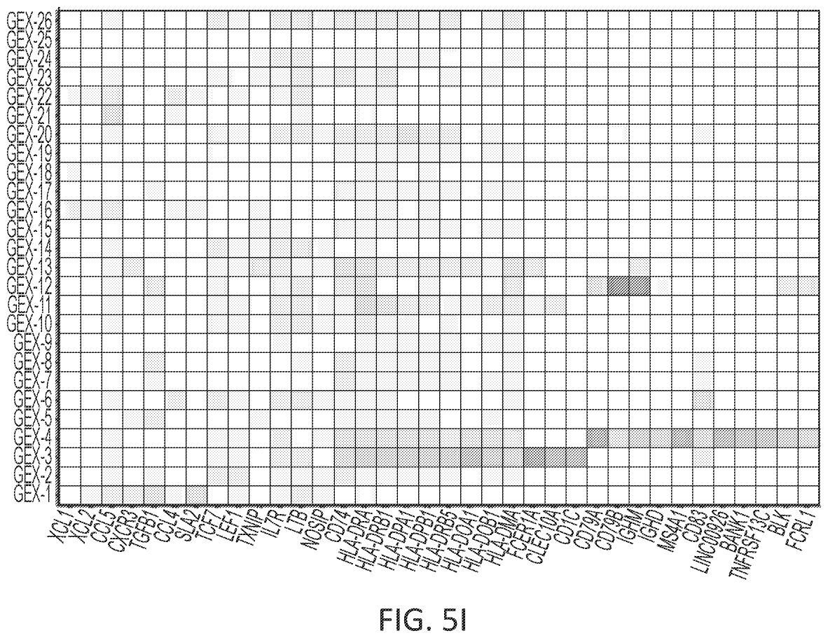

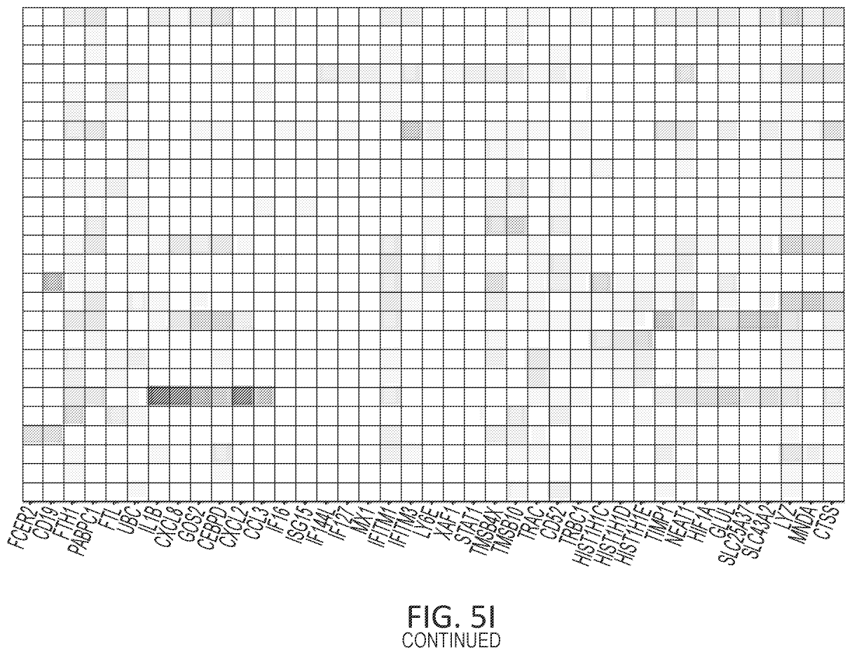

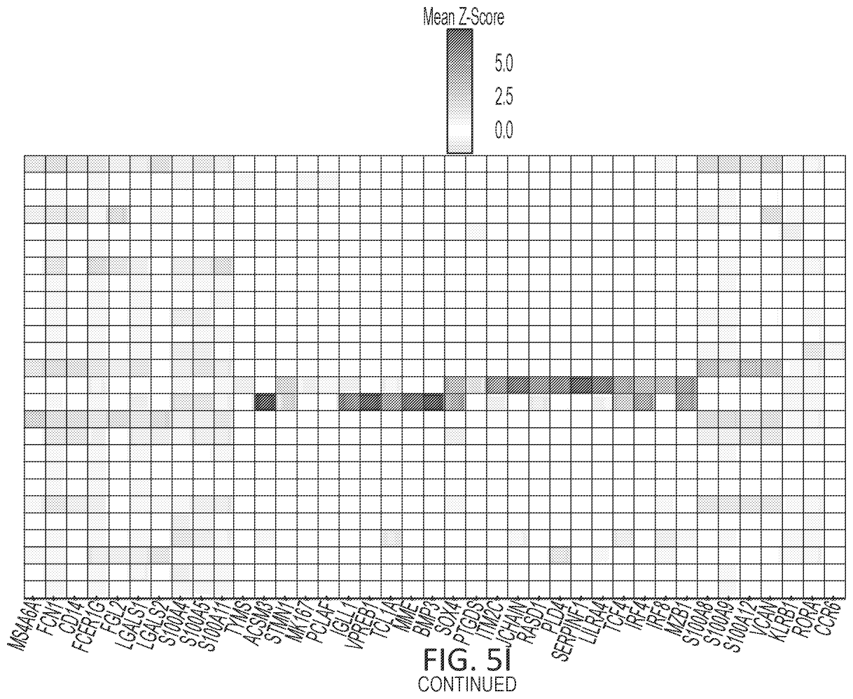

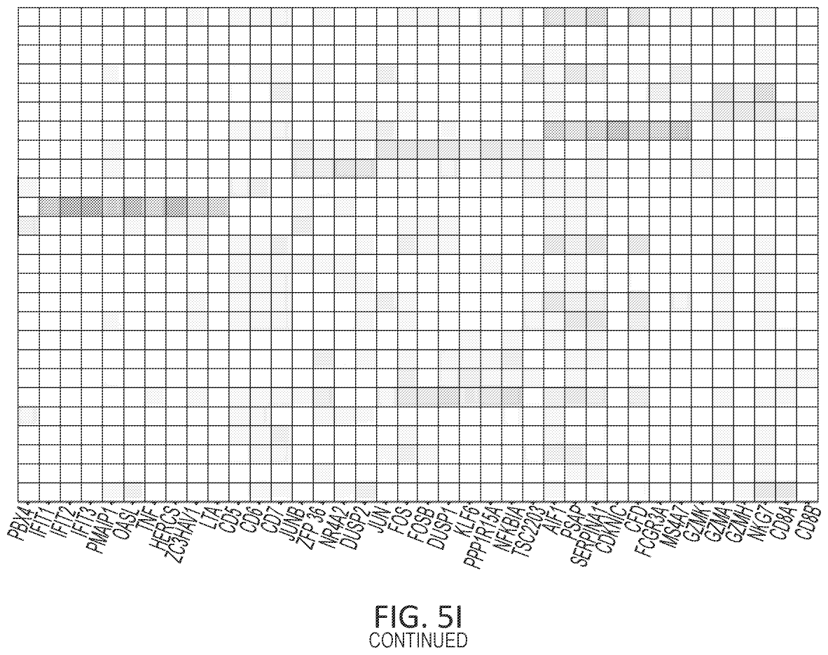

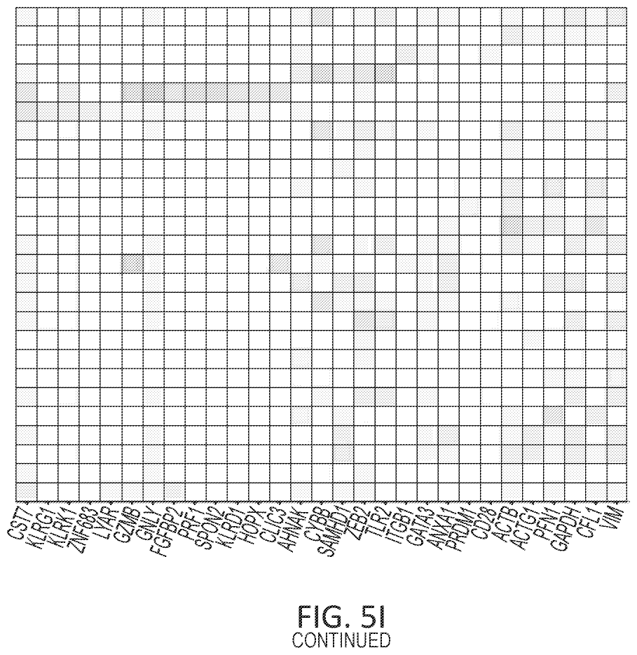

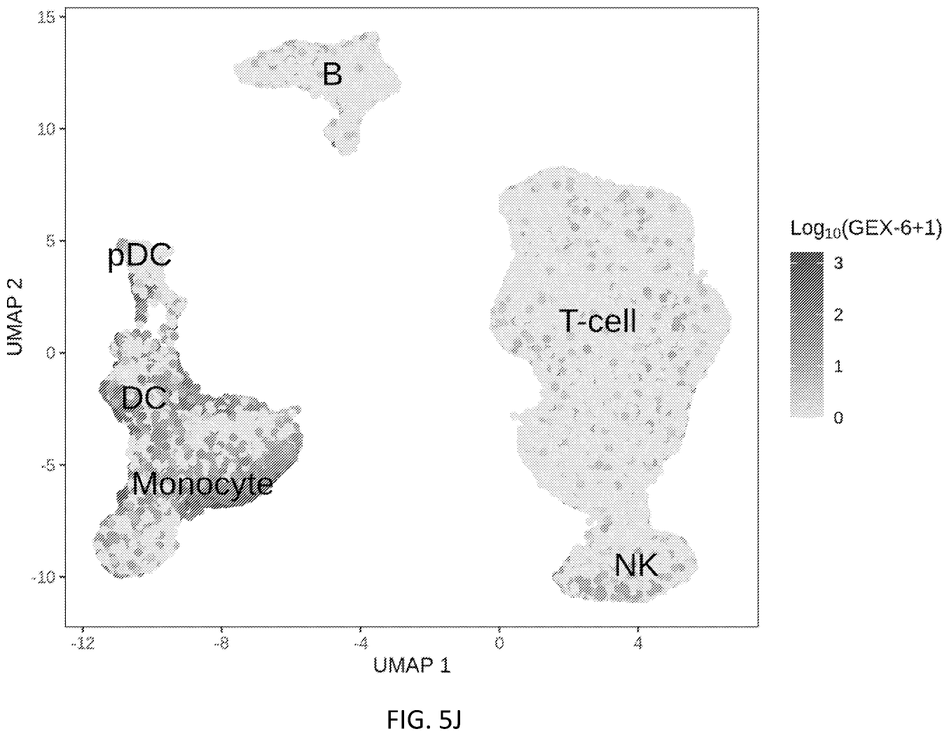

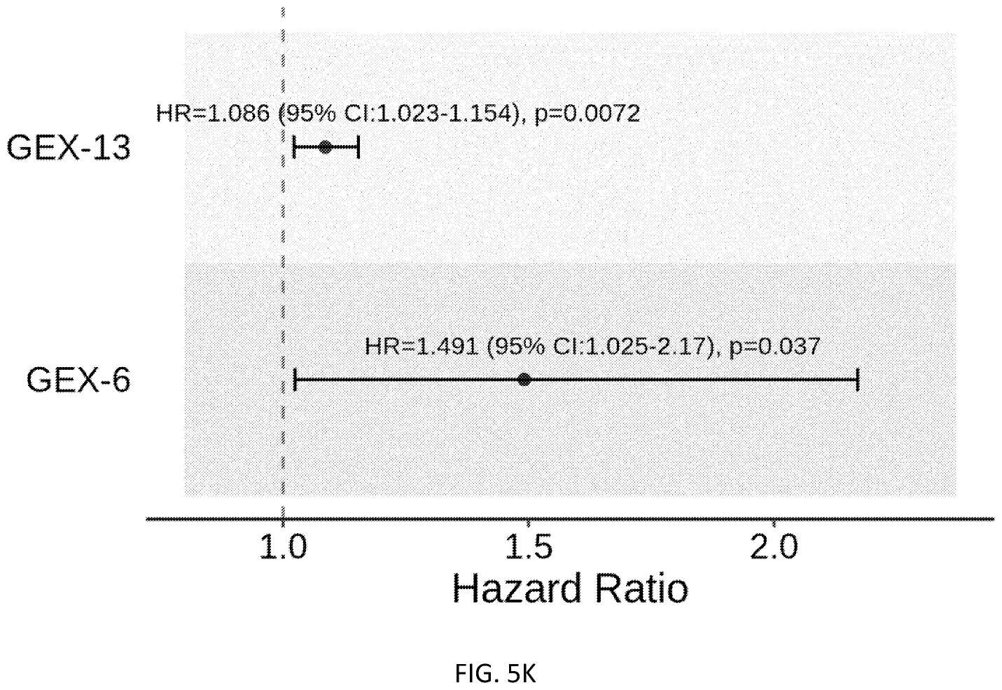

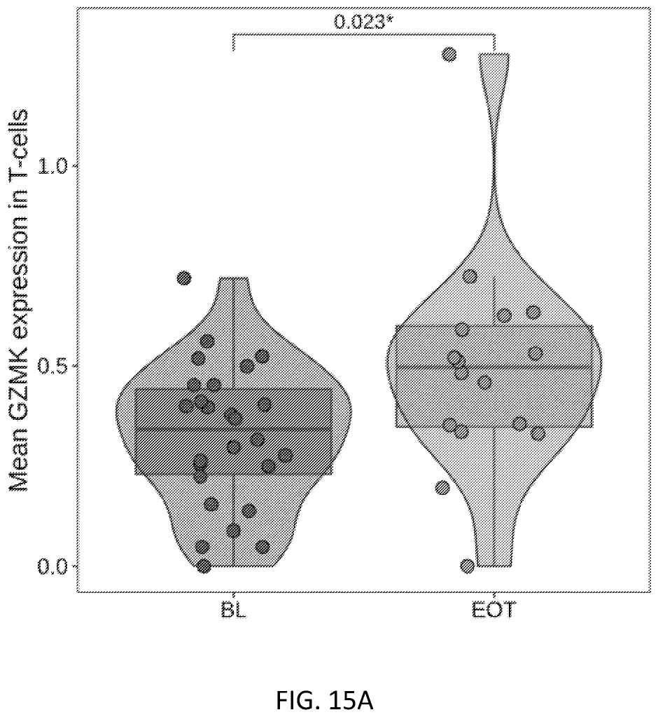

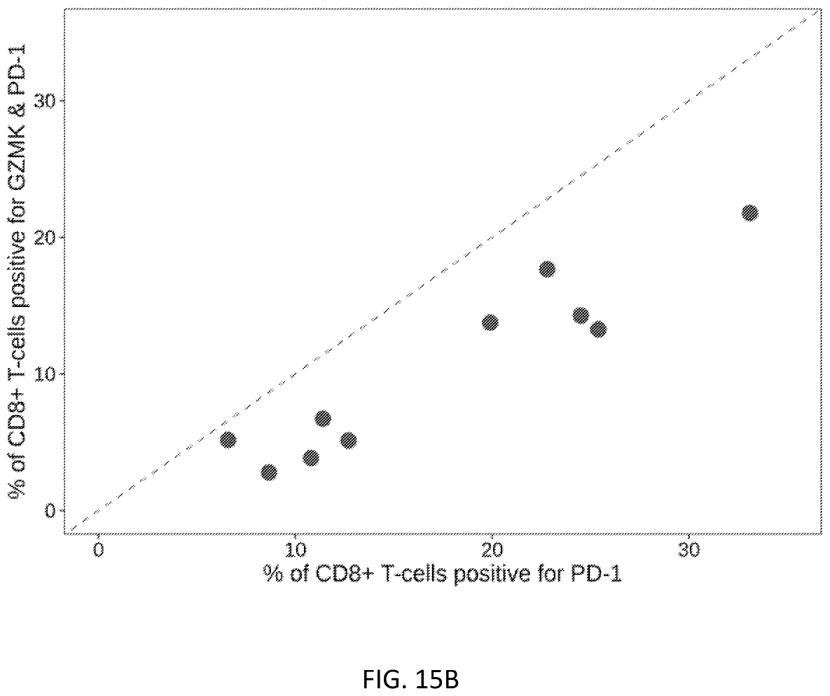

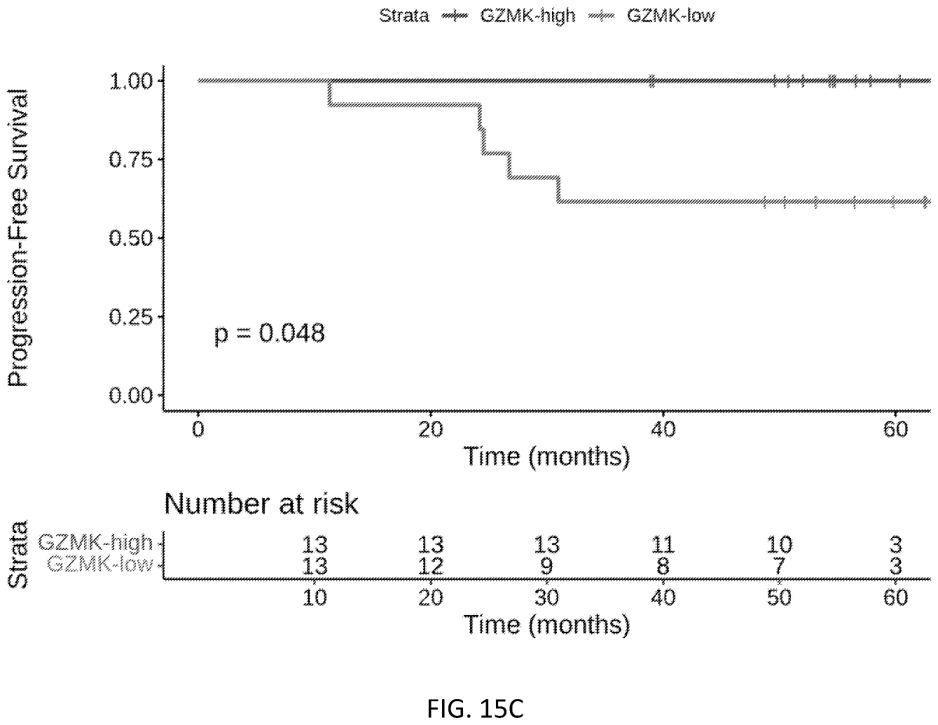

It has been shown that patients with HRSMM have decreased abundance of memory-like GZMK⁺ CD8⁺ TEM cells in favor of more mature GZMB⁺ effectors, and that lower levels of memory T-cells in vivo are associated with shorter overall survival. Therefore, it was hypothesized that higher levels of GZMK⁺ CD8⁺ TEM cells may be associated with prolonged PFS in patients. Patients with HRSMM showed a significant shift from GZMK⁺ to GZMB⁺ CD8⁺ TEM cells in the BM, with a significant decrease in the abundance of GZMK⁺ CD8⁺ TEM cells relative to all cytotoxic T-cells at baseline compared to healthy donors (FIG. 5A). However, the average levels of GZMK expression in patient T-cells increased significantly at EOT, reflecting a significant increase in GZMK-expressing cells, which was validated by CyTOF (FIG. 5B, FIG. 15A). In the PB, where changes in T-cell clonality at EOT were investigated, GZMK⁺ CD8⁺ TEM cells from EOT samples were more likely to belong to expanded clones (FIG. 5C). Together, these results indicate that this subpopulation plays a role in response to immunotherapy. Compared to GZMB⁺ CD8⁺ TEM cells in this cohort, GZMK⁺ CD8⁺ TEM cells showed higher expression of CXCR4 and lower expression of SIP receptors, indicating preferential retainment of GZMK⁺ CD8⁺ TEM cells within the BM (FIG. 5D). Indeed, by comparing the abundance of these cells between matched BM and PB patient samples, enrichment of GZMK⁺ CD8⁺ TEM cells was observed in the BM (FIG. 5E). This enrichment was also observed in BM samples from healthy donors, indicating that this may be a physiological bias. Furthermore, GZMK⁺ CD8⁺ TEM cells had higher expression of canonical exhaustion markers TOX, TIGIT, TNFRSF9 (encoding 4-1BB), and TNFSF9 (encoding 4-1BBL), as well as markers of long-lived memory effector cells, including TCF7 (encoding Tcf-1), CD) 27 and (D) 28, a phenotype most consistent with progenitor exhausted T-cells (T_{PEX}) (FIG. 5D). Indeed, at the protein level, GZMK⁺ CD8⁺ TEM cells were shown to be a major source of PD-1 expression within cytotoxic T-cells in patients (FIG. 5F, FIG. 15B), confirming the suspected Progenitor exhausted T-cells (T_{PEX} phenotype. Progenitor exhausted T-cells maintain their proliferative capacity, can differentiate into terminal effector cells, and are thought to mediate the clinical benefits observed in patients treated with immune checkpoint inhibitors. Patients with a higher abundance of GZMK⁺ CD8⁺ TEM cells had significantly longer PFS in response to immunotherapy (FIG. 5G). Similarly, patients with higher average GZMK expression across their T-cells had significantly longer PFS (FIG. 15C).

Collectively, these results suggest that GZMK⁺ CD8⁺ TEM cells are likely important for disease control and response to therapy in patients with SMM.

### Example 6: Higher Abundance of Memory B-Cells is Associated with Longer Progression Free Survival in Patients with High-Risk SMM Under Treatment

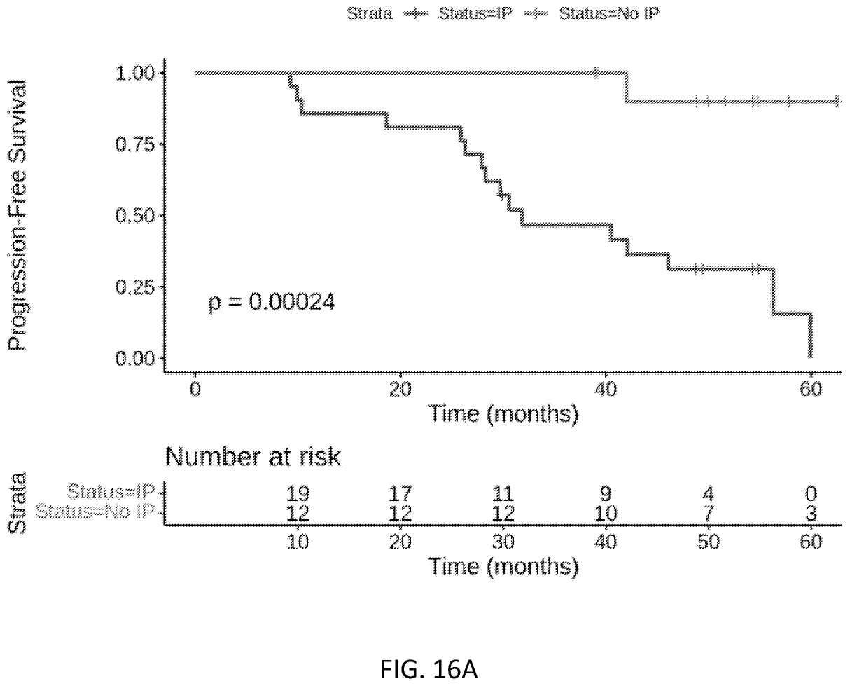

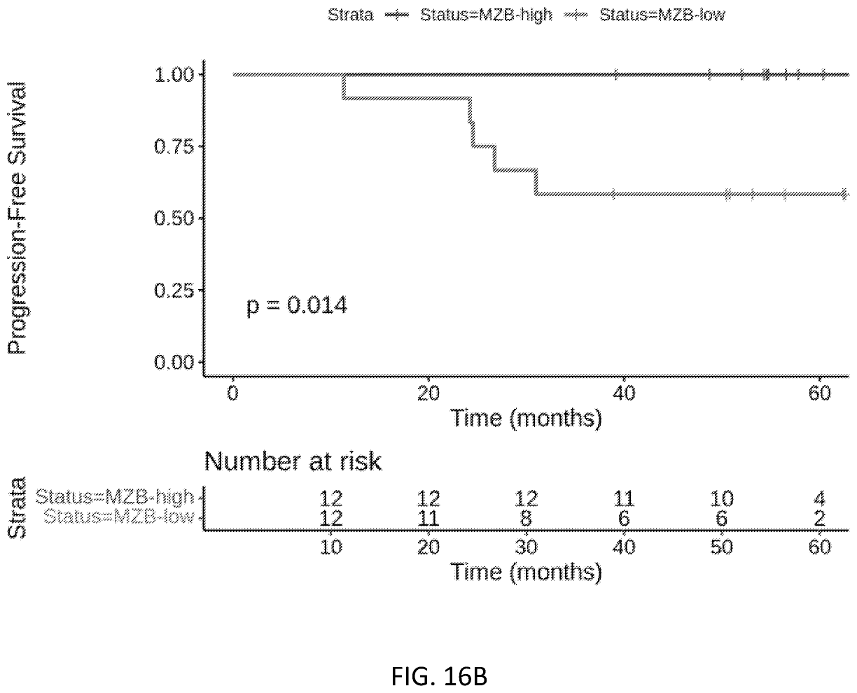

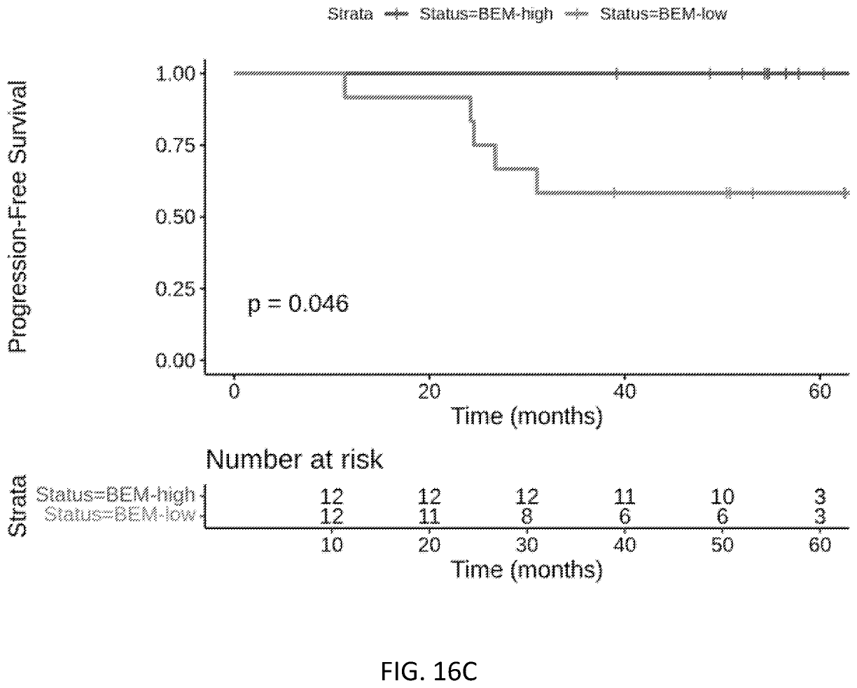

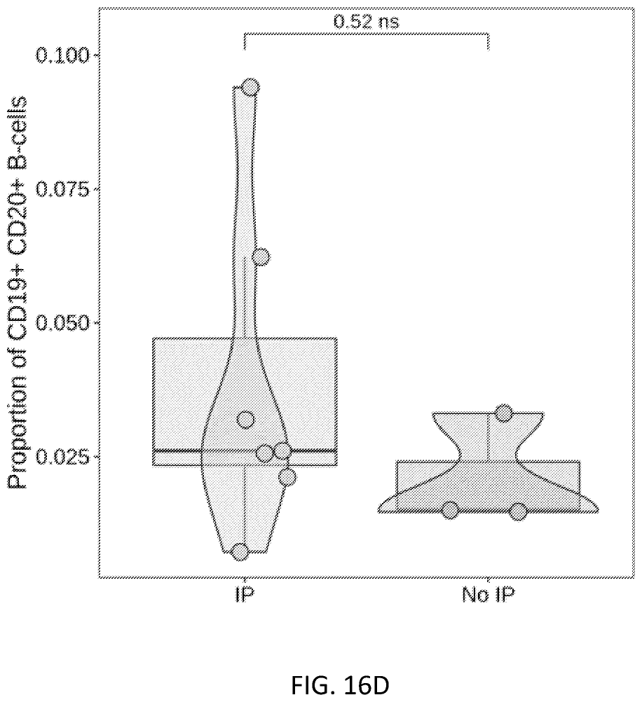

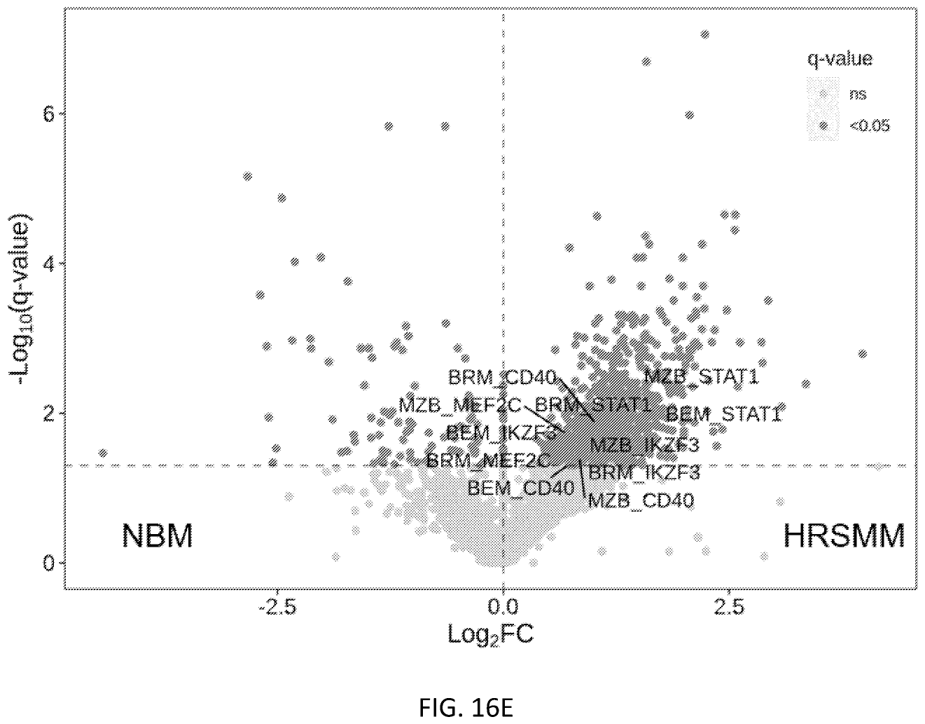

Patients with SMM and immunoparesis (i.e., abnormally low levels of serum immunoglobulin affecting at least one uninvolved isotype) are known to have significantly shorter progression free survival. This was also true in the present cohort (FIG. 16A). Therefore, it was hypothesized that patients with a higher abundance of memory B-cells may have significantly longer PFS. Indeed, in the present cohort, a higher abundance of IGHM-expressing marginal zone B-cells or class-switched memory B-cells were associated with prolonged progression free survival (FIGS. 16B and 16C). However, when the abundance of memory B-cells between patients with and without immunoparesis was compared, no association was observed at the RNA or protein level (FIG. 5H, FIG. 16D). This finding indicates that immunoparesis is not due to lower numbers of memory B-cells, but without intending to be bound by theory possibly due to a defect in normal plasma cell differentiation and/or immunoglobulin secretion. Without intending to be bound by theory, it also suggests that memory B-cells may be important for response to therapy due to mechanisms unrelated to the amount of immunoglobulins they produce. By comparing gene expression between patient and healthy donor B-cells, patient memory B-cells were found to express higher levels of (D) 40, a receptor important for interaction with follicular helper T-cells and class-switching; MEF2C, a transcription factor required for B-cell survival and proliferation following stimulation of the B-cell receptor (BCR); STAT1, a transcription factor that mediates responses to IFN-gamma and is involved in germinal center formation; and IKZF3 (which encodes Aiolos), a transcription factor that regulates memory B-cell formation and plasma cell differentiation (FIG. 16E). Collectively, these results indicate that memory B-cells play an important role in response to therapy in patients with SMM.

### Example 7: Higher Abundance of pDCs and Higher Activity of Pro-Inflammatory Signaling in Antigen-Presenting Cells is Associated with Shorter Progression Free Survival in Patients with High-Risk SMM Under Treatment

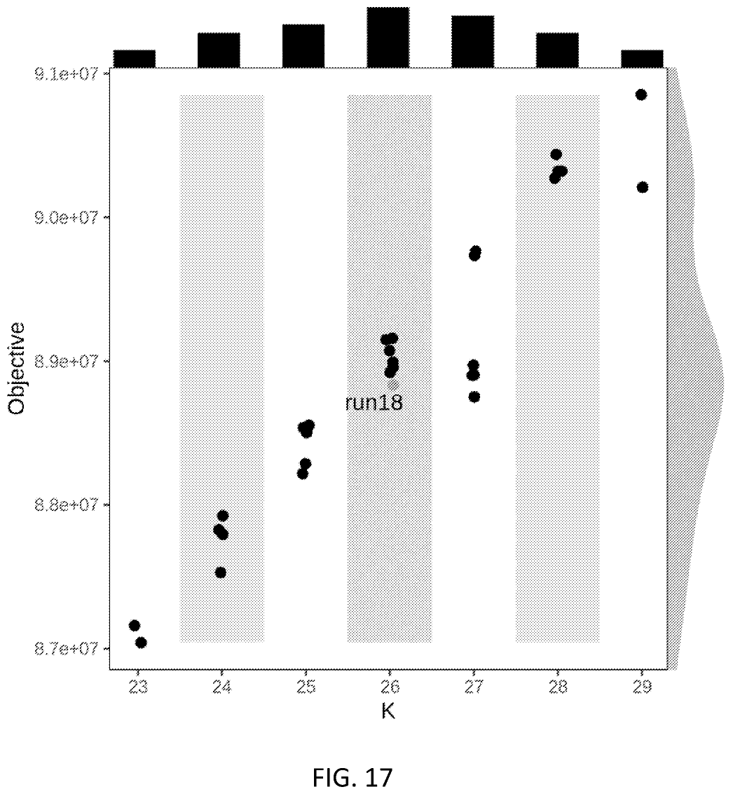

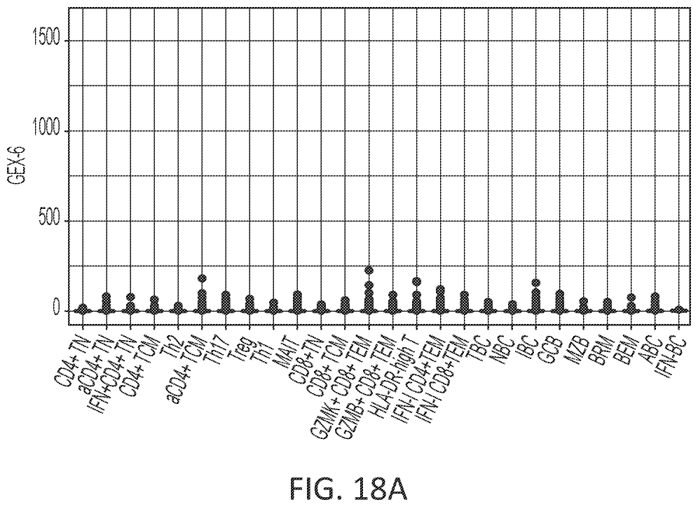

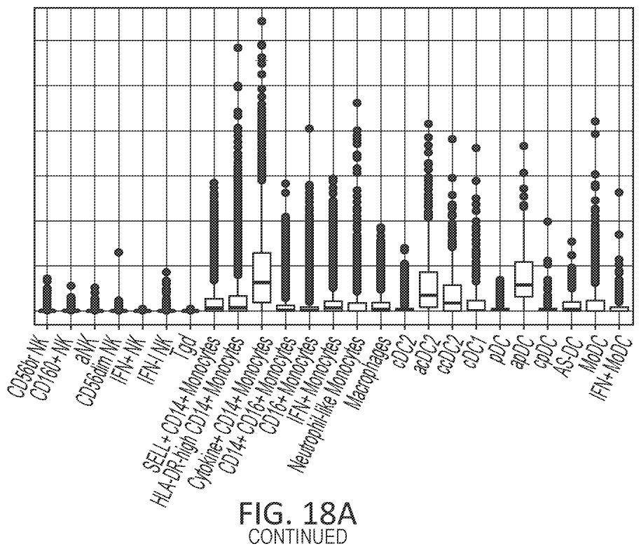

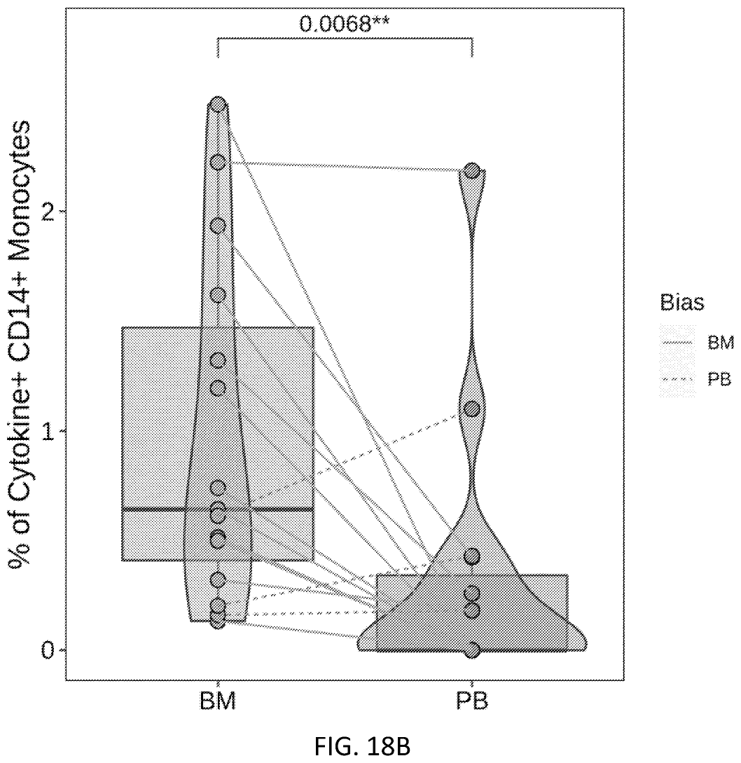

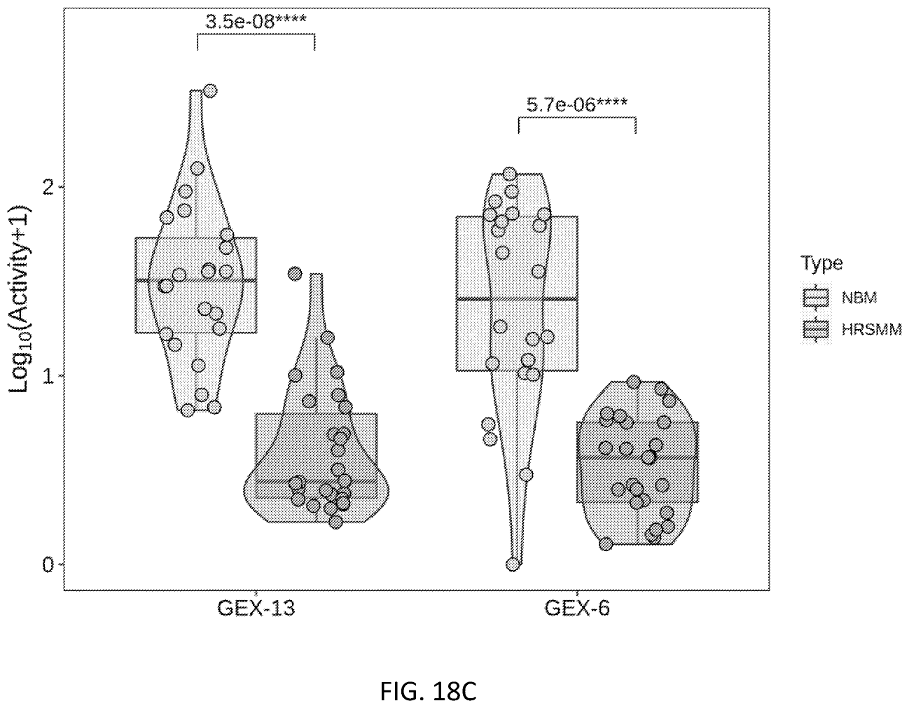

In order to assess the survival impact of expression programs that span multiple subpopulations, Bayesian NMF was performed on the gene expression matrix of lymphocytes and antigen-presenting cells, and 26 gene expression signatures were extracted (GEX) (FIG. 17). The signatures recovered corresponded to established cell-type-specific programs (for example, signature GEX-13 corresponded to plasmacytoid dendritic cells (pDCs), as well as cross-cell type programs like the interferon-gamma (IFN) and interferon type I (IFN-I) programs (FIG. 5I). Specifically, signature GEX-6 was associated with ILIB, CEBPD, CXCL8 (encoding IL-8), CXCL2, and CCL3 expression, which mediate neutrophil chemotaxis and pro-inflammatory signaling. Signature GEX-6 had higher activity in monocyte, cDC2, and pDC subpopulations expressing these same markers (FIG. 5J, FIG. 18A). As Cytokine⁺ CD14⁺ monocytes and pDCs were significantly enriched in patients classified as non-reactive and had two of the highest weights in our classification, it was hypothesized that higher activity of signatures GEX-6 and GEX-13 would be associated with shorter PFS, too. Plasmacytoid dendritic cells, and Cytokine⁺ CD14⁺ monocytes, the monocyte subpopulation with the highest GEX-6 activity, showed a tissue bias in patients and healthy individuals alike, with significant enrichment in the BM compared to PB (FIG. 18B). Consistent with the compositional changes observed before, the baseline activity of signatures GEX-6 and GEX-13 was significantly lower in the BM of patients compared to healthy donors (FIG. 18C). As expected, higher baseline activity of signatures GEX-6 and GEX-13 in patient BM was associated with significantly shorter PFS (FIG. 5K).

### Example 8: Peripheral Blood-Based Immune Profiling Accurately Detects Alterations in Immune Cell Composition and T-Cell Receptor Repertoire Diversity Observed in the Bone Marrow

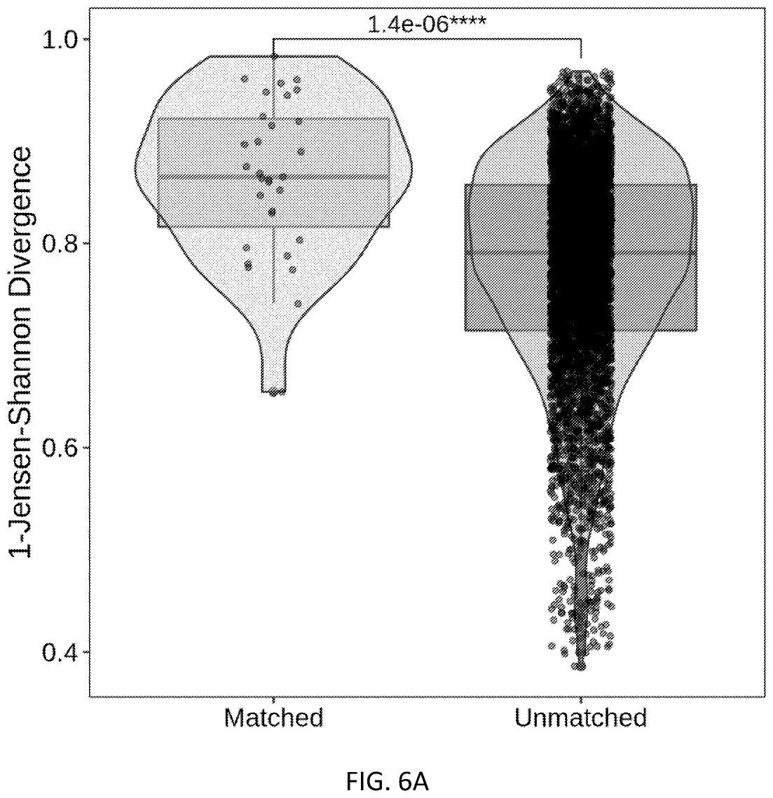

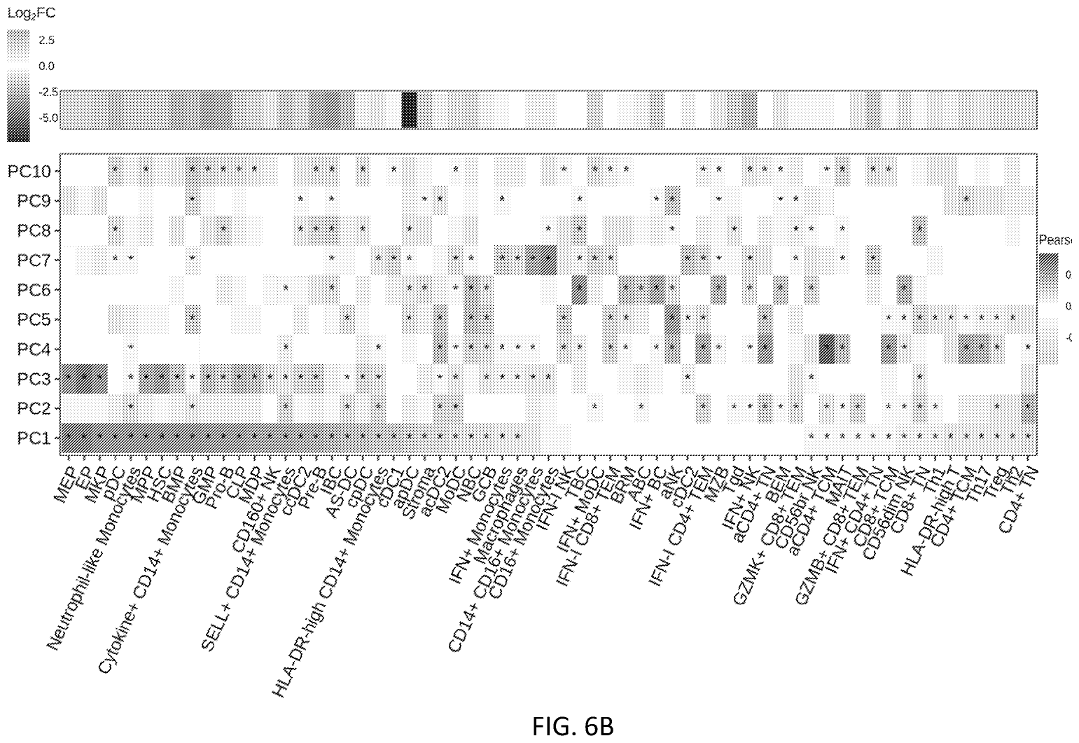

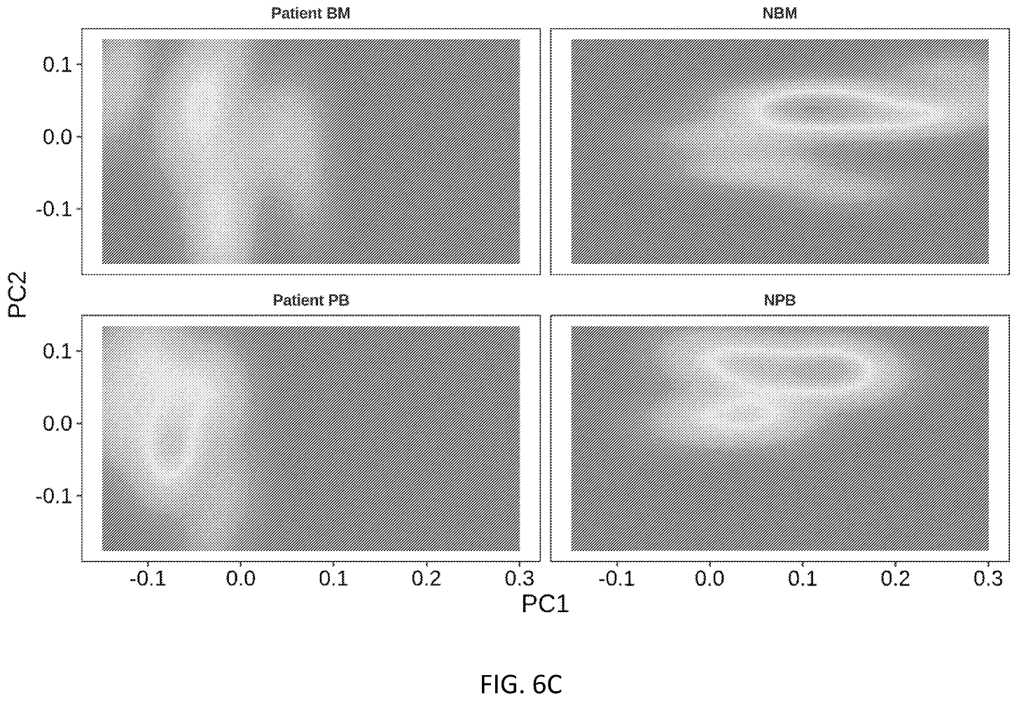

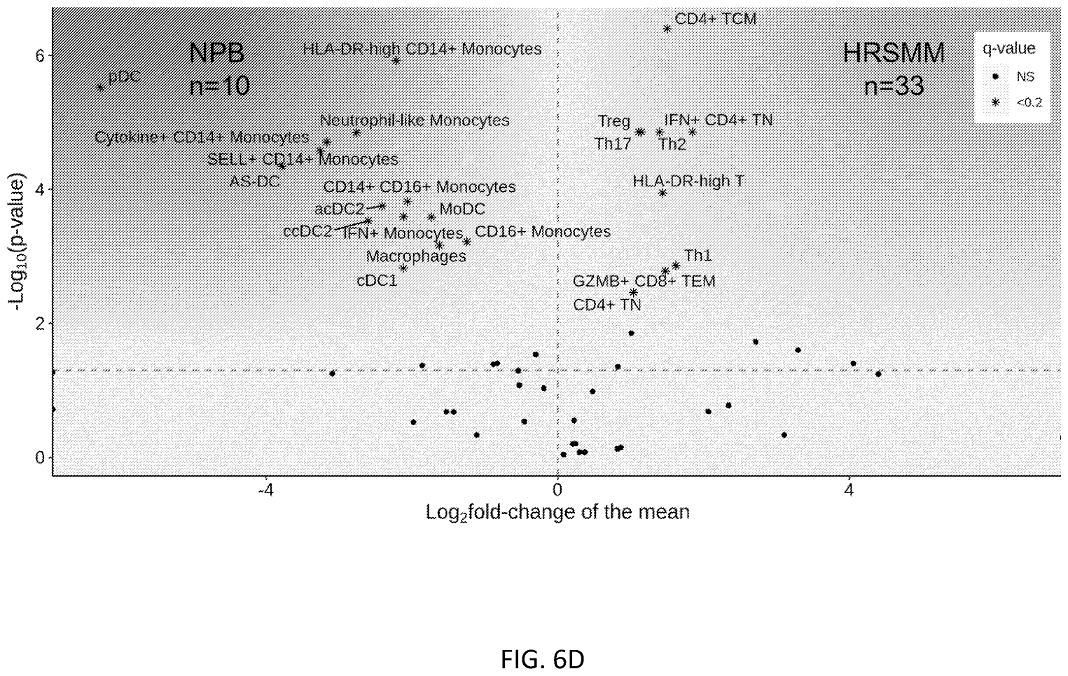

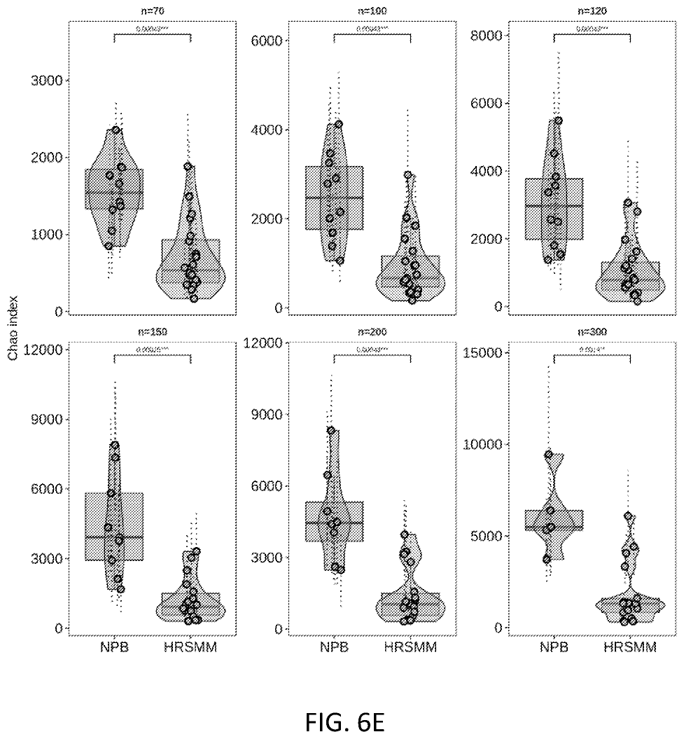

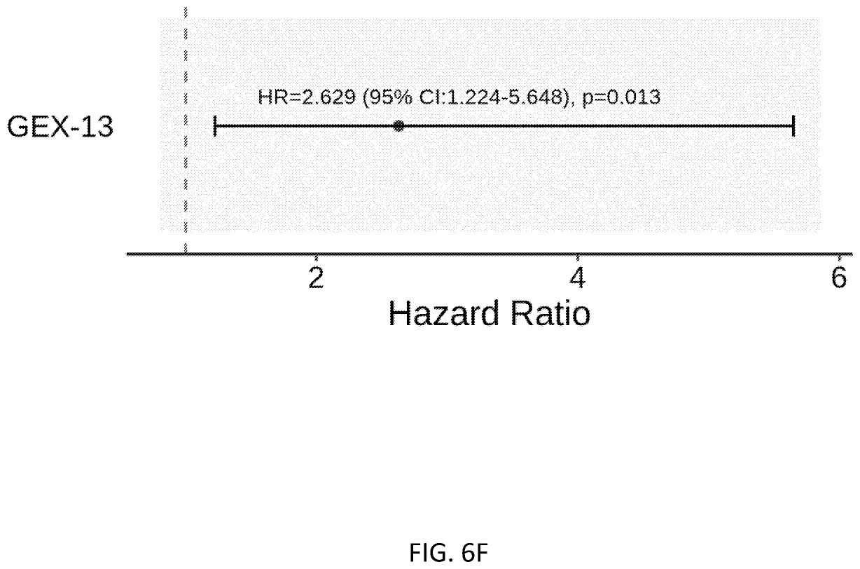

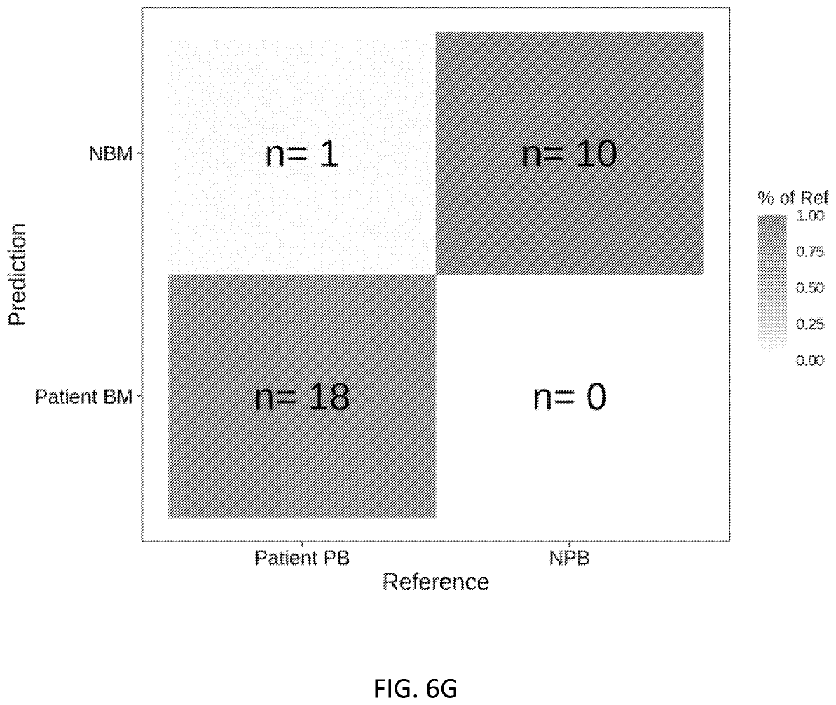

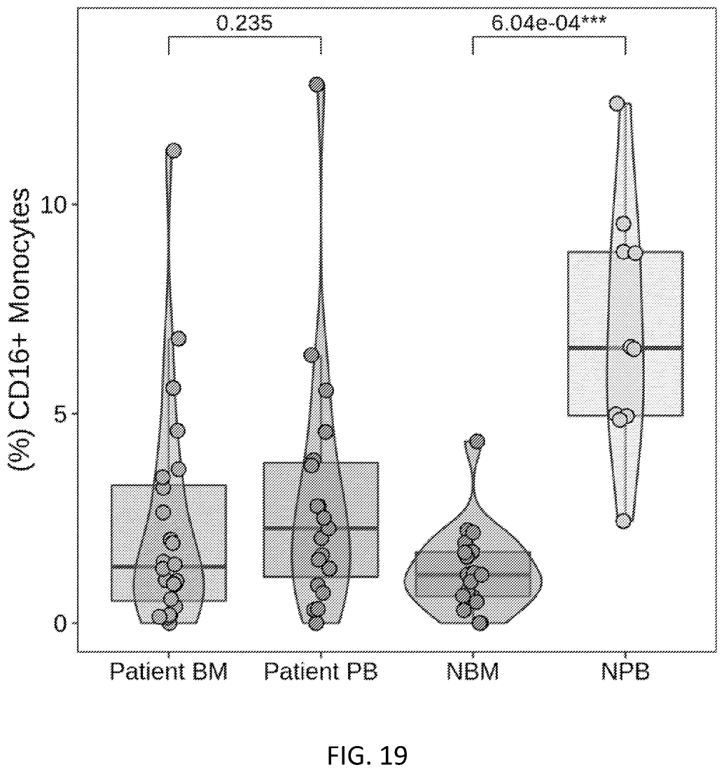

Next, experiments were undertaken to determine whether peripheral blood can be used reliably for immune profiling of patients with HRSMM, as bone marrow biopsies are invasive and carry risk, which precludes regular patient sampling. First, the Jensen-Shannon divergence of immune cell composition was compared between matched and unmatched PB and BM samples, and observed significantly lower divergence in matched pairs, which was also low overall, suggesting that matched PB samples reflect BM composition very well (FIG. 6A). In principle component analysis space, where PC1 captures the compositional changes observed in the bone marrow of patients (FIG. 6B), normal PB and BM samples clustered together on the right end of the axis, while patient PB and BM samples clustered together on the opposite side of PC1 (FIG. 6C), indicating that the compositional differences between patient and healthy PB are similar. Indeed, when PB samples were compared between patients and healthy donors, compositional changes previously discovered in our BM-based analysis were observed (FIG. 6D). Specifically, a significant increase in naïve and memory CD4⁺ T-cells (including Tregs, Th1, Th2 and Th17 cells), GZMB⁺ CD8⁺ TEM, and CD56_{dim} NK cells, and a significant decrease in CD14⁺ monocytes, and pDCs was observed. However, compared to healthy donor PB, patient PB showed significantly lower abundance of CD16⁺ monocytes, which are enriched in patient BM compared to healthy donor BM. Contrary to healthy individuals, where CD16⁺ monocytes are significantly enriched in the PB compared to the BM, in patients the abundance of CD16⁺ monocytes is not significantly different between the PB and the BM, indicating that these cells may be homing to the BM in patients (FIG. 19). Lastly, consistent with what was observed in patient BM, patient PB TCR repertoire showed significantly lower diversity, compared to healthy donors, indicating that the observed effect may not be constrained to the tumor microenvironment, but rather may be systemic (FIG. 6E) These results suggest that certain immune biomarkers related to SMM may be measurable in the PB. Indeed, consistent with what was seen in the BM, higher baseline levels of signature GEX-13 in patient PB were associated with significantly shorter (FIG. 6F). Of note, neither the bone marrow-biased signature GEX-6 nor immune reactivity were prognostic in PB, suggesting that PB-based immune profiling may not be able to replace BM-based profiling entirely.

Lastly, given the concordance of compositional changes between patient BM and PB, experiments were conducted to determine whether immune profiling of PB samples alone could enable the diagnosis of an individual with HRSMM. Remarkably, a classifier that was trained on BM composition data (this time excluding progenitor cells, which are not found in PB) to identify patients from healthy donors was able to correctly classify nearly all of the patient and normal PB samples with an accuracy of 97% (FIG. 6G). Collectively, these findings demonstrate that PB-based immune profiling accurately detects compositional changes observed in the BM, and may hold diagnostic and potential.

Identifying those patients who will benefit the most from treatment of SMM remains an unmet clinical need. Specifically, despite the availability of clinical and genomic biomarkers of progression, little is known about the role of BM immune dysregulation in disease progression and response to treatment or the diagnostic and prognostic utility of PB-based immune profiling. Here, a Phase II trial of the immunotherapeutic anti-SLAMF7 antibody, Elotuzumab, in combination with LenDex was conducted, to determine the utility and safety of early immunotherapy in patients with high-risk SMM, and correlative DNA sequencing and single-cell RNA and TCR-sequencing studies were performed to develop genomic and immune biomarkers for optimal patient selection and monitoring of response to treatment.

EloLenDex was safe and effective, with no evidence of aggressive subclone selection post-treatment in two patients who progressed. In fact, a regression of potentially aggressive subclones was observed in those patients harboring either Amplq or Dellp and a TP53 SNV. These results indicate that in some instances early treatment could alter the clonal dynamics in patients who progress, which could potentially result in improved overall survival in the long-term. Aggressive tumor biology, as determined by the presence of Del17p, was a significant predictor of inferior progression free survival in patients treated with EloLenDex in our cohort. Therefore, patients with high-risk SMM and Del17p may perhaps need more intensive regimens.

Patients with high-risk SMM showed increased abundance of naïve and memory CD4⁺ T-cells, GZMB⁺ CD8⁺ TEM cells and CD56dim NK cells, and decreased abundance of CD14⁺ monocytes, pDCs and progenitor cells compared to healthy donors. Furthermore, their T-cell receptor (TCR) repertoire diversity was significantly lower compared to healthy donors, due to broad low-level clonal expansion, which was mostly observed in GZMB⁺ CD8⁺ TEM cells and Tregs. Importantly, the similarity of a patient's bone marrow immune microenvironment to that of healthy donors was found to be useful for prognostication. At baseline, patients with the least normal-like immune cell composition, presumed to be reactive to the presence of the tumor, had significantly longer progression free survival under treatment with EloLenDex. Reactive patients had lower proportions of pro-inflammatory myeloid cells and pDCs, and more potent, long-lived GZMK⁺ CD8⁺ TEM cells. Granzyme K-expressing cells, which are normally enriched in the BM, had a phenotype most compatible with progenitor exhausted cells, and clonally expanded post-treatment, suggesting that they may play a role in response to therapy. Patients with higher abundance of GZMK⁺ CD8⁺ TEM cells in their BM showed significantly longer progression free survival. At the end of therapy, patients whose BM normalization scores increased post-therapy (Post-therapy Immune Normalization, PIN), had significantly longer progression free survival, indicating that immune profiling may be useful for monitoring of disease response and, is helpful in determining when to stop therapy.

Lastly, immune profiling currently requires a BM biopsy, which precludes its regular use for prognostication. Remarkably, by comparing patient PB to PB from healthy donors, nearly the same alterations in immune cell composition and TCR repertoire diversity were identified as those seen in BM-based comparisons. The similarities were so striking that a classifier trained to detect the presence of SMM based on BM immune profiling was able to correctly classify nearly all of the patient and healthy donor PB samples. These results indicate that PB-based immune profiling likely has diagnostic and prognostic utility.

Collectively, these results demonstrate the utility and safety of EloLenDex in patients with high-risk SMM, provide a comprehensive characterization of alterations in immune cell composition and TCR repertoire diversity in patient BM and PB, nominate novel immune biomarkers for optimal patient selection and assessment of response to treatment, and demonstrate that PB-based immune profiling have diagnostic and prognostic utility in patients with SMM.

### Example 8: Phase II Trial of Elotuzumab, Lenalidomide and Dexamethasone in Patients with High-Risk SMM (E-PRISM)

The results described herein above, were carried out using the following methods and materials.

### Study Design

The primary objective of this trial was to determine the proportion of high-risk SMM patients who were progression-free at 2 years post-treatment. Secondary objectives included safety and toxicity, time to progression, overall response rate, duration of response and overall survival.

Patients were enrolled at Dana Faber Cancer Institute, Boston, Massachusetts; Beth Israel Deaconess Medical Center, Boston, Massachusetts; Carolina HC Levine Cancer Institute, Charlotte, North Carolina; Colorado Blood Cancer Institute, Denver, Colorado; Eastern Maine Medical Center, Bangor, Maine; Karmanos Cancer Institute, Detroit, Michigan; Marlene and Stewart Greenbaum Cancer Center, Baltimore, Maryland; Massachusetts General Hospital, Boston, Massachusetts; Newton-Wellesley Hospital, Newton, Massachusetts; St. Francis Hospital, Hartford, Connecticut; and University of Chicago, Chicago, Illinois.

Patients were eligible for this trial if they were 18 years of age or older, had an Eastern Cooperative Oncology Group performance status of 0 to 2, and were classified as high-risk SMM. High-risk SMM was defined as in Rajkumar et al. (Blood 125, 3069-3075 (2015)) by the presence of bone marrow clonal plasma cells ≥10% (but less than 60%) and at least one of the following: serum M protein ≥3.0 mg/dL; IgA SMM; immunoparesis with reduction of two uninvolved immunoglobulin isotypes; serum involved/uninvolved free light chain ration ≥8 (but less than 100); evolving type of SMM, i.e., progressive increase in M protein level; bone marrow clonal plasma cells 50-60%; abnormal cell immunophenotype (>95% of bone marrow plasma cells are clonal) and reduction of one or more uninvolved immunoglobulin isotypes; t (4;14) or Del17p or Amplq; increased circulating plasma cells; MRI with diffuse abnormalities or 1 focal lesion (≥5 mm); PET-CT with one focal lesion (≥5 mm) with increased uptake without underlying osteolytic bone destruction. Furthermore, patients had to show no evidence of myeloma-defining events as described by the IMWG (Rajkumar, S. et al. The Lancet Oncology 15, e538-e548 (2014), and the following laboratory values had to be shown within 28 days prior to registration: Absolute Neutrophil Count ≥1000/mm³, Platelets (PLT)≥50,000/mm³, Total Bilirubin≤2.0 mg/dL, Aspartate and Alanine Aminotransferase (AST/ALT)≤3×ULN, and an estimated creatinine clearance >50 mL/min/1.73 m². Patients with other concurrent chemotherapy, immunotherapy, radiotherapy, or any ancillary therapy considered investigational, serious medical or psychiatric illness likely to interfere with participation, a diagnosis or treatment for another malignancy within 2 years of enrollment were excluded.

All patients provided informed consent. The review boards of all participating centers approved the study in accordance with the Declaration of Helsinki and the International Conference of Harmonization Guideline for Good Clinical Practice. This study was registered as a phase II study with ClinicalTrials.gov NCT02279394.

### Study Treatment

This phase II trial used a single-stage design to assess the efficacy of the combination of Elotuzumab and Lenalidomide with or without Dexamethasone in patients with high-risk SMM. Patients were randomized 1:1 to Arm A (EloLenDex) or Arm B (EloLen), based on stratification factors described below.

In the treatment schema a cycle is defined as 28 consecutive days. In cycles 1 and 2, 10 mg/kg of intravenous push Elotuzumab were administered on days 1, 8, 15, and 22. In cycles 3-4, it was administered on days 1 and 15. A 25 mg dose of oral Lenalidomide was administered on days 1-21 of each cycle. In Arm A, 40 mg of oral Dexamethasone were administered on days 1, 8, 15, and 22 in cycles 1 and 2 and on days 1, 8, and 15 in cycles 3-8. Patients on maintenance treatment (cycles 9-24) were administered 20 mg/kg of intravenous push Elotuzumab on day 1 and 15 mg oral Lenalidomide on days 1-21. All patients received thromboprophylaxis.

In consideration of Mateos et al. New England Journal of Medicine 369, 438-447 (2013), investigators were given the option to allow high-dose Dexamethasone to be resumed during maintenance due to biochemical progression (progressive increase in serum M-spike by at least 25% with an absolute increase of at least 0.5 g/dL, or urine M-spike by at least 25% with an absolute increase of at least 200 mg/24 hours, on two successive evaluations, as determined by the IMWG response criteria, or documented progression by serum free light chain criteria in the absence of serum or urine involvement). A total of 5 patients were given Dexamethasone during maintenance. The dosing schedule for this followed that of cycles 3-8, with 8 mg intravenous push and 32 mg oral Dexamethasone on days 1 and 15 and 40 mg oral Dexamethasone on day 8.

### Stratification Factors

Originally, the study aimed to secondarily determine whether Dexamethasone is beneficial or detrimental in this immunotherapy regimen for SMM. Therefore, the study was designed with two arms, meant to serve as two independent phase II studies to determine the activity of EloLen with or without Dexamethasone in the patient population. Patients were stratified by age (≥65 vs<65) and high-risk cytogenetics (t (4;14), t (14;16), Del17p, TP53 mutation, Amp1q) (high-risk, low-risk, FISH failure) before being randomly assigned to one of the two arms.

### Arm B Accrual

Arm B accrual was halted in April 2016 for two reasons: 1) treatment with Elotuzumab required premedication with high doses of steroids to avoid infusion reactions and the difference in dosing of Dexamethasone between the two arms (approximately 4 mg Dexamethasone per dosing day) for the first 2 cycles was not significant, and 2) a publication by Paiva et al. Blood 127, 1151-1162 (2016) demonstrated that once weekly high-dose Dexamethasone does not have a detrimental effect on the immune system in patients with SMM in a clinical trial of Lenalidomide and Dexamethasone. The patients who were originally enrolled in Arm B remained in Arm B until completion of therapy.

### Cohort Details

In total, 51 patients enrolled and received at least one dose of treatment. Three patients were deemed ineligible soon after they received treatment, due to misdiagnosis (overt myeloma with lesions on PET-CT, amyloidosis and light chain deposition disease), and were excluded from further analysis. Two patients received less than 2 full cycles of treatment and were excluded from our overall response assessment and correlative studies, but were included for toxicity assessment.

### Imaging

In order to assess the extent of disease involvement, imaging studies were performed at both baseline and EOT. All patients had either an MRI with skeletal survey or a PET/CT scan.

### Efficacy and Safety Assessments

Toxicities were monitored throughout the study and for up to 30 days after the last dose of the study drug. Adverse events (AEs) were graded according to the National Cancer Institute Common Terminology Criteria for Adverse Events (CTCAE; version 4.0; Bethesda, MD). In addition, neuropathy symptoms were assessed using the FACT/GOG-Neuropathy questionnaire (Version 4.0). Efficacy was measured using the IWMG criteria for response³¹.

### Statistical Analysis & Biomarker Discovery

Patient baseline characteristics were summarized as number of patients or median of values. Responses to study treatment were reported as proportions with 90% exact binomial confidence intervals.

The proportion of patients alive and progression-free at 2 years was compared to the previously published rate for high-risk SMM—a median time to progression of only 1.9 years according to the Kyle et al. model (New England Journal of Medicine 356, 2582-2590 (2007)). Therefore, for this study, a 2-year progression-free survival rate of 50% would not be considered promising; instead, a progression-free survival rate of 70% or higher would be a promising result.

This phase II study used a single-stage binomial design. With a sample size of 39 patients, the probability of concluding that the treatment is effective if the true rate is 50% was 0.10 and was >0.9 if the true rate is 70%. Assuming an ineligibility rate of 5%, it was an objective of this study to accrue 41 patients per Arm to the trial. Time to response (TTR) was measured from treatment initiation to the date the response was first observed. Duration of response was measured from response to progressive disease (PD) or death, censored at the date of last disease assessment for those who did not progress. Time-to-progression (TTP), and progression-free survival (PFS) were measured from the time of treatment initiation to event (PD for TTP; PD or death for PFS). Patients without event were censored at the date of last disease assessment for both TTP and PFS. If non-protocol therapy, excluding erythropoietin, was added prior to an event, patients were censored in the time-to-event analyses at the initiation of non-protocol therapy. The Kaplan-Meier method was used to estimate time to event and the log-rank test was used to compare time to event. Cox proportional hazards regression was employed to assess the significance of variable associations with outcome. Continuous variables, such as cell type proportions, were tested as such, and were dichotomized based on the median for Kaplan-Meier curves. Statistical analyses were performed using R version 4.0.2. All results with p-values<0.05 were considered statistically significant. The Benjamini-Hochberg method was used to correct for multiple hypothesis testing, when appropriate. Our data cut-off was Nov. 7, 2020.

### Treatment Discontinuation and Patient Disposition

The median duration of treatment was 24 cycles (range=1-24). In total, 40 of 51 patients received all 24 cycles. Of those, 30 (75%) were in arm A and 10 (25%) in arm B. Of the 10 patients in Arm A who did not complete all 24 cycles, 3 patients discontinued therapy during the induction cycles due to an incorrect initial diagnosis (one participant had myeloma lytic lesions on a PET scan that was originally misread at an outside institution and re-checked at DFCI afterwards; another participant had amyloidosis that was missed at the time of enrollment but was discovered after 3 cycles of therapy—the patient achieved VGPR on therapy before being removed from the protocol and placed on active amyloidosis therapy with a proteasome inhibitor-based approach; one final patient had light chain deposition disease, which was identified on a kidney biopsy after one cycle of therapy—the patient was then removed from the protocol to start active therapy). Six other patients discontinued therapy due to toxicity and withdrawal of consent or physician decision.

Overall, two deaths occurred and were unrelated to MM. One patient had Type I Diabetes and was hospitalized for Diabetic ketoacidosis (cycle 19). This was at a time when the patient was not receiving Dexamethasone. The patient developed diverticulitis, bowel perforation, sepsis and died due to sepsis. The second patient was on the last cycle of therapy when they experienced uncontrolled hypertension due to non-compliance with their hypertension medications. They then developed heart failure and died of hypertensive crisis.

### Safety and Tolerability

Treatment-related Grade 2 adverse events with at least 10% frequency and all Grade 3-5 events are summarized in Table 2 and FIG. 8A. Forty-nine patients experienced at least one toxicity while on study with worst grades of 1-mild (n=0), 2-moderate (n=11/51, 22%), and 3-severe (n=29/51, 57%), 4-life threatening (n=7/51, 14%), and 5-fatal (n=2/51, 4%). The most common toxicities of any grade were upper respiratory infection (n=22/51; 43%), fatigue (n=21/51; 41%), hypophosphatemia (n=21/51; 41%), neutropenia (n=15/51; 29%), and lymphocytopenia (n=14/51; 27%). Thromboembolic events occurred in 6 patients (12%), and no secondary leukemias were observed. Dexamethasone dose had no effect on the type or frequency of treatment-related toxicities.

### Time to Event Analysis

Median follow-up for all 51 patients was 50 months (range, 2-67). Median overall survival, time-to-progression, and progression-free survival have not been reached (FIG. 8B). Six total patients had progression-free survival events defined as progressive disease to myeloma defining events (n=4) or death (n=2). Another four patients (n=4) were treated based on worsening biomarkers and the physician's decision and were considered as progressors for the purposes of our correlative studies only.

### Impact of 20-2-20 Stratification Model on PES

The capacity of the 20-2-20 model to identify patients who exhibit superior PFS under treatment was explored. Although curve separation was observed and the hazard ratio confidence intervals were wide, indicating low power, there was no evidence that 20-2-20 was a significant predictor of progression in the present cohort (Intermediate-risk HR: 1.86 (95% CI: 0.3-11.53), High-risk HR: 4.38 (95% CI: 0.83-23.04), FIG. 9A).

ECOG Randomized Trial of Lenalidomide Versus Observation in Patients with High-Risk SMM

Due to the unavailability of patient-level information, survival data were recapitulated from published Kaplan-Meier (KM) plots in Lonial, et al Journal of Clinical Oncology 38, 1126-1137 (2020). The curves and at-risk tables were extracted as raster images, and their {x,y} coordinates were digitized using the commercial software DigitizeIt²³. Using the algorithm proposed by Guyot²⁴, the coordinates and number of patients at-risk during each time interval were used to estimate censored, time-to-event data. With the patient-level data, we were able to re-generate the survival curves, at-risk tables, log-rank tests, etc. and to compare with our own data. In particular, the KM method was used to explore the effect of the 20-2-20 risk stratification system on PFS in the Lenalidomide arm of the ECOG trial and the log-rank test to compute the p-value (FIG. 9B).

Whole-Exome Sequencing of Patients at Baseline and after Treatment

### Samples & Sequencing

Whole-Exome Sequencing was performed on baseline bone marrow aspirate specimens from 40 study participants. Tumor DNA was extracted from CD138⁺ cells from the patients' bone marrow. For germline control, DNA was obtained from PBMCs. Genomic DNA was extracted using the QIAamp DNA mini kit (QIAGEN) according to the manufacturer's protocols, and the DNA concentration was quantified using PicoGreen dsDNA Assay kit (Life Technologies). WES libraries were prepared by Agilent SureSelect XT2 Target Enrichment kit (V5⁺UTR probes). For three individuals, libraries for deep targeted sequencing were prepared using SureSelect XT Reagent Kits (Agilent) and an in-house bait set targeting 117 genes, comprising pan-cancer driver genes and frequently mutated genes in MM¹⁰. Lastly, for two out of four patients who progressed to overt myeloma, tumor samples were available at time of progression and were also studied by WES (Roche MedExome⁺) to characterize the landscape of clonal evolution under treatment. All sequencing was performed on the Illumina HiSeq 4000 platform at the Genomics Platform of the Broad Institute of MIT & Harvard. Six samples were excluded from analysis due to low tumor purity (<15%).

### WES Analysis

The output from Illumina software was processed by the Picard data processing pipeline to yield BAM files containing well-calibrated, aligned reads. We have utilized the Getz Lab CGA WES Characterization pipeline developed at the Broad Institute to call, filter and annotate somatic mutations and copy number variation. The pipeline employs the following tools: MuTect²⁵, ContEst²⁶, Strelka²⁷, Orientation Bias Filter²⁸, DeTiN²⁹, AllelicCapSeg³⁰, MAFPONFilter³¹, RealignmentFilter, ABSOLUTE³², GATK³³, PicardTools, Variant Effect Predictor³⁴, Oncotator³⁵. SNVs and CNVs were further cleaned with a custom PON made of matched normal samples³¹, and a bait bias filter was developed to correct for observed artifacts in the SNV data, as described before¹⁰. ABSOLUTE was applied to estimate sample purity, ploidy, and absolute somatic copy numbers, which were used to infer the cancer cell fractions of point mutations and CNVs from the WES data, following the framework previously described Carter et al., Nature Biotechnology 30, 413-421 (2012)).

### Assessment of Clonal Selection Upon Treatment

A primary concern with early treatment of patients with high-risk SMM is the potential for selection of aggressive subclones that carry high-risk genetic abnormalities. To explore if that was what drove progression in two (out of four) individuals in the cohort (patients #21 and #51) who developed overt MM and had available samples for testing, whole exome sequencing was performed on DNA extracted from bone marrow CD138⁺ cells taken at baseline and at EOT. Both individuals had high-risk genomic features at baseline: patient #21 had Del17p, while patient #51 had t (4;14), Dellp, TP53 & KRAS single nucleotide variants (SNVs). Clonal selection was assessed by cancer cell fractions (CCF) and their confidence intervals, as produced by ABSOLUTE³², which takes purity and ploidy into account. Considering established drivers in MM, as well as all arm-level CNVs, no evidence of aggressive subclone selection was observed (FIGS. 1B and 1C). In patient #21, several large-scale copy number variants (CNVs) were enriched after treatment, but none that is associated with an increased risk of progression or death. In both patients, we observed a contraction of potentially aggressive subclones that harbor Amp1q (patient #21) and Del1p and a TP53 SNV (patient #51), signified by non-overlapping cancer cell fraction confidence intervals.

### Single-Cell RNA-Sequencing of Patients at Baseline, C9D1, and End of Treatment

### Samples, Sequencing and Analysis Workflow

Bone marrow and peripheral blood mononuclear cells (BMMCs & PBMCs) were isolated using Red Blood Cell lysis buffer (ThermoFisher). Bone marrow mononuclear cells were further subjected to magnetic bead enrichment for CD138 (Miltenyi Biotec), in order to isolate CD138-negative immune cells. Overall, single-cell RNA-sequencing was performed on 149 CD138-negative BMMC and PBMC samples from 34 patients enrolled in the E-PRISM trial. Specifically, 60 CD138-negative BMMC samples were sequenced at baseline (n=28), cycle 9, Day 1 (C9D1, n=16) and EOT (n=16), 68 PBMC samples at baseline (n=33), C9D1 (n=14), and EOT (n=21)], 11 CD138-negative BMMC samples from age-matched healthy donors, and 10 PBMC samples from age-matched healthy donors. For 23 patients, baseline BM and PB samples were matched; for 8 patients, C9D1 BM and PB samples were matched; for 13 patients EOT BM and PB samples were matched. Of note, in contrast to the patient samples, healthy donor BM and PB samples were from different individuals.

For library preparation, Chromium Single Cell 3′ Gene Expression (n=40) and Chromium Single Cell 5′ Gene Expression kits (n=109) by 10× Genomics were used. Specifically, 3′ single-cell RNA-sequencing was performed on 40 CD138-negative BMMC samples drawn at baseline (n=11), C9D1 (n=13) or EOT (n=9), and PBMC samples drawn at baseline (n=4) and C9D1 (n=3). 5′ single-cell RNA-sequencing was performed on 98 CD138-negative BMMC samples drawn at baseline (n=17), C9D1 (n=3) or EOT (n=7), and PBMC samples drawn at baseline (n=29), C9D1 (n=11) or EOT (n=21). 5′ single-cell RNA-sequencing was performed on 11 CD138-negative BMMC samples and 10 PBMC samples from healthy donors. All 3′ libraries were sequenced on a HiSeq2500 instrument and all 5′ libraries were sequenced on a NovaSeq4000 instrument at the Genomics Platform of the Broad Institute of MIT & Harvard. CellRanger v.5.0.1. was used for FASTQ file extraction and generation of count matrices³⁶.

Ambient RNA correction with SoupX, doublet detection with Scrublet, scDblFinder and SCDS, normalization with Scran and integration with Harmony (correcting for sample ID and kit) was performed. Droplets were deemed to be doublets when at least 2 out of four methods classified them as such and they were only removed from consideration when they clustered together and co-expressed markers of multiple cell types. Droplets containing dying cells with more than 15% mitochondrial gene expression were removed before clustering. Clusters which presented with higher mitochondrial and ribosomal gene expression, clearly separated from well-annotated clusters and lacked interpretable expression markers were removed downstream. In total, 211,374 immune cells (˜1,419 cells/sample), comprising T-cells, NK cells, B-cells, Monocytes, Dendritic cells, and progenitor populations were profiled (FIGS. 2A-2L). Data from the E-PRISM study were processed together with our cohort of non-trial patients with MGUS (n=6), low-risk SMM (LRSMM, n=3), high-risk SMM (HRSMM, n=9), newly diagnosed MM (NDMM, n=9) and healthy donors (NBM, n=11). For each cell population (T-cells, NK cells, B-cells, Monocytes, Dendritic cells, progenitor cells), feature selection, dimensionality reduction and clustering were also performed separately, to identify cell subpopulations (see below for annotation strategy).

Cell type/subtype proportions were calculated as the fraction of cells belonging to said type/subtype out of all immune cells unless otherwise specified. For the purposes of cell type proportion comparisons, samples with fewer than 100 cells were removed from consideration. Proportion changes were assessed with Wilcoxon's rank-sum tests or paired t-tests when comparing serial timepoints within the same patients or matched BM and PB samples. When multiple hypotheses were tested, p-values were corrected using the Benjamini-Hochberg approach. For the purposes of survival analysis, cell type abundance was discretized based on the median.

Differential expression was performed using Wilcoxon's rank-sum tests at the single-cell level and DESeq2 at the pseudobulk level. Genes with more than 3 counts in at least 10 cells were considered for differential expression. For pseudobulk analyses, genes with more than 10 counts in at least 25% of samples were considered for differential expression. P-values were corrected for multiple hypotheses testing using the Benjamini-Hochberg approach.

### Cell Type and Subtype Annotation

Gene expression markers used for cell type and subtype annotation can be found in FIGS. 2B, 2D, 2F, 2H, 2J, and 2L. Scran-normalized expression values were Z-scored and the mean Z-score per cluster was used to plot the heatmaps.

Within the T-cell compartment, naïve CD8⁺ (CD8⁺ TN) and CD4⁺ T-cells (CD4-TN) (LEF1, TCF7, SELL, (CR7), central memory CD8⁺ (CD8⁺ TCM) and CD4⁺ T-cells (CD4⁺ TCM) (FAS, IL.7R, HNRNPLL), helper type 1 T-cells (Th1) (CD4, CXCR3, GZMA, GZMK, LYAR, C (15), helper type 2 T-cells (Th2) ((D) 4, GATA3, KRTI, (CR4), helper type 17 T-cells (Th17) (CD4, RORA, KLRB1, CCR6, TNFRSF4), regulatory T-cells (Treg) ((7) 4, IL.2RA, FOXP3, CTLA4, RTKN2, IKZF2), mucosa-associated invariant T-cells (MAIT) expressing a mixture of Th17 and Th1 markers, as well as the specific marker SI. (4A10 (MAIT annotation was confirmed by invariant TCR chain usage in overlapping TCR-sequencing data), two effector memory CD8⁺ T-cell subpopulations, GZMK⁺ CD8⁺ TEM cells (GZMK, CMC1, XCL1, XCL2) and terminal effector cells (GZMB⁺ CD8⁺ TEM) (GZMB, GZMH, FGFBP2, PRF1, FCGR3A, GNLY) were identified. Furthermore, we identified a subpopulation of CD4⁺ TN cells expressing genes related to response to interferon-gamma (IFN⁺ CD4⁺ TN) (MX1, ISG15, IF16, IFI44L), two separate subpopulations expressing genes related to response to interferon type-I (IFN-I CD4⁺ TEM & IFN-I CD8⁺ TEM) (IFIT1, IFIT2, IFIT3, TNF), and two activated CD4⁺ subpopulations expressing genes of the AP-1 module and general activation markers (aCD4⁺ TN & aCD4⁺ TCM) (JUN, FOS, FOSB, CD69, DUSP1, TS (221) 3). Lastly, a second CD4⁺ regulatory subpopulation, clustering adjacent to Tregs and expressing high levels of (′TLA4 and MHC-II-encoding genes, a profile most consistent with effector Tregs were identified, which was annotated descriptively as HLA-DR-high T-cells, due to non-specific expression levels of (D) 4, IL2RA and FOXP3.

Within the NK cell compartment, CD56bright NK cells (CD56br NK) (NCAMI), CD2, XC12, KLRC1, IL7R, SELL, GZMK), CD160⁺ NK cells expressing markers of CD56_{bright} NK cells as well as (D) 160, SPRY2, and TOX2, CD56_{dim} NK cells (CD56dim NK) (GZMB, GZMH, FGFBP2, PRF1, NKG7, CX3CR1, GNLY, B3GAT1) and gamma-delta T-cells (Tgd) ((D3D), TRGV9, TRIV2) were identified. Furthermore, a subpopulation of NK cells expressing genes related to response to interferon-gamma (IFN⁺ NK) (MX1, ISG15, IFI6, IFI44L), a separate subpopulation expressing genes related to response to interferon type-I (IFN-I NK) (IFIT1, IFIT2, IFIT3, TNF), and an activated subpopulation with characteristics of both CD56bright and CD56_{dim} NK cells (aNK), expressing genes of the AP-1 module and general activation markers (FOS, JUN, JUNB, CD69, CXCR4, DUSP1, NFKB1, NFKB2) were identified.

Within the B-cell compartment, immature B-cells (IBC) (MME, CD19, CD38, IGHM, SOX4, TOLIA, RAG1, RAG2, VPREB1, IGLL1), transitional B-cells (TBC) (CD) 19, MS4A1, (D) 38, IGHM, IGHD, TOLIA, ILAR, SELL), naïve B-cells (NBC) (CD19, MS4A1, IGHM, IGHD, IL4R, SELL, CCR7, FCER2, ABCB1), a population expressing higher levels of CXCR5, CD83, and TNFSF9 (encoding 4-1BBL), called germinal center B-cells (GBC), marginal zone B-cells (MZB) ((DIC, IGHM, PLD4, LINC01857), atypical B-cells (ABC) (TBX21, ITGAX, FCRL5, ENC1, TNFRSFIB, SOX5, MPP6), and two populations of class-switched memory B-cells: one expressed higher levels of T (F7 and was called resting memory B-cells (BRM), while the other expressed higher levels of FAS, CD86, ITGB1, S100A10, and TOX, and was called effector memory B-cells (BEM) were identified. Lastly, a subpopulation of B-cells expressing genes related to response to interferon-gamma (IFN⁺ BC) (MX1, ISG15, IF16, IF144L) was identified.

Within the monocyte compartment, three main subpopulations of classical (CD14⁺) monocytes were identified: a subpopulation expressing higher levels of SELL, VCAN, S100A8, S100A9, and S100A12 (SELL⁺ CD14⁺ Monocytes), a subpopulation expressing higher levels of genes encoding MHC-II peptides (HLA-DR-high CD14⁺ Monocytes), and a subpopulation expressing pro-inflammatory factors, such as IL1B, CXCL8, CXCL2, CCL3, (CL4, and CEBPD) (Cytokine⁺ CD14⁺ Monocytes). A fourth subpopulation expressed (D) 14, SELL, VCAN, S100A8, S100A9, S100A12, as well as MPO, AZU1, ELANE, PRIN3, and RNASE2, and were called Neutrophil-like Monocytes. Furthermore, we identified non-classical CD16⁺ Monocytes (F (GR3A, MS4A7, CSF1R, CDKN1C, RHOC) intermediated CD14⁺ CD16⁺ Monocytes expressing (D) 14, FCGR3A (which encodes CD16), and high levels of MHC-II-encoding genes, a CD16⁺ subpopulation of macrophages expressing high levels of complement factor Clq ((IQA, CIQB, CIQC, SELENOP, SD (3), and a population of monocytes expressing genes related to response to interferon-gamma (IFN⁺ Monocytes) (MX1, ISG15, IF16, IFI44L).

Within the dendritic cell compartment, canonical dendritic cells type 2 (cDC2) (FCER1A, CLEC10A, CD1C and MHC-II encoding genes), canonical dendritic cells type 1 (CDC1) (CLEC9A, C1orf54, IDO1, CADM1, CLMK, BATI3), monocyte-derived dendritic cells (MoDC) (FCER1A, CLEC10A, CD1C, CD14), plasmacytoid dendritic cells (pDCs) (LILRA4, IL3RA, GZMB, IRF4, SERPINF1), and AXL⁺ SIGLEC6⁺ dendritic cells (AS-DCs) (AXL, SIGLEC6, ADAM33, LTK) were identified. Furthermore, cycling subpopulations of pDCs and cDC2s (cpDC, ccDC2), activated subpopulations expressing the pro-inflammatory markers IL1B, CXCL8, and CCL3 (acDC2, apDC), and a MoDC subpopulation expressing genes related to response to interferon-gamma (IFN⁺ MoDC) (MX1, ISG15, IF16, IF144L) were identified.

Lastly, within the progenitor cell compartment, hematopoietic stem cells (HSC) ((D) 34, CD164, BEX1, BEX2, AVP, CRHBP, HLF), multi-potent progenitor cells (MPP) ((D) 34, CD33, MPO, FLT3, MZB1), granulocyte-monocyte progenitor cells (GMP) (MPO), AZUJ1, PRIN3, ELANE, LYZ), monocyte-dendritic cell progenitors (MDP) (MPO, LYZ, IRF8, LY86, RUNX2, LILRB4), megakaryocyte-erythroid progenitors (MEP) (GATA2, FCER1A, ITAG2B, CSF2RB), erythroid progenitors (EP) (GATA1, EPOR, CA1, CA2, EPCAM, KLF1, BLVRB, APOC1, APOE), megakaryocyte progenitors (MKP) (GATA2, FCERIA, ITGA2B, PLEK, GP1BB, PPBP, PF4, GP9), basophil-mast cell progenitors (BMP) (MS4A2, MS4A3, TPSAB1, TPSB2, HDC, CLC, PRG2), common lymphoid progenitors (CLP) (FLT3, TRBC2, MZB1, LTB, JCHAIN, ADA, BCL2), pro-B-cells (Pro-B) (DNTT, MME, PAX5, RAG1, RAG2), and pre-B-cells (Pre-B) (VPREB1, IGLL1, JCHAIN, TOL1A). Within this compartment we also identified a small population of stroma cells (CXCL12, LEPR, KITLG, VCAM1, COL1A1, COL1A2, (CL2) were identified.

### Gene Expression Signature Discovery & Annotation

A Bayesian version of Non-negative Matrix Factorization (NMF) was performed using SignatureAnalyzer^{44,45} on the count matrix of the top 5,000 highly variable genes for gene expression signature discovery. Half-normal priors were imposed for both the W (gene by signature) and the H matrix (cell by signature), as we reasoned that a single gene could contribute towards multiple expression programs and a single cell could be described by multiple expression programs. The tool was run 30 times and the run that resulted in a K (i.e., number of signatures) equal to the mode of the K distribution and had the lowest objective was selected for downstream analysis (FIG. 17). Gene expression signature markers were nominated by (i) multiplying the W matrix by the sum of signature activity across all cells in the H matrix, (ii) calculating the fraction of each signature's activity for each gene (matrix F) and (iii) ranking genes based on the product of W (i.e., how strongly each gene contributes to the signature) and F (i.e., how strongly each signature contributes to the gene). A total of 26 gene expression (GEX) signatures were extracted (FIG. 5I). GEX-1 corresponded to cytokine signaling associated with GZMK-expressing subpopulations (XCL1, XCL2, CCL3, CCL4); GEX-2 captured naïve T-cells (TCF7, LEF1, TXNIP, IL7R, LTB, NOSIP); GEX-3 captured dendritic cells (CLEC10A, FCER1A, CD74, HLA-DRA, HLA-DRB1); GEX-4 captured B-cells ((D) 19, MS4A1, IGHM, IGHD, CD79A, CD79B); GEX-5 was associated with ferritin heavy and light chain expression (FTH1, FTL) and was primarily active in myeloid cells; GEX-6 corresponded to pro-inflammatory signaling (IL1B, CXC18, CXCL2, (EBP)) and was primarily active in myeloid cells; GEX-7 corresponded to genes induced by interferon-gamma (MX1, STAT1, XAF1, IF16, IF144L., ISG15) and was active in T-cells, B-cells, NK cells, and monocytes; GEX-8 was associated with thymosin expression and the alpha and beta chains of the TCR (TMSB4X, TMSB10, TRAC, TRB (I), and was primarily active in T-cells; GEX-9 was associated with histone 1 gene expression (HIST1H1C, HIST1H1D, HIST1H1E) and was primarily active in dendritic cells; GEX-10 was associated with metabolic programming (GLUL, SLC43A2, SLC25A37) and hypoxia-related signaling (HIF1A, TIMP1, NEAT1), and was primarily active in myeloid cells; GEX-11 corresponded to CD14⁺ monocytes ((I) 14, MS4A6A, FCN1, LYZ); GEX-12 corresponded to immature B-cells (ACSM3, IGLL1, VPREB1, TOL1A, MME, SOX4); GEX-13 corresponded to pDCs (LILRA4, JCHAIN, SERPINF1, PTGDS, ITM2C, IRF4, IRF8); GEX-14 corresponded to a different program within classical monocytes (S100A8, S100A9, S100A12, VCAN); GEX-15 corresponded to Th17 cells and MAITs (RORA, KLRB1, CCR6); GEX-16 was associated with interferon type-I signaling (IFIT1, IFIT2, IFIT3, PMAIP1, OASL, INF, HERC5, ZC3HAV1); GEX-17 was associated with T-cell surface antigens (CD5, CD6, CD7); GEX-18 and GEX-19 were associated with genes of the AP-1 module (JUN, FOS, FOSB) and general activation markers (DUSP1, TS (22D) 3, NFKBIA); GEX-20 corresponded to CD16⁺ monocytes (FCGR3A, CDKN1C, SERPINA1, MS4A7); GEX-21 corresponded to cytotoxic T-cells (GZMK, GZMH, NKG7, CD8A, CD8B); GEX-22 corresponded to GZMB⁺ cytotoxic T-cells and NK cells (GZMB, GNLY, SPON2, CLIC3, FGFBP2); GEX-23 was associated with genes frequently expressed by myeloid cells (AHNAK, TLR2, CYBB) and was primarily active in monocytes; GEX-24 was associated with memory CD4⁺ T-cell expression markers (GATA3, CD) 28, PRDM1, ITGB1, ANXA1) and was primarily active in those populations; GEX-25 and GEX-26 were associated with cytoskeleton-related genes (ACTB, ACTG1, PFN1, CFL) and were primarily active in myeloid cells.

For the purposes of survival analysis, each sample was assigned its mean signature activity across cells, and mean activity was discretized based on the median.

### Naïve Bayes Classifier and Normalization Scores

A Naïve Bayes classifier was trained using the R package caret on a training set (n=41) comprising bone marrow samples from patients with high-risk SMM and healthy donors, using 10-fold cross-validation and 100 iterations, following a parameter sweep. The input to the classifier was the composition matrix of cell type proportions and included data on 63 subpopulations. The accuracy in the testing set (n=16) was 94% (95% CI: 69.8%-99.8%, p=0.0057) (FIG. 14). For each cell type, its importance to the classification using the varImp( ) function was computed, which employs an ROC approach, and signed it based on whether the cell type had higher mean abundance in disease (minus) or healthy donors (plus). Then the weighted sum of the product of each cell type's proportion and its signed importance (normalization score) was computed, and patients were classified as reactive (least normal-like) or non-reactive (most normal-like), based on the median normalization score of patients at baseline.

### Single-Cell TCR-Sequencing of Patients at Baseline, C9D1, and End of Treatment

### Samples, Sequencing and Analysis Workflow

Single-cell TCR-sequencing was performed on 86 samples with available single-cell RNA-sequencing data. Specifically, 65 BM and PB patient samples drawn before and after therapy were profiled [BM: 14 at BL, 2 at C9D1, 2 at EOT; PB: 22 at BL, 8 at C9D1, 17 at EOT], and 21 samples from healthy donors [BM: 11; PB: 10]. CellRanger v5.0.1 was used to extract FASTQ files and produce clonotype matrices. When multiple alignments were called for a single chain, the alignment with the most UMIs was selected, and when multiple chains were called for a single cell barcode, the top two chains in terms of UMI counts were selected.

To control for variability in T-cell numbers across samples, a range of T-cell numbers (n=75-500) from each sample was downsampled with 100 iterations, and averaged repertoire diversity estimates across iterations. Repertoire diversity was assessed using the Chao index, as implemented in the vegan R package. To estimate the proportion of clonotypes belonging to a given clone size (Rare: <=1%; Small: >1% and <5%; Medium: >=5% and <10%; Large: >=10%) in a given sample, we downsampled 100 T-cells from each sample with 100 iterations, converted clonotype counts into frequencies, computed the frequency of each clone size, and subsequently averaged the frequency of each clone size across iterations and renormalized those frequencies for plotting. To estimate the probability of a given T-cell subtype to be clonally expanded in patients or healthy donors, first was computed stable clone sizes for each clonotype in each sample, by downsampling 100 T-cells with 100 iterations, converting clonotype counts into frequencies, computing each clonotype's clone size, and subsequently assigning each clonotype the mode of its clone size distribution across all iterations. Next, for each T-cell subtype, 100 cells across all patients were randomly sampled (or healthy donors), and for each iteration, the frequency of rare (proportion of singletons) and expanded (1-rare) clonotypes was computed. For visualization purposes, the average frequencies were renormalized and plotted. Expansion frequencies across all iterations for a given T-cell subtype in patients were compared to those in healthy donors using Wilcoxon's rank-sum test.

### Cytometry by Time-of-Flight (CyTOF)

In-house conjugation of Granzyme K antibody was performed with Maxpar labeling kit per manufacturer instructions (Fluidigm). All antibodies were used per the manufacturer's recommendation (Fluidigm).

Cryopreserved patient BMMC (Bone Marrow Mononuclear Cells) samples were thawed, counted using AO/PI and pelleted by centrifugation at 400 g for 10 minutes. Cells were then incubated in 103Rh viability stain for 15 minutes, washed in CyFACS and incubated with undiluted Human TruStain FcX for 10 min for Fc receptor blocking. Antibodies mastermix was applied to cell suspension for 30 minutes, washed and fixed/permeabilized with FoxP3 Fixation/Permeabilization Concentrate and Diluent, prepared following manufacturer's guidelines (eBioscience). A mix of intracellular antibodies prepared with 1× Perm Wash was added to each sample and incubated for 30 minutes. Next, cells were washed with 1× Perm Wash, and incubated overnight at 4° C. in FoxP3 Fixation/Permeabilization Concentrate and Diluent, containing 191/193Ir DNA Intercalator. Prior to acquisition, samples were transferred to 5 mL round-bottom polystyrene tubes with cell strainer caps, washed and filtered with Cell Staining Buffer (CSB), Cell Acquisition Solution (CAS), and resuspended in CAS supplemented with EQ Four Element Calibration Beads (1:10).

All mass cytometry data was collected on a Helios™ Mass Cytometer (Fluidigm). The instrument was tuned using CyTOF Tuning Solution according to the Helios User Guide (Fluidigm, p. 60-68). A brief overview of tuning steps includes Pre-XY Optimization, Mass Calibration, XY Optimization, DV Calibration, Dual Calibration, Gases/Current Calibration, and QC report. EQ Four Element Calibration Beads (1:10 in CAS) were used according to the manufacturer protocol before and during acquisition. The data was normalized using the FCS Processing tab of the Fluidigm CyTOF Software 7.0.8493.

Data analysis was manually performed using FlowJo 10.7.1. For initial data clean-up cell events were gated to remove dead cells and debris through biaxial plots of Time vs. Event Length, Beads (for removal of the EQ Calibration Beads), and Gaussian-derived parameters (Residual, Width, Offset). The viability stain 103Rh, was used to gate out dead cells on PBMC populations. All viable cells were backgated on both DNA parameters (191Ir and 193Ir) to ensure no doublets were included. Please refer to Hallisey M, Dennis J, Abrecht C, Pistofidis R, Schork A, Lightbody E et al. Mass cytometry staining for human bone marrow clinical samples. STAR Protocols. 2022; 3 (1): 101163 for detailed protocol.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-29. (canceled)

30. A method for monitoring combination therapy comprising an immunotherapeutic or immunomodulatory agent in a subject having a hematological malignancy or precursor condition, the method comprising characterizing normalization scores during the course of therapy, wherein an increase in immune normalization score relative to a baseline normalization score indicates that the combination therapy is effective.

31. A method for characterizing a subject being treated for a hematological malignancy or precursor condition at end of treatment, the method comprising characterizing normalization scores at end of treatment, wherein a significant increase in post-therapy immune normalization score characterizes the subject as having a good prognosis, and no significant change in post-therapy immune normalization score characterizes the subject as having a poor prognosis.

32-33. (canceled)

34. The method of claim 31, wherein characterizing post-therapy immune normalization (PIN) comprises determining a threshold based on the distribution of change in normalization scores.

35. The method of claim 31, further comprising detecting the presence of Del17p in a biological sample of the subject, wherein such detection indicates a need for more aggressive treatment.

36. The method of claim 31, further comprising characterizing the subject's bone marrow microenvironment by detecting an increase in naïve and memory CD4+ T-cells, GZMB+ CD8+ effector memory T-cells and CD56dim NK cells, and a reduction in CD14+ monocytes, pDCs and progenitor cells in a biological sample relative to a healthy control.

37. (canceled)

38. The method of claim 36, wherein detecting at end of treatment a bone marrow microenvironment that closely resembles the bone marrow microenvironment of a healthy control indicates that the treatment was effective and may be discontinued.

39-59. (canceled)

60. A method for identifying a human subject having smoldering multiple myeloma (SMM), Monoclonal Gammopathy of Undetermined Significance (MGUS), or Multiple Myeloma (MM) that would benefit from treatment, the method comprising:
determining that mononuclear cells obtained from a blood sample, CD138-negative (CD138−) mononuclear cells obtained from a bone marrow sample or a blood sample, or a bone marrow tissue section obtained from the human subject have one or more biomarkers selected from:
(i) an increased or decreased abundance of granzyme K positive (GZMK⁺) T cells relative to a control abundance of GZMK⁺ T cells;
(ii) an increased or decreased abundance of GZMK⁺ natural killer (NK) cells relative to a control abundance of GZMK⁺ NK cells;
(iii) an increased or decreased abundance of Th17 cells relative to a control abundance of Th17 cells;
(iv) an increased or decreased abundance of plasmacytoid dendritic cells (pDCs) relative to a control abundance of pDCs;
(v) an increased or decreased abundance of hematopoietic stem cells (HSCs) relative to a control abundance of HSCs;
(vi) an increased or decreased abundance of mature B-cells, which include both naïve and memory B-cells, relative to a control abundance of mature B-cells;
(vii) an increased or decreased activity of GZMK-associated signaling relative to a control activity of GZMK-associated signaling;
(viii) an increased or decreased activity of Th17-associated signaling relative to a control activity of Th17-associated signaling;
(ix) an increased or decreased activity of a compositional signature that captures an abundance of mature B-cells relative to a control activity of a compositional signature that captures an abundance of mature B-cells;
(x) an increased or decreased activity of a compositional signature that captures an abundance of HSCs relative to a control activity of a compositional signature that captures an abundance of HSCs; and
(xi) an increased or decreased activity of an immune reactivity score relative to a control activity of an immune reactivity score.

61. The method of claim 30, wherein the subject is human and the human subject is undergoing treatment for SMM, MGUS, or MM.

62-99. (canceled)

100. The method of claim 60 further comprising identifying a human subject having smoldering multiple myeloma (SMM), Monoclonal Gammopathy of Undetermined Significance (MGUS), or Multiple Myeloma (MM) that would benefit from termination or modification of treatment for SMM, MGUS, or MM, the method comprising:
determining that CD138-negative (CD138−) mononuclear cells obtained from a bone marrow sample or blood sample, mononuclear cells obtained from a blood sample, or a bone marrow tissue section obtained from the human subject have an immune cell composition similar to a control immune cell composition, wherein the control immune cell composition is an immune cell composition for a panel of healthy human subjects.

101-117. (canceled)

118. The method of claim 60 comprising:
determining that mononuclear cells obtained from a blood sample, CD138-negative (CD138−) mononuclear cells obtained from a bone marrow sample or a blood sample, or a bone marrow tissue section obtained from the human subject have:
an increased abundance of granzyme K positive (GZMK⁺) CD8+ T cells relative to a control abundance of GZMK⁺ CD8+ T cells.

119. The method of claim 60, wherein the sample obtained from the subject is a peripheral blood sample.

120. The method of claim 60, wherein the sample obtained from the subject is a bone marrow sample.

121. The method of claim 30, wherein the hematological malignancy or precursor condition is any one of smoldering multiple myeloma (SMM), Monoclonal Gammopathy of Undetermined Significance (MGUS), or Multiple Myeloma (MM).

122. The method of claim 30, wherein the normalization scores are determined using single cell RNA sequencing, targeted single-cell RNA-sequencing, RNA-sequencing, immunohistochemistry, immunofluorescence, flow cytometry, mass cytometry, mass spectrometry, quantitative PCR, immunoblotting, or an imaging-based method.

123. The method of claim 30, wherein characterizing normalization scores during the course of therapy comprises:
(a) providing single-cell RNA sequencing data for a sample obtained from the subject;
(b) determining a composition matrix of cell type proportions based on the single-cell RNA sequencing data;
(c) inputting the composition matrix into a Naïve Bayes classifier and computing the weighted sum of the product of each cell type's proportion to determine a normalization score; and
(d) classifying the sample based on the median normalization score, wherein the Naïve Bayes classifier was trained on a training set comprising:
(i) samples from subjects with smoldering multiple myeloma (SMM), Monoclonal Gammopathy of Undetermined Significance (MGUS), and/or Multiple Myeloma (MM), and
(ii) samples from normal healthy control subjects
wherein the subject is determined to be:
immune reactive if the cell-type proportions in the sample of the subject are least normal-like, and
non-immune reactive if the cell-type proportions in the sample of the subject are most-normal like.

124. The method of claim 123, wherein the subject is determined to be immune reactive based on an increased proportion of granzyme K positive (GZMK+) CD8+ T cells.

125. The method of claim 30 further comprising administering to the subject an immunotherapeutic treatment comprising elotuzumab, lenalidomide, and dexamethasone.

126. The method of claim 31, wherein the hematological malignancy or precursor condition is any one of smoldering multiple myeloma (SMM), Monoclonal Gammopathy of Undetermined Significance (MGUS), or Multiple Myeloma (MM).

127. The method of claim 31, wherein the subject is undergoing treatment for SMM, MGUS, or MM.

128. The method of claim 31, wherein the normalization scores are determined using single cell RNA sequencing, targeted single-cell RNA-sequencing, RNA-sequencing, immunohistochemistry, immunofluorescence, flow cytometry, mass cytometry, mass spectrometry, quantitative PCR, immunoblotting, or an imaging-based method.
